(19) United States
(12) Patent Application Publication
(10) Pub. No.: US 2023/0032988 A1
(43) Pub. Date: Feb. 2, 2023

(54) INFLUENZA ARRAYS AND USE THEREOF

(71) Applicant: NATIONAL INSTITUTE FOR BIOTECHNOLOGY IN THE NEGEV LTD., Beer-Sheva (IL)

(72) Inventors: Tomer HERTZ, Omer (IL); Lilach FRIEDMAN, Rishon Le Zion (IL)

(21) Appl. No.: 17/784,016

(22) PCT Filed: Dec. 8, 2020
(86) PCT No.: PCT/IL2020/051270

§ 371 Date: Jun. 9, 2022

Related U.S. Application Data
(60) Provisional application No. 62/945,178, filed on Dec. 8, 2019.
(60) Provisional application No. 62/945,179, filed on Dec. 8, 2019.

(51) Int. Cl. G01N 33/569

(52) U.S. Cl. CPC G01N 33/56983; G01N 2800/50; G01N 2333/11; G01N 2469/20; G01N 2800/52

(57) ABSTRACT

Arrays comprising probes comprising probes from more than one influenza strain or subtype are provided. Methods of using the arrays, as well as kits and systems comprising the arrays are also provided.

### CROSS REFERENCE TO RELATED APPLICATIONS

This application claims the benefit of priority of U.S. Provisional Patent Application Nos. 62/945,179, filed Dec. 8, 2019, and No. 62/945,178, filed Dec. 8, 2019, the contents of which are all incorporated herein by reference in their entirety.

### FIELD OF INVENTION

The present invention is in the field of influenza vaccination and array design.

### BACKGROUND OF THE INVENTION

The adaptive immune system provides protection against previously encountered pathogens via memory T cells and B cells. Vaccination, the most cost-effective public health intervention, stimulates the immune system to generate protective memory responses. A variety of factors impact an individual's heterogeneity in vaccine induced immune responses, such as age and gender. One important and understudied factor is ‘immunological history’—the individual's memory antibody repertoire to previously encountered pathogens and vaccines. This lack of knowledge is due to the lack of a systematic approach to quantify immunological history and study its effects.

Seasonal influenza viruses are a significant threat to public health and are estimated to cause up to 650,000 deaths worldwide per year. Viruses from the influenza A H1N1 and H3N2 subtypes as well as two B lineages (Victoria and Yamagata) are currently co-circulating in the human population, causing seasonal epidemics and occasional pandemics. Due to high mutation rate, new distinct strains emerge each year. It is estimated that 5-10% of adults and 20-30% of children are infected yearly around the globe. Vaccination is currently the most effective strategy for preventing influenza. While there are currently many influenza vaccines available on the market, the trivalent inactivated influenza vaccine (TIV) and the live attenuated influenza vaccine (LAIV) are the most frequently used. Both vaccines include three (trivalent) or four (quadrivalent) influenza strains from the A/H1N1, A/H3N2 subtypes and one or two B viruses. The vaccines are administered annually and are reformulated in response to antigenic drift that occurs due to the constant genetic evolution of influenza viruses. Influenza vaccine efficacy varies by year and by person, depending on the antigenic match between the vaccine and circulating strains as well as other factors such as age, and prior vaccination history.

The FLUVACS study was a double blind, placebo-controlled trial comparing the efficacious of inactivated and live-attenuated influenza vaccines in adults during the 2007-2008 influenza season (PubmedID:19776407). The study reported that the trivalent inactivated vaccine (Fluzone) significantly reduced the risk of influenza infection with an estimated Vaccine Efficacy (VE) of 68%, whereas the trivalent live-attenuated vaccine (Flumist) was not significantly efficacious (VE=36%). More recently, the CIRN study compared the two types of vaccines in children during the seasons of 2015-2016 and reported significant VE for both types of vaccines (PubmedID:29971427).

In the quest for improving influenza vaccine efficacy, there is a high interest to identify immune markers that correlate with protection against infection or disease following vaccination or natural infection. Correlates of protection (CoPs) have generally been associated with the antibody response in protected and unprotected subjects as well as in vaccine efficacy trials.

There are two classical serological assays that have been widely studied as correlates of protection for influenza vaccines: (1) the hemagglutination-inhibition assay (HAI); and (2) the microneutralization assay (MN). HAI and MN assays are used to define seroconversion, or surrogate correlates of protection, by a fourfold or greater rise in antibody titer following a recent influenza infection or response to vaccination. It is widely accepted that a serum of HAI antibody titer of 1:40 protects 50% of individuals from influenza infection. However, both of these assays have several clear limitations: (1) recently circulating H3N2 viruses fail to agglutinate red blood cells preventing the use of the HAI assay; (2) The MN assay which is a functional assay in tissue cultures is harder to standardize across different labs. More recently the neuraminidase inhibition assay (NAI) has been explored as a correlate of protection, as well as HA (head and stalk) and NA ELISA assays. A clear limitation of all existing correlates of protection assays is that they can be measured for a limited number of influenza strains and require sufficient sample volumes (>50 microliters). A simple, effective assay for all influenza strains, that requires a small amount of sample and can be standardized and run at point of care locations is greatly needed.

### SUMMARY OF THE INVENTION

The present invention provides arrays comprising probes comprising peptides from more than one influenza strain or subtype. Methods of using the arrays of the invention, as well as kits and systems comprising the arrays are also provided.

According to a first aspect, there is provided an array comprising a plurality of probes each immobilized at a discrete location on the array, wherein the plurality of probes comprises a probe from a first influenza strain or subtype and a probe from a second influenza strain or subtype.

According to another aspect, there is provided a method of determining risk of a subject to be symptomatically infected by influenza, determining the suitability of a subject in need thereof to receive an influenza vaccination or for predicting effectiveness of an influenza vaccine in a subject in need thereof, the method comprising
a. providing a biological sample from the subject comprising antibodies;
b. contacting the sample to an array of the invention in conditions sufficient for antibody binding to the probes;
c. detecting the binding of the antibodies to discrete locations on the array indicating the presence in the sample of antibodies to probes located at the detected discrete locations; and
d. generating an influenza immune score from the detected binding, wherein the magnitude of the immune score is proportional to the subject's suitability to receive an influenza vaccine and to effectiveness of an influenza vaccine in the subject;
thereby determining the suitability of a subject to receive an influenza vaccination, determining risk of symptomatic infection of a subject by an influenza virus or predicting effectiveness of an influenza vaccine in a subject.

According to another aspect, there is provided a method of predicting the effectiveness of an influenza vaccine, the method comprising:
a. providing a solution comprising antibodies from immune cells contacted by the influenza vaccine;
b. contacting the solution to an array of the invention in conditions sufficient for antibody binding to the probes;
c. detecting the binding of the antibodies to discrete locations on the array indicating the presence in the solution of antibodies to probes located at the detected discrete locations; and
d. generating an influenza immune score from the detected binding, wherein the magnitude of the immune score is proportional to the influenza vaccine's effectiveness;
thereby predicting the effectiveness of an influenza vaccine.

According to another aspect, there is provided a kit comprising an array of the invention, and labeled secondary antibodies configured for detection of antibodies bound to the array.

According to another aspect, there is provided a system comprising an array of the invention, and a detector configured to detect binding of antibodies to probes immobilized on the array.

According to another aspect, there is provided a method of determining risk of infection by influenza, the method comprising providing a biological sample from the subject comprising antibodies; measuring levels of IgA antibodies against influenza where in a level of anti-influenza IgA antibodies below a predetermined threshold indicates the subject is it at increased of infection by influenza.

According to some embodiments, the plurality of probes comprises an amino acid sequence of a surface protein from a first strain or subtype of influenza and an amino acid sequence of a surface protein from a second strain or subtype of influenza.

According to some embodiments, the plurality of probes comprises at least two probe from each strain or subtype of influenza.

According to some embodiments, the plurality of probes comprises a peptide from a hemagglutinin (HA) protein, a peptide from a neuraminidase (NA) protein or both.

According to some embodiments, the influenza subtype is selected from the group consisting of: H1N1 influenza, H3N2 influenza, and influenza B.

According to some embodiments, the influenza subtype is selected from the subtypes listed in Table 1 and Table 5.

According to some embodiments, the plurality of probes is selected from a whole virus, a lysed virus, a virus-like particle (VLP), a whole recombinant protein and a peptide.

According to some embodiments, the plurality of probes comprises a probe from an influenza subtype or strain from a first year and a probe comprising a peptide from the influenza subtype or strain from a second year.

According to some embodiments, the probe from an influenza subtype from a first year and the probe from an influenza subtype from a second year are from the same protein, from the same region of a protein or both.

According to some embodiments, the plurality of probes comprises a peptide probe from each of the influenza strains or subtypes.

According to some embodiments, the plurality of probes comprises a peptide probe from a hemagglutinin (HA) protein from each of the influenza strains or subtypes.

According to some embodiments, the plurality of probes comprises a peptide probe from a neuraminidase (NA) protein from each of the influenza strains or subtypes.

According to some embodiments, the peptide probe comprises between 10 and 60 consecutive amino acids from an influenza protein.

According to some embodiments, the plurality of probes comprises a recombinant protein from each of the influenza strains or subtypes.

According to some embodiments, the recombinants protein is a recombinant surface protein.

According to some embodiments, the plurality of probes comprises an inactivated form of each of the influenza strains or subtypes.

According to some embodiments, the plurality of probes comprises a virus-like particle (VLP) of each of the influenza strains or subtypes.

According to some embodiments, the plurality of probes comprises lysate from a cell infected by each of the influenza strains or subtypes.

According to some embodiments, the peptide comprises at least 10 consecutive amino acids from an influenza protein.

According to some embodiments, the plurality of probes further comprises an inactivated influenza virus.

According to some embodiments, the plurality of probes further comprises a virus-like particle (VLP).

According to some embodiments, the plurality of probes further comprises lysate from a cell infected by an influenza virus.

According to some embodiments, the array of the invention is a human array, and the plurality of probes is selected from Table 1 and SEQ ID NO: 1-1390.

According to some embodiments, the array of the invention is a non-human array, and the plurality of probes is selected from Table 5 and SEQ ID NO: 598-995.

According to some embodiments, the array of the invention is for use in predicting the risk of symptomatic infection of a subject by an influenza virus.

According to some embodiments, the array of the invention is for use in determining the suitability of a subject to receive an influenza vaccine.

According to some embodiments, the array of the invention is for use in predicting the effectiveness of influenza vaccination in a subject.

According to some embodiments, the array of the invention is for use in detecting previous influenza infection or vaccination of a subject.

According to some embodiments, the array of the invention is for use in determining the strain or subtype of influenza that had previously infected or been vaccinated against in the subject.

According to some embodiments, the subject has previously been vaccinated against influenza or previously been infected by an influenza virus.

According to some embodiments, the influenza is selected from the group consisting of: H1N1 influenza, H3N2 influenza, and influenza B.

According to some embodiments, the influenza subtype is selected from the subtypes listed in Table 1 and Table 5.

According to some embodiments, the biological sample is a peripheral blood sample, a plasma sample or a serum sample.

According to some embodiments, the detecting comprises contacting the array with bound antibodies with labeled secondary antibodies against the antibodies in the biological sample.

According to some embodiments, the secondary antibodies are directed against IgG, IgA, or both and wherein antibodies against IgG and IgA comprise distinct labels.

According to some embodiments, the influenza immune score is generated from IgA binding.

According to some embodiments, the influenza is a specific influenza strain or subtype and the immune score is generated from detected binding to probes from the specific strain or subtype.

According to some embodiments, the detecting further comprises scanning the array with a detector configured to detect the labeled secondary antibodies and producing an output of the discrete locations where antibody was detected.

According to some embodiments, a higher immune score indicates a greater suitability to receive an influenza vaccination, decreased risk of symptomatic infection by an influenza or greater likelihood of effectiveness of an influenza vaccine, and wherein a lower immune score indicates a lesser suitability to receive an influenza vaccination, increased risk of symptomatic infection by an influenza or a lower likelihood of effectiveness of an influenza vaccine.

According to some embodiments, an immune score above a predetermined threshold indicates the subject is suitable to receive an influenza vaccination, is at reduced risk of symptomatic infection by an influenza or the influenza vaccine is likely to be effective.

According to some embodiments, the subject is a human subject, and the plurality of probes is selected from Table 1 and SEQ ID NO: 1-1390 or the subject is a non-human subject and the plurality of probes is selected from Table 5 and SEQ ID NO: 598-995.

According to some embodiments, the detector is configured to detect labeled secondary antibodies.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1Y: TIV but not LAIV induced a significant rise in IgG and IgA levels. (1A, 1C, 1E, 1G) Boxplots of the pre-vaccination (left boxes) and post-vaccination (right boxes) median fluorescence intensity (MFI) for IgG (1A, 1E) and IgA (1C, 1G) to the whole inactivated virus (1A, 1C) or recombinant hemagglutinin protein (rHA) (1E, 1G) of the H3N2 vaccine strain A/Wisconsin/67/2005 (Wis05) in serum samples from individuals vaccinated with placebo, inactivated (TIV) or live-attenuated (LAIV) trivalent influenza vaccines. (1B, 1F, 1D, 1H) Boxplots of MFI fold change of IgG (1B, 1F) and IgA (1D, 1H) to Wis05 whole virus (1B, 1D) or rHA (1F, 1H) antigens at 30 days following TIV or LAIV compared with pre-vaccination antibody levels. Horizontal lines represent the median values, and the box limits correspond to the 25th and the 75th percentiles. The whiskers represent 1.5 times the interquartile range. Statistical significance (p<0.05) was determined using the Wilcoxon signed rank test for comparison between pre- and post-vaccination timepoints, and the Wilcoxon ranksum test for fold-rise differences comparing the TIV and LAIV vaccine groups. (1I-1P) The same results are shown for the H1N1 vaccine strain (A/Solomon Island/3/2006). (1Q-1X) The same results are shown for the B vaccine strain (B/Malaysia/2506/2004). (1Y) Schematic diagram of the protocol.

FIGS. 2A-2JJ: TIV and LAIV induced significant rises in the magnitudes and breadths of IgG and IgA to a panel of influenza strains, and post-vaccination magnitudes were correlated with the baseline magnitudes. The overall magnitude was defined as the sum of antibody levels, measured by median fluorescence intensity (MFI) values, across all spotted antigens from the same subtype (2A-2DD). The breadth was defined as the total number of antigens from a certain subtype with an antibody response above a predefined threshold (MFI>2000 for proteins and MFI>200 for whole viruses) (2EE-2JJ). (2A-2H) Magnitude of antibodies to H3N2 whole viruses and rHA proteins. (2A, 2C, 2E, 2G) Boxplots of the H3N2 magnitude for baseline and post-vaccination IgG (2A, 2E) and IgA (2C, 2G) binding to panels of 15 H3N2 whole inactivated viruses (WIV) from 1979-2013 (2A, 2C), and 9 H3N2 recombinant HA (rHA) antigens from 1989-2013 (2E, 2G). (2B, 2D, 2F, 2H) Boxplots of the magnitude fold change of IgG (2B, 2F) and IgA (2D, 2H) to 15 H3N2 WIV and 9 rHA proteins 30 days post TIV or LAIV vaccination compared with pre-vaccination levels. Horizontal lines represent the median values, and the box limits correspond to the 25th and the 75th percentiles. The whiskers represent 1.5 times the interquartile range. Statistical significance (p<0.05) was determined using the Wilcoxon signed rank test for comparison between pre and post timepoints and the Wilcoxon ranksum test for fold-rise differences. (2I-2P) The same results are shown for the H1N1 antigens. Whole virus magnitudes were computed against 12 H1N1 whole inactivated viruses from 1933-2010, and rHA magnitudes were computed against 10 H1N1 recombinant HA antigens from 1918-2009. (2Q-2X) The same results are shown for the B strain antigens. Whole virus magnitudes were computed against 8 B whole inactivated viruses from 1940-2013, and rHA magnitudes were computed against seven B recombinant HA antigens from 1988-2013. (2Y-2DD) Post-vaccination antibody magnitudes to antigens are correlated with the individuals baseline magnitudes. Pairwise Spearman correlations between baseline (x-axis) and post-vaccination (y-axis) magnitude of IgG (upper panels) and IgA (lower panels) levels to the whole inactivated viruses (WIV; 2Y, 2AA, 2CC) and recombinant HA proteins (rHA; 2Z, 2BB, 2DD) of strains from the (2Y-2Z) H3N2 subtype, (2AA-2BB) H1N1 subtype and (2CC-2DD) B subtype. (2EE-2JJ) Boxplots of the breadth for baseline and post-vaccination IgG (2EE, 2GG, 2II) and IgA (2FF, 2HH, 2JJ) binding to whole virus and rHA antigens of subtypes H3N2 (2EE-2FF), H1N1 (2GG-2HH) and B (2II-2JJ). TIV induced a broader repertoire of antibodies for both whole virus and recombinant HA antigens. Horizontal bars represent the median response of each group. Box limits represent the 25th and 75th percentiles, and the whiskers denote the 1.5 times the interquartile range. Individuals are plotted as circles. Statistical significance was determined using Wilcoxon signed rank test.

FIGS. 3A-3P: Comparing baseline and post-vaccination IgG and IgA antibody binding to H3N2 antigens in cases vs. control participants. Eighty-six subjects from Fluvacs trial that were symptomatically infected by influenza A/H3N2, as confirmed by real-time reverse transcription polymerase chain reaction (RT-PCR) from throat swab samples (cases), were compared with 79 randomly selected controls without influenza illness from the same trial. (3A-3P) Boxplots of IgG (3A-3D, 3I-3L) and IgA (3E-3H, 3M-3P) antibody MFI levels at baseline (3A, 3C, 3E, 3G, 3I, 3K, 3M, 3O) and post-vaccination (3B, 3D, 3F, 3H, 3J, 3L, 3N, 3P) to the rHA (3A-3B, 3E-3F) and whole virus antigen (3I-3J, 3M-3N) of the Wis05 H3N2 vaccine strain; the magnitude of antibodies to 9 H3N2 recombinant rHA antigens from 1989-2013 (3C-3D, 3G-H); and the magnitude of antibodies to 15 H3N2 whole inactivated viruses from 1979-2013 (3K-3L, 3O-3P). For post-vaccination time point, only TIV and LAIV groups are shown. Horizontal bars represent the median response of each group. Box limits represent the 25th and 75th percentiles, and the whiskers denote the 1.5 times the interquartile range. Individual responses are plotted as circles. Responses above the whiskers are outliers. P-values and q-values are from the regression analyses of whether the antibody marker is associated with Wis05 infection. Significant p-values (<0.05) and q-values (<0.1) are represented for the relevant group. X axis labels: − controls; + cases.

FIGS. 4A-4P. Comparing baseline and post-vaccination IgG and IgA binding to rHA antigens of H1N1 (4A-4H) and B (4I-4P) influenza strains in 86 cases vs. 79 control participants. All the cases were infected with the H3N2 vaccine strain. (4A-4H) Boxplots of the IgG (4A-4D) and IgA (4E-4H) binding to the rHA antigen of the H1N1 vaccine strain (4A-4B, 4E-4F) and overall magnitude of antibodies to 10 H1N1 rHA proteins (4C-4D, 4G-41I) in the placebo, TIV and LAIV groups, comparing cases (+) vs. control (−) subjects (4I-4P) Boxplots of the IgG (4I-4L) and IgA (4M-4P) binding to the rHA antigen of the B vaccine strain (4I-4J, 4M-4N) and overall magnitude of antibodies to 7 B rHA proteins (4K-4L, 4O-4P). For post-vaccination time point, only TIV and LAIV groups are shown (Placebo had no changes). Horizontal bars represent the median response of each group. Box limits represent the 25th and 75th percentiles, and the whiskers denote the 1.5 times the interquartile range. Individual responses are plotted as circles. Responses above the whiskers are outliers. P-values and q-values are from regression analyses. Significant p-values (<0.05) and q-values (<0.1) are represented for the relevant group.

FIGS. 5A-5D. High heterogeneity in IgA and IgG baseline and post-vaccination influenza immune history profiles. (5A) A histogram of the baseline IgA median fluorescence intensity (MFI) levels to H3N2 rHA (rH3) antigens for each individual in the TIV group (n=51), sorted by the overall magnitude to a panel of 9 recombinant HA proteins from H3N2 subtype. Strains were ordered by time; vaccine strain is colored in orange; historical strains are in blue; and cross-reactive antibodies to future strains are colored in green. Following sorting the individuals from the highest to lowest baseline rH3 IgA magnitude, the individuals were divided to four quartiles. The quartile with the highest magnitudes, termed high-baseline immune history (BIH), is labeled by a darkest gray background. The quartile with the lowest magnitudes, termed Low-BIH, is labeled by a white background. The residual half of individuals, termed Mid-BIH, is labeled by a lighter gray background. This histogram is given as an example. The same sorting was done for all the three groups, for magnitudes of rHA and whole viruses of all the 3 vaccine subtypes. (5B-5D) Spider charts of IgA and IgG antibody profiles to a panel of recombinant HA proteins for H3N2, H1N1 and B types (9, 7 and 5 strains, respectively) in the TIV vaccinated group. Individuals were sorted by their baseline immune history (BIH) for the magnitude of rH3 proteins using median fluorescent intensity (MFI) level (as shown in 5A). Spider charts for representative individuals from the low-BIH (5B), high-BIH (5C) and mid-BIH (5D) quartiles are shown. Both IgA and IgG repertoires are shown. PCR-confirmed H3N2 symptomatically infected cases and uninfected controls are represented for each quartile. The colored spider area represents the breadth and magnitude of the antibody response pre- and post-vaccination; Blue area: baseline response, orange area: vaccine induced response. H3N2, H1N1 and B rHA protein antigens are ring-colored by red, purple and light blue, respectively. The year when viruses were first isolated is denoted by its last two digits. The three vaccine strains are denoted by colored dots; H3N2 A/Wisconsin/65/2005 (red dot), H1N1 A/Solomon Island/3/2006 (purple dot) and B/Malaysia/2506/2004 (light blue dot). The number above each spider charts is the individual ID number.

FIGS. 6A-6D. Baseline and post-vaccination serum IgA but not IgG immune-history profiles are correlates of risk of influenza infection. (6A) Histograms of the baseline IgA median fluorescence intensity (MFI) levels sorted by the overall magnitude to a panel of 9 recombinant HA proteins from H3N2 subtype for each of the 165 individuals in the trial. Strains were ordered by time; vaccine strain is colored in orange; historical strains are in blue; and cross-reactive antibodies to future strains are colored in green. Subjects within each treatment arm (Placebo, TIV and LAIV) were divided into quartiles based on the overall BIH magnitude for the IgA: low BIH (lowest quartile), high BIH (highest quartile) and mid-BIH (the two middle quartiles). (6B-6D) BIH ranking according to the baseline magnitude of IgA (6B-6C) or IgG (6D) antibody binding to rHA proteins of 9 H3N2 influenza stains. The infection rates within each BIH group are presented for the three treatment arms of the study. (6B) Bar graphs of the infection rates within each IgA-BIH group. (6C) Subjects in the Mid-BIH TIV group (n=25) and Mid-BIH LAIV group (n=28), according to IgA-BIH sorting, were further divided into quartiles based on the post-vaccination (baseline subtracted) rise in IgA titer to the panel of H3N2 rHA proteins. Bar graphs show infection rates in each group by percentage. (6D) Bar graphs of infection rates within each IgG-BIH group. P-values and q-values were computed using a logisticare regression analyses adjusted for baseline covariates. Significant p-values (<0.05) and q-values (<0.1) are represented for the relevant group.

FIGS. 7A-7B. PCR-confirmed Wis05 infection rates are correlated with baseline IgA immune history ranking to the Wis05 recombinant HA protein. (7A-7B) Individuals were sorted by their baseline immune history (BIH) median fluorescence intensity (MFI) for the vaccine strain A/Wisconsin/65/2005 (Wis05) rHA antigen only, for each of three treatment arms of the study separately (Placebo, TIV, LAIV). Subjects within each group were divided into quartiles by BIH, and infection rates within each group were computed. (7A) Bar graphs of infection rates by IgA-BIH to Wis05 rHA. (7B) Bar graphs of infection rates by IgG-BIH Wis05 rHA. P-values comparing infection rates of high vs. low BIH were computed using a logistic regression model adjusted for baseline covariates.

FIGS. 8A-8D. PCR-confirmed Wis05 infection rates are correlated with post-vaccination ranking according to antibody binding to H3N2 rHA proteins. Individuals were sorted by their post-vaccination magnitude of antibody binding to rHA proteins of 9 H3N2 influenza stains (8A, 8C), or by their median fluorescence intensity (MFI) of antibody binding to the A/Wisconsin/65/2005 rHA antigen only, for the two immunized arms of the study (TIV, LAIV). Following sorting, subjects within each vaccine arm were divided into quartiles: low-responders (lowest quartile), high-responders (highest quartile) and mid-responders (the two middle quartiles), and the infection rate within each group was computed. Bar charts present the infection rates of quartile responder groups based on IgA (8A-8B) or IgG (8C-8D) post-vaccination magnitude of antibodies to 9 recombinant HA proteins of H3N2 subtypes (8A, 8C), or post-vaccination staining of the Wis05 rHA protein alone (8B, 8D). P-values comparing infection rates of high vs. low responders were computed using a logistic regression model adjusted for baseline covariates.

FIG. 9A-BB: Comparing the serum IgG and IgA antibody repertoires of obese and healthy-weight individuals. Baseline and post-vaccination serum samples from 89 healthy-weight (HW) and 100 obese (Ob) subjects were hybridized with two types of antigen microarrays for profiling of IgG and IgA binding: proteins and viruses microarrays spotted with 34 BPL-inactivated influenza viruses and 26 recombinant HA (rHA) proteins that included the three vaccine strains used in the study; and peptides microarrays spotted with 20mer amino acid peptides with 15aa overlap spanning the HA and NA proteins of the pandemic H1N1 A/California/7/2009 (Cal09) vaccine strain, that is known as inducing less effective immunization in obese individuals. Responses were summarized using breadth and magnitude. (9A-9L) IgG (9A-9F) and IgA (9G-9L) repertoires to the H1N1 Cal09 and historical H1N1 strains. Obese individuals have lower levels of IgG to Cal09 virus and rHA protein both at baseline and post-vaccination, compared with healthy-weight individuals, and higher levels of IgA to Cal09 virus and rHA protein only at the post-vaccination time point. In addition, obese individuals have a wider repertoire of IgG antibodies and a narrower IgA repertoire to Cal09 HA and NA peptides compared with healthy-weight individuals, both at baseline and post-vaccination. (9A-9B, 9G-9H) IgG (9A-9B) and (9G-9H) IgA binding to whole viruse antigen (9A, 9G) or a recombinant HA protein (rHA; 9B, 9H) of the Cal09 vaccine strain. (9C-9D, 9I-9J) Boxplots comparing the magnitude of (9C-9D) IgG and (9I-9J) IgA antibodies to a panel of (9C, 9I) 11 whole virus H1N1 strains and to a panel of (9D, 9J) 8 recombinant HA antigens of H1N1 strains. (9E-9F, 9K-9L) Boxplots of (9E-9F) IgG and (9K-9L) IgA breadth to Cal09 H1 and N1 peptides (9M-9T) Baseline and post-vaccination IgG (9M-9P) and IgA (9Q-9T) repertoires to H3N2 strains. (9M, 9Q) IgG and IgA binding to the whole virus antigen of the H3N2 vaccine strain A/Perth/16/2009. (9N, 9R) IgG and IgA binding to the recombinant HA of the H3N2 A/Perth/16/2009 vaccine strain. (9O-9P, 9S-9T) Boxplots comparing the magnitude of (9O-9P) IgG and (9S-9T) IgA antibodies to a panel of (9O, 9S) 15 whole virus H3N2 strains and to a panel of (9P, 9T) 10 recombinant H3 proteins. (9U-9BB) Baseline and post-vaccination IgG (9U-9X) and IgA (9Y-9BB) repertoires to B influenza strains. (9U, 9Y) IgG and IgA binding to whole virus antigen of the B/Brisbane/60/2008 vaccine strain. (9V, 9Z) IgG and IgA binding to the recombinant HA of the B/Brisbane/60/2008 vaccine strain. (9W-9X, 9AA-9BB) Boxplots comparing the magnitude of (9W-9X) IgG and (9AA-9BB) IgA antibodies to a panel of (9W, 9AA) 8 whole virus B strains and to a panel of (9X, 9BB) recombinant HA antigens of 5 B strains. Left panels: boxplots of baseline (Pre; HW: light green, Ob: light blue) and post-vaccination (d30, Post; HW: dark green, Ob: dark blue) antibody binding. Right panels: boxplots of fold rise responses (HW: green, Ob: blue). Lines represent the median fluorescence intensity (MFI), the boxes denote the 25th and 75th percentiles, and the error bars represent 1.5 times the interquartile range. Statistical significance was assessed using the Wilcoxon signed rank test (pre vs. post) and the Wilcoxon rank-sum test (HW vs. Ob). * p<0.05, ** p<0.005, *** p<0.0005.

FIGS. 10A-10R: (10A-10B) Cumulative distribution plots comparing the baseline and post-vaccination total level of (10A) IgG and (10B) IgA in the serum, measured by sandwich. ELISA. A white dot represents the median titer. (10C-10N) Left panels: Box plots comparing the breadth of (10C-10H) IgG or (10I-10N) IgA antibodies to a panel of (10C-10E, 10I-10K) whole inactivated viruses and (10F-H, 10M-N) recombinant HA proteins, including: (10C, 10I) 11 whole virus H1N1 influenza strains, (10D, 10J) 15 whole virus H3N2 strains, (10E, 10K) 8 whole B viruses, (10F, 10L) recombinant HA (rHA) proteins of 8 H1N1 strains, (10G, 10M) rHA proteins of 10 H3N2 strains, and (10H, 10N) rHA proteins of 5 B strains. Right panels: Boxplots of IgG or IgA breadth at baseline divided from post-vaccination breadth (fold rise of breadth; HW: green, Ob: light blue). Lines represent the median breadth. (100-10R) Box plots comparing the magnitude (left panels) and baseline-adjusted magnitude (right panels) of (100-10P) IgG and (10Q-10R) IgA antibodies to linear peptides spanning the (10O, 10Q) HA and (10P, 10R) NA proteins of the H1N1 California/07/2009 vaccine strain. Lines represent the median magnitude. The boxes in 10C-10R denote the 25th and 75th percentiles, and the error bars represent 1.5 times the interquartile range. Statistical significance was determined using the Wilcoxon rank sum test. * p<0.05, ** p<0.005, and *** p<0.0005.

FIGS. 11A-11L: Extensive heterogeneity of baseline and post-vaccination influenza immune history profiles, for both IgG and IgA. (11A) All subjects in the cohort were sorted by their baseline IgG magnitude (baseline immune history—BIH) to recombinant HA proteins of the H1N1 subtype strains (rH1, listed in the color key, the vaccine strain in pink). Subjects were divided into quartiles (low-BIH: n=47, mid-BIH: n=95 and high-BIH: n=47). (11B-11E) To visualize the antibody repertoire of each subject to the panel of influenza strains we generated spider plots in which each vertex represents the normalized binding of IgG or IgA to a single influenza strain rHA. Strains were organized by subtype (H1N1: purple, H3N2: red and B: light blue), the numbers denote the year each strain was isolated, and the vaccine strains are denoted by the colored dots at the circle perimeters. Baseline and post-vaccination antibody repertoires are denoted by blue and orange spider plots, respectively. Subject ID, BMI and age of each subject are presented above each graph. Representative spider plots of (11B-11C) 6 HW and (11D-11E) 6 obese subjects from the (11B, 11D) IgG low-BIH group and (11C, 11E) IgG high-BIH group are presented. (11F) Extensive heterogeneity of influenza baseline IgA immune history profiles. All subjects in the cohort were sorted by their baseline IgA magnitude to recombinant HA proteins of the H1N1 subtype strains. Subjects were divided into quartiles (low-BIH: n=44, mid-BIH: n=86 and high-BIH: n=44). (11G-11L) High correlation between baseline and post-vaccination antibody binding to H1N1 antigens. Scatter plots comparing the baseline and post vaccination IgG and IgA antibody levels to H1N1 antigens: (11G-11H) IgG and IgA to Cal09 whole virus; (11I-11J) IgG and IgA levels to Cal09 rHA protein (11K-11L); and magnitude of responses to recombinant H1N1 HA proteins. The Spearman correlation coefficients are presented in each panel in FIGS. 11G-11L.

FIGS. 12A-12H: The repertoires of serum IgG and IgA to influenza whole virus and rHA antigens are weakly correlated to one another. (12A-12D) Scatter plots comparing the binding of IgG and IgA to the (12A-12B) whole virus antigen or (12C-12D) rHA protein of the H1N1 Cal09 vaccine strain in (12A, 12C) HW and (12B, 12D) obese subjects at the two timepoints. The Spearman correlation coefficient and p value are presented in each panel of FIGS. 12A-12D. (12E-12H) Pairwise Spearman correlations between magnitudes of IgG and IgA to influenza subtypes, as measured by binding to whole viruses and rHA proteins at (12E, 12G) baseline and (12F, 12H) post-vaccination. P-values were computed for each pairwise correlation, and multiplicity adjustment was applied over the entire p-values set. Family-wise error rates (FWER) were calculated using the Bonferroni-Holm method and are presented in each figure using asterisks, with ***, **, and * indicating p<0.0005, 0.005, and 0.05, respectively.

FIGS. 13A-13L: Obesity is associated with a particular baseline immune history (BIH) profile to influenza H1N1 antigens. Obesity was associated with low IgG-BIH to H1N1 viruses and rH1 proteins, high IgA-BIH to rH1 proteins, and high IgG-BIH to peptides of the Cal09 H1 and N1 proteins. The distributions of subjects' BMI within the low-BIH group (n=47, purple) and the high-BIH group (n=47, green) were plotted when sorting subjects into groups by their IgG and IgA responses to H1N1 antigens as follows: (13A) IgG Magnitude against H1N1 viruses; (13B) IgG Magnitude to rH1 proteins; (13C) IgA magnitude to H1N1 viruses; (13D) IgA magnitude to rH1 proteins; (13E) IgG breadth to H1 peptides; (13F) IgG breadth to N1 peptides; (13G) IgA breadth to H1 peptides; (13H) IgA breadth to N1 peptides; (13I) IgG magnitude to H1 peptides; (13J) IgG magnitude to N1 peptides; (13K) IgA magnitude to H1 peptides; (13L) IgA magnitude to N1 peptides. BMI distributions of the low-BIH and high-BIH groups were compared using the Wilcoxon ranksum test. Our cohort did not include any overweight subjects (25<BMI<30).

FIGS. 14A-14X: Distributions of BMI and age in subjects with low and high post-vaccination responses to H1N1 antigens. Baseline antibody binding to each antigen was subtracted from the post-vaccination antibody binding to the same antigen. Subjects were ranked according to these baseline-subtracted post-vaccination responses and divided into quartiles. Individuals belonging to the upper quartile were defined as high responders (n=47, broken green lines) and participants belonging to the lower quartile were defined as low responders. (14A-14P) The distributions of subjects' BMI and age within the low-responders and high-responders groups when sorting subjects into groups by their IgG and IgA responses to Cal09 H1N1 peptides. (14A-14H) The distributions of subjects' BMI within the low-responders and the high-responders were plotted when sorting subjects into groups by their IgG and IgA responses to Cal09 H1N1 peptides: (14A) H1 IgG breadth; (14B) N1 IgG breadth; (14C) H1 IgA breadth; (14D) N1 IgA breadth; (14E) H1 IgG magnitude; (14F) N1 IgG magnitude; (14G) H1 IgA magnitude; (14H) N1 IgA magnitude. The percentages of obese subjects in the low-responders and high-responders quartiles are listed. (14I-14P) The distributions of subjects' age within the low-responders and the high-responders were plotted when sorting subjects into groups by their IgG and IgA responses to Cal09 H1N1 peptides: (14I) H1 IgG breadth; (14J) N1 IgG breadth; (14K) H1 IgA breadth; (14L) N1 IgA breadth; (14M) H1 IgG magnitude; (14N) N1 IgG magnitude; (140) H1 IgA magnitude; (14P) N1 IgA magnitude. The percentages of elderlies (>65 years old) subjects in the low-responders and high-responders quartiles are listed. (14Q-14X) The distributions of subjects' BMI and age within the low-responders and high-responders groups when sorting subjects into groups by their IgG and IgA magnitudes to H1N1 whole viruses or rH1 proteins. The subjects were ranked based on magnitude of (14Q-14R, 14U-14V) IgG or (14S-14T, 14W-14X) IgA baseline-subtracted responses to whole viruses or recombinant HA proteins. The proportion of (14Q-14T) obese and healthy-weight and (14U-14X) distribution of ages were measured in the low and high responders for IgG and IgA, for both whole viruses and recombinant HA proteins. P values of the differences between the distributions of BMI and ages in low and high responders were compared using the Wilcoxon rank-sum test. Our cohort did not include any overweight subjects (25<BMI<30).

FIGS. 15A-15L: Age is associated with a particular baseline immune history (BIH) profile to influenza H1N1 antigens. Elderly age was associated with high IgA-BIH to H1N1 viruses and H1 proteins, high IgG-BIH to H1N1 viruses, low IgG- and IgA-BIH to Cal09 H1 peptides, and low IgG-BIH (but not IgA-BIH) to Cal09 N1 peptides. The distributions of subjects' Age within the low-BIH group (n=47, purple) and the high-BIH group (n=47, green) were plotted when sorting subjects into groups by their IgG and IgA responses to H1N1 antigens as follows: (15A) IgG Magnitude against H1N1 viruses; (15B) IgG Magnitude to rH1 proteins; (15C) IgA magnitude to H1N1 viruses; (15D) IgA magnitude to rH1 proteins; (15E) IgG breadth to H1 peptides; (15F) IgG breadth to N1 peptides; (15G) IgA breadth to H1 peptides; (5H) IgA breadth to N1 peptides; (15I) IgG magnitude to H1 peptides; (15J) IgG magnitude to N1 peptides; (15K) IgA magnitude to H1 peptides; (15L) IgA magnitude to N1 peptides. Age distributions of the low-BIH-1 and high-BIH groups were compared using the Wilcoxon ranksum test.

FIGS. 16A-16B: Baseline antibody profiles can predict post-vaccination antibody profiles. (16A-16B) Six logistic regression models were trained to predict the IgA and IgG post-vaccination levels to the Cal09 HA protein and Cal09 whole virus, as well as the magnitudes of IgG and IgA levels to H1N1 proteins and viruses. (16A) The Spearman correlation coefficient between the measured and predicted values for each of the six models. All models were trained on n=196 subjects using the following features: baseline IgG and IgA levels to the Cal09 vaccine strain, baseline IgG and IgA magnitudes to H1N1 viruses, baseline IgG and IgA levels to the H1 protein of the Cal09 vaccine strain, and the baseline IgG and IgA magnitudes to H1 proteins. Error bars represent the 95% confidence interval. (16B) Heatmap of the odds ratios (model weights) of each of the features (y-axis) for each prediction model (x-axis).

FIGS. 17A-17D: Obese and healthy-weight subjects target different domains in the Cal09 HA protein. (17A) The repertoire of IgG and IgA antibodies to Cal09 peptides can be used to predict obesity. Four logistic regression models were trained using the entire cohort (n=205). Models were trained on baseline (Pre) or baseline+post-vaccination (Pre & Post) levels of IgG and IgA antibodies to Cal09 H1 and N1 peptides. Each bar portrays the area under the receiver operating characteristic (ROC) curve for each model, computed using leave one sample out cross validation. (17B) Four logistic regression models for predicting obesity status were trained using part of the cohort, the three n=68 models were constructed using the same 68 samples for which MN (microneutralization) and HAI (hemagglutinin inhibition) were available. The fourth model AM (the antigen microarray of the invention) using all the cohort n=165. Each bar portrays the area under the receiver operating characteristic (ROC) curve for each model, calculated for all predicted values using leave one sample out cross validation. Predicted values over all folds were collected, and then AUC (area under the curve) was calculated over all values. (17C-17D) Differences between obese and healthy-weight subjects in the baseline (17C) IgG and (17D) IgA antibody repertoires to Cal09 H1 peptides. The weights of peptides used to train the logistic regression model was used to score individual amino acids on the HA protein based on the maximal weight they were assigned within the model. HA residues were divided into 3 groups: obese (blue)—residues assign with positive weights values, HW (orange)—residues assigned with negative weight values and unweighted (green) residues for positions who had neutral or low contribution to the regression model (see method for more details). Then residues were assigned into their structural domains (x axis) within the HA protein, including HAL HA2, fusion peptide, the receptor binding site (RBS), antigenic sites (Sa, Sb, Ca1, Ca2, Cb), glycosylation sites (Gly site—general glycosylation in HA proteins, cal09 gly site-specific glycosylation in HA protein of cal09 strain), the esterase domain, and the three binding loops. The proportion of obese, HW and unweighted associated residues within each domain are shown to explore the enrichment of these groups, statistically significant enrichments are marked with asterisk.

FIG. 18: Image of a representative influenza array containing whole virus and proteins probes. The array was hybridized with human serum from an elderly subject and stained with anti-human IgG-Alexa 647.

### DETAILED DESCRIPTION OF THE INVENTION

The present invention, in some embodiments, provides arrays comprising a plurality of probes, each probe is immobilized at a discrete location on the array, and the plurality of probes comprises a probe from a first influenza strain or subtype and a probe from a second influenza strain or subtype. Kits and systems comprising the arrays of the invention are also provided, as are methods of using the arrays, kits and systems of the invention for determining the suitability of a subject to receive an influenza vaccine, or for predicting the effectiveness of an influenza vaccine.

The present invention is based on the surprising finding that influenza immune-history antibody profiles correlate with the risk of future infection and vaccination efficacy. That is by assaying specific peptides, proteins and whole virus antigens from various influenza subtypes and strains, an influenza immune score can be generated for an individual or a vaccine. This immune score can predict the likelihood of future infection, with and without vaccination, as well as the likelihood that an individual will produce an effective immune response upon vaccination. It was further surprisingly found that IgA levels were more predictive than IgG levels. Previous studies reported that local and systemic serum antibody production following inactivated influenza vaccines is dominated by IgG antibodies (particularly IgG1), with lower concentration of IgM and IgA. IgA levels are usually lower in blood and therefore harder to measure, and the arrays of the invention allow for accurate and precise IgA readings, as well as for repeatable measures across individuals and testing sites.

Using a case-control set of 165 subjects from the FLUVACS trial for which serum samples were collected at baseline and post-vaccination we studied (1) baseline immune-history antibody profiles as correlates of risk for influenza disease in each of the placebo, TIV and LAIV treatment groups; (2) post-vaccination profiles as correlates of risk in the TIV and LAIV treatment groups; and (3) baseline immune-history antibody profiles as modifiers/correlates of VE for each of the TIV and LAIV treatment groups. An extensive high-dimensional influenza antigen microarray for profiling influenza immune history was developed. The arrays were spotted with influenza antigens including whole inactivated viruses (n=47) and recombinant HA (n=27) and NA proteins (n=7) from the H1N1, H3N2 and B subtypes that represent the antigenic diversity of influenza strains during a period of over 100 years (1918-2018), with a focus on the last 35 years (1986-2018). These arrays were used to generate IgG and IgA antibody profiles for each of the subjects at both baseline and post-vaccination. By comparing the profiles of infected vs. uninfected subjects we found IgG responses post-vaccination that were associated with protection in the TIV group, while both baseline and post-vaccination IgA responses were associated with protection in all three treatment arms of the study. We were also able to find significant differences in the risk of infection in subjects with weak and narrow immune-history profiles as compared to subjects with strong and broad immune-history profiles.

The use of an array of probes allows for a quick and accurate assessment of the influenza immune history of a subject. Further, the array only requires a small amount of sample from an individual. This way, a single blood draw can be used to evaluate a subject influenza history, across multiple subtypes and multiple strains simultaneously.

Influenza Arrays

By a first aspect, there is provided a solid support comprising a plurality of probes, wherein the plurality of probes comprises a probe from a first influenza and a probe from a second influenza.

By a first aspect, there is provided an array comprising a plurality of probes, wherein the plurality of probes comprises a probe peptide from a first influenza and a probe peptide from a second influenza.

In some embodiments, the solid support is an array. In some embodiments, the solid support is a chip. As used herein, the term “array” refers to a solid support with regularly spaced probes attached to distinct and defined locations. In some embodiments, an array is an array of probes. In some embodiments, the support or array comprises probes at known locations. Thus, the location of each probe is known and so binding to a given probe can be correlated to the probe itself based on its position on the support or array. In some embodiments, an array is a single solid support with probes arrayed thereupon. In some embodiments, an array is a plurality of solid supports with probes arrayed thereupon. In some embodiments, each probe is on a separate solid support. In some embodiments, an array is an array of beads. In some embodiments, an array is an array of solid supports. Methods of making arrays and in particular protein and peptide arrays are well known in the art. Any method of making an array such as described herein may be employed. One such method is provided hereinbelow in the Materials and Methods section. Non-limiting examples of methods of producing protein/peptide arrays include U.S. Pat. No. 5,143,854, U.S. Patent Application Publication Nos. 2007/0154946, 2007/0122841, 2007/0122842, and 2008/0108149 and International Patent Application Publication No. WO/2000/003307.

The solid support, or support, refers to a material or group of materials having a rigid or semi-rigid surface or surfaces. In some embodiments, at least one surface of the solid support will be substantially flat, although in some embodiments it may be desirable to physically separate synthesis regions for different molecules with, for example, wells, raised regions, beads, pins, etched trenches, or the like. In certain embodiments, the solid support may be porous. In some embodiments, the solid support is glass. In some embodiments, the solid support is coated. In some embodiments, the solid support is uncoated. In some embodiments, the coating adheres amines. In some embodiments, the coating adheres lysine residues. In some embodiments, the coating adheres amino termini of proteins. In some embodiments, the coating adheres 6-His sequences. In some embodiments, the coating adheres biotin residues.

Support materials useful in embodiments of the present invention include, for example, silicon, bio-compatible polymers such as, for example poly(methyl methacrylate) (PMMA) and polydimethylsiloxane (PDMS), glass, SiO2 (such as, for example, a thermal oxide silicon wafer such as that used by the semiconductor industry), quartz, silicon nitride, functionalized glass, gold, platinum, and aluminum. Functionalized surfaces include for example, amino-functionalized glass, carboxy functionalized glass, and hydroxy functionalized glass. Additionally, a support may optionally be coated with one or more layers to provide a surface for molecular attachment or functionalization, increased or decreased reactivity, binding detection, or other specialized application. Support materials and or layer(s) may be porous or non-porous. For example, a support may be comprised of porous silicon. Additionally, the support may be a silicon wafer or chip such as those used in the semiconductor device fabrication industry. In the case of a wafer or chip, a plurality of arrays may be synthesized on the wafer. A person skilled in the art would know how to select an appropriate support material.

In some embodiments, the plurality of probes is immobilized on the array. In some embodiments, the plurality of probes is linked to the array. In some embodiments, the immobilization is via linkage. In some embodiments, the plurality of probes is directly linked to the array. In some embodiments, the plurality of probes is indirectly linked to the array. In some embodiments, the linking is via a linker. In some embodiments, the linker is an amino acid linker. In some embodiments, the linker is at least one lysine residue. In some embodiments, the linker is a plurality of lysine residues. In some embodiments, the linker is two lysine residues. In some embodiments, the linker is KK. In some embodiments, the linker is at least one histidine residue. In some embodiments, the linker is a plurality of histidine residues. In some embodiments, the linker is six histidine residues. In some embodiments, the linker is HHHHHH (SEQ ID NO: 1391). In some embodiments, the linker is a biotin residue. In some embodiments, the linker is an N-terminal linker. In some embodiments, the linker is a C-terminal linker. In some embodiments, the linker is an N-terminal or C-terminal linker.

The peptides, proteins and viruses present on the array may be linked covalently or non-covalently to the array and can be attached to the array support (e.g., silicon or other relatively flat material) by cleavable linkers. A linker molecule can be a molecule inserted between the support and peptide that is being synthesized, and a linker molecule may not necessarily convey functionality to the resulting peptide, such as molecular recognition functionality, but instead elongates the distance between the support surface and the peptide functionality to enhance the exposure of the peptide functionality on the surface of the support. Preferably a linker should be about 4 to about 120 atoms long to provide exposure. In some embodiments, a linker is at least 40 atoms long. In some embodiments, a linker is at least 48 atoms long. In some embodiments, a linker is between 40 and 150 atoms long. In some embodiments, a linker is about 48 atoms long. In some embodiments, a linker is about 120 atoms long. The linker molecules may be, for example, aryl acetylene, ethylene glycol oligomers containing 2-10 monomer units (PEGs), diamines, diacids, amino acids, biotin, among others, and combinations thereof. A person skilled in the art would know how to design appropriate linkers. In some embodiments, a probe is immobilized on the array but not linked. In some embodiments, a virus or protein is immobilized but not linked. In some embodiments, a link is reversible. In some embodiments, linking is printing the probe on the array. In some embodiments, a peptide is printed. In some embodiments, a recombinant protein is printed.

In some embodiments, each probe is located at a discrete location on a support. In some embodiments, each probe is located at a discrete location on an array. In some embodiments, each probe is immobilized at a discrete location. In some embodiments, each probe is distinctly immobilized. It will be understood by a skilled artisan that each probe must be able to be uniquely detected such that upon reading/scanning the array, the precise probe bound by an antibody can be determined. In some embodiments, each probe is immobilized on a separate support. In some embodiments, each probe is immobilized in a separate region of a support or array. In some embodiments, each probe is located or immobilized such that they can be uniquely measured or detected. In some embodiments, each probe is located or immobilized such that an antibody binding to the probe can be uniquely measured or detected.

In some embodiments, the plurality of probes comprises at least one probe from a first influenza. In some embodiments, the plurality of probes comprises at least one probe from a second influenza. In some embodiments, the plurality of probes comprises at least one probe from a third influenza. In some embodiments, the plurality of probes comprises at least one probe from at least 2, 3, 4, 5, 6, 7, 8, 9 or 10 influenzas. Each possibility represents a separate embodiment of the invention. In some embodiments, the first and second influenza are different influenzas. In some embodiments, the first and second influenzas are different types of influenza. In some embodiments, the first and second influenzas are different subtypes of influenza. In some embodiments, the first and second influenzas are different strains of influenza. In some embodiments, the first and second influenzas are the same subtype of influenzas and different strains of influenza. As used herein a “subtype” refers to a specific influenza defined by the proteins expressed by the influenza. There are three types of influenza, influenza type A, influenza type B and influenza type C. Influenzas from different types are automatically from different subtypes as well. Subtypes of influenza A are defined by their variants of two proteins: hemagglutinin (HA) and neuraminidase (NA). There are 18 different HA subtypes and 11 different NA subtypes (H1 through H18 and N1 through N11, respectively). There are three subtypes currently circulating in the human population: H1N1, H3N2 and influenza B. Table 1 provides human subtypes from the last 100 years. Other subtypes are found in various animal species. Table 5 provides several non-human subtypes. Table 6 provides H1N1, H3N2 and influenza B strains spotted on exemplary arrays. In some embodiments, the influenza is an influenza circulating in the human population. In some embodiments, the influenza is an avian influenza. In some embodiments, the influenza is a zoonotic influenza. In some embodiments, the zoonotic influenza is selected from H5N1 and H7N9. In some embodiment, the zoonotic influenza is selected from H2N2, H7N2, H7N3, H7N7, H7N9, H9N2, and H5N1. In some embodiment, the zoonotic influenza is selected from H2N2, H7N2, H7N3, H7N7, H7N9, H9N2, H4N4, H4N6, H5N8, H6N4, H8N4, H10N3, H10N8, H11N9, H13N6 and H5N1. In some embodiments, a zoonotic influenza comprises increased risk of severe disease. In some embodiments, severe disease comprises an increased death rate.

As used herein, a “strain” refers to a mutational genetic variant. In some embodiments, a strain is from a given year. In some embodiments, a strain is from a given location. An influenza virus can be named in the following way: type/place isolated/strain number/year isolated(subtype). For example: A/Solomon Islands/3/2006 (H1N1) refers to an influenza type A that was isolated in the Solomon Islands in 2006. It has a strain number of 3 and is a H1N1 subtype. In some embodiments, the strain is selected from a strain provided in Table 1. In some embodiments, the strain is selected from a strain provided in Table 6. In some embodiments, the strain is selected from a strain provided in Table 5. In some embodiments, the strain is selected from a strain provided in Table 4. In some embodiments, the strain is selected from A/California/07/2009 (H1N1), A/Wisconsin/67/2005 (H3N2), A/Puerto-Rico/8/1934 (H1N1), A/Shanghai/01/2013 (H7N9), A/Vietnam/1203/2004 (H5N1), A/Victoria/361/2011 (H3N2), and B/Wisconsin/01/2010. In some embodiments, the strain is selected from A/California/07/2009 (H1N1), A/Wisconsin/67/2005 (H3N2).

In some embodiments, the influenza is an influenza currently circulating in the world. In some embodiments, the influenza is an avian influenza. In some embodiments, the influenza is a mammalian influenza. In some embodiments, the influenza is a human influenza. In some embodiments, a human influenza is selected from the influenzas provided in Table 1. In some embodiments, a human influenza is selected from the influenzas provided in Table 6. In some embodiments, the influenza is a non-human influenza. In some embodiments, a non-human influenza is selected from the influenzas provided in Table 5. In some embodiments, the influenza is an influenza currently circulating in a human population. In some embodiments, the influenza types are selected from influenza A, influenza B and influenza C. In some embodiments, the influenza types are selected from influenza A, and influenza B. In some embodiments, the influenza subtypes are selected from influenza A/H1N1, influenza A/H3N2 and influenza B. In some embodiments, the influenza is selected from influenza A/H1N1, influenza A/H3N2 and influenza B. In some embodiments, the influenza is selected from influenza A/H1N1, A/H3N2, B, A/H2N2, A/H4N4, A/H4N6, A/H5N1, A/H5N8, A/H6N4, A/H7N2, A/H7N3, A/H7N2, A/H7N3, A/H7N7, A/H7N9, A/H8N4, A/H9N2, A/H10N3, A/H10N8, A/H11N9, and A/H13N6. In some embodiments, the influenza is selected from influenza H1N1, H3N2, B, H7N9, and H5N1. In some embodiments, the influenza is selected from influenza H1N1, H2N2, H3N2, B, H7N2, H7N3, H7N7, H7N9, H9N2, and H5N1. In some embodiments, the influenza is selected from an influenza listed in Table 1. In some embodiments, the influenza is selected from an influenza listed in Table 1 and Table 5. In some embodiments, the influenza is selected from an influenza listed in Table 5. In some embodiments, the influenza is selected from an influenza listed in Table 6.

TABLE 5
List of HA and/or NA proteins that were spotted on an exemplary non-
human influenza array.

| Influenza strain name | Subtype | Species | HA or HA1 protein | NA protein |
|---|---|---|---|---|
| A/mallard/Minnesota/Sg-00194/2007 | H10N3 | avian | X | |
| A/duck/Guangdong/E1/2012 | H10N8 | avian | X | X |
| A/Jiangxi-Donghu/346/2013 | H10N8 | avian | | X |
| A/mallard/Alberta/294/1977 | H11N9 | avian | X | |
| A/black-headedgull/Sweden/1/1999 | H13N6 | avian | X | |
| A/greyteal/Australia/2/1979 | H4N4 | avian | | X |
| A/mallard/Ohio/657/2002 | H4N6 | avian | X | |
| A/Bar-headedgoose/Qingi/14/2008 | H5N1 | avian | X | |
| A/chicken/Yamaguchi/7/2004 | H5N1 | avian | X | |
| A/duck/Hunan/795/2002 | H5N1 | avian | X | |
| A/duck/Laos/3295/2006 | H5N1 | avian | X | |
| A/chicken/Netherlands/14015531/2014 | H5N8 | avian | | X |
| A/chicken/HongKong/17/1977 | H6N4 | avian | X | |
| A/pintailduck/Alberta/114/1979 | H8N4 | avian | X | |
| A/Chicken/HongKong/G9/1997 | H9N2 | avian | X | X |
| A/duck/HongKong/448/1978 | H9N2 | avian | X | |
| A/HongKong/1073/1999 | H9N2 | avian | X | X |
| A/HongKong/35820/2009 | H9N2 | avian | X | |
| A/turkey/Wisconsin/1/1966 | H9N2 | avian | X | X |
| A/Guinea fowl/Hong Kong/WF10/1999 | H9N2 | guinea_pig | X | |

In some embodiments, the influenza B is selected from B/Brisbane/60/2008, B/Colorado/06/2017, B/Florida/4/2006, B/HongKong/330/2001, B/Jiangsu/10/2003, B/Lee/1940, B/Malaysia/2506/2004, B/Massachusetts/02/2012, B/Phuket/3073/2013, and B/Yamagata/16/1988. In some embodiments, the H1N1 influenza is selected from A/Beijing/262/1995, A/Brazil/11/1978, A/Brisbane/02/2018, A/Brisbane/59/2007, A/California/04/2009, A/California/07/2009, A/Chile/1/1983, A/Christchurch/16/2010, A/Michigan/45/2015, A/NewCaledonia/20/1999, A/PuertoRico/8/1934, A/Singapore/6/1986, A/SolomonIslands/3/2006, A/SouthCarolina/1/1918, A/USSR/90/1977, and A/Wisconsin/1933. In some embodiments, the H2N2 influenza is A/Japan/305/1957. In some embodiments, the H3N2 influenza is selected from A/Aichi/2/1968, A/Babol/36/2005, A/Bangkok/1/1979, A/Brisbane/10/2007, A/California/07/2004, A/Fujian/411/2002, A/Guizhou/54/1989, A/Hawaii/07/2009, A/HongKong/4801/2014, A/Kansas/14/2017, A/Leningrad/360/1986, A/Maryland/26/2014, A/NewYork/55/2004, A/Panama/2007/1999, A/Perth/16/2009, A/Portchalmers/1/1973, A/Shandong/9/1993, A/Singapore/INFIMH-16-0019/2016, A/Switzerland/9715293/2013, A/Sydney/5/1997, A/Texas/50/2012, A/Uruguay/716/2007, A/Victoria/210/2009, A/Victoria/361/2011, A/Wisconsin/67/2005, and A/X-31/1968. In some embodiments, the H5N1 influenza is selected from A/Anhui/1/2005, A/HongKong/483/1997, A/Hubei/1/2010, A/Indonesia/5/2005, A/Thailand/1 (KAN-1)/2004, and A/Vietnam/1203/2004. In some embodiments, the H7N2 influenza is A/NewYork/107/2003. In some embodiments, the H7N3 influenza is A/Canada/444/2004. In some embodiments, the H7N7 influenza is A/Netherlands/219/2003. In some embodiments, the H7N9 influenza is selected from A/Anhui/1/2013 and A/Shenghai/02/2013. In some embodiments, the H9N2 influenza is A/HongKong/33982/2009.

In some embodiments, the non-human is avian. In some embodiments, the non-human is rodent. In some embodiments, the non-human is porcine. In some embodiments, the porcine is swine. In some embodiments, the non-human influenza is selected form the influenzas provided in Table 5. In some embodiments, the zoonotic influenza is selected form the influenzas provided in Table 5. In some embodiments, the non-human influenza is selected from H4N4, H4N6, H5N1, H5N8, H6N4, H8N4, and H9N2. In some embodiments, the non-human influenza is selected from H1N1, H2N2, H4N4, H4N6, H5N1, H5N8, H6N4, H7N2, H7N3, H7N7, H7N9, H8N4, H9N2, H10N3, H10N8, H11N9, H13N6 and B. In some embodiments, the zoonotic influenza is selected from H1N1, H2N2, H4N4, H4N6, H5N1, H5N8, H6N4, H7N2, H7N3, H7N7, H7N9, H8N4, H9N2, H10N3, H10N8, H11N9, H13N6 and B In some embodiments, the non-human influenza is selected from A/mallard/Minnesota/Sg-00194/2007, A/duck/Guangdong/E1/2012, A/Jiangxi-Donghu/346/2013, A/mallard/Alberta/294/1977, A/black-headedgull/Sweden/1/1999, A/greyteal/Australia/2/1979, A/mallard/Ohio/657/2002, A/Bar-headedgoose/Qingi/14/2008, A/chicken/Yamaguchi/7/2004, A/duck/Hunan/795/2002, A/duck/Laos/3295/2006, A/chicken/Netherlands/14015531/2014, A/chicken/HongKong/17/1977, A/pintailduck/Alberta/114/1979, A/Chicken/HongKong/G9/1997, A/duck/HongKong/448/1978, A/HongKong/1073/1999, A/HongKong/35820/2009, A/turkey/Wisconsin/1/1966, and A/Guinea fowl/Hong Kong/WF10/1999.

As used herein the term “probe” refers to a part from a virus or a whole virus that contains at least one epitope that can be bound by an antibody. In some embodiments, a probe is a whole virus. In some embodiments, the probe is a virus-like particle (VLP). In some embodiments, a probe is a lysed virus. In some embodiments, a probe is a fraction from a lysed virus. In some embodiments, a probe is a whole protein. In some embodiments, the protein is a recombinant protein. In some embodiments, a probe is a portion of a protein. In some embodiments, the portion comprises a functional domain. In some embodiments, the protein is a peptide. In some embodiments, the probe comprises amino acids. In some embodiments, the probe comprises viral protein. In some embodiments, the probe comprises a viral epitope. In some embodiments, the epitope is an immunological epitope. In some embodiments, the probe is selected from a whole virus, a lysed virus, a VLP, a whole recombinant protein and a peptide.

As used herein, the terms “peptide”, and “polypeptide” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid. In some embodiments, the probe comprises a peptide. It will be understood that even a full virus will inherently comprise a peptide and it will comprise an amino acid. Similarly, a VLP and a recombinant protein must comprise a peptide.

In some embodiments, the peptide is a purified peptide. In some embodiments, the peptide is an isolated peptide. In some embodiments, the peptide is a recombinant peptide. In some embodiments, the peptide is a synthetic peptide. As used herein, the term “isolated peptide” refers to a peptide that is essentially free from contaminating cellular components, such as carbohydrate, lipid, or other proteinaceous impurities associated with the peptide in nature. Typically, a preparation of isolated peptide contains the peptide in a highly purified form, i.e., at least about 80% pure, at least about 90% pure, at least about 95% pure, greater than 95% pure, or greater than 99% pure. In some embodiments, a synthetic peptide is at least 99% pure. In some embodiments, a synthetic peptide is 100% pure.

In some embodiments, the peptide is a fragment of a influenza protein. In some embodiments, the peptide is a protein fragment that retains an immunogenic epitope. In some embodiments, the peptide is a linear peptide. In some embodiments, the peptide is a conformational peptide. In some embodiments, the peptide contains three-dimensional structure. In some embodiments, a cysteine residue of a peptide has been mutated to another amino acid. Removal of cysteines removes disulfide bridges that may change the conformation of the peptide or render it non-linear. In some embodiments, the cysteine is mutated to a non-charged amino acid. In some embodiments, the cysteine is mutated to a non-charged amino acid. In some embodiments, the cysteine is mutated to a non-polar amino acid. In some embodiments, the cysteine is mutated to a methionine. In some embodiments, all cysteines are mutated. In some embodiments, the influenza protein is a surface protein.

In some embodiments, the peptide comprises a domain from an influenza protein. In some embodiments, the peptide comprises a functional domain from an influenza protein. In some embodiments, the peptide comprises a motif from an influenza protein. In some embodiments, the peptide comprises sufficient amino acids to retain a secondary structure found in an intact protein. In some embodiments, the secondary structure is a three-dimensional structure. In some embodiments, the peptide comprises a functional fragment of the protein. In some embodiments, the probe is functional. In some embodiments, the probe comprises a 3D functional epitope. In some embodiments, the probe comprises a conformational epitope. In some embodiments, the probe comprises a linear epitope.

In some embodiments, the probe is a full protein. In some embodiments, the protein is a recombinant protein. In some embodiments, the protein is an isolated protein. In some embodiments, the protein comprises a post-translational modification. In some embodiments, the post-translational modification is glycosylation. In some embodiments, the probe is a full virus. In some embodiments, the probe is a lysed virus. In some embodiments, the virus is an inactivated virus. It will be understood by a skilled artisan that full proteins and full viruses are likely to be properly folded and thus provide 3D, conformational epitopes, while peptide may or may not have conformational epitope and not just linear epitopes.

In some embodiments, a peptide comprises at least 5, 7, 10, 12, 14, 15, 16, 18, 20 or 25 amino acids. Each possibility represents a separate embodiment of the invention. In some embodiments, a peptide comprises at least 10 amino acids. In some embodiments, a peptide comprises at least 14 amino acids. In some embodiments, a peptide comprises at least 20 amino acids. In some embodiments, a peptide is not a complete protein. In some embodiments, a peptide comprises between 5 and 200, 5 and 150, 5 and 100, 5 and 90, 5 and 90, 5 and 70, 5 and 60, 5 and 58, 5 and 50, 10 and 200, 10 and 150, 10 and 100, 10 and 90, 10 and 90, 10 and 70, 10 and 60, 10 and 58, 10 and 50, 12 and 200, 12 and 150, 12 and 100, 12 and 90, 12 and 90, 12 and 70, 12 and 60, 12 and 58, 12 and 50, 14 and 200, 14 and 150, 14 and 100, 14 and 90, 14 and 90, 14 and 70, 14 and 60, 14 and 58, 14 and 50, 15 and 200, 15 and 150, 15 and 100, 15 and 90, 15 and 90, 15 and 70, 15 and 60, 15 and 58, or 15 and 50 amino acids. Each possibility represents a separate embodiment of the invention. In some embodiments, a peptide comprises between 5 and 60 amino acids. In some embodiments, a peptide comprises between 10 and 60 amino acids. In some embodiments, a peptide comprises between 14 and 60. In some embodiments, a peptide comprises between 5 and 58 amino acids. In some embodiments, a peptide comprises between 10 and 58 amino acids. In some embodiments, a peptide comprises between 14 and 58. In some embodiments, a peptide comprises at most 50, 58, 60, 70, 80, 90, 100, 125, 150, 175, 200 or 250 amino acids. Each possibility represents a separate embodiment of the invention. In some embodiments, a peptide comprises at most 60 amino acids. In some embodiments, a peptide comprises at most 100 amino acids. In some embodiments, the amino acids are consecutive amino acids from an influenza protein.

In some embodiments, a peptide is a protein. In some embodiments, a peptide is a part of a protein. In some embodiments, a peptide comprises a functional domain of a protein. In some embodiments, a peptide is a complete protein. In some embodiments, a complete protein is a whole protein. In some embodiments, a complete protein comprises a signal peptide. In some embodiments, a complete protein lacks a signal peptide. In some embodiments, the protein is a recombinant protein. In some embodiments, the probe is a complete protein. Recombinant proteins can be produced by any method known in the art, or can be purchased for commercial supplies, such as for example Sino Biological and The Native Antigen Company.

In some embodiments, the plurality of probes comprises a probe comprising an amino acid sequence of an influenza protein. In some embodiments, the plurality of probes comprises a probe consisting of an amino acid sequence of an influenza protein. In some embodiments, the plurality of probes comprises a protein probe from a first influenza. In some embodiments, the plurality of probes comprises a protein probe from a second influenza. In some embodiments, the protein is a surface protein. In some embodiments, the protein is an influenza HA protein. In some embodiments, the protein is an influenza HAI protein. In some embodiments, the protein is an influenza NA protein. In some embodiments, the protein is a recombinant protein. In some embodiments, the protein is a secreted protein. It will be understood by a skilled artisan that by using a protein, secondary structures and intramolecular bonds and interactions will be preserved. In some embodiments, a probe comprises a whole influenza protein. In some embodiments, a probe consists of a whole influenza protein. In some embodiments, the protein is selected from an HA protein and a NA protein. In some embodiments, the plurality of probes comprises a first probe that consists of a whole protein and a second probe that consists of a whole protein.

In some embodiments, the plurality of probes comprises at least 1, 2, 3, 4, 5, 6, 7, 8, 9 or 10 peptides from an influenza. Each possibility represents a separate embodiment of the invention. In some embodiments, the plurality of probes comprises at least 2 peptides from an influenza. In some embodiments, the plurality of probes comprises 2 peptides from an influenza. In some embodiments, the plurality of probes comprises probes from at least 2 different influenza proteins. In some embodiments, the two proteins are an HA protein and a NA protein. In some embodiments, the plurality of probes comprises at least 1, 2, 3, 4, 5, 6, 7, 8, 9 or 10 peptides from each influenza. Each possibility represents a separate embodiment of the invention. In some embodiments, the plurality of probes comprises at least 2 peptides from each influenza. In some embodiments, the plurality of probes comprises 2 peptides from each influenza. In some embodiments, at least 2 is 2. In some embodiments, the plurality of probes comprises at least a peptide from an HA protein from each influenza. In some embodiments, the plurality of probes comprises at least a peptide from a NA protein from each influenza. In some embodiments, the plurality of probes comprises at least the HA protein from each influenza. In some embodiments, the plurality of probes comprises at least the NA protein from each influenza. In some embodiments, the peptide is the whole protein.

In some embodiments, an array comprises probes to at least 2 influenzas. In some embodiments, an array comprises probes to at least 3 influenzas. In some embodiments, an array comprises at least 10, 20, 30, 40, 50, 60, 70, 80, 90, 100, 150, 200, 250, 300, 350, 400, 450, 500, 550 or 600 probes. Each possibility represents a separate embodiment of the invention. In some embodiments, an array comprises at least 10 probes. In some embodiments, an array comprises at least 50 probes. In some embodiments, an array comprises at least 90 probes. In some embodiments, an array comprises at most 250, 300, 400, 500, 600, 700, 750, 800, 900 or 1000 probes. Each possibility represents a separate embodiment of the invention. In some embodiments, an array comprises at most 600 probes. In some embodiments, an array comprises 2-800, 10-800, 50-800, 90-800, 100-800, 200-800, 2-700, 10-700, 50-700, 90-700, 100-700, 200-70, 2-650, 10-650, 50-650, 90-650, 100-650, 200-650, 2-600, 10-600, 50-600, 90-600, 100-600, 200-600, 2-550, 10-550, 50-550, 90-550, 100-550, 200-550, 2-500, 10-500, 50-500, 90-500, 100-500, 200-500, 2-400, 10-400, 50-400, 90-400, 100-400, 200-400, 2-300, 10-300, 50-300, 90-300, 100-300, 200-300, 2-250, 10-250, 50-250, 90-250, 100-250, or 200-250. Each possibility represents a separate embodiment of the invention. In some embodiments, an array comprises 90-600 probes. In some embodiments, an array comprises 90-250 probes.

In some embodiments, a peptide is a protein. In some embodiments, a peptide is a complete protein. In some embodiments, a complete protein comprises a signal peptide. In some embodiments, a complete protein lacks a signal peptide. In some embodiments, the plurality of probes comprises an amino acid sequence of an influenza protein. In some embodiments, the plurality of probes comprises a protein probe from a first influenza. In some embodiments, the plurality of probes comprises a protein probe from a second influenza. In some embodiments, the protein is a surface protein. In some embodiments, the protein is an influenza envelope protein. In some embodiments, the protein is selected from a hemagglutinin (HA) and neuraminidase (NA). In some embodiments, the protein is a recombinant protein. It will be understood by a skilled artisan that by using a protein secondary structures and intramolecular bonds and interactions will be preserved.

In some embodiments, the plurality of probes comprises at least 1, 2, 3, 4, 5, 6, 7, 8, 9 or 10 peptides from an influenza. Each possibility represents a separate embodiment of the invention. In some embodiments, the plurality of probes comprises at least 2 peptides from an influenza. In some embodiments, the plurality of probes comprises 2 peptides from an influenza. In some embodiments, the two proteins are HA and NA. In some embodiments, the plurality of probes comprises at least 1, 2, 3, 4, 5, 6, 7, 8, 9 or 10 peptides from each influenza. Each possibility represents a separate embodiment of the invention. In some embodiments, the plurality of probes comprises at least 2 peptides from each influenza. In some embodiments, the plurality of probes comprises 2 peptides from each influenza. In some embodiments, the plurality of probes comprises at least 2 peptides from each subtype of influenza. In some embodiments, the plurality of probes comprises at least 2 peptides from each strain of influenza. In some embodiments, at least 2 is 2. In some embodiments, the plurality of probes comprises at least a peptide from HA from each subtype. In some embodiments, the plurality of probes comprises at least a peptide from HA from each strain. In some embodiments, the plurality of probes comprises at least the HA protein from each subtype. In some embodiments, the plurality of probes comprises at least the HA protein from each strain. In some embodiments, the plurality of probes comprises at least a peptide from NA from each subtype. In some embodiments, the plurality of probes comprises at least a peptide from NA from each strain. In some embodiments, the plurality of probes comprises at least the NA protein from each subtype. In some embodiments, the plurality of probes comprises at least the NA protein from each strain.

In some embodiments, the plurality of probes comprises probes from influenzas from different years. In some embodiments, the plurality of probes comprises a probe from an influenza from a first year. In some embodiments, the plurality of probes comprises a probe from an influenza from a second year. In some embodiments, the first and second years are different years. In some embodiments, the plurality of probes comprises the same probe from different years. In some embodiments, the plurality of probes comprises a probe from a given strain from a first year, and a probe from the same strain from a second year. In some embodiments, different strains are by definition from different years. In some embodiments, the plurality of probes comprises a probe from a given subtype from a first year, and a probe from the same subtype from a second year. In some embodiments, the probe from different years are the same region of a peptide or protein but comprise different amino acid sequences. In some embodiments, it is the same protein from different years. It will be understood that in different years due to genetic drift there will be introduced mutations into a given amino acids sequence. Thus, the same peptide from one year to another, may be recognizable as the same peptide even though the sequence may be altered. Similarly, a given protein may be recognized as the same protein even if mutations have been generated in the amino acid sequence.

In some embodiments, the plurality of probes further comprises an influenza virus. In some embodiments, the influenza virus is an inactivated virus. As used herein, the term “inactivated virus” refers to a virulent virus that has been made non-infectious. In some embodiments, an inactivated virus is a killed virus. In some embodiments, an inactivated virus is a virus comprising a mutation that reduces virulence. In some embodiments, an inactivated virus is a virulent virus some of whose proteins have been transferred to a backbone of a less virulent or non-virulent virus. In some embodiments, the influenza is a lysed virus. In some embodiments, the virus is a virion. In some embodiments, the lysed virus is a lysed cell infected by the virus. In some embodiments, the lysed virus is media from infected cells containing virus. In some embodiments, the virus is a virus-like particle. In some embodiments, the plurality of probes comprises a virus-like particle (VLP). In some embodiments, the VLP is an influenza VLP. As used herein, the term “virus-like particle” refers to a multiprotein structure that mimics the organization and conformation of an authentic native virus but lacks the viral genome. In some embodiments, the plurality of probes comprises a lysate from a cell infected by an influenza virus. In some embodiments, the lysate is mixed with spotting buffer before immobilization on the array or support. It will be appreciated by a skilled artisan that by using whole virus, VLPs or cell lysate viral epitopes will be provided in their natural confirmation. In some embodiments, the plurality of probes comprises at least two VLP probes. In some embodiments, the plurality of probes comprises at least two lysed virus probes. In some embodiments, the plurality of probes comprises at least two virus probes that are different influenzas.

In some embodiments, the probes are present on the array or support at a concentration sufficient for antibody binding. In some embodiments, the probes are present on the array or support at a concentration sufficient for detectable antibody binding. In some embodiments, the concentration is at least 0.00001, 0.00005, 0.0001, 0.0005, 0.001, 0.005, 0.01, 0.05, 0.1, 0.5, 1, 2, 3, 4, 5, 10, 100, 1000, 10000, 100000, 1000000, 10000000, 100000000, or 1000000000 ng per spot of probe. Each possibility represents a separate embodiment of the invention. In some embodiments, the concentration is at least 0.00001, 0.00005, 0.0001, 0.0005, 0.001, 0.005, 0.01, 0.05, 0.1, 0.5, 1, 2, 3, 4, 5, 10, 100, 1000, 10000, 100000, 1000000, 10000000, 100000000, or 1000000000 ng/ml. Each possibility represents a separate embodiment of the invention. In some embodiments, the concentration is at most 0.00001, 0.00005, 0.0001, 0.0005, 0.001, 0.005, 0.01, 0.05, 0.1, 0.5, 1, 2, 3, 4, 5, 10, 100, 1000, 10000, 100000, 1000000, 10000000, 100000000, or 1000000000 ng per spot of probe. Each possibility represents a separate embodiment of the invention. In some embodiments, the concentration is at most 0.00001, 0.00005, 0.0001, 0.0005, 0.001, 0.005, 0.01, 0.05, 0.1, 0.5, 1, 2, 3, 4, 5, 10, 100, 1000, 10000, 100000, 1000000, 10000000, 100000000, or 1000000000 ng/ml. Each possibility represents a separate embodiment of the invention. In some embodiments, the concentration of whole influenza virus antigen is 2-4 HAU/ul. In some embodiments, the concentration of whole influenza virus antigen is 4-8 ugHA/ul. In some embodiments, the concentration of protein is 8-32 ug/ml. In some embodiments, the concentration of peptide is −1 mg/ml. In some embodiments, the volume of the spot is at least 0.00001, 0.00005, 0.0001, 0.0005, 0.001, 0.005, 0.01, 0.05, 0.1, 0.5, 1, 2, 3, 4, 5, 10, 100, 1000, 10000, 100000, 1000000, 10000000, 100000000, or 1000000000 nL. Each possibility represents a separate embodiment of the invention. In some embodiments, the volume of the spot or probe is at most 0.00001, 0.00005, 0.0001, 0.0005, 0.001, 0.005, 0.01, 0.05, 0.1, 0.5, 1, 2, 3, 4, 5, 10, 100, 1000, 10000, 100000, 1000000, 10000000, 100000000, or 1000000000 nL. Each possibility represents a separate embodiment of the invention. In some embodiments, the volume of the spot or probe is ˜370 pL. In some embodiments, the spotted mass of whole recombinant proteins is between 3-20 picograms. In some embodiments, the spotted mass of peptides is between 300-400 picograms. It will be understood that shorter peptides will tend to have a lower mass and longer peptide a larger mass. In some embodiments, the spotted mass of peptide is ˜370 picograms.

In some embodiments, the plurality of probes is selected form the probes provided in Table 1. In some embodiments, the plurality of probes comprises the probes provided in Table 1. In some embodiments, the plurality of probes consists of the probes provided in Table 1. In some embodiments, the plurality of probes is selected form the probes provided in Table 6. In some embodiments, the plurality of probes comprises the probes provided in Table 6. In some embodiments, the plurality of probes consists of the probes provided in Table 6. In some embodiments, the plurality of probes is selected form the probes provided in Table 5. In some embodiments, the plurality of probes comprises the probes provided in Table 5. In some embodiments, the plurality of probes consists of the probes provided in Table 5. In some embodiments, the plurality of probes is selected form the probes provided in Table 5 and SEQ ID NO: 598-995. In some embodiments, the plurality of probes comprises the probes provided in Table 5 and SEQ ID NO: 598-995. In some embodiments, the plurality of probes consists of the probes provided in Table 5 and SEQ ID NO: 598-995. In some embodiments, the plurality of probes is selected from the probes provided in Table 4. In some embodiments, the plurality of probes comprises the probes provided in Table 4. In some embodiments, the plurality of probes consists of the probes provided in Table 4. In some embodiments, the plurality of probes is selected form the probes provided in SEQ ID NOs: 1-399. In some embodiments, the plurality of probes comprises the probes provided in SEQ ID NOs: 1-399. In some embodiments, the plurality of probes consists of the probes provided in SEQ ID NOs: 1-399. In some embodiments, the plurality of probes is selected form the probes provided in SEQ ID NOs: 1-1390. In some embodiments, the plurality of probes comprises the probes provided in SEQ ID NOs: 1-1390. In some embodiments, the plurality of probes consists of the probes provided in SEQ ID NOs: 1-1390. In some embodiments, the whole virus probes are selected from the probes provided in Table 1. In some embodiments, the VLP probes are selected from the probes provided in Table 1. In some embodiments, the virus lysate probes are selected from the probes provide in Table 1. In some embodiments, the whole protein probes are selected from the probes provided in Table 1. In some embodiments, the whole protein probes are selected from the probes provided in Table 5. In some embodiments, the whole virus probes are selected from the probes provided in Table 6. In some embodiments, the VLP probes are selected from the probes provided in Table 6. In some embodiments, the virus lysate probes are selected from the probes provide in Table 6. In some embodiments, the whole protein probes are selected from the probes provided in Table 6. In some embodiments, the whole NA probes are selected from the probes provide in Table 1. In some embodiments, the whole NA probes are selected from the probes provide in Table 5. In some embodiments, the whole NA probes are selected from the probes provide in Table 6. In some embodiments, the whole HA probes are selected from the probes provided in Table 1. In some embodiments, the whole HA probes are selected from the probes provided in Table 5. In some embodiments, the whole HA probes are selected from the probes provided in Table 6. In some embodiments, the peptide probes are selected from the probes provided in Table 4. In some embodiments, the HA peptide probes are selected from the HA probes provided in Table 4. In some embodiments, the NA probes are selected from the NA probes provided in Table 4. In some embodiments, the peptide probes are selected from SEQ ID NOs: 1-399. In some embodiments, the peptide probes are selected from SEQ ID NOs: 1-1390. In some embodiments, the peptide probes comprise peptides selected from SEQ ID NOs: 1-399. In some embodiments, the peptide probes comprise peptides selected from SEQ ID NOs: 1-1390. In some embodiments, the peptide probes comprise at least 2 peptides selected from SEQ ID NOs: 1-399. In some embodiments, the peptide probes comprise at least 2 peptides selected from SEQ ID NOs: 1-1390. In some embodiments, the peptide probes comprise at least 2, 3, 4, 5, 6, 7, 8, 9, 10, 15, 20, 25, 30, 35, 40, 45, 50, 60, 70, 80, 90, 100, 110, 120, 125, 130, 140, 150, 160, 170, 175, 180, 190, 195 or 200 peptides selected from SEQ ID NOs: 1-1390. Each possibility represents a separate embodiment of the invention. In some embodiments, the peptide probes comprise at least 2, 3, 4, 5, 6, 7, 8, 9, 10, 15, 20, 25, 30, 35, 40, 45, 50, 60, 70, 80, 90, 100, 110, 120, 125, 130, 140, 150, 160, 170, 175, 180, 190, 195 or 200 peptides selected from SEQ ID NOs: 1-399. Each possibility represents a separate embodiment of the invention. In some embodiments, the peptide probes comprise at least 2 peptides selected from SEQ ID NOs: 1-1390. Each possibility represents a separate embodiment of the invention. In some embodiments, the peptide probes comprise at least 2 peptides selected from SEQ ID NOs: 1-399. In some embodiments, the peptide probes comprise at least 10 peptides selected from SEQ ID NOs: 1-1390. In some embodiments, the peptide probes comprise at least 10 peptides selected from SEQ ID NOs: 1-399. In some embodiments, the peptide probes comprise at least 2, 3, 4, 5, 6, 7, 8, 9, 10, 15, 20, 25, 30, 35, 40, 45, 50, 60, 70, 80, 90, 100, 110, 120, 125, 130, 140, 150, 160, 170, 175, 180, 190, 195 or 199 peptides selected from SEQ ID NOs: 1-399. Each possibility represents a separate embodiment of the invention. In some embodiments, the peptide probes comprise at least 1 peptide selected from SEQ ID NOs: 1-199. In some embodiments, the peptide probes comprise at least 2 peptides selected from SEQ ID NOs: 1-199. In some embodiments, the peptide probes comprise at least 10 peptides selected from SEQ ID NOs: 1-199. In some embodiments, the peptide probes comprise at least 2, 3, 4, 5, 6, 7, 8, 9, 10, 15, 20, 25, 30, 35, 40, 45, 50, 60, 70, 80, 90, 100, 110, 120, 125, 130, 140, 150, 160, 170, 175, 180, 190, 195 or 200 peptides selected from SEQ ID NOs: 200-399. Each possibility represents a separate embodiment of the invention. In some embodiments, the peptide probes comprise at least 1 peptide selected from SEQ ID NOs: 200-399. In some embodiments, the peptide probes comprise at least 2 peptides selected from SEQ ID NOs: 200-399. In some embodiments, the peptide probes comprise at least 10 peptides selected from SEQ ID NOs: 200-399. In some embodiments, the at least 2 peptides comprise 1 HA at least one HA peptide and at least one NA peptide. In some embodiments, peptide probes against HA are selected from SEQ ID NOs: 1-109, 200-310, 400-508, 598-707, 799-908, 996-1106, and 1191-1304. In some embodiments, peptide probes against NA are selected from SEQ ID NOs: 110-199, 311-399, 509-597, 708-798, 909-995, 1107-1190, and 1305-1390.

In some embodiments, the probes are selected from the probes provided in Tables 1, 4 and 5. In some embodiments, the plurality of probes is selected form the probes provided in Tables 1, 4 and 5. In some embodiments, the plurality of probes comprises the probes provided in Tables 1, 4 and 5. In some embodiments, the plurality of probes consists of the probes provided in Tables 1, 4 and 5. In some embodiments, the probes are selected from the probes provided in Tables 1 and 5. In some embodiments, the plurality of probes is selected form the probes provided in Tables 1 and 5. In some embodiments, the plurality of probes comprises the probes provided in Tables 1 and 5. In some embodiments, the plurality of probes consists of the probes provided in Tables 1 and 5. In some embodiments, the probes are selected from the probes provided in Tables 1 and 4. In some embodiments, the plurality of probes is selected form the probes provided in Tables 1 and 4. In some embodiments, the plurality of probes comprises the probes provided in Tables 1 and 4. In some embodiments, the plurality of probes consists of the probes provided in Tables 1 and 4.

In some embodiments, the plurality of probes comprises at least 10 probes selected from Table 1, 4, 5 or 6. In some embodiments, the plurality of probes comprises at least 10 probes selected from Tables 1 and 4. In some embodiments, the plurality of probes comprises at least 5 probes from Table 1 and at least 5 probes from Table 4. In some embodiments, the plurality of probes comprises at least 5 probes from Table 1 and at least 5 probes from SEQ ID NO: 1-1390. In some embodiments, the plurality of probes comprises at least 5 probes from Table 1 and SEQ ID NO: 1-1390. In some embodiments, the plurality of probes comprises at least 20 probes selected from Table 1, 4, 5 or 6. In some embodiments, the plurality of probes comprises at least 20 probes selected from Tables 1 and 4. In some embodiments, the plurality of probes comprises at least 20 probes selected from Table 1 and SEQ ID NO: 1-1390. In some embodiments, the plurality of probes comprises at least 10 probes from Table 1 and at least 10 probes from Table 4. In some embodiments, the plurality of probes comprises at least 10 probes from Table 1 and at least 10 probes from SEQ ID NO: 1-1390. In some embodiments, the plurality of probes comprises at least 20 probes selected from Table 1. In some embodiments, the plurality of probes comprises at least 20 probes selected from Table 6. In some embodiments, the plurality of probes comprises at least 20 probes selected from Table 4. In some embodiments, the plurality of probes comprises at least 20 probes selected from SEQ ID NO: 1-1390. In some embodiments, the plurality of probes comprises at least 20 probes selected from Table 5. In some embodiments, the plurality of probes comprises at least 20 probes selected from Table 5 and SEQ ID NO: 598-995. In some embodiments, the plurality of probes comprises at least 10 probes selected from Table 1. In some embodiments, the plurality of probes comprises at least 10 probes selected from Table 6. In some embodiments, the plurality of probes comprises at least 10 probes selected from Table 4. In some embodiments, the plurality of probes comprises at least 10 probes selected from SEQ ID NO: 1-1390. In some embodiments, the plurality of probes comprises at least 10 probes selected from Table 5. In some embodiments, the plurality of probes comprises at least 10 probes selected from Table 5 and SEQ ID NO: 598-995. In some embodiments, the plurality of probes comprises at least 30 probes selected from Table 1 and Table 4. In some embodiments, the plurality of probes comprises at least 30 probes selected from Table 1 and SEQ ID NO: 1-1390. In some embodiments, the plurality of probes comprises at least 40 probes selected from Table 1 and Table 4. In some embodiments, the plurality of probes comprises at least 40 probes selected from Table 1 and SEQ ID NO: 1-1390. In some embodiments, the plurality of probes comprises at least 50 probes selected from Table 1 and Table 4. In some embodiments, the plurality of probes comprises at least 50 probes selected from Table 1 and SEQ ID NO: 1-1390. In some embodiments, the plurality of probes comprises at least 100 probes selected from Table 1 and Table 4. In some embodiments, the plurality of probes comprises at least 100 probes selected from Table 1 and SEQ ID NO: 1-1390. In some embodiments, the plurality of probes comprises all the probes from Table 1 and Table 4. In some embodiments, the plurality of probes comprises all the probes from Table 1. In some embodiments, the plurality of probes comprises all the probes from Table 4. In some embodiments, the plurality of probes comprises all the probes from Table 4 of a particular subtype. In some embodiments, the plurality of probes comprises at least 190 probes from Table 4. In some embodiments, the plurality of probes comprises all the Wis05 probes in Table 4. In some embodiments, the plurality of probes comprises all the Cal probes in Table 4. In some embodiments, the plurality of probes comprises SEQ ID NO: 1-199. In some embodiments, the plurality of probes comprises SEQ ID NO: 200-399. In some embodiments, the plurality of probes comprises SEQ ID NO: 200-399. In some embodiments, the plurality of probes comprises SEQ ID NO:400-597. In some embodiments, the plurality of probes comprises SEQ ID NO: 598-798. In some embodiments, the plurality of probes comprises SEQ ID NO: 799-995. In some embodiments, the plurality of probes comprises SEQ ID NO: 996-1190. In some embodiments, the plurality of probes comprises SEQ ID NO: 1191-1390.

In some embodiments, the plurality of probes comprises peptides selected from Table 4. In some embodiments, the plurality of probes consists of peptides selected from Table 4. In some embodiments, the plurality of probes consists of the peptides of Table 4. In some embodiments, the plurality of probes comprises peptides, wherein the peptides consist of the peptides of Table 4. In some embodiments, the plurality of probes consists of peptides selected from Table 4. Peptides covering the HA protein are provide, as are peptides covering the NA protein. In some embodiments, the plurality of probes comprises peptides spanning at least 10, 20, 25, 30, 40, 50, 60, 70, 75, 80, 90, 95, 99 or 100% of an influenza protein. Each possibility represents a separate embodiment of the invention. In some embodiments, the plurality of probes comprises peptides covering 100% of an influenza protein. In some embodiments, the plurality of probes comprises probes selected from SEQ ID NO: 1-199. In some embodiments, the plurality of probes comprises probes selected from SEQ ID NO: 200-399. In some embodiments, the plurality of probes comprises SEQ ID NO: 200-399. In some embodiments, the plurality of probes comprises probes selected from SEQ ID NO:400-597. In some embodiments, the plurality of probes comprises SEQ ID NO: 598-798. In some embodiments, the plurality of probes comprises probes selected from SEQ ID NO: 799-995. In some embodiments, the plurality of probes comprises probes selected from SEQ ID NO: 996-1190. In some embodiments, the plurality of probes comprises probes selected from SEQ ID NO: 1191-1390.

In some embodiments, the plurality of probes comprises viruses, VLPs or both selected from Table 1. In some embodiments, the plurality of probes consists of virus, VLPs or both selected from Table 1. In some embodiments, the plurality of probes consists of the viruses, VLPs or both of Table 1. In some embodiments, the plurality of probes comprises viruses, wherein the viruses consist of the viruses, VLPs or both of Table 1. In some embodiments, the plurality of probes consists of viruses, VLPs or both selected from Table 1. In some embodiments, the plurality of probes comprises viruses, VLPs or both selected from Table 6. In some embodiments, the plurality of probes consists of virus, VLPs or both selected from Table 6. In some embodiments, the plurality of probes consists of the viruses, VLPs or both of Table 6. In some embodiments, the plurality of probes comprises viruses, wherein the viruses consist of the viruses, VLPs or both of Table 6. In some embodiments, the plurality of probes consists of viruses, VLPs or both selected from Table 6.

In some embodiments, the plurality of probes comprises proteins selected from Table 1. In some embodiments, the plurality of probes consists of proteins selected from Table 1. In some embodiments, the plurality of probes consists of the proteins of Table 1. In some embodiments, the plurality of probes comprises proteins, wherein the proteins consist of the proteins of Table 1. In some embodiments, the plurality of probes consists of proteins selected from Table 1. In some embodiments, the plurality of probes consists of probes selected from Table 1. In some embodiments, the plurality of probes comprises proteins selected from Table 6. In some embodiments, the plurality of probes consists of proteins selected from Table 6. In some embodiments, the plurality of probes consists of the proteins of Table 6. In some embodiments, the plurality of probes comprises proteins, wherein the proteins consist of the proteins of Table 6. In some embodiments, the plurality of probes consists of proteins selected from Table 6. In some embodiments, the plurality of probes consists of probes selected from Table 6.

In some embodiments, the plurality of probes comprises proteins selected from Table 5. In some embodiments, the plurality of probes consists of proteins selected from Table 5. In some embodiments, the plurality of probes consists of the proteins of Table 5. In some embodiments, the plurality of probes comprises proteins, wherein the proteins consist of the proteins of Table 5. In some embodiments, the plurality of probes consists of proteins selected from Table 5. In some embodiments, the plurality of probes consists of probes selected from Table 5. In some embodiments, the plurality of probes consists of probes selected from Table 5 and SEQ ID NO: 598-995.

In some embodiments, the support or array consists of the plurality of probes. In some embodiments, the only probes on the array/support are the plurality of probes. In some embodiments, the solid support or array further comprises control probes. In some embodiments, control probes are probes that are bound by known antibodies found in all subjects. In some embodiments, control probes are probes that bind known antibodies found in all subjects. In some embodiments, control probes comprise secondary antibodies to human antibodies. In some embodiments, the secondary antibodies are selected from anti-human IgG, anti-human IgA, and anti-human IgM. In some embodiments, the secondary antibodies are selected from anti-human IgG, anti-human IgA, anti-avian IgY and anti-human IgM. In some embodiments, the secondary antibodies are anti-avian IgY. In some embodiments, control probes are peptides or proteins used to generate a vaccine. In some embodiments, the plurality of probes comprises control probes. In some embodiments, a control is cell lysate from a cell uninfected by an influenza.

Table 1 lists the viruses, and HA and/or NA proteins that have been spotted on arrays of the invention. It contains 19 NA proteins and 47 HA proteins, as well as 39 whole viruses.

TABLE 1
Whole virus and recombinant protein probes for human influenza: The table
specifies the strain, vaccine year of the strain, and the type of antigen spotted on the array
(Whole inactivated virus, recombinant HA or NA proteins).
Internal proteins were also spotted for some of the strains.

| | | | whole | HA or | |
|---|---|---|---|---|---|
| | | Vaccine | inactivated | HAI | NA |
| Influenza strain name | Subtype | Year | virus | protein | protein |
| A/Beijing/262/1995 | H1N1 | 1998-2000 | X | X | |
| A/Brazil/11/1978 | H1N1 | | X | | |
| A/Brisbane/02/2018 | H1N1 | 2019-2020 | | X | X |
| A/Brisbane/59/2007 | H1N1 | 2008-2010 | X | X | |
| A/California/04/2009 | H1N1 | 2010-2016 | X | X | |
| A/California/07/2009 | H1N1 | 2010-2017 | X | X | X |
| A/Chile/1/1983 | H1N1 | 1984-1987 | X | | |
| A/Christchurch/16/2010 | H1N1 | 2010-2016 | X | | |
| A/Michigan/45/2015 | H1N1 | | | X | X |
| A/NewCaledonia/20/1999 | H1N1 | 2000-2007 | X | X | |
| A/PuertoRico/8/1934 | H1N1 | | X | X | X |
| A/Singapore/6/1986 | H1N1 | 1987-1997 | X | | |
| A/SolomonIslands/3/2006 | H1N1 | 2007-2008 | X | X | |
| A/SouthCarolina/1/1918 | H1N1 | | | X | |
| A/USSR/90/1977 | H1N1 | | X | X | X |
| A/Wisconsin/1933 | H1N1 | | X | X | |
| A/Japan/305/1957 | H2N2 | | | X | |
| A/Aichi/2/1968 | H3N2 | | | X | X |
| A/Babol/36/2005 | H3N2 | | | | X |
| A/Bangkok/1/1979 | H3N2 | | X | | |
| A/Brisbane/10/2007 | H3N2 | 2008-2010 | X | X | |
| A/California/07/2004 | H3N2 | 2005-2006 | X | X | |
| A/Fujian/411/2002 | H3N2 | | | X | |
| A/Guizhou/54/1989 | H3N2 | 1990-1991 | X | X | |
| A/Hawaii/07/2009 | H3N2 | | | X | |
| A/HongKong/4801/2014 | H3N2 | | | X | X |
| A/Kansas/14/2017 | H3N2 | 2019-2020 | | X | X |
| A/Leningrad/360/1986 | H3N2 | 1987-1988 | X | | |
| A/Maryland/26/2014 | H3N2 | | | X | |
| A/NewYork/55/2004 | H3N2 | 2005-2006 | X | X | |
| A/Panama/2007/1999 | H3N2 | 2000-2004 | X | | |
| A/Perth/16/2009 | H3N2 | 2010-2012 | X | X | |
| A/Portchalmers/1/1973 | H3N2 | | | | |
| A/Shandong/9/1993 | H3N2 | 1994-1995 | X | | |
| A/Singapore/INFIMH-16- | H3N2 | 2018-2019 | | X | |
| 0019/2016 | | | | | |
| A/Switzerland/9715293/2013 | H3N2 | 2015-2016 | X | X | |
| A/Sydney/5/1997 | H3N2 | 1998-2000 | X | X | |
| A/Texas/50/2012 | H3N2 | 2013-2015 | X | X | |
| A/Uruguay/716/2007 | H3N2 | 2008-2010 | X | | |
| A/Victoria/210/2009 | H3N2 | 2011 | X | X | |
| A/Victoria/361/2011 | H3N2 | 2012-2013 | X | X | |
| A/Wisconsin/67/2005 | H3N2 | 2006-2008 | X | X | X |
| A/X-31/1968 | H3N2 | | X | X | |
| A/Anhui/l/2005 | H5N1 | | | X | |
| A/HongKong/483/1997 | H5N1 | | | X | |
| A/Hubei/1/2010 | H5N1 | | | | X |
| A/Indonesia/5/2005 | H5N1 | | | X | |
| A/Thailand/1(KAN-1)/2004 | H5N1 | | | | X |
| A/Vietnam/1203/2004 | H5N1 | | X | X | |
| A/NewYork/107/2003 | H7N2 | | | X | |
| A/Canada/444/2004 | H7N3 | | | | X |
| A/Netherlands/219/2003 | H7N7 | | | X | X |
| A/Anhui/l/2013 | H7N9 | | | X | X |
| A/Shenghai/02/2013 | H7N9 | | | X | |
| A/HongKong/33982/2009 | H9N2 | | | X | |
| B/Brisbane/60/2008 | B | 2010-2017 | X | X | |
| B/Colorado/06/2017 | B | 2018-2020 | | X | X |
| B/Florida/4/2006 | B | 2008-2009 | X | X | X |
| B/HongKong/330/2001 | B | | | | X |
| B/Jiangsu/l0/2003 | B | 2005-2006 | X | | |
| B/Lee/1940 | B | | X | | |
| B/Malaysia/2506/2004 | B | 2006-2008 | X | X | |
| B/Massachusetts/02/2012 | B | 2014-2015 | X | | |
| B/Phuket/3073/2013 | B | 2015-2021 | X | X | X |
| B/Yamagata/16/1988 | B | | X | X | |

In some embodiments, the plurality of probes is selected form the probes provided in Table 4. In some embodiments, the plurality of probes comprises the probes provided in Table 4. In some embodiments, the plurality of probes consists of the probes provided in Table 4. In some embodiments, the peptide probes are selected from the probes provided in Table 4. In some embodiments, the plurality of probes is selected form the probes provided in Tables 1 and 4. In some embodiments, the plurality of probes comprises the probes provided in Tables 1 and 4. In some embodiments, the plurality of probes consists of the probes provided in Tables 1 and 4.

In some embodiments, peptide probes are probes against Wis05-A/Wisconsin/67/2005 (H3N2). In some embodiments, peptide probes against Wis05 are selected from SEQ ID NOs: 1-199. In some embodiments, SEQ ID NOs: 1-109 are peptide probes from HA proteins. In some embodiments, SEQ ID NOs: 110-199 are peptide probes from NA proteins. In some embodiments, peptide probes are probes against Cal09-A/California/07/2009 (H1N1). In some embodiments, peptide probes against Cal09 are selected from SEQ ID NOs: 200-399. In some embodiments, SEQ ID NOs: 200-310, are peptide probes from HA proteins. In some embodiments, SEQ ID NOs: 311-399 are peptide probes from NA proteins. In some embodiments, peptide probes are probes against PR8-A/Puerto-Rico/8/1934 (H1N1). In some embodiments, peptide probes against PR8 are provided in SEQ ID NOs: 400-597. In some embodiments, SEQ ID NOs: 400-508 are peptide probes from HA proteins. In some embodiments, SEQ ID NOs: 509-597 are peptide probes from NA proteins. In some embodiments, peptide probes are probes against SHA-A/Shanghai/01/2013 (H7N9). In some embodiments, peptide probes against SHA are provided in SEQ ID NOs: 598-798. In some embodiments, SEQ ID NOs: 598-707 are peptide probes from HA proteins. In some embodiments, SEQ ID NOs: 708-798 are peptide probes from NA proteins. In some embodiments, peptide probes are probes against SHA-A/Shanghai/01/2013 (H7N9). In some embodiments, peptide probes are probes against Veit-A/VietNam/1203/2004 (H5N1). In some embodiments, peptide probes against Veit are provided in SEQ ID NOs: 799-995. In some embodiments, SEQ ID NOs: 799-908 are peptide probes from HA proteins. In some embodiments, SEQ ID NOs: 909-995 are peptide probes from NA proteins. SHA and Viet are lethal avian influenza strains that have infected humans. In some embodiments, peptide probes are probes against Vic-A/Victoria/361/2011 (H3N2). In some embodiments, peptide probes against Vic are provided in SEQ ID NOs: 996-1190. In some embodiments, SEQ ID NOs: 996-1106 are peptide probes from HA proteins. In some embodiments, SEQ ID NOs: 1107-1190 are peptide probes from NA proteins. In some embodiments, peptide probes are probes against BWis-B/Wisconsin/01/2010. In some embodiments, peptide probes against BWis are provided in SEQ ID NOs: 1191-1390. In some embodiments, SEQ ID NOs: 1191-1304 are peptide probes from HA proteins. In some embodiments, SEQ ID NOs: 1305-1390 are peptide probes from NA proteins. In some embodiments, the plurality of probes is selected from SEQ ID NOS: 1-1390. In some embodiments, the peptide probes are selected from SEQ ID NOS: 1-1390. In some embodiments, the plurality of probes is selected from Table 4 and SEQ ID NO: 400-1390. In some embodiments, the peptide probes are selected from Table 4 and SEQ ID NO: 400-1390. In some embodiments, SEQ ID NOs: 1-109, 200-310, 400-508, 598-707, 799-908, 996-1106, and 1191-1304 are peptide probes from HA proteins. In some embodiments, SEQ ID NOs: 110-199, 311-399, 509-597, 708-798, 909-995, 1107-1190, and 1305-1390 are peptide probes from NA proteins. In some embodiments, peptide probes from zoonotic influenza are selected from SEQ ID NO: 598-995.

TABLE 4
List of peptides spotted on exemplary arrays. Short peptides from the HA
and NA proteins from various strains. Peptides for Wis05 (Wis05-
A/Wisconsin/67/2005 (H3N2)) are provide in SEQ ID NOs: 1-199. Peptides for
Cal09 (A/California/07/2009 (H1N1)) are provided in SEQ ID NOs: 200-399.

| | | SEQ | | | SEQ |
|---|---|---|---|---|---|
| | | ID | | | ID |
| Peptide name | Sequence | NO: | Peptide name | Sequence | NO: |
| Wis05_HA_1 | KTIIALSYILCL | 1 | Cal_HA_1 | MKAILVVLLYTF | 200 |
| | VFAQKLP | | | ATANADTLKK | |
| Wis05_HA_2 | LSYILCLVFAQ | 2 | Cal_HA_2 | VVLLYTFATAN | 201 |
| | KLPGNDNS | | | ADTLCIGYH | |
| Wis05_HA_3 | CLVFAQKLPGN | 3 | Cal_HA_3 | TFATANADTLCI | 202 |
| | DNSTATLC | | | GYHANNST | |
| Wis05_HA_4 | QKLPGNDNSTA | 4 | Cal_HA_4 | NADTLCIGYHA | 203 |
| | TLCLGHHA | | | NNSTDTVDT | |
| Wis05_HA_5 | NDNSTATLCLG | 5 | Cal_HA_5 | CIGYHANNSTDT | 204 |
| | HHAVPNGT | | | VDTVLEKN | |
| Wis05_HA_6 | ATLCLGHHAVP | 6 | Cal_HA_6 | ANNSTDTVDTV | 205 |
| | NGTIVKTI | | | LEKNVTVTH | |
| Wis05_HA_7 | GHHAVPNGTIV | 7 | Cal_HA_7 | DTVDTVLEKNV | 206 |
| | KTITNDQI | | | TVTHSVNLL | |
| Wis05_HA_8 | PNGTIVKTITND | 8 | Cal_HA_8 | VLEKNVTVTHS | 207 |
| | QIEVTNA | | | VNLLEDKHN | |
| Wis05_HA_9 | VKTITNDQIEVT | 9 | Cal_HA_9 | VTVTHSVNLLED | 208 |
| | NATELVQ | | | KHNGKLCK | |
| Wis05_HA_10 | NDQIEVTNATE | 10 | Cal_HA_10 | SVNLLEDKHNG | 209 |
| | LVQSSSTG | | | KLCKLRGVA | |
| Wis05_HA_11 | VTNATELVQSS | 11 | Cal_HA_11 | EDKHNGKLCKL | 210 |
| | STGGICDS | | | RGVAPLHLG | |
| Wis05_HA_12 | ELVQSSSTGGIC | 12 | Cal_HA_12 | GKLCKLRGVAP | 211 |
| | DSPHQIL | | | LHLGKCNIA | |
| Wis05_HA_13 | SSTGGICDSPHQ | 13 | Cal_HA_13 | LRGVAPLHLGK | 212 |
| | ILDGENC | | | CNIAGWILG | |
| Wis05_HA_14 | ICDSPHQILDGE | 14 | Cal_HA_14 | PLHLGKCNIAG | 213 |
| | NCTLIDA | | | WILGNPECE | |
| Wis05_HA_15 | HQILDGENCTLI | 15 | Cal_HA_15 | KCNIAGWILGNP | 214 |
| | DALLGDP | | | ECESLSTA | |
| Wis05_HA_16 | GENCTLIDALL | 16 | Cal_HA_16 | GWILGNPECESL | 215 |
| | GDPQCDGF | | | STASSWSY | |
| Wis05_HA_17 | LIDALLGDPQC | 17 | Cal_HA_17 | NPECESLSTASS | 216 |
| | DGFQNKKW | | | WSYIVETP | |
| Wis05_HA_18 | LGDPQCDGFQN | 18 | Cal_HA_18 | SLSTASSWSYIV | 217 |
| | KKWDLFVE | | | ETPSSDNG | |
| Wis05_HA_19 | CDGFQNKKWD | 19 | Cal_HA_19 | SSWSYIVETPSS | 218 |
| | LFVERSKAY | | | DNGTCYPG | |
| Wis05_HA_20 | NKKWDLFVER | 20 | Cal_HA_20 | IVETPSSDNGTC | 219 |
| | SKAYSNCYP | | | YPGDFIDY | |
| Wis05_HA_21 | LFVERSKAYSN | 21 | Cal_HA_21 | SSDNGTCYPGDF | 220 |
| | CYPYDVPD | | | IDYEELRE | |
| Wis05_HA_22 | SKAYSNCYPYD | 22 | Cal_HA_22 | TCYPGDFIDYEE | 221 |
| | VPDYASLR | | | LREQLSSV | |
| Wis05_HA_23 | NCYPYDVPDY | 23 | Cal_HA_23 | DFIDYEELREQL | 222 |
| | ASLRSLVAS | | | SSVSSFER | |
| Wis05_HA_24 | DVPDYASLRSL | 24 | Cal_HA_24 | EELREQLSSVSSF | 223 |
| | VASSGTLE | | | ERFEIFP | |
| Wis05_HA_25 | ASLRSLVASSG | 25 | Cal_HA_25 | QLSSVSSFERFEI | 224 |
| | TLEFNDES | | | FPKTSSW | |
| Wis05_HA_26 | LVASSGTLEFN | 26 | Cal_HA_26 | SSFERFEIFPKTS | 225 |
| | DESFNWTG | | | SWPNHDS | |
| Wis05_HA_27 | GTLEFNDESFN | 27 | Cal_HA_27 | FEIFPKTSSWPN | 226 |
| | WTGVTQNG | | | HDSNKGVT | |
| Wis05_HA_28 | NDESFNWTGVT | 28 | Cal_HA_28 | KTSSWPNHDSN | 227 |
| | QNGTSSSC | | | KGVTAACPH | |
| Wis05_HA_29 | NWTGVTQNGT | 29 | Cal_HA_29 | PNHDSNKGVTA | 228 |
| | SSSCKRRSN | | | ACPHAGAKS | |
| Wis05_HA_30 | TQNGTSSSCKR | 30 | Cal_HA_30 | NKGVTAACPHA | 229 |
| | RSNNSFFS | | | GAKSFYKNL | |
| Wis05_HA_31 | SSSCKRRSNNSF | 31 | Cal_HA_31 | AACPHAGAKSF | 230 |
| | FSRLNWL | | | YKNLIWLVK | |
| Wis05_HA_32 | RRSNNSFFSRL | 32 | Cal_HA_32 | AGAKSFYKNLI | 231 |
| | NWLTHLKF | | | WLVKKGNSY | |
| Wis05_HA_33 | SFFSRLNWLTH | 33 | Cal_HA_33 | FYKNLIWLVKK | 232 |
| | LKFKYPAL | | | GNSYPKLSK | |
| Wis05_HA_34 | LNWLTHLKFK | 34 | Cal_HA_34 | IWLVKKGNSYP | 233 |
| | YPALNVTMP | | | KLSKSYIND | |
| Wis05_HA_35 | HLKFKYPALNV | 35 | Cal_HA_35 | KGNSYPKLSKSY | 234 |
| | TMPNNEKF | | | INDKGKEV | |
| Wis05_HA_36 | YPALNVTMPN | 36 | Cal_HA_36 | PKLSKSYINDKG | 235 |
| | NEKFDKLYI | | | KEVLVLWG | |
| Wis05_HA_37 | VTMPNNEKFD | 37 | Cal_HA_37 | SYINDKGKEVLV | 236 |
| | KLYIWGVHH | | | LWGIHHPS | |
| Wis05_HA_38 | NEKFDKLYIWG | 38 | Cal_HA_38 | KGKEVLVLWGI | 237 |
| | VHHPVTDN | | | HHPSTSADQ | |
| Wis05_HA_39 | KLYIWGVHHPV | 39 | Cal_HA_39 | LVLWGIHHPSTS | 238 |
| | TDNDQIFL | | | ADQQSLYQ | |
| Wis05_HA_40 | GVHHPVTDND | 40 | Cal_HA_40 | IHHPSTSADQQS | 239 |
| | QIFLYAQAS | | | LYQNADAY | |
| Wis05_HA_41 | VTDNDQIFLYA | 41 | Cal_HA_41 | TSADQQSLYQN | 240 |
| | QASGRITV | | | ADAYVFVGS | |
| Wis05_HA_42 | QIFLYAQASGRI | 42 | Cal_HA_42 | QSLYQNADAYV | 241 |
| | TVSTKRS | | | FVGSSRYSK | |
| Wis05_HA_43 | AQASGRITVST | 43 | Cal_HA_43 | NADAYVFVGSS | 242 |
| | KRSQQTVI | | | RYSKKFKPE | |
| Wis05_HA_44 | RITVSTKRSQQT | 44 | Cal_HA_44 | VFVGSSRYSKKF | 243 |
| | VIPNIGS | | | KPEIAIRP | |
| Wis05_HA_45 | TKRSQQTVIPNI | 45 | Cal_HA_45 | SRYSKKFKPEIAI | 244 |
| | GSRPRIR | | | RPKVRDQ | |
| Wis05_HA_46 | QTVIPNIGSRPRI | 46 | Cal_HA_46 | KFKPEIAIRPKVR | 245 |
| | RNIPSR | | | DQEGRMN | |
| Wis05_HA_47 | NIGSRPRIRNIPS | 47 | Cal_HA_47 | IAIRPKVRDQEG | 246 |
| | RISIYW | | | RMNYYWTL | |
| Wis05_HA_48 | PRIRNIPSRISIY | 48 | Cal_HA_48 | KVRDQEGRMNY | 247 |
| | WTIVKP | | | YWTLVEPGD | |
| Wis05_HA_49 | IPSRISIYWTIVK | 49 | Cal_HA_49 | EGRMNYYWTLV | 248 |
| | PGDILL | | | EPGDKITFE | |
| Wis05_HA_50 | SIYWTIVKPGDI | 50 | Cal_HA_50 | YYWTLVEPGDK | 249 |
| | LLINSTG | | | ITFEATGNL | |
| Wis05_HA_51 | IVKPGDILLINS | 51 | Cal_HA_51 | VEPGDKITFEAT | 250 |
| | TGNLIAP | | | GNLVVPRY | |
| Wis05_HA_52 | DILLINSTGNLI | 52 | Cal_HA_52 | KITFEATGNLVV | 251 |
| | APRGYFK | | | PRYAFAME | |
| Wis05_HA_53 | NSTGNLIAPRG | 53 | Cal_HA_53 | ATGNLVVPRYA | 252 |
| | YFKIRSGK | | | FAMERNAGS | |
| Wis05_HA_54 | LIAPRGYFKIRS | 54 | Cal_HA_54 | VVPRYAFAMER | 253 |
| | GKSSIMR | | | NAGSGIIIS | |
| Wis05_HA_55 | GYFKIRSGKSSI | 55 | Cal_HA_55 | AFAMERNAGSGI | 254 |
| | MRSDAPI | | | IISDTPVH | |
| Wis05_HA_56 | RSGKSSIMRSD | 56 | Cal_HA_56 | RNAGSGIIISDTP | 255 |
| | APIGKCNS | | | VHDCNTT | |
| Wis05_HA_57 | SIMRSDAPIGKC | 57 | Cal_HA_57 | GIIISDTPVHDCN | 256 |
| | NSECITP | | | TTCQTPK | |
| Wis05_HA_58 | DAPIGKCNSECI | 58 | Cal_HA_58 | DTPVHDCNTTC | 257 |
| | TPNGSIP | | | QTPKGAINT | |
| Wis05_HA_59 | CNSECITPNGSI | 59 | Cal_HA_59 | DCNTTCQTPKG | 258 |
| | PNDKPF | | | AINTSLPFQ | |
| Wis05_HA_60 | CITPNGSIPNDK | 60 | Cal_HA_60 | CQTPKGAINTSL | 259 |
| | PFQNVNR | | | PFQNIHPI | |
| Wis05_HA_61 | GSIPNDKPFQN | 61 | Cal_HA_61 | GAINTSLPFQNIH | 260 |
| | VNRITYGA | | | PITIGKC | |
| Wis05_HA_62 | DKPFQNVNRIT | 62 | Cal_HA_62 | SLPFQNIHPITIG | 261 |
| | YGACPRYV | | | KCPKYVK | |
| Wis05_HA_63 | NVNRITYGACP | 63 | Cal_HA_63 | NIHPITIGKCPKY | 262 |
| | RYVKQNTL | | | VKSTKLR | |
| Wis05_HA_64 | TYGACPRYVK | 64 | Cal_HA_64 | TIGKCPKYVKST | 263 |
| | QNTLKLATG | | | KLRLATGL | |
| Wis05_HA_65 | PRYVKQNTLKL | 65 | Cal_HA_65 | PKYVKSTKLRLA | 264 |
| | ATGMRNVP | | | TGLRNIPS | |
| Wis05_HA_66 | QNTLKLATGM | 66 | Cal_HA_66 | STKLRLATGLRN | 265 |
| | RNVPEKQTR | | | IPSIQSRG | |
| Wis05_HA_67 | LATGMRNVPE | 67 | Cal_HA_67 | LATGLRNIPSIQS | 266 |
| | KQTRGIFGA | | | RGLFGAI | |
| Wis05_HA_68 | RNVPEKQTRGI | 68 | Cal_HA_68 | RNIPSIQSRGLFG | 267 |
| | FGAIAGFI | | | AIAGFIE | |
| Wis05_HA_69 | KQTRGIFGAIA | 69 | Cal_HA_69 | IQSRGLFGAIAGF | 268 |
| | GFIENGWE | | | IEGGWTG | |
| Wis05_HA_70 | IFGAIAGFIENG | 70 | Cal_HA_70 | KKLFGAIAGFIE | 269 |
| | WEGMVDG | | | GGWTGMVDGW | |
| | | | | KK | |
| Wis05_HA_71 | AGFIENGWEG | 71 | Cal_HA_71 | AGFIEGGWTGM | 270 |
| | MVDGWYGFR | | | VDGWYGYHH | |
| Wis05_HA_72 | NGWEGMVDG | 72 | Cal_HA_72 | GGWTGMVDGW | 271 |
| | WYGFRHQNSE | | | YGYHHQNEQG | |
| Wis05_HA_73 | MVDGWYGFRH | 73 | Cal_HA_73 | MVDGWYGYHH | 272 |
| | QNSEGIGQA | | | QNEQGSGYAA | |
| Wis05_HA_74 | YGFRHQNSEGI | 74 | Cal_HA_74 | YGYHHQNEQGS | 273 |
| | GQAADLKS | | | GYAADLKST | |
| Wis05_HA_75 | QNSEGIGQAAD | 75 | Cal_HA_75 | QNEQGSGYAAD | 274 |
| | LKSTQAAI | | | LKSTQNAID | |
| Wis05_HA_76 | IGQAADLKSTQ | 76 | Cal_HA_76 | SGYAADLKSTQ | 275 |
| | AAINQING | | | NAIDEITNK | |
| Wis05_HA_77 | DLKSTQAAINQI | 77 | Cal_HA_77 | DLKSTQNAIDEI | 276 |
| | NGKLNRL | | | TNKVNSVI | |
| Wis05_HA_78 | QAAINQINGKL | 78 | Cal_HA_78 | QNAIDEITNKVN | 277 |
| | NRLIGKTN | | | SVIEKMNT | |
| Wis05_HA_79 | QINGKLNRLIG | 79 | Cal_HA_79 | EITNKVNSVIEK | 278 |
| | KTNEKFHQ | | | MNTQFTAV | |
| Wis05_HA_80 | LNRLIGKTNEK | 80 | Cal_HA_80 | VNSVIEKMNTQF | 279 |
| | FHQIEKEF | | | TAVGKEFN | |
| Wis05_HA_81 | GKTNEKFHQIE | 81 | Cal_HA_81 | EKMNTQFTAVG | 280 |
| | KEFSEVEG | | | KEFNHLEKR | |
| Wis05_HA_82 | FHQIEKEFSEVE | 82 | Cal_HA_82 | QFTAVGKEFNH | 281 |
| | GRIQDL | | | LEKRIENLN | |
| Wis05_HA_83 | EKEFSEVEGRIQ | 83 | Cal_HA_83 | GKEFNHLEKRIE | 282 |
| | DLEKYVE | | | NLNVDD | |
| Wis05_HA_84 | EVEGRIQDLEK | 84 | Cal_HA_84 | HLEKRIENLNVD | 283 |
| | YVEDTKID | | | DGFLDI | |
| Wis05_HA_85 | IQDLEKYVEDT | 85 | Cal_HA_85 | IENLNVDDGFLD | 284 |
| | KIDLWSYN | | | IWTYNA | |
| Wis05_HA_86 | KYVEDTKIDLW | 86 | Cal_HA_86 | VDDGFLDIWTY | 285 |
| | SYNAELLV | | | NAELLVL | |
| Wis05_HA_87 | TKIDLWSYNAE | 87 | Cal_HA_87 | GFLDIWTYNAEL | 286 |
| | LLVALENQ | | | LVLLENER | |
| Wis05_HA_88 | WSYNAELLVA | 88 | Cal_HA_88 | WTYNAELLVLL | 287 |
| | LENQHTIDL | | | ENERTLDYH | |
| Wis05_HA_89 | ELLVALENQHT | 89 | Cal_HA_89 | ELLVLLENERTL | 288 |
| | IDLTDSEM | | | DYHDSNVK | |
| Wis05_HA_90 | LENQHTIDLTD | 90 | Cal_HA_90 | LENERTLDYHDS | 289 |
| | SEMNKLFE | | | NVKNLYEK | |
| Wis05_HA_91 | TIDLTDSEMNK | 91 | Cal_HA_91 | TLDYHDSNVKN | 290 |
| | LFERTKKQ | | | LYEKVRSQL | |
| Wis05_HA_92 | DSEMNKLFERT | 92 | Cal_HA_92 | DSNVKNLYEKV | 291 |
| | KKQLRENA | | | RSQLKNNAK | |
| Wis05_HA_93 | KLFERTKKQLR | 93 | Cal_HA_93 | NLYEKVRSQLK | 292 |
| | ENAEDMGN | | | NNAKEIGNG | |
| Wis05_HA_94 | TKKQLRENAED | 94 | Cal_HA_94 | VRSQLKNNAKEI | 293 |
| | MGNGCFKI | | | GNGCFEFY | |
| Wis05_HA_95 | RENAEDMGNG | 95 | Cal_HA_95 | KNNAKEIGNGCF | 294 |
| | CFKIYHKCD | | | EFYHKCDN | |
| Wis05_HA_96 | DMGNGCFKIYH | 96 | Cal_HA_96 | EIGNGCFEFYHK | 295 |
| | KCDNACIG | | | CDNTCMES | |
| Wis05_HA_97 | CFKIYHKCDNA | 97 | Cal_HA_97 | CFEFYHKCDNTC | 296 |
| | CIGSIRNG | | | MESVKNGT | |
| Wis05_HA_98 | HKCDNACIGSI | 98 | Cal_HA_98 | HKCDNTCMESV | 297 |
| | RNGTYDHD | | | KNGTYDYPK | |
| Wis05_HA_99 | ACIGSIRNGTYD | 99 | Cal_HA_99 | TCMESVKNGTY | 298 |
| | HDVYRDE | | | DYPKYSEEA | |
| Wis05_HA_100 | IRNGTYDHDVY | 100 | Cal_HA_100 | VKNGTYDYPKY | 299 |
| | RDEALNNR | | | SEEAKLNRE | |
| Wis05_HA_101 | YDHDVYRDEA | 101 | Cal_HA_101 | YDYPKYSEEAK | 300 |
| | LNNRFQIKG | | | LNREEIDGV | |
| Wis05_HA_102 | YRDEALNNRFQ | 102 | Cal_HA_102 | YSEEAKLNREEI | 301 |
| | IKGVELKS | | | DGVKLEST | |
| Wis05_HA_103 | LNNRFQIKGVE | 103 | Cal_HA_103 | KLNREEIDGVKL | 302 |
| | LKSGYKDW | | | ESTRIYQI | |
| Wis05_HA_104 | QIKGVELKSGY | 104 | Cal_HA_104 | EIDGVKLESTRIY | 303 |
| | KDWILWIS | | | QILAIYS | |
| Wis05_HA_105 | ELKSGYKDWIL | 105 | Cal_HA_105 | KLESTRIYQILAI | 304 |
| | WISFAISC | | | YSTVASS | |
| Wis05_HA_106 | YKDWILWISFAI | 106 | Cal_HA_106 | KRIYQILAIYSTV | 305 |
| | SCFLLCV | | | ASSLVLVV | |
| Wis05_HA_107 | LWISFAISCFLL | 107 | Cal_HA_107 | KLAIYSTVASSL | 306 |
| | CVALLGF | | | VLVVSLGAI | |
| Wis05_HA_108 | AISCFLLCVALL | 108 | Cal_HA_108 | KTVASSLVLVVS | 307 |
| | GFIMWAC | | | LGAISFWMC | |
| Wis05_HA_109 | LLCVALLGFIM | 109 | Cal_HA_109 | KLVLVVSLGAIS | 308 |
| | WACQKGNI | | | FWMCSNGSL | |
| Wis05_NA_1 | NPNQKIITIGSV | 110 | Cal_HA_110 | KSLGAISFWMCS | 309 |
| | SLTISTI | | | NGSLQCRIC | |
| Wis05_NA_2 | IITIGSVSLTISTI | 111 | Cal_HA_111 | SFWMCSNGSLQ | 310 |
| | CFFMQ | | | CRICI | |
| Wis05_NA_3 | SVSLTISTICFF | 112 | Cal_NA_1 | MNPNQKIITIGSV | 311 |
| | MQIAILI | | | CMTIGMA | |
| Wis05_NA_4 | ISTICFFMQIAIL | 113 | Cal_NA_4 | KTIGMANLILQI | 312 |
| | ITTVTL | | | GNIISIWIS | |
| Wis05_NA_5 | FFMQIAILITTV | 114 | Cal_NA_5 | KNLILQIGNIISI | 313 |
| | TLHFKQY | | | WISHSIQL | |
| Wis05_NA_6 | AILITTVTLHFK | 115 | Cal_NA_6 | KIGNIISIWISHSI | 314 |
| | QYEFNSP | | | QLGNQNQ | |
| Wis05_NA_7 | TVTLHFKQYEF | 116 | Cal_NA_7 | KSIWISHSIQLGN | 315 |
| | NSPPNNQV | | | QNQIETCN | |
| Wis05_NA_8 | FKQYEFNSPPN | 117 | Cal_NA_8 | KHSIQLGNQNQI | 316 |
| | NQVMLCEP | | | ETCNQSVIT | |
| Wis05_NA_9 | FNSPPNNQVML | 118 | Cal_NA_9 | KGNQNQIETCN | 317 |
| | CEPTIIER | | | QSVITYENNT | |
| Wis05_NA_10 | NNQVMLCEPTII | 119 | Cal_NA_10 | KIETCNQSVITYE | 318 |
| | ERNITEI | | | NNTWVNQT | |
| Wis05_NA_11 | LCEPTIIERNITE | 120 | Cal_NA_11 | QSVITYENNTW | 319 |
| | IVYLTN | | | VNQTYVNIS | |
| Wis05_NA_12 | IIERNITEIVYLT | 121 | Cal_NA_12 | YENNTWVNQTY | 320 |
| | NTTIEK | | | VNISNTNFA | |
| Wis05_NA_13 | ITEIVYLTNTTIE | 122 | Cal_NA_13 | KWVNQTYVNIS | 321 |
| | KEICPK | | | NTNFAAGQSV | |
| Wis05_NA_14 | YLTNTTIEKEIC | 123 | Cal_NA_14 | YVNISNTNFAAG | 322 |
| | PKLAEYR | | | QSVVSVKL | |
| Wis05_NA_15 | TIEKEICPKLAE | 124 | Cal_NA_15 | NTNFAAGQSVV | 323 |
| | YRNWSKP | | | SVKLAGNSS | |
| Wis05_NA_16 | ICPKLAEYRNW | 125 | Cal_NA_16 | AGQSVVSVKLA | 324 |
| | SKPQCNIT | | | GNSSLCPVS | |
| Wis05_NA_17 | AEYRNWSKPQ | 126 | Cal_NA_17 | VSVKLAGNSSLC | 325 |
| | CNITGFAPF | | | PVSGWAIY | |
| Wis05_NA_18 | WSKPQCNITGF | 127 | Cal_NA_18 | AGNSSLCPVSG | 326 |
| | APFSKDNS | | | WAIYSKDNS | |
| Wis05_NA_19 | CNITGFAPFSKD | 128 | Cal_NA_19 | LCPVSGWAIYSK | 327 |
| | NSIRLSA | | | DNSVRIGS | |
| Wis05_NA_20 | FAPFSKDNSIRL | 129 | Cal_NA_20 | GWAIYSKDNSV | 328 |
| | SAGGDIW | | | RIGSKGDVF | |
| Wis05_NA_21 | DNSIRLSAGGDI | 130 | Cal_NA_21 | SKDNSVRIGSKG | 329 |
| | WVTREP | | | DVFVIREP | |
| Wis05_NA_22 | RLSAGGDIWVT | 131 | Cal_NA_22 | VRIGSKGDVFVI | 330 |
| | REPYVSCD | | | REPFISCS | |
| Wis05_NA_23 | GDIWVTREPYV | 132 | Cal_NA_23 | KGDVFVIREPFIS | 331 |
| | SCDPDKCY | | | CSPLECR | |
| Wis05_NA_24 | TREPYVSCDPD | 133 | Cal_NA_24 | VIREPFISCSPLE | 332 |
| | KCYQFALG | | | CRTFFLT | |
| Wis05_NA_25 | VSCDPDKCYQF | 134 | Cal_NA_25 | FISCSPLECRTFF | 333 |
| | ALGQGTTL | | | LTQGALL | |
| Wis05_NA_26 | DKCYQFALGQ | 135 | Cal_NA_26 | PLECRTFFLTQG | 334 |
| | GTTLNNVHS | | | ALLNDKHS | |
| Wis05_NA_27 | FALGQGTTLNN | 136 | Cal_NA_27 | TFFLTQGALLND | 335 |
| | VHSNDTVH | | | KHSNGTIK | |
| Wis05_NA_28 | GTTLNNVHSND | 137 | Cal_NA_28 | QGALLNDKHSN | 336 |
| | TVHDRTPY | | | GTIKDRSPY | |
| Wis05_NA_29 | NVHSNDTVHD | 138 | Cal_NA_29 | NDKHSNGTIKDR | 337 |
| | RTPYRTLLM | | | SPYRTLMS | |
| Wis05_NA_30 | DTVHDRTPYRT | 139 | Cal_NA_30 | NGTIKDRSPYRT | 338 |
| | LLMNELGV | | | LMSCPIGE | |
| Wis05_NA_31 | RTPYRTLLMNE | 140 | Cal_NA_31 | DRSPYRTLMSCP | 339 |
| | LGVPFHLG | | | IGEVPSPY | |
| Wis05_NA_32 | TLLMNELGVPF | 141 | Cal_NA_32 | RTLMSCPIGEVP | 340 |
| | HLGTKQVC | | | SPYNSRFE | |
| Wis05_NA_33 | ELGVPFHLGTK | 142 | Cal_NA_33 | CPIGEVPSPYNSR | 341 |
| | QVCIAWSS | | | FESVAWS | |
| Wis05_NA_34 | FHLGTKQVCIA | 143 | Cal_NA_34 | VPSPYNSRFESV | 342 |
| | WSSSSCHD | | | AWSASACH | |
| Wis05_NA_35 | KQVCIAWSSSS | 144 | Cal_NA_35 | NSRFESVAWSAS | 343 |
| | CHDGKAWL | | | ACHDGINW | |
| Wis05_NA_36 | AWSSSSCHDGK | 145 | Cal_NA_36 | KSVAWSASACH | 344 |
| | AWLHVCVT | | | DGINWLTIGI | |
| Wis05_NA_37 | SCHDGKAWLH | 146 | Cal_NA_37 | ASACHDGINWL | 345 |
| | VCVTGDDKN | | | TIGISGPDN | |
| Wis05_NA_38 | KAWLHVCVTG | 147 | Cal_NA_38 | DGINWLTIGISGP | 346 |
| | DDKNATASF | | | DNGAVAV | |
| Wis05_NA_39 | VCVTGDDKNA | 148 | Cal_NA_39 | LTIGISGPDNGA | 347 |
| | TASFIYNGR | | | VAVLKYNG | |
| Wis05_NA_40 | DDKNATASFIY | 149 | Cal_NA_40 | SGPDNGAVAVL | 348 |
| | NGRLVDSI | | | KYNGIITDT | |
| Wis05_NA_41 | TASFIYNGRLV | 150 | Cal_NA_41 | GAVAVLKYNGII | 349 |
| | DSIVSWSK | | | TDTIKSWR | |
| Wis05_NA_42 | YNGRLVDSIVS | 151 | Cal_NA_42 | LKYNGIITDTIKS | 350 |
| | WSKEILRT | | | WRNNILR | |
| Wis05_NA_43 | VDSIVSWSKEIL | 152 | Cal_NA_43 | IITDTIKSWRNNI | 351 |
| | RTQESEC | | | LRTQESE | |
| Wis05_NA_44 | SWSKEILRTQES | 153 | Cal_NA_44 | IKSWRNNILRTQ | 352 |
| | ECVCING | | | ESECACVN | |
| Wis05_NA_45 | ILRTQESECVCI | 154 | Cal_NA_45 | NNILRTQESECA | 353 |
| | NGTCTVV | | | CVNGSCFT | |
| Wis05_NA_46 | ESECVCINGTCT | 155 | Cal_NA_46 | TQESECACVNGS | 354 |
| | VVMTDGS | | | CFTVMTDG | |
| Wis05_NA_47 | CINGTCTVVMT | 156 | Cal_NA_47 | KCACVNGSCFT | 355 |
| | DGSASGKA | | | VMTDGPSNGQ | |
| Wis05_NA_48 | CTVVMTDGSAS | 157 | Cal_NA_48 | GSCFTVMTDGPS | 356 |
| | GKADTKIL | | | NGQASYKI | |
| Wis05_NA_49 | TDGSASGKADT | 158 | Cal_NA_49 | VMTDGPSNGQA | 357 |
| | KILFIEEG | | | SYKIFRIEK | |
| Wis05_NA_50 | SGKADTKILFIE | 159 | Cal_NA_50 | PSNGQASYKIFRI | 358 |
| | EGKIVHT | | | EKGKIVK | |
| Wis05_NA_51 | TKILFIEEGKIV | 160 | Cal_NA_51 | ASYKIFRIEKGKI | 359 |
| | HTSTLSG | | | VKSVEMN | |
| Wis05_NA_52 | IEEGKIVHTSTL | 161 | Cal_NA_52 | FRIEKGKIVKSV | 360 |
| | SGSAQHV | | | EMNAPNYH | |
| Wis05_NA_53 | IVHTSTLSGSAQ | 162 | Cal_NA_53 | GKIVKSVEMNA | 361 |
| | HVEECSC | | | PNYHYEECS | |
| Wis05_NA_54 | TLSGSAQHVEE | 163 | Cal_NA_54 | SVEMNAPNYHY | 362 |
| | CSCYPRYL | | | EECSCYPDS | |
| Wis05_NA_55 | AQHVEECSCYP | 164 | Cal_NA_55 | APNYHYEECSC | 363 |
| | RYLGVRCV | | | YPDSSEITC | |
| Wis05_NA_56 | ECSCYPRYLGV | 165 | Cal_NA_56 | YEECSCYPDSSEI | 364 |
| | RCVCRDNW | | | TCVCRDN | |
| Wis05_NA_57 | PRYLGVRCVCR | 166 | Cal_NA_57 | CYPDSSEITCVC | 365 |
| | DNWKGSNR | | | RDNWHGSN | |
| Wis05_NA_58 | VRCVCRDNWK | 167 | Cal_NA_58 | SEITCVCRDNWH | 366 |
| | GSNRPIVDI | | | GSNRPWVS | |
| Wis05_NA_59 | RDNWKGSNRPI | 168 | Cal_NA_59 | VCRDNWHGSNR | 367 |
| | VDINIKDY | | | PWVSFNQNL | |
| Wis05_NA_60 | GSNRPIVDINIK | 169 | Cal_NA_60 | WHGSNRPWVSF | 368 |
| | DYSIVSS | | | NQNLEYQIG | |
| Wis05_NA_61 | IVDINIKDYSIVS | 170 | Cal_NA_61 | RPWVSFNQNLE | 369 |
| | SYVCSG | | | YQIGYICSG | |
| Wis05_NA_62 | IKDYSIVSSYVC | 171 | Cal_NA_62 | FNQNLEYQIGYI | 370 |
| | SGLVGDT | | | CSGIFGDN | |
| Wis05_NA_63 | IVSSYVCSGLV | 172 | Cal_NA_63 | EYQIGYICSGIFG | 371 |
| | GDTPRKND | | | DNPRPND | |
| Wis05_NA_64 | VCSGLVGDTPR | 173 | Cal_NA_64 | YICSGIFGDNPRP | 372 |
| | KNDSSSSS | | | NDKTGSC | |
| Wis05_NA_65 | VGDTPRKNDSS | 174 | Cal_NA_65 | IFGDNPRPNDKT | 373 |
| | SSSHCLDP | | | GSCGPVSS | |
| Wis05_NA_66 | RKNDSSSSSHC | 175 | Cal_NA_66 | PRPNDKTGSCGP | 374 |
| | LDPNNEEG | | | VSSNGANG | |
| Wis05_NA_67 | SSSSHCLDPNN | 176 | Cal_NA_67 | KTGSCGPVSSNG | 375 |
| | EEGGHGVK | | | ANGVKGFS | |
| Wis05_NA_68 | CLDPNNEEGGH | 177 | Cal_NA_68 | GPVSSNGANGV | 376 |
| | GVKGWAFD | | | KGFSFKYGN | |
| Wis05_NA_69 | NEEGGHGVKG | 178 | Cal_NA_69 | NGANGVKGFSF | 377 |
| | WAFDDGNDV | | | KYGNGVWIG | |
| Wis05_NA_70 | HGVKGWAFDD | 179 | Cal_NA_70 | VKGFSFKYGNG | 378 |
| | GNDVWMGRT | | | VWIGRTKSI | |
| Wis05_NA_71 | WAFDDGNDVW | 180 | Cal_NA_71 | FKYGNGVWIGR | 379 |
| | MGRTISEKL | | | TKSISSRNG | |
| Wis05_NA_72 | GNDVWMGRTI | 181 | Cal_NA_72 | GVWIGRTKSISS | 380 |
| | SEKLRSGYE | | | RNGFEMIW | |
| Wis05_NA_73 | MGRTISEKLRS | 182 | Cal_NA_73 | RTKSISSRNGFE | 381 |
| | GYETFKVI | | | MIWDPNGW | |
| Wis05_NA_74 | SEKLRSGYETF | 183 | Cal_NA_74 | SSRNGFEMIWDP | 382 |
| | KVIEGWSN | | | NGWTGTDN | |
| Wis05_NA_75 | SGYETFKVIEG | 184 | Cal_NA_75 | FEMIWDPNGWT | 383 |
| | WSNPNSKL | | | GTDNNFSIK | |
| Wis05_NA_76 | FKVIEGWSNPN | 185 | Cal_NA_76 | DPNGWTGTDNN | 384 |
| | SKLQINRQ | | | FSIKQDIVG | |
| Wis05_NA_77 | GWSNPNSKLQI | 186 | Cal_NA_77 | TGTDNNFSIKQD | 385 |
| | NRQVIVDR | | | IVGINEWS | |
| Wis05_NA_78 | NSKLQINRQVI | 187 | Cal_NA_78 | NFSIKQDIVGINE | 386 |
| | VDRGNRSG | | | WSGYSGS | |
| Wis05_NA_79 | INRQVIVDRGN | 188 | Cal_NA_79 | QDIVGINEWSGY | 387 |
| | RSGYSGIF | | | SGSFVQHP | |
| Wis05_NA_80 | IVDRGNRSGYS | 189 | Cal_NA_80 | INEWSGYSGSFV | 388 |
| | GIFSVEGK | | | QHPELTGL | |
| Wis05_NA_81 | NRSGYSGIFSVE | 190 | Cal_NA_81 | GYSGSFVQHPEL | 389 |
| | GKSCINR | | | TGLDCIRP | |
| Wis05_NA_82 | SGIFSVEGKSCI | 191 | Cal_NA_82 | FVQHPELTGLDC | 390 |
| | NRCFYVE | | | IRPCFWVE | |
| Wis05_NA_83 | VEGKSCINRCF | 192 | Cal_NA_83 | ELTGLDCIRPCF | 391 |
| | YVELIRGR | | | WVELIRGR | |
| Wis05_NA_84 | CINRCFYVELIR | 193 | Cal_NA_84 | DCIRPCFWVELI | 392 |
| | GRKEETE | | | RGRPKENT | |
| Wis05_NA_85 | FYVELIRGRKE | 194 | Cal_NA_85 | CFWVELIRGRPK | 393 |
| | ETEVLWTS | | | ENTIWTSG | |
| Wis05_NA_86 | IRGRKEETEVL | 195 | Cal_NA_86 | LIRGRPKENTIW | 394 |
| | WTSNSIVV | | | TSGSSISF | |
| Wis05_NA_87 | EETEVLWTSNSI | 196 | Cal_NA_87 | PKENTIWTSGSSI | 395 |
| | VVFCGTS | | | SFCGVNS | |
| Wis05_NA_88 | LWTSNSIVVFC | 197 | Cal_NA_88 | KIWTSGSSISFCG | 396 |
| | GTSGTYGT | | | VNSDTVGW | |
| Wis05_NA_89 | SIVVFCGTSGTY | 198 | Cal_NA_89 | KSSISFCGVNSD | 397 |
| | GTGSWPD | | | TVGWSWPDG | |
| Wis05_NA_90 | CGTSGTYGTGS | 199 | Cal_NA_90 | KCGVNSDTVGW | 398 |
| | WPDGADIN | | | SWPDGAELPF | |

| | | |
|---|---|---|
| Cal_NA_91 | DTVGWSWPDGA | 399 |
| | ELPFTIDK | |

Kits and Systems

According to another aspect, there is provided a kit comprising an array or support of the invention.

According to another aspect, there is provided a system comprising an array or support of the invention.

In some embodiments, the kit further comprises a detecting agent. In some embodiments, the kit further comprises at least one detecting agent. In some embodiments, the detecting agent is a labeled detecting agent. In some embodiments, the detecting agent is for detecting binding of an antibody to a probe of the array or support. In some embodiments, the detecting agent is for detecting antibodies. In some embodiments, the detecting agent is for detecting antibodies from a subject. In some embodiments, the detecting agent is for detecting human antibodies. In some embodiments, the detecting agent is for detecting IgG, IgA, IgM, IgY or a combination thereof. In some embodiments, the detecting agent is for detecting IgA. In some embodiments, the detecting agent is at least one labeled secondary antibody. In some embodiments, the secondary antibody is configured for detection of antibodies bound to the array or support. In some embodiments, the secondary antibody is an anti-human secondary antibody. Antibodies against any organism for which sample is to be tested can be included in the kit. In some embodiments, the secondary antibody is an anti-IgG antibody. In some embodiments, the secondary antibody is an anti-IgA antibody. In some embodiments, the secondary antibody is an anti-IgM antibody. In some embodiments, the secondary antibody is an anti-IgY antibody. In some embodiments, agents for detecting IgM, IgA, IgY and IgG comprise distinct labels.

In some embodiments, the kit comprises at least 1, 2, 3, 4, 5, 6, 7, 8, 9, or 10 secondary antibodies. Each possibility represents a separate embodiment of the invention. In some embodiments, the kit comprises at least 2 secondary antibodies. In some embodiments, the kit comprises an anti-IgG and an anti-IgA antibody. In some embodiments, the kit comprises an anti-IgA antibody. In some embodiments, the kit comprises an anti-IgG. In some embodiments, the kit comprises an anti-IgM antibody. antibody. In some embodiments, the kit comprises an anti-IgY antibody. In some embodiments, each secondary antibody comprises a uniquely detectable label. As such, the binding of each secondary antibody can be measured separately, or simultaneously but distinctly identified.

In some embodiments, the label is a fluorescent label. In some embodiments, the label is a radioactive label. Detectable labeled are well known in the art and any uniquely detectable labeled may be used.

In some embodiments, the system comprises a detector or sensor configured to detect binding of antibodies to probes of the array or support. In some embodiments, the detector or sensor is configured to detect labeled secondary antibodies. In some embodiments, the detector or support is configured to detect fluorescence. In some embodiments, the detector or sensor is configured to detect binding at specific locations on the array or support. In some embodiments, the detector or sense is configured to detect binding of antibodies to probes immobilized on the array or support. In some embodiments, the detector is a laser scanner.

In some embodiments, the array or support is for use in predicting the risk of symptomatic infection by an influenza virus. In some embodiments, the array or support is for use in predicting the risk of symptomatic infection of a subject by an influenza virus. n some embodiments, the array or support is for use in determining suitability of a subject to receive an influenza vaccine. In some embodiments, the array or support is for use in predicting the effectiveness of influenza vaccination of a subject. In some embodiments, the array or support is for use in predicting the effectiveness of an influenza vaccine in a subject. In some embodiments, the array or support is for use in detecting previous influenza infection of a subject. In some embodiments, the array or support is for use in detecting previous vaccination of a subject.

Methods of Use

In some embodiments, the array or support is for use in determining suitability of a subject to receive an influenza vaccine. In some embodiments, the array or support is for use in predicting the effectiveness of an influenza vaccination in a given subject. In some embodiments, the array or support is for use in detecting previous influenza infection in a subject. In some embodiments, the array or support is for use in detecting vaccination in a subject. In some embodiments, the array or support is for use in determining the strain of influenza that had previously infected a subject. In some embodiments, the array or support is for use in determining the subtype of influenza that had previously infected a subject. In some embodiments, the array or support is for use in determining the strain of influenza that a subject had previously been vaccinated against. In some embodiments, the array or support is for use in determining the subtype of influenza that a subject had previously been vaccinated against. In some embodiments, the array or support is for use in determining the potency of an influenza vaccine. In some embodiments, the array or support is for use in determining the efficacy of an influenza vaccine. In some embodiments, the array or support is for use in predicting risk of infection by influenza. It will be understood by a skilled artisan that any use for which the array or support can be used, so too a kit or system of the invention can also be used.

According to another aspect, there is provided a method of determining the suitability of a subject in need thereof to receive an influenza vaccination, the method comprising providing a sample from the subject, contacting the sample to an array or support of the invention, detecting binding of an antibody from the sample to a discrete location on the array or support, and generating an influenza immune score from the detected binding, thereby determining the suitability of a subject to receive an influenza vaccination.

According to another aspect, there is provided a method of determining risk of symptomatic infection of a subject by an influenza virus, the method comprising providing a sample from the subject, contacting the sample to an array or support of the invention, detecting binding of an antibody from the sample to a discrete location on the array or support, and generating an influenza immune score from the detected binding, thereby determining risk of symptomatic infection of a subject by an influenza virus.

According to another aspect, there is provided a method of predicting effectiveness of an influenza vaccine in a subject in need thereof, the method comprising providing a sample from the subject, contacting the sample to an array or support of the invention, detecting binding of an antibody from the sample to a discrete location on the array or support, and generating an influenza immune score from the detected binding, thereby predicting effectiveness of an influenza vaccine in a subject.

According to another aspect, there is provided a method of predicting the effectiveness of an influenza vaccine, the method comprising: providing a solution comprising antibodies from immune cells contacted by the influenza vaccine; contacting the solution to an array or support of the invention; detecting binding of the antibodies to locations on the array or support, and generating an influenza immune score from the detected binding; thereby predicting the effectiveness of an influenza vaccine.

According to another aspect, there is provided a method of determining risk of infection by influenza in a subject in need thereof, the method comprising providing a biological sample from the subject comprising antibodies; measuring levels of IgA antibodies against influenza where in a level of anti-influenza IgA antibodies below a predetermined threshold indicates the subject is it at increased risk of infection by influenza.

According to another aspect, there is provided a method of predicting disease severity following infection in a subject in need thereof, the method comprising providing a biological sample from the subject comprising antibodies; measuring levels of IgA antibodies against influenza where in a level of anti-influenza IgA antibodies below a predetermined threshold indicates the subject is at increased risk of high disease severity after infection by influenza.

In some embodiments, the subject is a human. In some embodiments, the subject is avian. In some embodiments, the subject is a mammal. In some embodiments, the subject is a non-human. In some embodiments, the subject is avian. In some embodiments, the subject is a veterinary animal. In some embodiments, the subject is a farm animal. In some embodiments, the subject is a wild animal. In some embodiments, the wild animal is a wild bird. Birds and avian animals are well known in the art and include for example, chickens, mallards, ducks, turkeys, pheasants, among many others. In some embodiments, the subject is at risk for developing influenza. In some embodiments, the subject is at risk for contracting influenza. In some embodiments, the subject has previously been vaccinated against influenza. In some embodiments, the subject is high-risk for complications of influenza. In some embodiments, the subject is at risk for influenza related complications. In some embodiments, the subject has previously been infected by influenza. In some embodiments, the subject is naïve to influenza. In some embodiments, the subject is an infant. In some embodiments, the subject is elderly. In some embodiments, the subject is sick with a non-influenza infection. In some embodiments, the subject is a child. In some embodiments, the subject is an adult. In some embodiments, the subject is pregnant or at risk of becoming pregnant.

In some embodiments, the sample is a biological sample. In some embodiments, the sample is a bodily fluid. In some embodiments, the bodily fluid is selected from: blood, serum, gastric fluid, intestinal fluid, saliva, bile, breast milk, nasal swab, oral swab urine, interstitial fluid, and stool. In some embodiments, the bodily fluid is blood. In some embodiments, the bodily fluid is serum. In some embodiments, the bodily fluid is plasma. In some embodiments, the blood is peripheral blood. In some embodiments, the bodily fluid is selected from blood and serum. In some embodiments, the bodily fluid is selected from blood, plasma and serum. In some embodiments, the bodily fluid is saliva. In some embodiments, the bodily fluid is selected from blood, saliva and serum. In some embodiments, the sample is from the subject. In some embodiments, the sample is a sample comprising antibodies. In some embodiments, the sample comprises antibodies from the subject. In some embodiments, the sample is not a nasal swab. In some embodiments, the sample is not a nasal sample.

In some embodiments, the sample comprises at least 1, 2, 3, 4, 5, 6, 7, 8, 9, or 10 microliters of fluid. Each possibility represents a separate embodiment of the invention. In some embodiments, the sample comprises at most 10, 15, 20, 25, 30, 35, 40, 45, 50, 55, 60, 65, 70, 75, 80, 85, 90, 95, 100, 200, 400 or 500 microliters of fluid. Each possibility represents a separate embodiment of the invention. In some embodiments, the sample comprises sufficient liquid to cover the array or support. In some embodiments, the sample comprises sufficient liquid to cover the plurality of probes. In some embodiments, the sample is diluted in buffer. In some embodiments, the buffer is binding buffer.

In some embodiments, the contacting is in conditions sufficient for antibody binding to the probes. In some embodiments, the contacting is in conditions sufficient for antibody binding to the plurality of probes. Conditions for antibody binding will be known by one skilled in the art. Further, optimization of binding conditions can be determined by a skilled artisan. In some embodiments, the contacting produces an array or support with bound antibodies. In some embodiments, the contacting produces antibodies bound to the array or support.

In some embodiments, the detecting comprises detecting binding of an antibody to a probe. In some embodiments, the binding indicates the presence in the sample of an antibody to a probe located at the detected location. In some embodiments, the binding indicates the presence in the sample of an antibody to a given peptide, protein, or virus. The identity of the virus, peptide or protein to which there are antibodies in the sample is determined by the known locations of each probe.

In some embodiments, the detecting comprises contacting the array or support with bound antibodies with a labeled detecting agent. In some embodiments, the detecting agent is a secondary antibody. In some embodiments, the detecting agent detects antibodies from the sample. In some embodiments, the detecting agent detects binding of antibodies to a target. In some embodiments, the detecting agent detects binding of antibody from the sample to a probe of the array or support.

In some embodiments, generating an influenza immune score comprises summing the magnitude of binding of antibodies to given probes. In some embodiments, given probes are all the probes for a specific influenza. In some embodiments, the influenza immune score is virus specific. In some embodiments, the influenza immune score is informative for a plurality of influenzas. In some embodiments, the influenza immune score is informative for all influenzas. In some embodiments, generating an influenza immune score comprises summing the magnitude of binding of antibodies to all probes. In some embodiments, generating a influenza immune score comprises performing an algorithm as provided herein below. In some embodiments, binding to specific probes is weighted and the sum of the magnitudes of binding comprises these weights. In some embodiments, binding to specific probes is weighted such that binding to certain probes has a greater impact on the immune score and binding to other probes has a lesser impact on immune score. For example, for three probes x, y, and z an immune score may be compiled by summing binding to x+binding to y+binding to z. Alternatively, x, y and z may be given weights a, b and c respectively and thus the immune score would be calculated by a*x+b*y+c*z. In some embodiments, an influenza immune score is generated only from IgA binding. In some embodiments, an influenza immune score is generated only from IgG binding. In some embodiments, an influenza immune score is generated from binding to peptides. In some embodiments, an influenza immune score is generated from binding to proteins. In some embodiments, an influenza immune score is generated from binding to virus. In some embodiments, an influenza immune score is generated from binding to VLPs.

In some embodiments, the immune score is proportional to the subject's suitability to receive an influenza vaccine. In some embodiments, the immune score is inversely proportional to the subject's risk of symptomatic disease upon influenza infection. In some embodiments, the immune score is inversely proportional to the subject's risk of symptomatic influenza infection. In some embodiments, the immune score is inversely proportional to the subject's risk of developing a symptomatic influenza infection. In some embodiments, the immune score is the magnitude of the immune score. In some embodiments, the immune score is a numerical value. In some embodiments, immune score is proportional to the effectiveness of an influenza vaccine in the subject. In some embodiments, immune score is proportional to the effectiveness of an influenza vaccine on the subject. In some embodiments, effectiveness is predicted effectiveness. In some embodiments, a subject with low predicted effectiveness is not suitable to receive the vaccination. In some embodiments, a predicted effectiveness below a predestined threshold indicates the subject is not suitable to receive the vaccine. In some embodiments, a subject not at risk for symptomatic influenza infection is a subject with an immune score above a predetermined threshold. In some embodiments, a subject not at risk for symptomatic influenza infection is a subject not suitable to receive an influenza vaccination. In some embodiments, at risk is at high risk.

In some embodiments, a higher immune score indicates a greater suitability to receive an influenza vaccine. In some embodiments, a higher immune score indicates a greater likelihood of effectiveness of an influenza vaccine. In some embodiments, a higher immune score indicates a greater likelihood of effectiveness of an influenza vaccine in the subject. In some embodiments, a higher immune score indicates a lower likelihood of influenza infection. In some embodiments, a higher immune score indicates a lower likelihood of severe disease after infection by influenza. In some embodiments, influenza infection is future infection. In some embodiments, the higher and lower is as compared to a predetermined threshold. In some embodiments, the higher and lower is as compared to a subject with an average immune score in a population. In some embodiments, a predetermined threshold is a predetermined level. In some embodiments, the predetermined threshold is an immune score of a healthy subject. In some embodiments, the predetermined threshold is an average immune score of a population. In some embodiments, a population is a non-vaccinated population. In some embodiments, a population is a previously vaccinated population.

In some embodiments, the threshold for probe binding is a mean fluorescence intensity (MFI) above 200. In some embodiments, the threshold for probe binding is an MFI above 2000. In some embodiments, the probe is a peptide probe and the threshold for probe binding is an MFI above 2000. In some embodiments, the probe is a whole protein probe and the threshold for probe binding is an MFI above 2000. In some embodiments, the probe is a whole virus probe and the threshold for probe binding is an MFI above 200. In some embodiments, the threshold is binding to a predetermined number of probes. In some embodiments, the predetermined number is at least 1, 2, 3, 4, 5, 6, 7, 8, 9, 10, 15, 20, 25, 30, 35, 40, 45, 50 or 100 probes. Each possibility represents a separate embodiment of the invention. In some embodiments, the number of probes that must be bound is known, and each probe is multiplied by the MFI threshold for binding and the sum of the products is the immune score threshold.

In some embodiments, a lower immune score indicates a lesser suitability to receive an influenza vaccine. In some embodiments, a lower immune score indicates a lower likelihood of effectiveness of an influenza vaccine. In some embodiments, a lower immune score indicates a lower likelihood of effectiveness of an influenza vaccine in the subject. In some embodiments, a lower immune score indicates a greater likelihood of infection by influenza. In some embodiments, a lower immune score indicates a greater likelihood of severe disease after infection by influenza. In some embodiments, a higher immune score indicates a previous influenza infection. In some embodiments, a higher immune score indicates a previous influenza vaccination. In some embodiments the probes bound that result in the higher immune score indicate the particular influenza that caused the infection or that was vaccinated against.

In some embodiments, an immune score above a predetermined threshold indicates the subject is suitable to receive an influenza vaccination. In some embodiments, an immune score above a predetermined threshold indicates an influenza vaccine is likely to be effective. In some embodiments, an immune score above a predetermined threshold indicates an influenza vaccine is likely to be effective in the subject. In some embodiments, an immune score above a predetermined threshold indicates a decreased risk of infection by influenza. In some embodiments, an immune score above a predetermined threshold indicates a decreased risk of severe disease upon infection by influenza. In some embodiments, an immune score above a predetermined threshold indicates a subject not at risk for infection by influenza.

In some embodiments, an immune score below a predetermined threshold indicates the subject is unsuitable to receive an influenza vaccination. In some embodiments, an immune score below a predetermined threshold indicates an influenza vaccine is unlikely to be effective. In some embodiments, an immune score below a predetermined threshold indicates an influenza vaccine is unlikely to be effective in the subject. In some embodiments, an immune score below a predetermined threshold indicates a subject at risk for infection by influenza. In some embodiments, an immune score below a predetermined threshold indicates a subject at increased risk for infection by influenza. In some embodiments, an immune score below a predetermined threshold indicates a subject at increased risk for severe disease upon infection by influenza. In some embodiments, likely comprises a chance of occurring of at least 50, 60, 70, 75, 80, 85, 90, 95, 97, 99 or 100%. In some embodiments, likely comprises a chance of occurring of at least 70%. Each possibility represents a separate embodiment of the invention. In some embodiments, unlikely comprises a chance of occurring of at most 1, 5, 10, 15, 20, 25, 30, 40 or 50%. Each possibility represents a separate embodiment of the invention. In some embodiments, unlikely comprises a chance of occurring of at most 30%.

In some embodiments, the method further comprises contacting immune cells with the influenza vaccine. In some embodiments, the contacting is done in vitro. In some embodiments, in vitro is in cell culture. In some embodiments, the immune cells are T cells. In some embodiments, the immune cells are B cells. In some embodiments, the immune cells are a mixed lymphocyte reaction. In some embodiments, the immune cells are peripheral blood mononuclear cells PBMCs. Contacting the immune cells with the vaccine can be done in any way known in the art. In some embodiments, the vaccine is added to the culture. In some embodiments, the vaccine is transferred into the cytosol of the immune cells. Methods of transfer such as transfection, nucleofection, and viral transfer (lentiviral etc.) are known in the art and any such method may be used.

In some embodiments, the immune cells are incubated for at least 1, 2, 3, 4, 5, 6, 7, 8, 9, 10, 11, 12, 13 or 14 days after contact with the influenza vaccine. Each possibility represents a separate embodiment of the invention. In some embodiments, the immune cells are incubated for a sufficient time to produce antibodies against influenza. In some embodiments, media from the immune cells is the solution. In some embodiments, antibodies are isolated from the media. In some embodiments, proteins are isolated from the media.

As used herein, the term “symptomatic infection”, “symptomatic disease” and “symptomatic influenza” are synonymous and used interchangeably and refer to a disease that is apparent due to the presence of an influenza symptom. It will be understood by a skilled artisan that a subject can become infected by influenza and would be found positive by a PCR test or other sensitive test, but still be asymptomatic. Examples of influenza symptoms include, but are not limited to, fever, chills, cough, sore throat, runny or stuffy nose, muscle aches, headache, fatigue, vomiting and diarrhea. In some embodiments, symptomatic infection comprises at least one symptom.

In some embodiments, the method further comprises administering an influenza vaccine to a subject at risk of influenza infection. In some embodiments, the method further comprises administering prophylactic treatment of a subject at risk of influenza infection. In some embodiments, the method further comprises instructing the subject in methods of avoiding influenza infection.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

Methods and Materials

Initial Array Study

Antigen microarray design and spotting. To study anti-influenza antibody repertoires, two types of antigen microarrays were designed and spotted: (1) An influenza viruses and proteins (VP) microarray was spotted with a panel of 35 whole inactivated influenza viruses, 23-64 recombinant HA proteins, and up to 26 recombinant NA proteins, that were selected to represent 85 influenza strains from 19 subtypes of influenza viruses from 1918 to 2020. This wide panel of whole virus and protein antigens includes all seasonal H1N1, H3N2 and B vaccine strains that were circulated from 1984 to 2019 in humans, and additional H1N1, H3N2 and B strains from 1918, to represent the antigenic diversity of vaccine and historical human influenza strains of the H1N1, H3N2 and B subtypes. For each trial a VP array with selected part of these antigens was spotted. (2) For each trial a peptide microarray was also spotted with ˜205 partially overlapping 20 aa peptides (with 15 aa overlap) spanning the full-length HA and NA proteins of a selected influenza strain (see below). KK linkers were added to the peptides design. Microarrays were spotted using a Scienion Sx spotter (Scienion, Germany) on Hydrogel-coated slides that bind amine groups (H slides, Schott, Germany). VP and peptide slides were printed separately, and all slides used for profiling the whole cohort were printed in a single batch to avoid potential batch effects. Each antigen was spotted in triplicate. To reduce costs and sample volumes, each slide was spotted with 8 or 16 identical arrays.

Influenza Antigens

Whole inactivated viruses (WIV): β-propiolactone (BPL)-inactivated whole influenza viruses were obtained from two resources: (1) Influenza viruses that were propagated in chicken embryonic eggs, BPL-inactivated, purified on sucrose columns, and whose concentrations were determined by the hemagglutinin assay. Virus concentration was measured by standard hemagglutination assay (WHO Laboratory Biosafety Manual, 2007 edition, WHO manual for the laboratory diagnosis and virological surveillance of human influenza viruses) and lack of virus infectivity was verified by a plaque assay. (2) Viruses with a known concentration prepared and inactivated by the NIBSC (UK) for use as reference antigens for the single radial diffusion influenza potency assay. The arrays included 35 WIV strains of type A (H1N1, H3N2) and B that were isolated between 1933-2013 (Table 6). The viruses were stored at −80° C. until spotting and were spotted in a 0.0025% spotting buffer (phosphate-buffered saline (PBS) with 0.0025% Triton X-100) or PBS at a concentration of 2 HAU/ul for viruses propagated in embryonated eggs, and 4 μgHA/ml for viruses produced by NIBSC.

Recombinant surface glycoproteins: recombinant HA (rHA) and NA (rNA) proteins were purchased from Sino biological (USA) or Native Antigens (UK) as purified His-tagged proteins or obtained from IRR CDC repository. Most of the recombinant proteins were produced in human HEK293 cell cultures, and some in Baculovirus-Insect Cells. The proteins were spotted in a 0.01% spotting buffer (PBS with 0.01% Triton X-100) or in sciSPOT Protein D1 buffer (Scienion) at a final concentration of 8, 16.25 or 32.5 ug/ml.

Peptides: Synthetic 20 amino acids (aa) peptides were synthesized at >90% purity by CPC scientific (CA USA) or Mimotopes (Australia). Each peptide included a KK tag as an amine group source for binding to the coated slides. —205 peptides were spotted per each strain, dissolved in 20-60% dimethyl sulfoxide (DMSO), diluted to 10-30% DMSO concentration and spotted in 0.0025% Triton X-100 at a final concentration of ˜1 mg/ml. Peptides from the Wis05-A/Wisconsin/67/2005 (H3N2) strain HA and NA proteins were spotted on the peptide array, and are provided in Table 4, SEQ ID NOs: 1-199.

Normalization of serum concentration. Since the concentrations of different serum samples from the same individual may be different due to a variety of reasons at different timepoints, serum concentrations were normalized by measuring the total protein concentration in the serum using spectrophotometer and diluting all the samples to the same total protein concentration by PBS (the average concentration/10). These diluted samples were considered as diluted 1:10, and additional dilutions were done from them.

Antigen Microarray (AM) hybridization assay: The total protein concentration of each serum sample was measured by spectrophotometer, and then diluted 1:10 to get the same total concentration. Normalized Serum samples were diluted 1:100-1:300 for IgA profiling or 1:1000-1:3000 for IgG profiling in a hybridization buffer (PBS containing 0.025% tween-20 and 1% bovine serum albumin). AMs were blocked with a blocking buffer (50 mM borate, 50 mM ethanolamine in 0.1M Tris, pH 9.0) for 1 h at room temperature (RT) with light rocking agitation, and then washed twice with PBST washing buffer (PBS with 0.05% tween-20), twice with PBS, and once with double-deionized water (DDW). The AMs were then dried by centrifugation at 800 g for 5 min and hybridized with the serum samples diluted in a hybridization buffer for 2 h at RT with light rocking agitation in 16-array PEPperCHIP® incubation trays 3/16 (PepperPrint). After hybridization, arrays were rinsed twice with PBST, twice with PBS (as mentioned above) and incubated with a fluor-conjugated secondary antibody (Alexa Fluor 647 Donkey anti-human IgG (H+L), diluted 1:1000 or 1:2000, or Alexa Fluor 647 goat anti-human serum IgA diluted 1:3000 or 1:6000 (Jackson ImmunoResearch, West Grove, Pa.) for 45 min at RT with light rocking agitation. Following another set of washes, (2×PBST, 2×PBS, and once with DDW) arrays were thoroughly dried by centrifugation at 800 g for 5 min in microscope slide racks. Slides were scanned on a 4-laser GenePix 4400A scanner (Molecular Devices). Images were analyzed by GenePix Pro 7.0 to obtain the mean fluorescence intensity (0≤MFI≤65,000), and local background was subtracted for each spot (MFI-B). FIG. 18 provides a scan image from an exemplary array.

Baseline and post-vaccination samples from the same individual were hybridized at the same day on the same slide and processed together. Negative control arrays were hybridized with a hybridization buffer only at each experiment day and were used for background subtraction of non-specific staining. The median background-subtracted MFI (median MFI-B) was selected for each triplicate of the same antigen. The MFI-B of negative controls from the same experiment was subtracted, and values below 20 were considered as zeros. Data analysis was conducted using an in-house pipeline written in Python. The vaccine-induced change in the antibodies level to a given antigen was calculated as the fold-change rise from the baseline level, or as a baseline-subtracted response.

Analysis of microarray results. Scanned slides were annotated using GenePix Pro version 7 (Molecular Devices) to obtain the mean fluorescence intensity. The local background fluorescence intensity was subtracted from each spot MFI. The median was selected for each triplicate of the same antigen. Data analysis was conducted using an in-house pipeline written in Python. The vaccine-induced change in the antibodies level to a given antigen was calculated as the fold-change rise from the baseline level, or as a baseline-subtracted response.

Magnitude and breadth of summary statistics: The antibody profile generated by the antigen microarrays are a multidimensional measurement of the antibody responses to a large set of antigens. To compare the antibody responses of each sample as measured by these antigen microarrays across time and groups, we defined the magnitude and breadth of responses to a given set of antigens. We denote the normalized array measurements by x_{i,p,ap} where: i—subject, =1, . . . , N; p—pathogen (influenza strain or HHV species), p=1, . . . , P; aₚ—antigen from pathogen, =1, . . . , Nₚ. zᵢ—denotes treatment assignment/group (vaccine/placebo, lean/obese) of subject i. yᵢ—denotes outcome of subject i (vaccine-induced Ab titer/infection status/disease status). The observed data for each subject are (zᵢ, yᵢ, z_{i,p,ap}), for i=1, . . . . N; p=1, P; and aₚ=1, . . . , Nₚ. For a given subject we define the ‘breadth’ and ‘magnitude’ of responses to each pathogen as follows:
1.

$${m}_{i , p} = {\sum }_{{a}_{p} = 1}^{{N}_{p}} {x}_{i , p , {a}_{p}}$$

—denotes the magnitude of responses to all antigens of pathogen.
2.

$${b}_{i , p} = {\sum }_{{a}_{p} = 1}^{{N}_{p}} I ( {x}_{i , p , {a}_{p}} > 0 )$$

—denotes breadth of response to antigens from pathogen.
   We consider the following data representations for use in our statistical models:
1. x_{i,p,ap}—normalized array measurements.
1

$${d}_{i , p} = f ( i , p \vert {a}_{{p}_{1}} , … , {a}_{{p}_{{N}_{p}}} ) ⟶ [ 0 , 1 ] \_$$

where f( ) is some function of the antigen measurements for pathogen p that maps to the binary indicator of whether the subject i has a detectable Ab response to pathogen: e.g.

$$f ( i , p \vert {a}_{{p}_{1}} , … , {a}_{{p}_{{N}_{p}}} ) = 1 ( {b}_{l , p} )$$

is 1 if a response is detected to at least one antigen from pathogen p, and 0 otherwise.
3. mᵢ=Σₚ₌₁^{p}m_{i,p}—denotes the magnitude of responses to all antigens.
4. bᵢ=Σᵢ₌₁^{p}d_{i,p}—denotes the breadth of responses to pathogens.

In order to characterize the immune history profile for H3N2, H1N1 and B strains, the magnitude and breadth of antibody binding to each of the subtypes were calculated. Magnitude was defined as the sum of the overall signal intensity (MFI) for all antigens from the same subtype. Breadth was defined as the total number of antigens with MFI above a predefined threshold (e.g., MFI>2000 for proteins and MFI>200 for whole viruses).

In order to summarize the repertoire of antibodies to peptides of HA or NA protein of a specific influenza strain, the magnitude and breadth of antibody binding to this set of peptides was calculated. Magnitude was defined as the sum of the overall signal intensity (MFI) for all peptides from the same protein. Breadth was defined as the total number of peptides with an MFI above a predefined threshold (e.g., MFI>2000).

Baseline immune history (BIH) ranking. All subjects in each trial were ranked according to the baseline magnitude or breadth to whole viruses or rHA proteins to a selected subtype, or magnitude of HA or NA peptides of a selected strain, or MFI of the rHA or whole virus antigen of the selected strain. For each ranking, the subjects were divided into quartiles, and the two extreme quartiles of highest and lowest scores, termed high-BIH and low-BIH, respectively, were compared.

Vaccine responders ranking. For each individual, the baseline-adjusted response to the vaccine was computed for each antigen, by subtracting the baseline binding of antibodies to this antigen from the post-vaccination antibody binding. All subjects in each trial were ranked according to the magnitude or breadth of baseline-adjusted responses to HA or NA peptides of a selected strain, or magnitudes of whole viruses or rHA proteins of selected subtype. The subjects were divided into quartiles, and the two extreme quartiles of highest and lowest baseline-adjusted responses, termed high-responders and low-responders, respectively, were compared.

Statistical analysis. A 2-sided hypothesis tests (Wilcoxon rank-sum and Fisher's exact test) was used to test for differences between the distribution of breadth and magnitude scores defined above. Differences between baseline and post-vaccination responses within each group were tested using the Wilcoxon signed rank test.

To predict the group of subjects based on the immune-history profiles, a logistic regression model was used, and a generalized linear model (GLM) was used to predict the vaccine-induced immune responses using a logistic model for binary variables and a linear model for continuous. All models were trained using leave-one-out cross validation. Predicted values were collected over all folds for computing the area under the curve (AUC) summary stat. All continuous variables were standardized prior to training. Regularization was implemented using the elastic net package with different alpha and L 1 weight parameters for each model. All models were trained using the statsmodels python package.

FLUVACS Study

Clinical dataset: Clinical samples from the FLUVACS study, a randomized, double-blinded, placebo-controlled, community-based clinical trial, conducted in healthy adults aged 18-49 during the 2007-08 influenza season (Monto et al., 2009, “Comparative efficacy of inactivated and live attenuated influenza vaccines”, N. Engl. J. Med., September 24; 361(13):1260-7)) were used. Subjects were randomly divided into three treatment arms: (1) Vaccination with the trivalent inactivated influenza vaccine (TIV, Fluzone); (2) Vaccination with the trivalent live-attenuated influenza vaccine (LAIV, Flumist); or (3) Placebo administered either intramuscularly or intranasally. The vaccines contained the following strains: A/Solomon Islands/3/2006 (H1N1), A/Wisconsin/67/2005 (H3N2), and B/Malaysia/2506/2004 (B/Victoria lineage). Herein serum samples collected at two timepoints from 165 subjects were analyzed: baseline (pre-vaccination), and post (approximately 30 days post vaccination). The cohort analyzed here was composed of a case-control set including 86 cases—subjects that were symptomatically infected by the influenza A/H3N2 A/Wisconsin/67/2005 vaccine strain, as confirmed by real-time reverse transcription polymerase chain reaction (RT-PCR) from throat swab samples, and 79 randomly selected controls without influenza illness during the 2007-08 influenza season, along 3 months post vaccination (Table 2). The trial was approved by the institutional review board at the University of Michigan Medical School. Written informed consents were obtained from all participants prior to enrollment.

FLUVACS study microarrays: Viruses and proteins antigen microarrays included 35 inactivated influenza whole-viruses and 26 influenza recombinant HA proteins (Table 6). In addition, HA and NA peptide arrays of the Wis05 (H3N2) HA and NA proteins were spotted.

Dilutions of samples and secondary antibodies for array hybridization: Normalized Serum samples were diluted 1:100 for IgA profiling or 1:1000 for IgG profiling. Secondary antibodies: Alexa Fluor 647 Donkey anti-human IgG (H+L), diluted 1:1000, or Alexa Fluor 647 goat anti-human serum IgA diluted 1:6000 (Jackson ImmunoResearch, West Grove, Pa.).

Thresholds for breadth calculations: MFI>2000 for proteins and peptides, MFI>200 for whole viruses.

TABLE 2
Clinical data set

| | | Inactivated | Live | |
|---|---|---|---|---|
| | Placebo | (TIV) | (LAIV) | Total |
| Num of participant (n) | 56 | 51 | 58 | 165 |
| Cases/Controls | 29/27 | 27/24 | 30/28 | 86/79 |
| Gender (M/F) | 19/37 | 17/34 | 21/37 | 57/108 |
| Caucasian (%) | 49 (87%) | 49 (96%) | 48 (83%) | 146 (88%) |
| Age, years (median) | 18-45 | 18-47 | 18-49 | 18-49 |
| | (21.7) | (23.8) | (25.8) | (23.8) |

Data was analyzed using a computational in-house pipeline written in Python. Statistical significance for comparisons between baseline and post-vaccination antibody levels within each treatment group, as well as between influenza PCR-confirmed H3N2 cases (Flu Pos) vs. uninfected control (Flu Neg) participants was performed using the Wilcoxon signed rank test. All cases were infected with the vaccine H3N2 strain Wis05. Comparisons between the categorical infection rates of low and high baseline immune-history groups were computed using a logistic regression model adjusted for all baseline covariates.

Correlates of risk and correlates of protection: Pre-vaccination (baseline) and post-vaccination (Day 30) immunological markers as correlates of risk (CoRs) of symptomatic H3N2 influenza infection were assessed. Baseline CoRs were defined as pre-vaccination markers that are associated with risk of H3N2 illness during the flu season in one or more of the three treatment arms (TIV, LAIV, placebo). While a CoR nomenclature was used because these types of correlates can be directly assessed based on randomized efficacy trial data without requiring strong unverifiable assumptions, CoRs are linked to the common correlate of protection (CoP) terminology, where a post-baseline CoP can be defined as a post-vaccination immunological marker that either (1) is a common CoR in the vaccine and placebo groups and mediates a substantial fraction of vaccine efficacy (VE) against influenza illness, or (2) that modifies VE. Previous studies have supported Day 30 HAI titer as a partially valid CoP of both types, with titer 1:40 associated with about 50% protection and mediating more than 50% of VE. A baseline (pre-vaccination) CoP has the same two definitions, except mediation no longer applies because mediators, in capturing ‘pathways’ of a vaccine's effect, are defined post-baseline. All variance estimates were robust sandwich variance estimates except for the breadth variables in which model-based variance estimates were used since the breadth variables have very unbalanced distributions and the sandwich variance estimates are not stable.

The correlates of risk (CoR) models used herein were based on regression binary outcome on immune response predictors within each treatment arm. The post-vaccination (Day 30) models included both baseline and post-vaccination measurements for each variable.

TABLE 3
List of all variables ordered by the analysis primary and secondary plan
tiers. Variables included a single antigen or summary statistic-magnitude and breadth-for
each subtype (H3N2, H1N1, B). Vaccine years represent circulated strains that were also
administrated in the seasonal vaccine. All measurements were done twice for IgG and IgA
antibodies to whole inactivated viruses (WIVs) or recombinant HA/NA (rHA/rNA) protein
(as listed in the antigen type column).

| | Analyzed variable | | | |
|---|---|---|---|---|
| | (8 variables) | Subtype | Vaccine year | Variable type |
| Primary | H3N2 proteins magnitude | H3N2 | 1990-2016 | Proteins magnitude |
| | H3N2 virus magnitude | H3N2 | 1987-2016 | Viruses magnitude |
| | Wisconsin/67/2005 | H3N2 | 2007/8 | rHA |
| | Wisconsin/67/2005 | H3N2 | 2007/8 | WIV |
| | Analyzed variable | | | |
| | (36 variables) | Subtype | Vaccine year | Antigen type |
| Secondary | H3N2 proteins breadth | H3N2 | 1990-2016 | Proteins Breadth |
| | H3N2 proteins magnitude | H3N2 | 1990-2016 | Proteins Magnitude |
| | H3N2 virus breadth | H3N2 | 1987-2016 | Viruses Breadth |
| | H3N2 virus magnitude | H3N2 | 1987-2016 | Viruses Magnitude |
| | Wisconsin/67/2005 | H3N2 | 2007/8 | rHA |
| | Wisconsin/67/2005 | H3N2 | 2007/8 | WIV |
| | H1N1 proteins breadth | H1N1 | 1978-2016 | Proteins Breadth |
| | H1N1 proteins magnitude | H1N1 | 1978-2016 | Proteins Magnitude |
| | H1N1 virus breadth | H1N1 | 1978-2016 | Viruses Breadth |
| | H1N1 virus magnitude | H1N1 | 1978-2016 | viruses Magnitude |
| | Solomon/islands/3/2006 | H1N1 | 2007/8 | rHA |
| | Solomon/islands/3/2006 | H1N1 | 2007/8 | WIV |
| | B proteins breadth | B | 1989-2017 | Proteins Breadth |
| | B proteins magnitude | B | 1989-2017 | Proteins Magnitude |
| | B virus breadth | B | 1989-2016 | Viruses Breadth |
| | B virus magnitude | B | 1989-2016 | viruses Magnitude |
| | Malaysia/2506/2004 | B | 2007/8 | rHA |
| | Malaysia/2506/2004 | B | 2007/8 | WIV |

Statistical analysis plan: The proteins and viruses microarrays included a set of 61 whole virus and rHA influenza antigens that were analyzed. Summary statistics were computed for groups of antigens (such as subtype specific magnitude and breadth, described herein). A correlates of risk framework (described herein) could be used to analyze the predictive power of each individual antigen as well as their summary statistics at both baseline and post-vaccination. To preserve statistical power, a statistical analysis plan was devised prior to the analysis of the data, in which two analysis tiers were defined: primary and secondary (See Table 3). Briefly, since all of the cases in the dataset were infected with the A/Wisconsin/67/2005 (Wis05) H3N2 strain, the primary tier only included the analysis of IgG and IgA antibodies to the Wis05 strain, and the overall H3N2 magnitude, which represents the H3N2 specific immune-history. A total of 8 variables were included in the primary tier. The secondary tier included all the vaccine strains, as well as the magnitude and breadth to H1N1, and B subtypes, yielding a total of 36 variables. A cutoff of P-values<0.05 and q<0.1 were used to determine significance.

Baseline immune history (BIH) and vaccine responders ranking: All subjects (n=165) were ranked according to the baseline magnitude or breadth to H3N2 whole viruses, or rHA proteins of H3N2 strains, or magnitude or breadth of Wis05 H1 or N1 peptides, or the WI of Wis05 rHA or whole virus.

Obesity Study

Clinical dataset. Participants were recruited as a part of a prospective observational study carried out at the University of North Carolina at Chapel Hill Family Medicine Center, an academic outpatient primary care facility in Chapel Hill, N.C. Recruitment criteria for this study included adults 18 years of age and older receiving the seasonal trivalent inactivated influenza vaccine (TIV) for the years 2010-2011 that included the following strains: A/H1N1/California/7/2009, A/H3N2/Perth/16/2009, and B/Brisbane/60/08. The study cohort included both obese (body mass index bigger than 30 kg/m2, n=104) and healthy-weight (18.5≤BMI≤24.9, n=101) individuals. Sera samples were collected prior to vaccination (baseline—day 0) and one-month (28-35 days) post vaccination. All procedures were approved by the Biomedical Institutional Review Board at the University of North Carolina at Chapel Hill.

Obesity study microarrays: Viruses and proteins antigen microarrays included 34 inactivated influenza whole-viruses (the same as for Fluvacs but with A/California/04/2009—H1N1 removed) and 23 influenza recombinant HA proteins. In addition, HA and NA peptide arrays of the Cal09 (H1N1) HA and NA proteins (SEQ ID NOs: 200-399) were spotted.

Dilutions of samples and secondary antibodies for array hybridization: Normalized Serum samples were diluted 1:300 for IgA profiling or 1:3000 for IgG profiling. Secondary antibodies: Alexa Fluor 647 labeled polyclonal anti-human IgG antibody at 1:1000 dilution (Jackson ImmunoResearch cat #709-605-149) or Alexa Fluor 488-conjugated polyclonal anti-human IgA antibody at 1:6000 dilution (Jackson ImmunoResearch cat #109-545-011).

Thresholds for breadth calculations: MFI>2000 for proteins and peptides, MFI>4000 for whole viruses.

The magnitude and breadth were calculated for each subject at each timepoint and also for the post-vaccination baseline-subtracted responses, for the following sets of antigens: Cal09 H1 peptides, Cal09 N1 peptides, HA proteins of subtype B strains, HA proteins of subtype H1N1 strains, HA proteins of subtype H3N2 strains, whole viruses of subtype B strains, whole viruses of subtype H1N1 strains, and whole viruses of subtype H3N2 strains.

Baseline immune history (BIH) and vaccine responders ranking: All subjects (n=189) were ranked according to the baseline magnitude or breadth to H1N1 whole viruses, or rHA proteins of H1N1 strains (rH1), or magnitude or breadth of Cal09 H1 or N1 peptides, or the MFI of Cal09 rHA or whole virus antigens.

Human IgG and IgA ELISA Quantitation. Commercial enzyme linked immunosorbent assay (ELISA) kits (Bethyl Laboratories, USA, cat #E80-104 for human IgG and cat #E80-1026 for human IgA) were used to quantify the total human IgG and IgA concentrations in the normalized sera, using the manufacturer's instructions with the following modifications. The normalized serum samples were diluted 1:24375 for human IgA ELISA and 1:243750 for human IgG ELISA. The ELISA assays were performed in 384-well white MaxiSorp Nunc plates (cat #460372). The wells were coated with 17 μl/well of ELISA coating antibody in coating buffer, blocking and all washes were performed with 100 μl/well, and 30 μl/well diluted sera and standards were added in triplicates. Following washes, 30 μl/well HRP-conjugated detection antibody was also added. Instead of TMB, we used 30 μl/well of the SuperSignal™ West Pico PLUS Chemiluminescent Substrate (Thermo Scientific, cat #34579, the two reagents were mixed at 1:1 ration before adding to the plate). Plates were read using a standard luminometer (TECAN infinite M200 PRO) at 600 nm. The average of each triplicate was calculated, and total antibody concentration was concluded from the standard curve.

### Example 1: TIV Significantly Increases IgG and IgA Levels to the Vaccine Strains

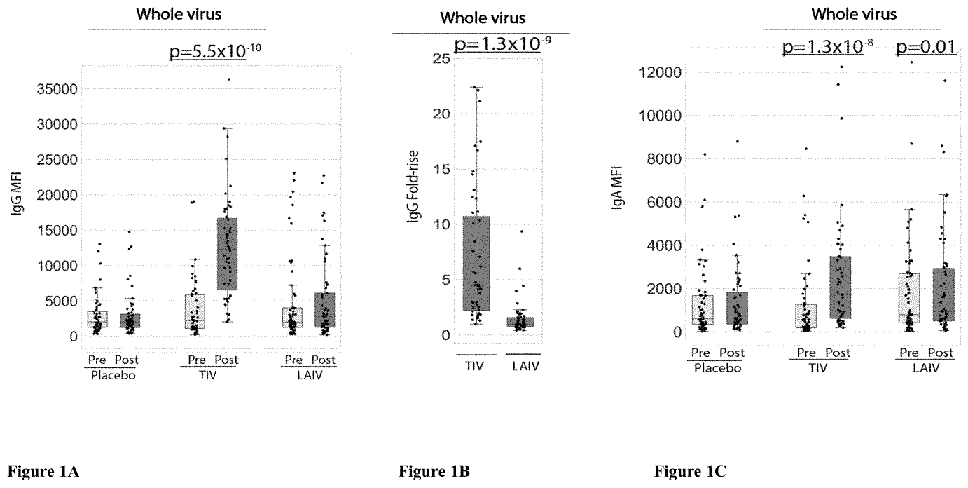

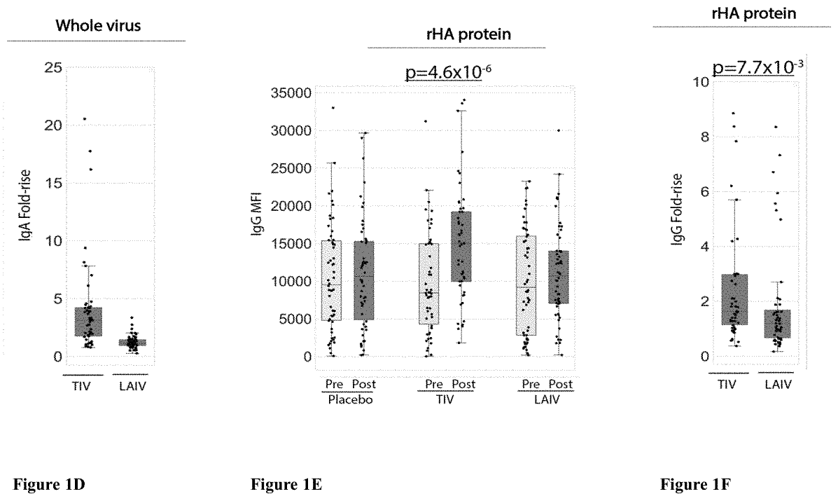

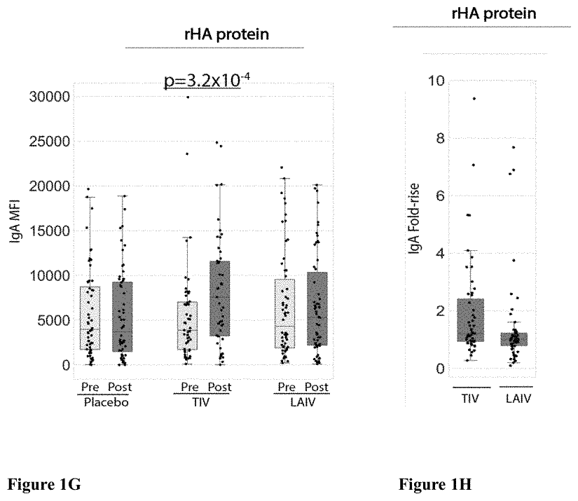

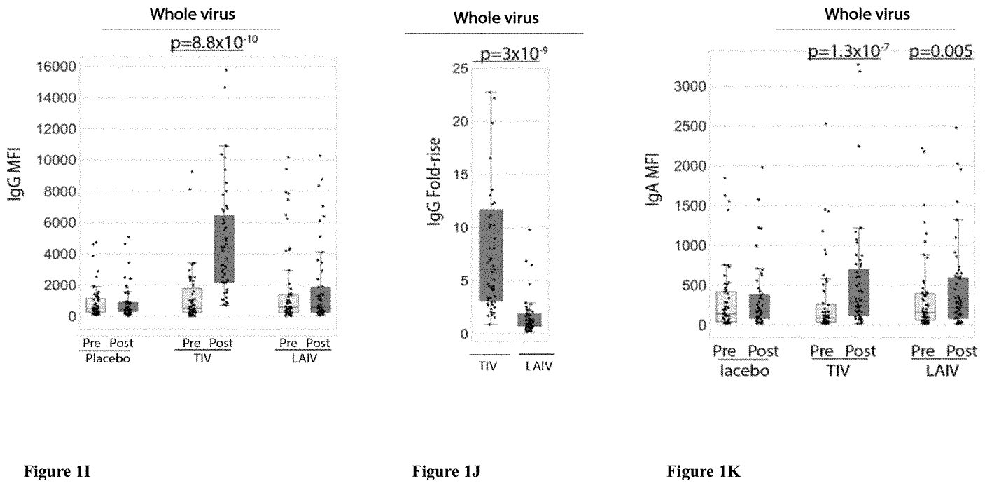

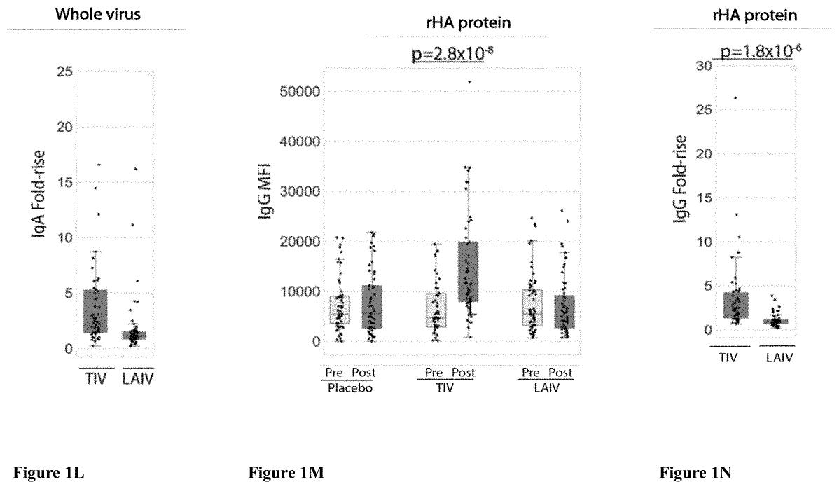

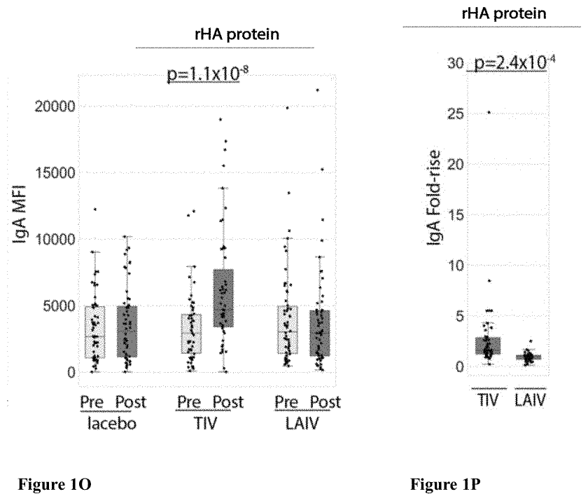

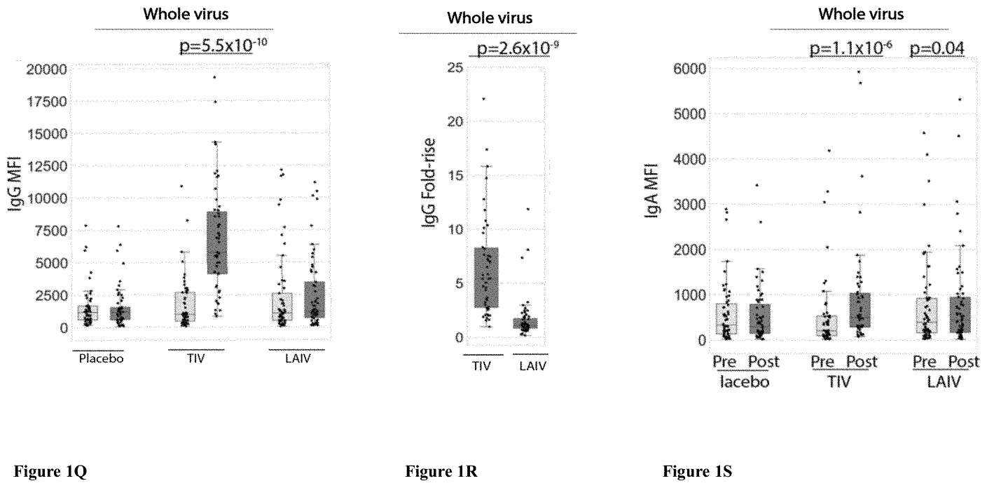

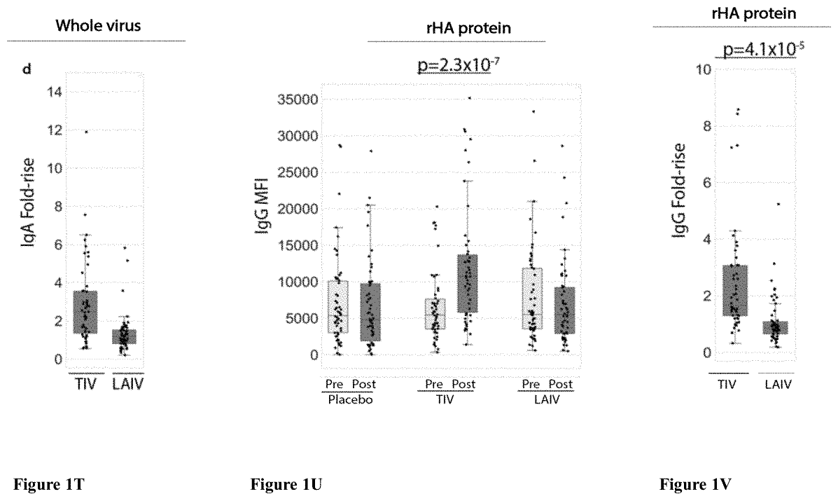

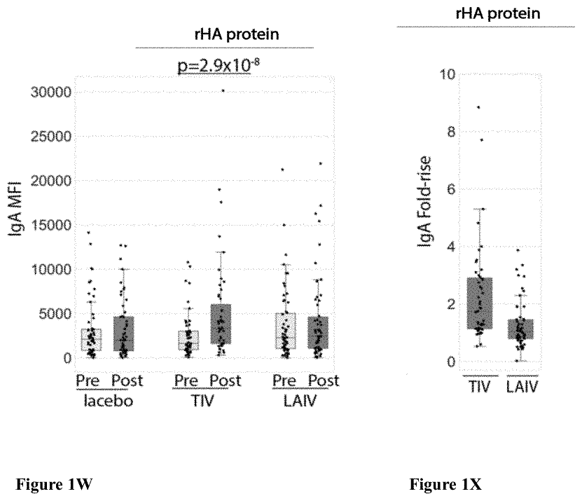

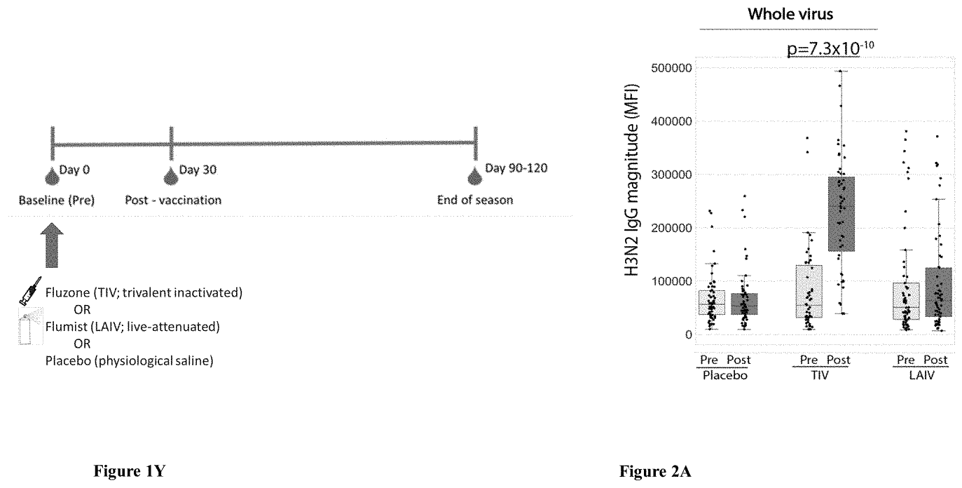

To study the baseline and post-vaccination anti-influenza IgG and IgA antibody levels in the three treatment arms of the FLUVACS study, a case-control set of 165 subjects was used (see Table 2 and FIG. 1Y). First, antibodies levels to each of the seasonal influenza vaccine strains used in the study were compared: A/wisconsin/65/2005 (Wis05; H3N2), A/Solomon Islands/3/2006 (H1N1) and B/Malaysia/2506/2004 (Victoria lineage). Each of the vaccine strains was represented on the VP-arrays by two types of antigens: whole inactivated viruses (WIV), and recombinant HA (rHA) proteins. A significant rise in both IgG and IgA antibody levels to the H3N2 vaccine strain was observed following vaccination with TIV for both WIV (p=5.5×10⁻¹⁰, p=1.3×10⁻⁹, FIG. 1A, 1C) and rHA antigens (p=4.6×10⁻⁶, p=3.2×10⁻⁴, FIG. 1E, 1G). A weaker, but still significant, rise in antibody levels in the LAIV group was observed only for IgA against the WIV antigen (p=0.01, FIG. 1C). TIV vaccination induced significantly higher IgG MFI fold-rise as compared to the LAIV vaccination for both the WIV and rHA antigens (p=1.3×10⁻⁹, p=7.7×10⁻³, FIG. 1B, 1F). Differences in IgA fold-rise were not significantly higher in the TIV group (FIG. 1D, 1H). A significant rise in IgG and IgA antibody levels was also observed in the TIV group for the H1N1 (FIG. 1I-P) and B (FIG. 1Q-X) vaccine strains. Similar to H3N2, significant differences in the IgG fold-rise distributions were found for both WIV and rHA antigens (FIG. 1J, 1N, 1R, 1V), and were also observed for IgA against the rHA of the H1N1 vaccine strain (FIG. 1P).

### Example 2: TIV and LAIV Significantly Increase the Magnitude and Breadth of IgG and IgA to Influenza Strains Circulating from 1933 to 2017

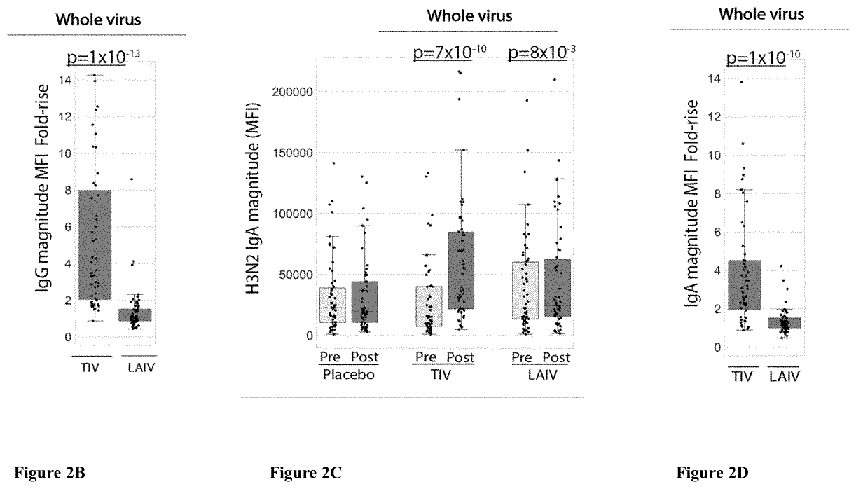

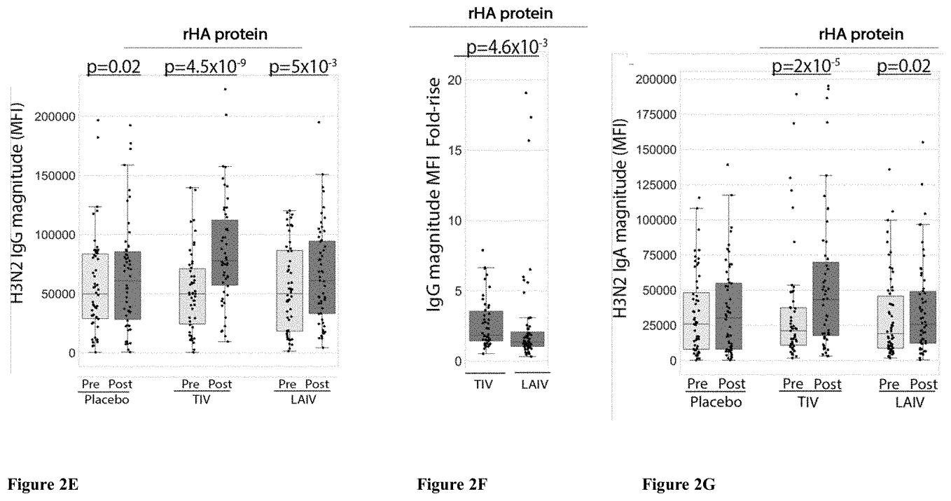

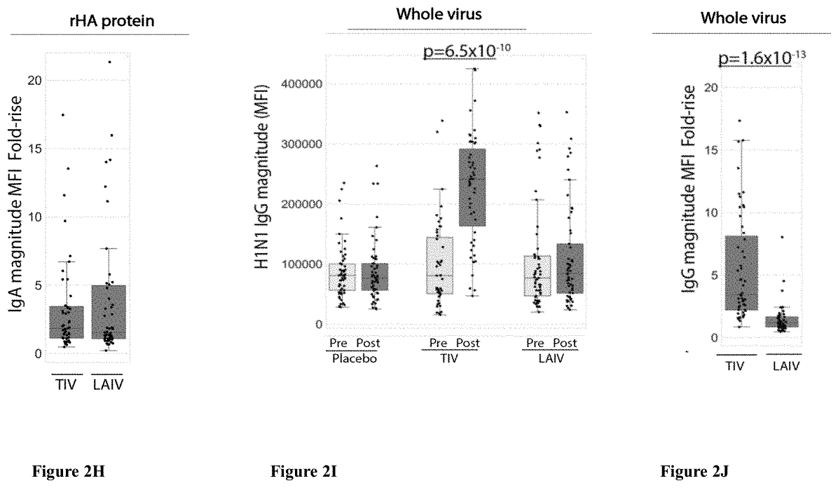

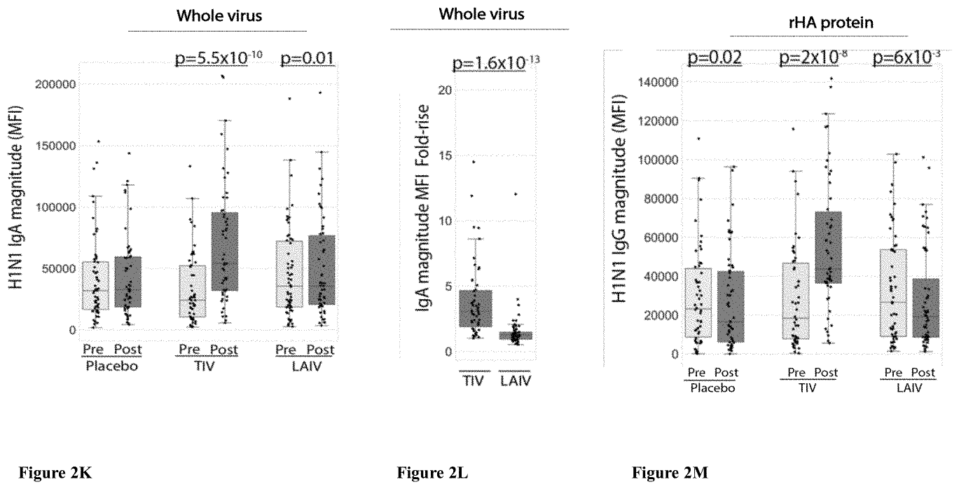

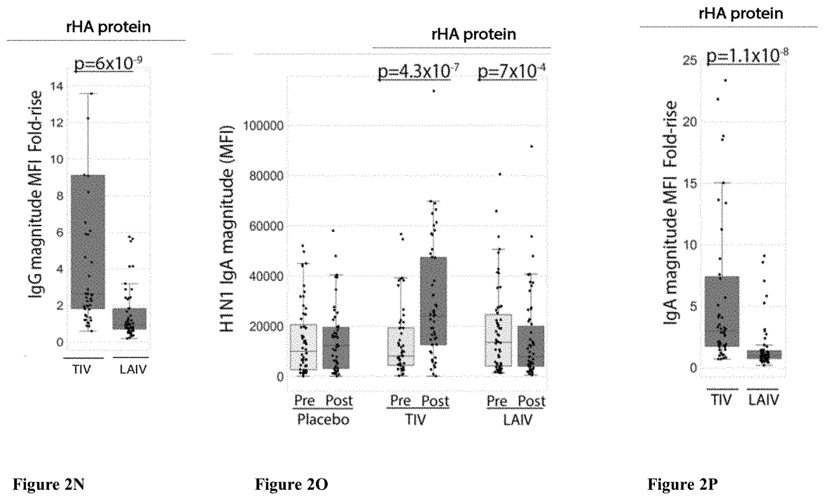

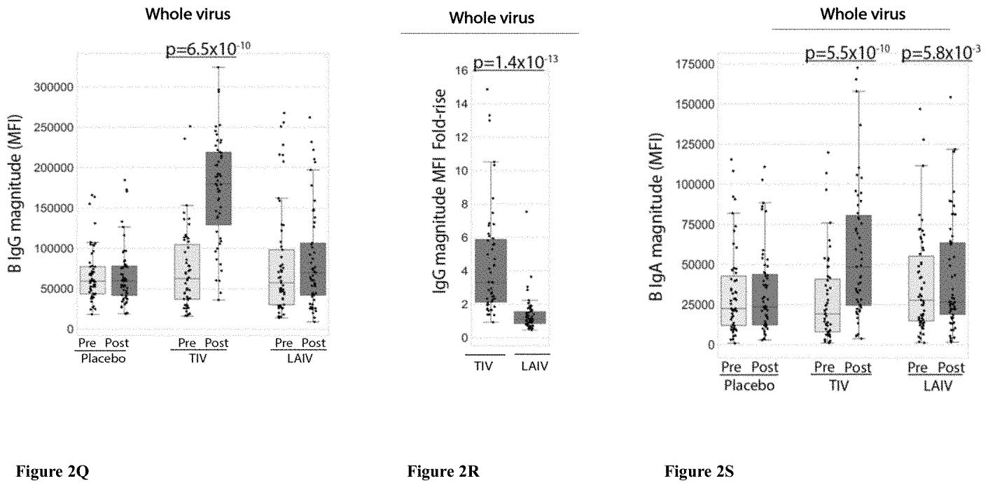

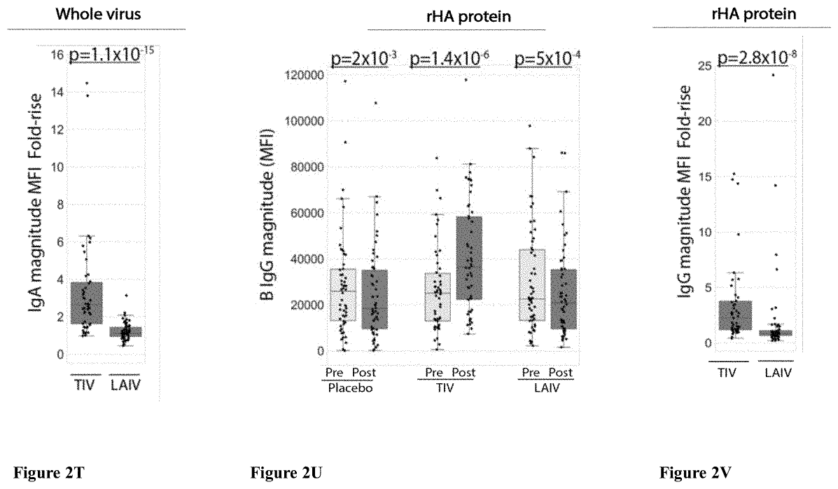

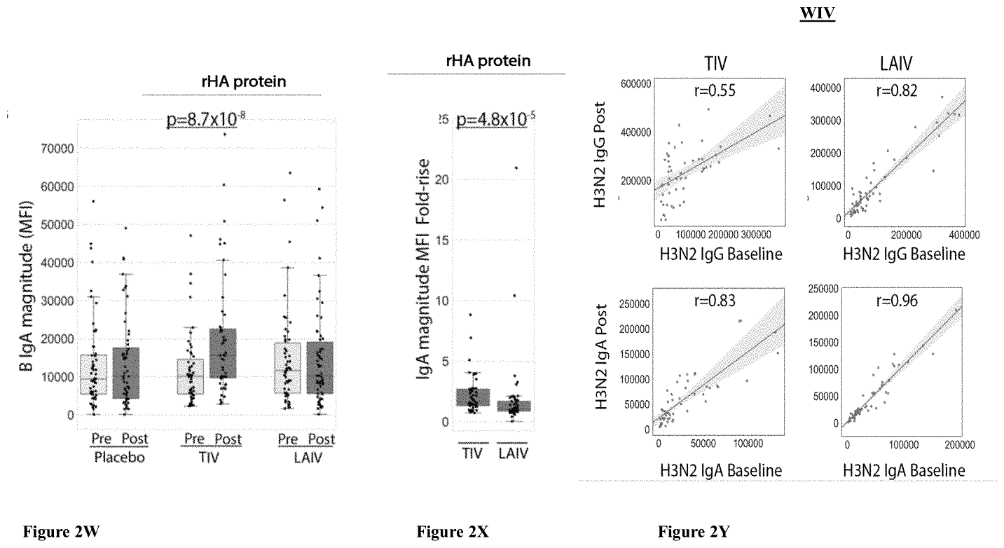

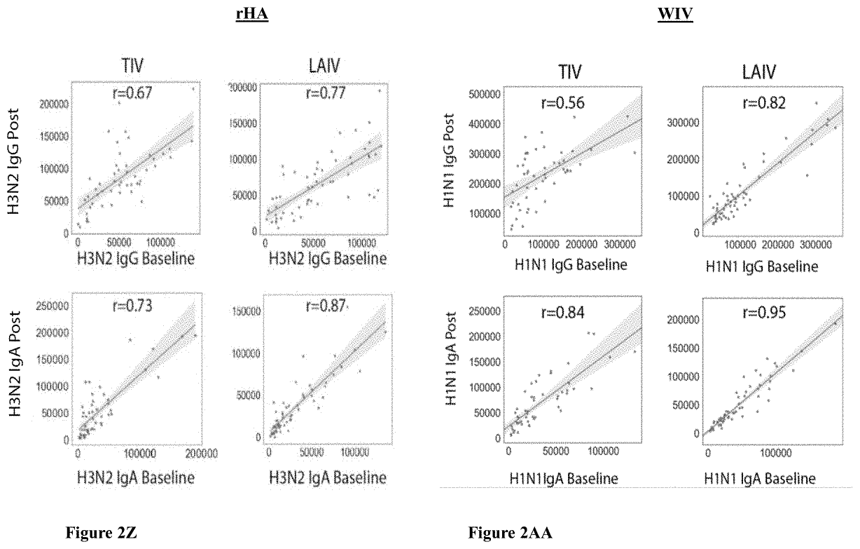

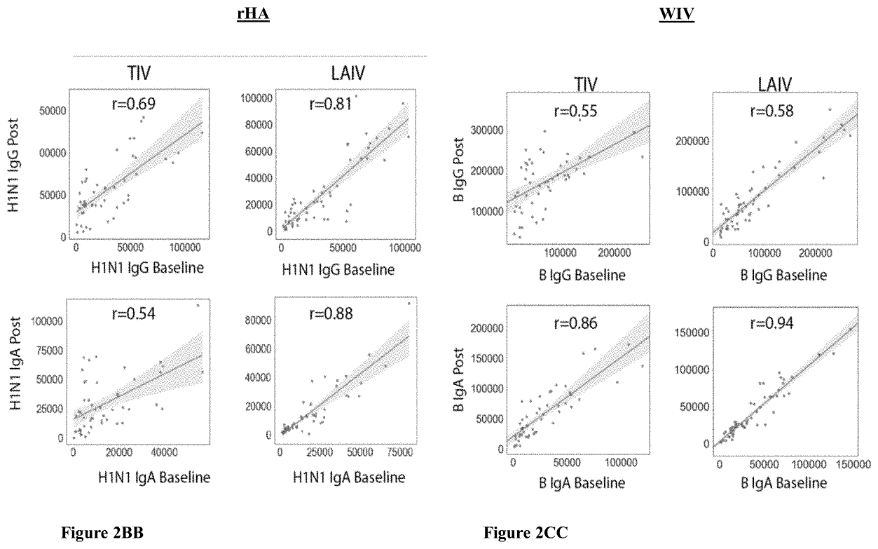

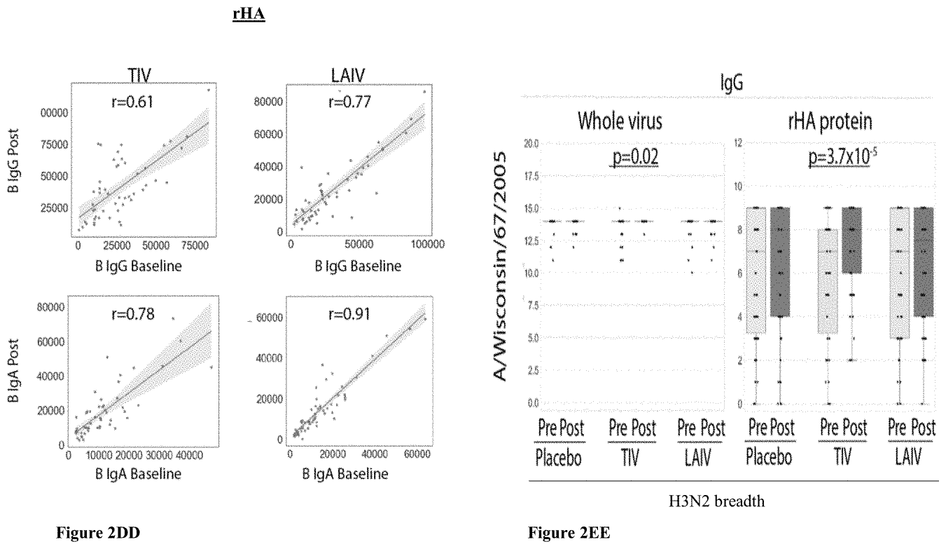

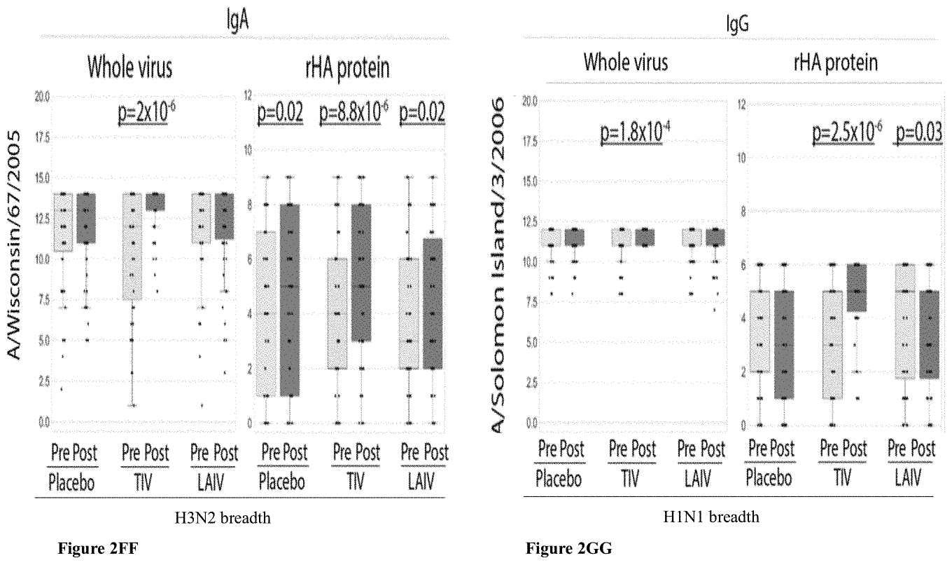

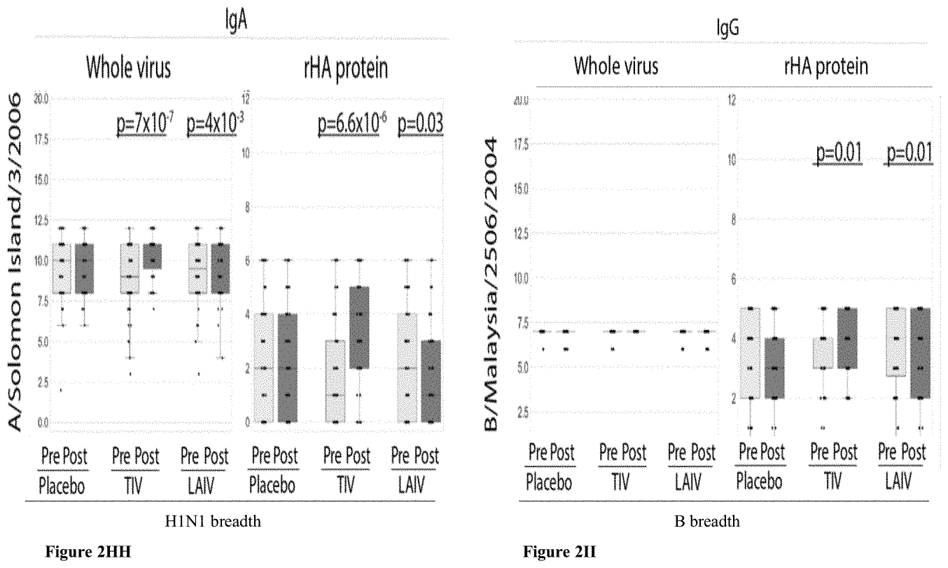

To capture influenza immune history the antigen arrays included 35 whole inactivated influenza viruses (WIV) and 26 recombinant HA (rHA) proteins representing a total of 39 different strains circulating from 1933-2017 (Table 6), including strains isolated after the FLUVACS trial (2009-2017), allowing for the quantification of purely cross-reactive responses to these strains. There was found a significant rise in the magnitude of IgG (FIG. 2A, 2B, 2E, 2F) and IgA (FIG. 2C, 2D, 2G, 2H) antibodies for both the WIV and rHA antigens of the H3N2 subtype post-vaccination in both the TIV and LAIV groups. The MFI fold-rises for antibody magnitudes to H3N2 viruses and rHA proteins were significantly higher in the TIV group as compared to the LAIV group (FIG. 2B, 2D, 2F), except for the IgA magnitude fold-rise to the rHA proteins set (FIG. 2H). Even more highly significant post-vaccination rise in antibody magnitudes in the TIV group were observed for the H1N1 subtype (FIG. 2I-2O) and B strains (FIG. 2Q-2X). Similarly, highly significant increases in the IgG and IgA breadth to all three vaccine strains, were observed only in the TIV group (FIG. 2EE-JJ).

In order to examine the association between the baseline and post-vaccination magnitude of IgG and IgA antibodies, their Spearman correlation was calculated. Positive correlation between baseline and post-vaccination IgG and IgA antibody levels to H3N2 viruses (0.55≤r≤0.96, FIG. 2Y) and HA proteins (0.67≤r≤0.87, FIG. 2Z) were found in both the TIV and the LAIV groups. However, correlations in the LAIV group were overall stronger as compared to the TIV group. Significant correlations were also found for IgG and IgA levels to the H1N1 (FIG. 2AA-BB) and B (FIG. 2CC-DD) subtypes with stronger correlation in the LAIV group as compared to the TIV group. In both TIV and LAIV groups, the baseline and post-vaccination IgA magnitudes to whole viruses from all the three subtypes were more strongly correlated than IgG magnitude (FIG. 2Y, 2AA, 2CC).

TABLE 6
Probes on an exemplary array: Antigen list for H1N1, H3N2, and B
subtypes. The table is specifying the strain, vaccine year of this strain, and the type of antigen
spotted on the array (Whole inactivated virus, recombinant HA or NA proteins).
A/California/04/2009-H1N1 was not included in the array used for the obesity study.

| | | | | HA | NA |
|---|---|---|---|---|---|
| Strain | Subtype | Vaccine year | Virus | Protein | Protein |
| A/South Carolina/1/1918 | H1N1 | None | | + | |
| A/WSN/1933 | H1N1 | None | + | + | |
| A/Puerto Rico/8/1934 | H1N1 | None | | + | |
| A/USSR/90/1977 | H1N1 | None | + | + | |
| A/Brazil/11/1978 | H1N1 | None | + | | |
| A/Chile/1/1983 | H1N1 | 1984-87 | + | | |
| A/Singapore/6/1986 | H1N1 | 1987-1997 | + | | |
| A/Beijing/262/1995 | H1N1 | 1998-2000 | + | + | |
| A/New Caledonia/20/1999 | H1N1 | 2000-07 | + | + | |
| A/Solomon lslands/3/2006 | H1N1 | 2007-08 | + | + | |
| A/Brisbane/59/2007 | H1N1 | 2008-10 | + | + | |
| A/California/04/2009 | H1N1 | 2010-16 | + | + | + |
| A/California/7/2009 | H1N1 | 2010-16 | + | + | |
| A/Christchurch/16/2010 | H1N1 | 2010-16 | + | | |
| Total H1N1 antigens | 14 | | 12 | 10 | 1 |
| A/Aichi/2/1968 | H3N2 | None | | | + |
| A/Bangkok/1/1979 | H3N2 | None | + | | |
| A/Leningrad/360/1986 | H3N2 | 1987-1988 | + | | |
| A/Guizhou/54/1989 | H3N2 | 1990-1991 | + | + | |
| A/Shandong/9/1993 | H3N2 | 1994-95 | + | | |
| A/Sydney/5/1997 | H3N2 | 1998-2000 | + | + | |
| A/Panama/2007/1999 | H3N2 | 2000-2004 | + | | |
| A/New York/55/2004 | H3N2 | 2005-2006 | + | | |
| A/California/07/2004 | H3N2 | 2005-2006 | + | | |
| A/Wisconsin/67/2005 | H3N2 | 2006-2008 | + | + | |
| A/Brisbane/10/2007 | H3N2 | 2008-2010 | + | + | |
| A/Uruguay/716/2007 | H3N2 | 2008-2010 | + | | |
| A/Victoria/210/2009 | H3N2 | 2011 | | + | |
| A/Perth/16/2009 | H3N2 | 2010-12 | + | + | |
| A/Victoria/361/2011 | H3N2 | 2012-13 | + | + | |
| A/Texas/50/2012 | H3N2 | 2013-15 | + | + | |
| A/Switzerland/9715293/2013 | H3N2 | 2015-16 | + | + | |
| Total H3N2 antigens | 17 | | 15 | 9 | 1 |
| B/Lee/1940 | B | None | + | | |
| B/Yamagata/16/1988 | B | None | + | + | |
| B/Jiangsu/10/2003 | B | 2005-06 | + | + | |
| B/Malaysia/2506/2004 | B | 2006-2008 | + | + | |
| B/Florida/4/2006 | B | 2008-09 | + | + | |
| B/Brisbane/60/2008 | B | 2010-17 | + | + | |
| B/Massachusetts/2/2012 | B | 2014-15 | + | + | |
| B/Phuket/3073/2013 | B | 2015-16 | + | + | |
| Total B antigens | 8 | | 8 | 7 | 0 |
| Grand Total | 39 | | 35 | 26 | 2 |

### Example 3: Baseline and Post-Vaccination Anti-H3N2 IgG and IgA Levels are Associated with Influenza Disease

To assess whether IgG and IgA levels are associated with the risk to be symptomatically infected with influenza, the distributions of Ab levels between influenza negative and PCR-confirmed influenza positive subjects was compared. Since RT-PCR tests for influenza were only performed for individuals with influenza-like symptoms, only symptomatic influenza cases were identified. This comparison was done for both baseline and post-vaccination measurements in each of the treatment arms: placebo, TIV and LAIV. The analysis followed a pre-defined statistical analysis plan that defined two analysis tiers in order to preserve statistical power (see Table 3).

Since all of the infections in the case-control dataset were H3N2 positive and infected with the A/Wisconsin/67/2005 (Wis05) strain, the primary analysis focused on H3N2 antibody levels to the (1) Wis05 vaccine strain; and (2) H3N2 magnitude, which represents the immune-history antibody repertoire to subtype H3N2. The secondary tier analysis included responses to the H1N1 and B vaccine strains, as well as the overall magnitude to these subtypes (see Table 3). Statistical significance was determined using a logistic regression model adjusted for all baseline covariates.

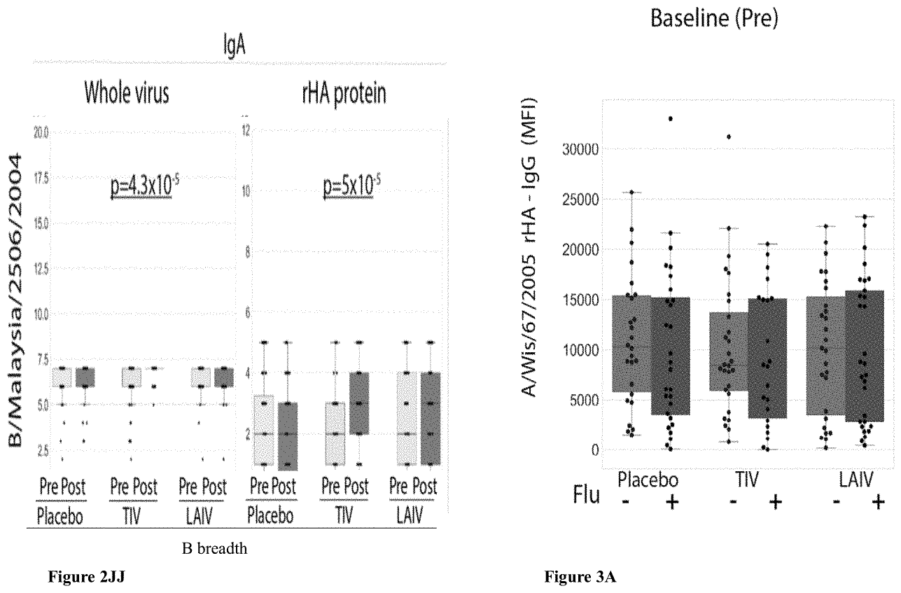

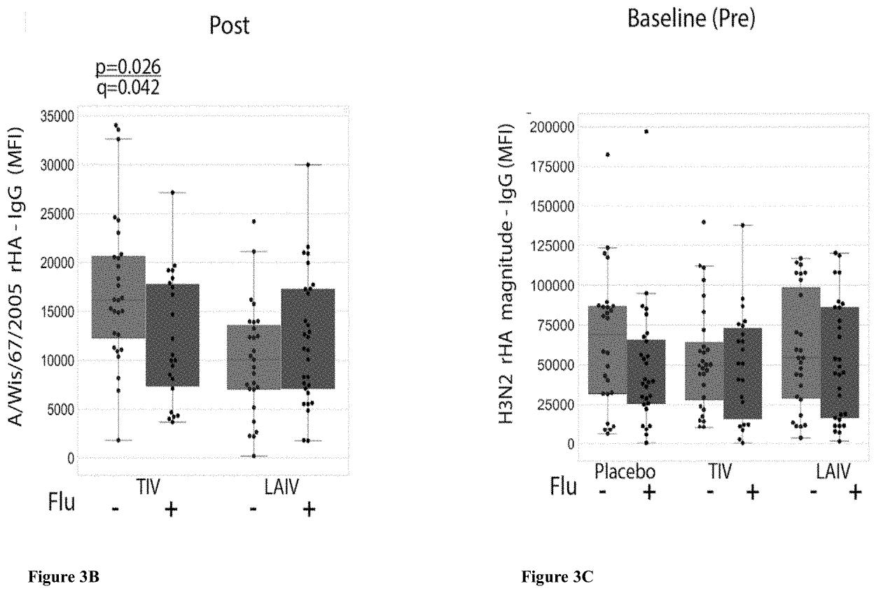

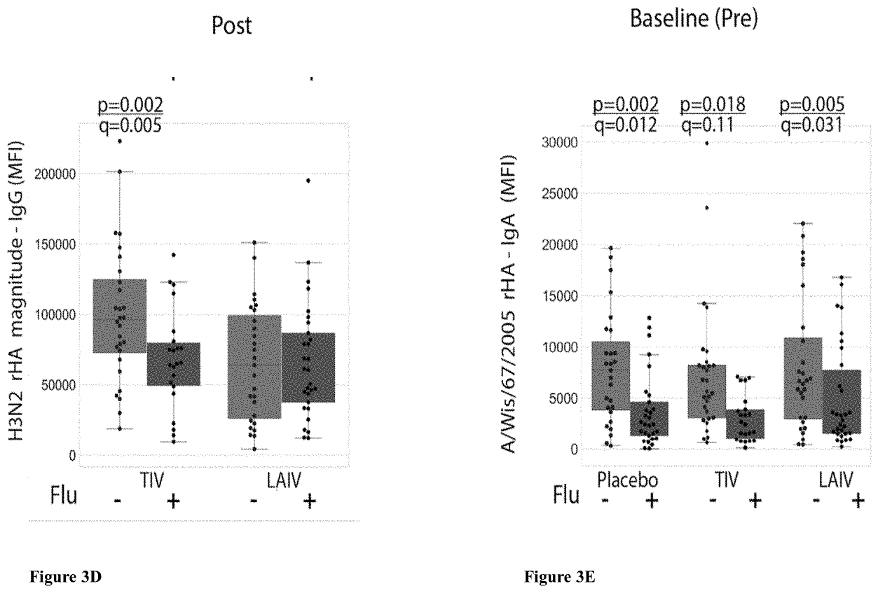

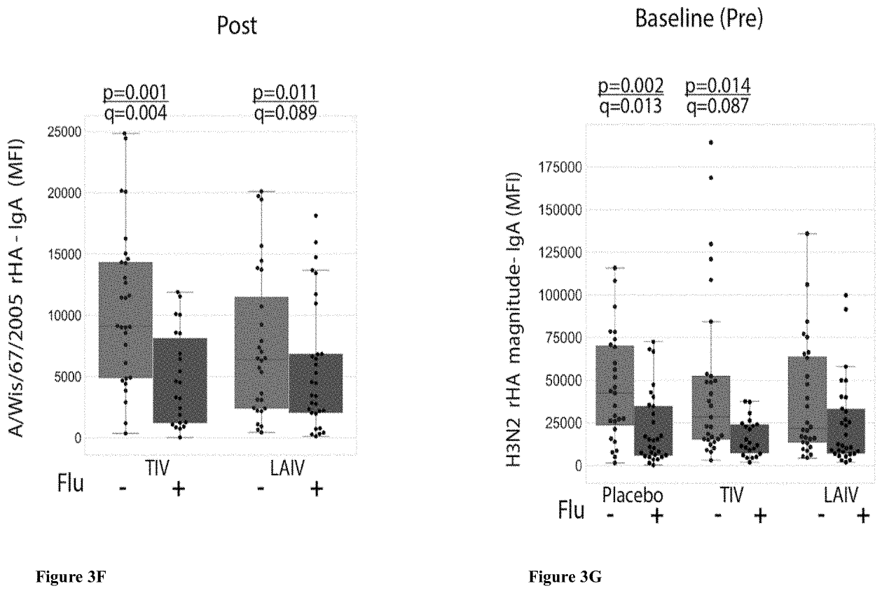

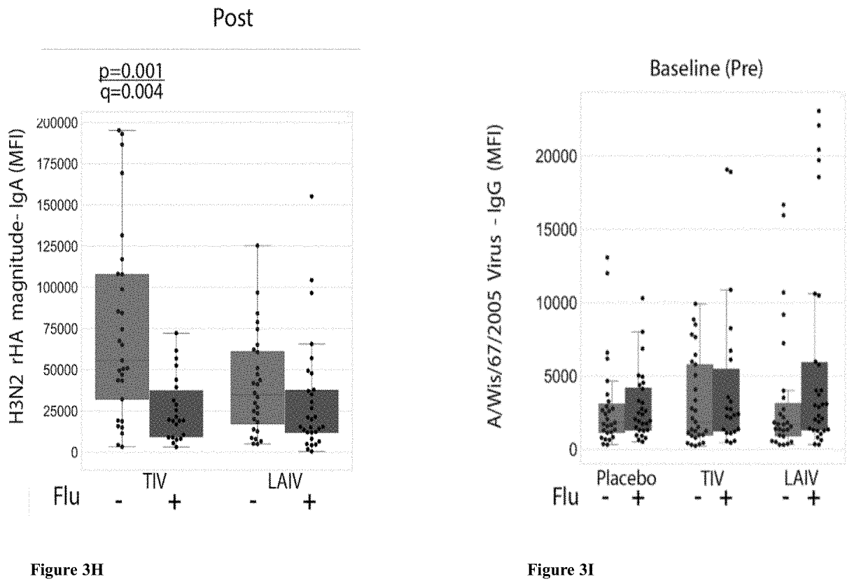

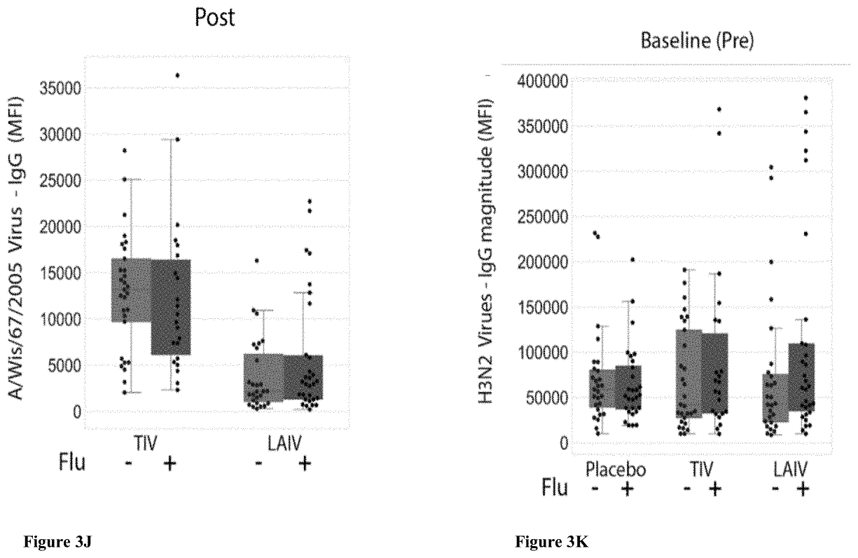

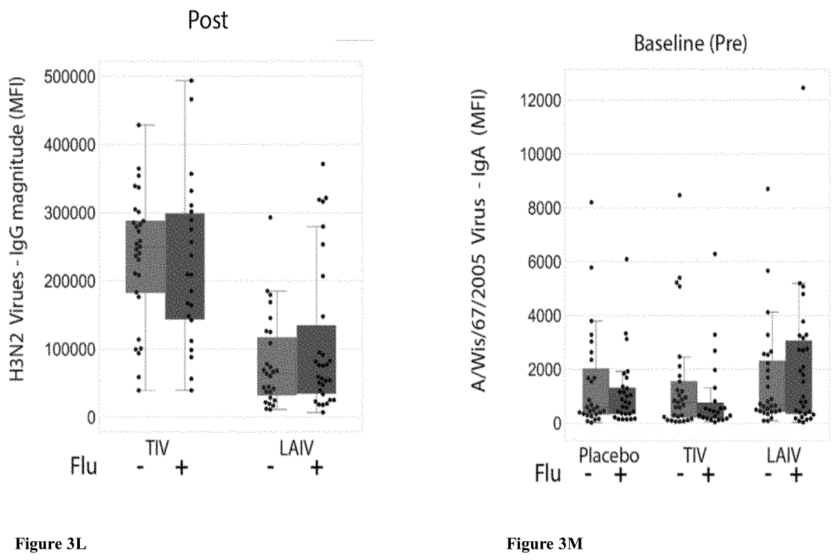

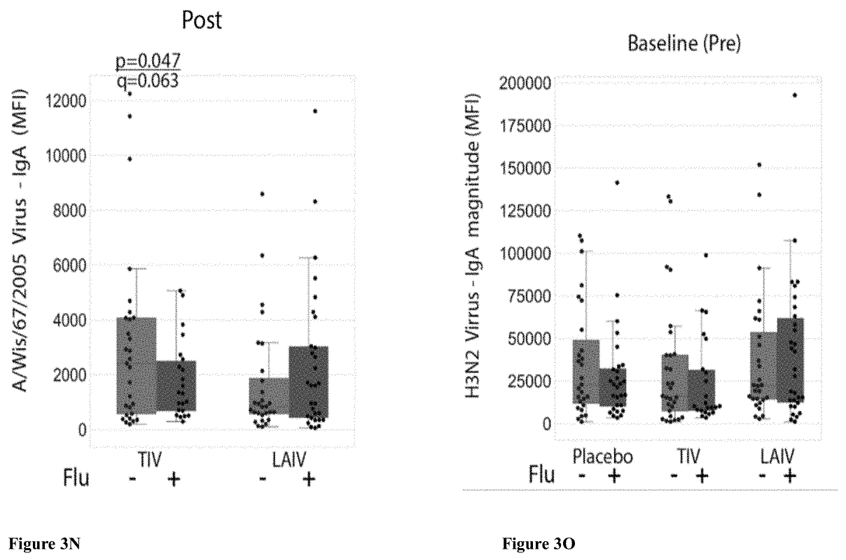

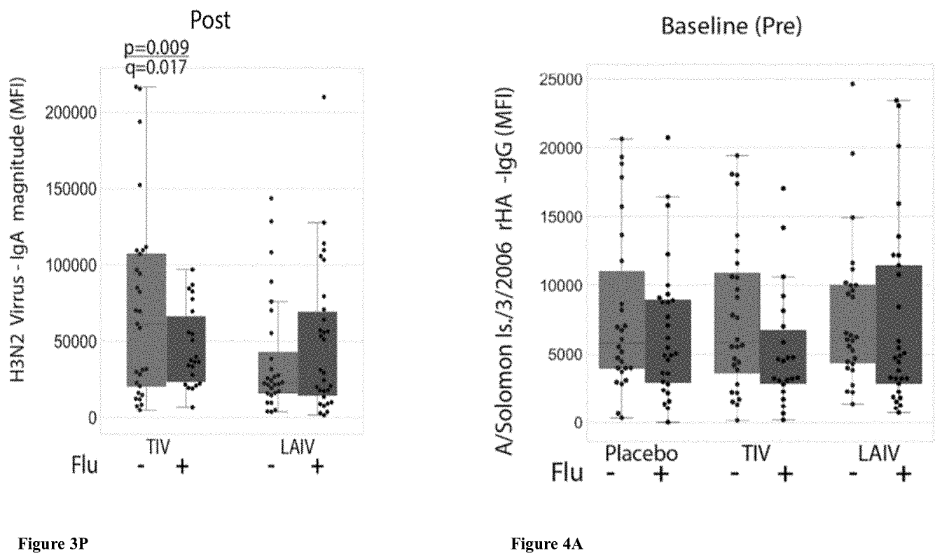

It was found that baseline IgG levels to H3N2 rHA of the vaccine strain (A/Wisconsin/67/2005) were not significantly associated with infection status in any of the groups (FIG. 3A). In contrast, post-vaccination levels were significantly associated in the TIV group only (p=0.026, q=0.042) (FIG. 3B). Similarly, IgG magnitude to H3N2 rHA proteins was not associated with influenza disease at baseline but was significantly associated only in the TIV group post-vaccination (p=0.002, q=0.005; FIG. 3C-3D). No significant associations were found between IgG antibody levels to H3N2 WIV vaccine strain or overall H3N2 WIV magnitude (FIG. 3I-3L).

Next, the associations of infection status with IgA antibody levels to the rHA of the H3N2 vaccine strain and total IgA magnitude to H3N2 rHA proteins were analyzed. Significant associations between infection status and baseline IgA levels to the H3N2 rHA protein of the vaccine strain were found in the placebo group (p=0.002, q=0.012), the TIV group (p=0.018, q=0.11) and in the LAIV group (p=0.005, q=0.031) (FIG. 3E). Post-vaccination IgA levels to Wis05 rHA were associated with infection status in both TIV and LAIV groups (TIV: p=0.001, q=0.004; LAIV: p=0.011, q=0.089; FIG. 3F). Similar significant associations were also observed for the IgA magnitude to H3N2 rHA antigens at baseline for the placebo group (p=0.002 q=0.013) and the TIV group (p=0.014, q=0.087), and were trending toward significance in the LAIV group (p=0.07) (FIG. 3G). The post-vaccination IgA magnitude to H3N2 strains was also significantly associated with infection status in the TIV group (p=0.001, q=0.004), and were trending toward significance in the LAIV group (p=0.09) (FIG. 3H). However, baseline IgA levels to the H3N2 WIV antigens were not associated with infection status, while both post-vaccination IgA level to Wis05 WIV and IgA magnitude to H3N2 whole viruses were associated with influenza disease only in the TIV group (Wis05 WIV: p=0.047 q=0.063, FIG. 3N; IgA magnitude to H3N2 viruses: p=0.009 q=0.017, FIG. 3P).

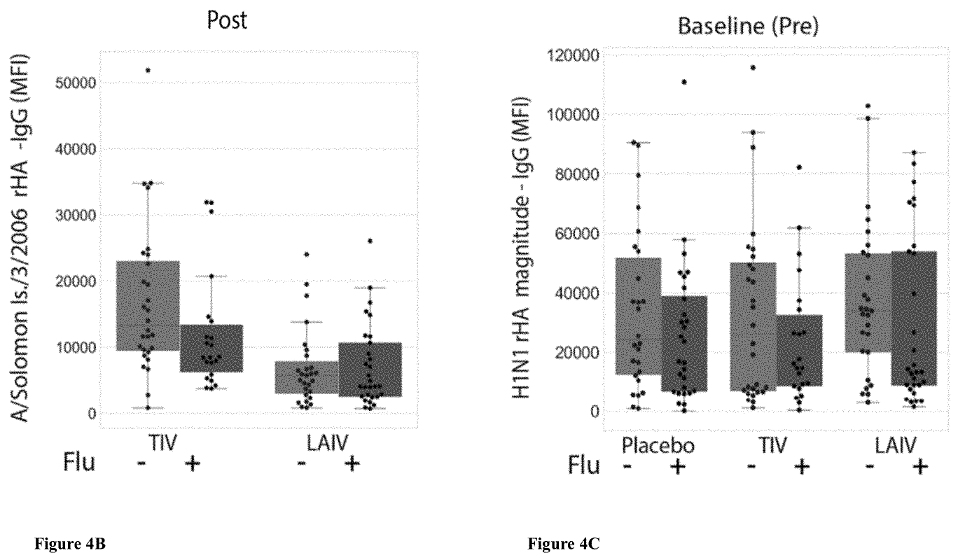

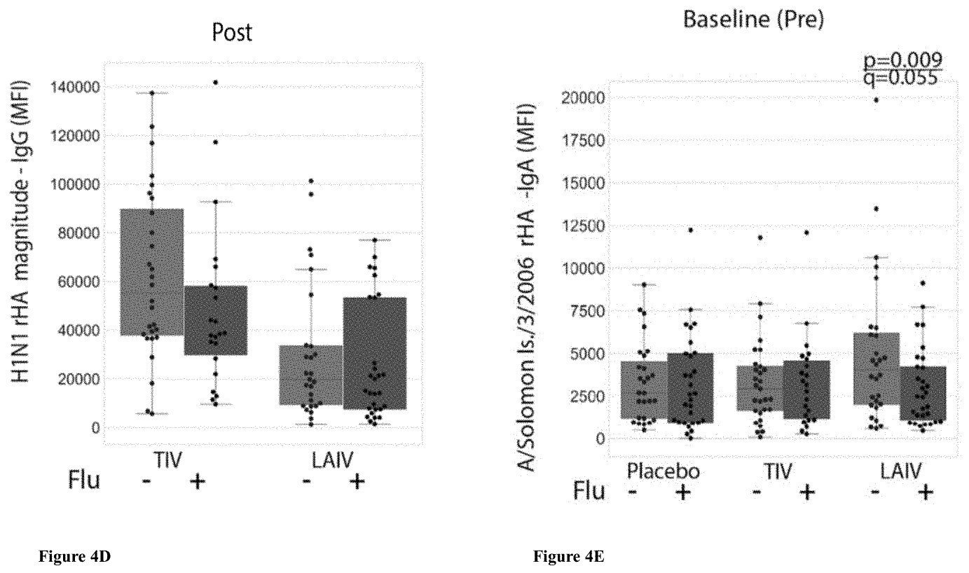

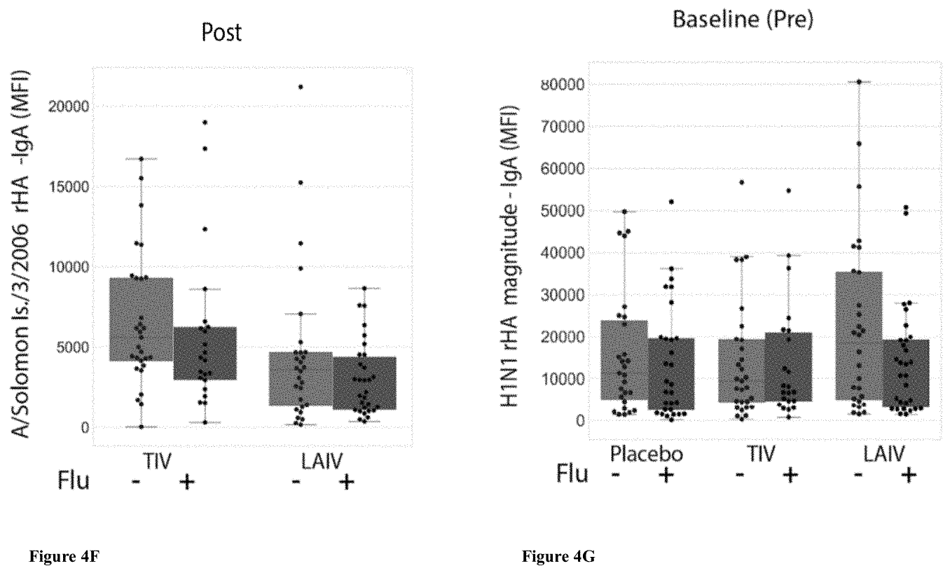

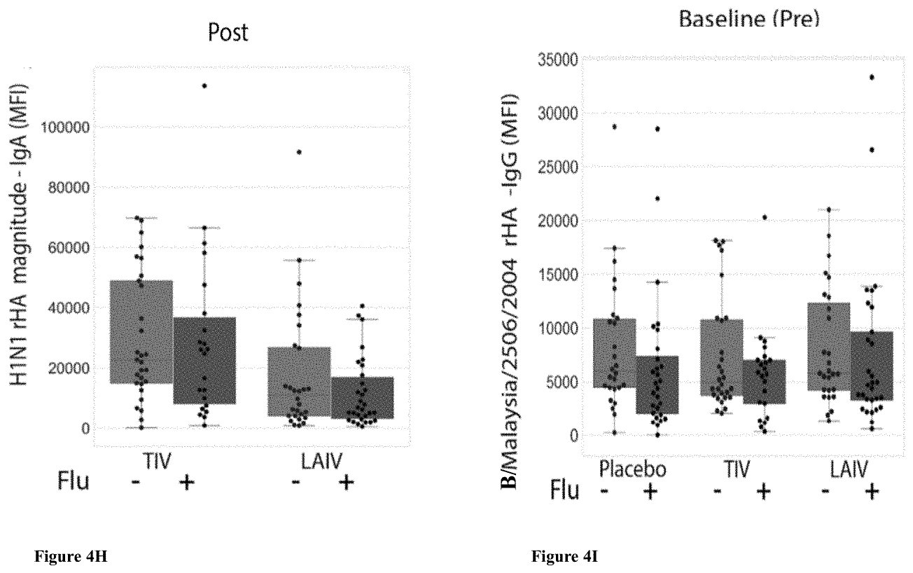

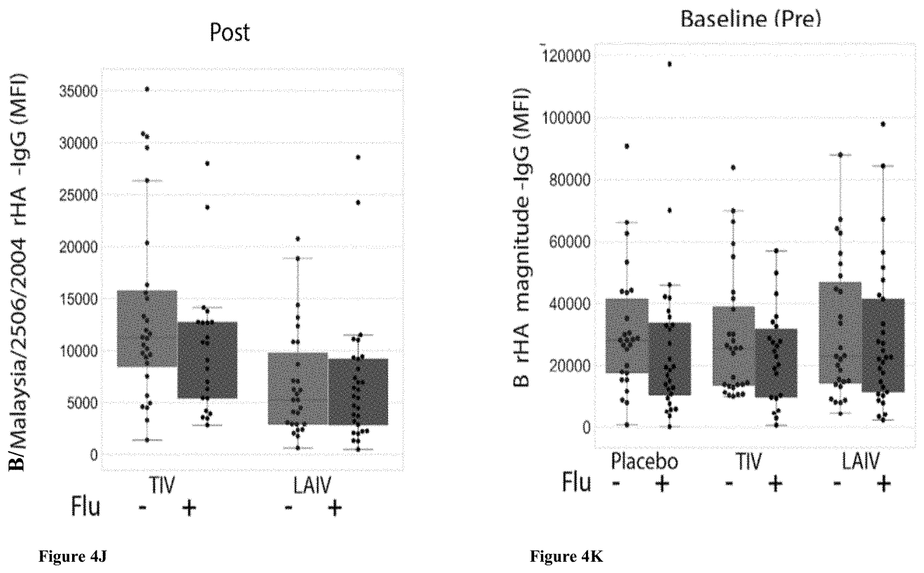

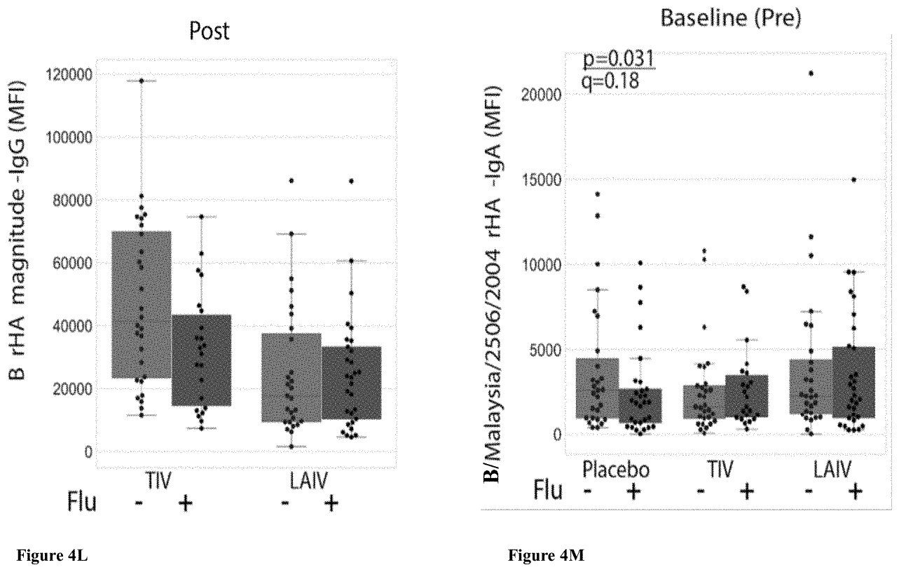

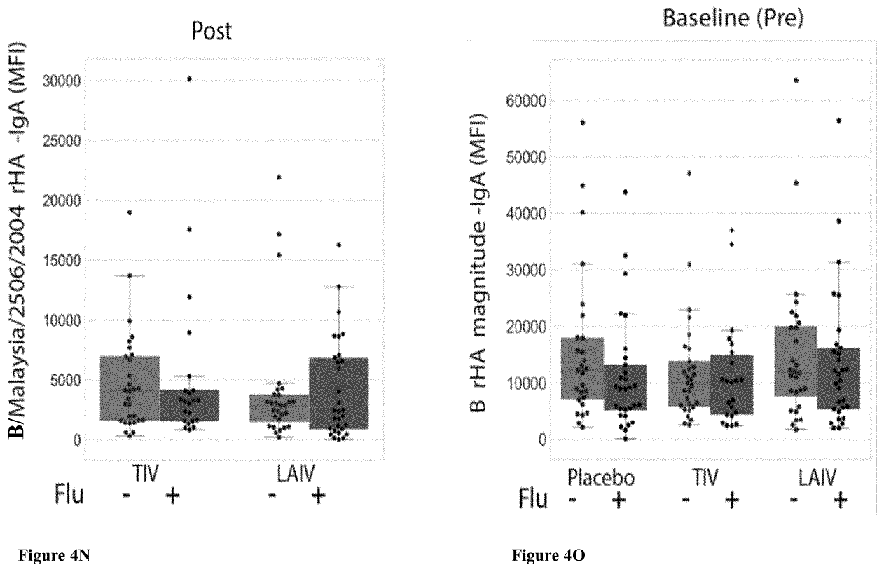

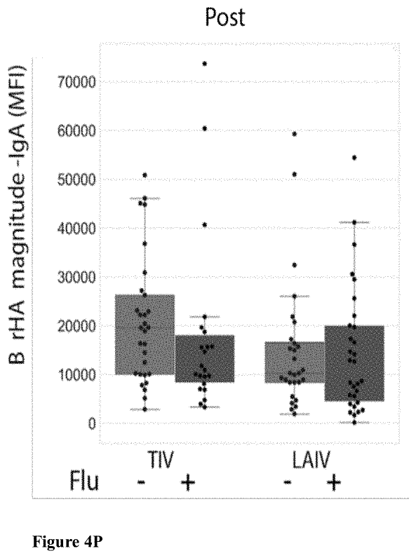

The analysis of all secondary tier variables which included the IgG and IgA binding to rHA proteins of the H1N1 and B vaccine strains and magnitude to H1N1 and B subtype rHA antigens (FIG. 4A-4P), identified only a single association with influenza disease—IgA levels to the H1N1 rHA vaccine strain (p=0.009 q=0.055, FIG. 4E). No significant association between antibody levels to H1N1 and B WIV antigens with influenza disease were found (data not shown).

### Example 4: Baseline IgA and IgG Immune History Profiles Exhibited Marked Differences

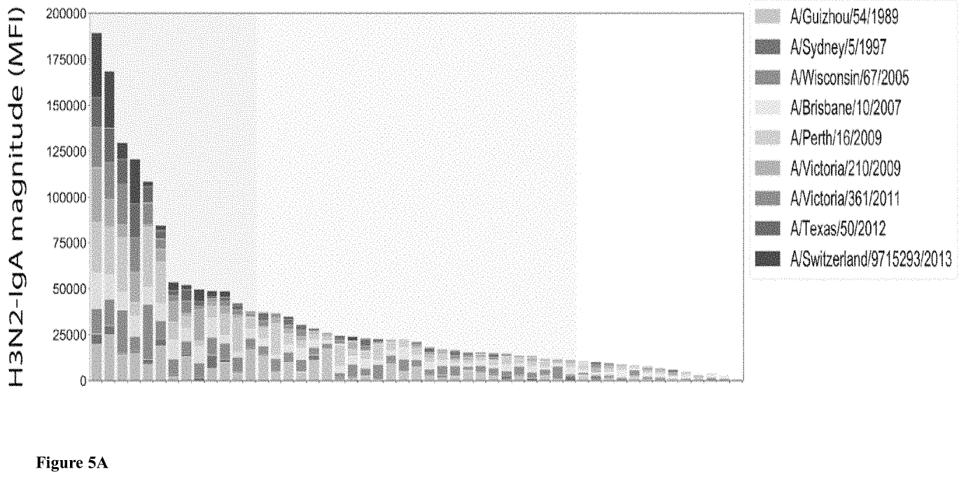

The magnitude of antibodies to subtype antigens demonstrated extensive heterogeneity. For example, while some individuals from the TIV group had high baseline IgA levels to rHA proteins of many H3N2 strains, others had very low baseline IgA levels to the same antigens (FIG. 5A). We defined a magnitude of antibodies to a certain type of antigen (rHA or WIV or peptides) from a certain influenza subtype as a feature of the baseline immune-history (BIH) profile of the individual (for example, IgA-BIH to H3N2 rHA proteins). Since significant associations were only found between baseline IgA antibody levels and influenza disease, but not IgG antibody levels, the IgG and IgA baseline immune-history (BIH) antibody profiles of each individual subject were further investigated. First, all subjects from the TIV group were ranked by their IgA-NIH to H3N2 rHA proteins and divided into quartiles: subjects in the lowest quartile were denoted as low-BIH-1 group; subjects in the highest quartile were denoted as high-BIH group and the remaining two middle quartiles were denoted as the mid-BIH group. This ranking was generally retained post-vaccination (FIG. 5B-5D) as suggested by the relatively high correlations between baseline and post-vaccination IgG and IgA for H3N2 antigens magnitude responses (FIG. 2Y-2Z). Four representative subjects were selected from the IgA low-, mid- and high-BIH groups and their IgG and IgA immune history antibody profiles were plotted (FIG. 5B-5D). Significant differences in the IgA vs. IgG anti-influenza antibody repertoires were observed within subjects; some subjects which were ranked as low IgA-BIH had high IgG levels (e.g., subjects 80385, 40862 and 40703 in FIG. 5B), while others which were ranked as high IgA-BIH had low IgG levels (e.g., subject 40779 in FIG. 5D). Furthermore, some subjects had low IgA levels across all three subtypes (e.g., subjects 40862, 80385 in FIG. 5B), while others had low levels for H3 strains, but high levels for the H1 and B subtypes post-vaccination (e.g., subject 80392, in FIG. 5B). Comparing the baseline and post-vaccination antibody profiles of subjects, it was observed that in most cases the post-vaccination antibody repertoires were similar in shape to those observed at baseline, suggesting that the majority of vaccine-induced antibodies boosted their baseline memory repertoire (e.g., subjects 40878 and 80392, in FIG. 5B-5C). However, in some subjects, changes to the repertoire were observed in both magnitude and breadth (e.g., subjects 71065 and 40669, in FIG. 5C). Some subjects generated a broad IgG response across all subtypes, but a narrower IgA response post-vaccination (e.g., subject 90379, in FIG. 5D). Interestingly, all four subjects that were subsequently infected (subjects 40862, 80385, 40882 and 40878, in FIG. 5B-5C) had low or mid IgA BIH to the H3 subtype, but high IgG MEI to the H3 subtype.

### Example 5: Baseline and Post-Vaccination Anti-H3N2 IgA Profiles are Correlates of Risk for Influenza Disease

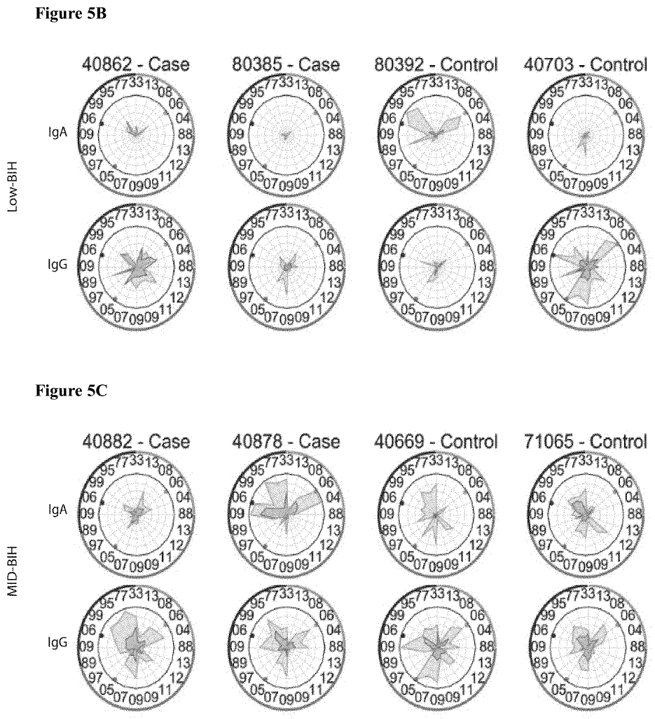

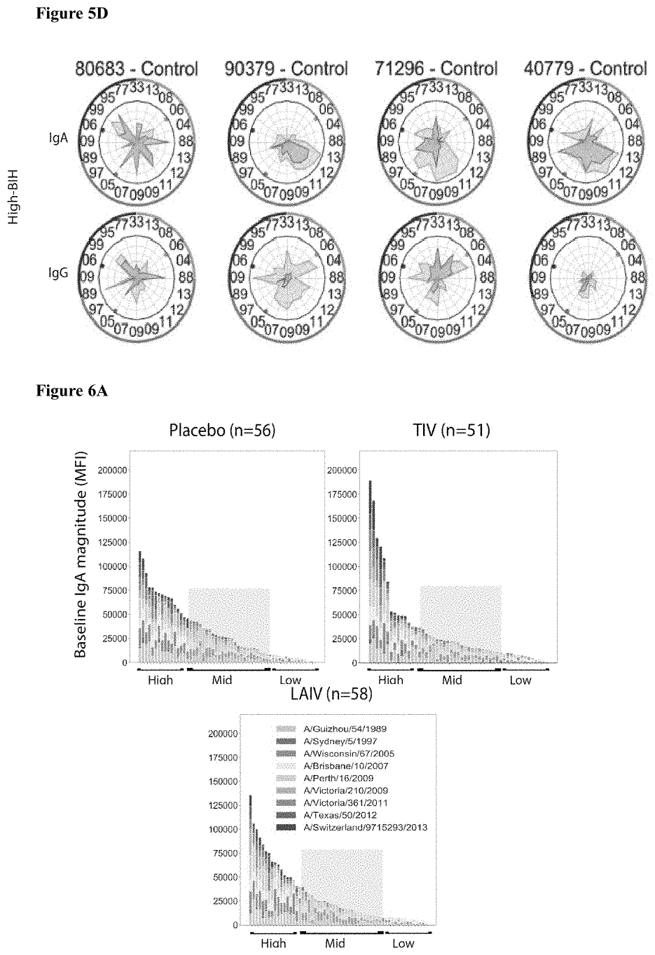

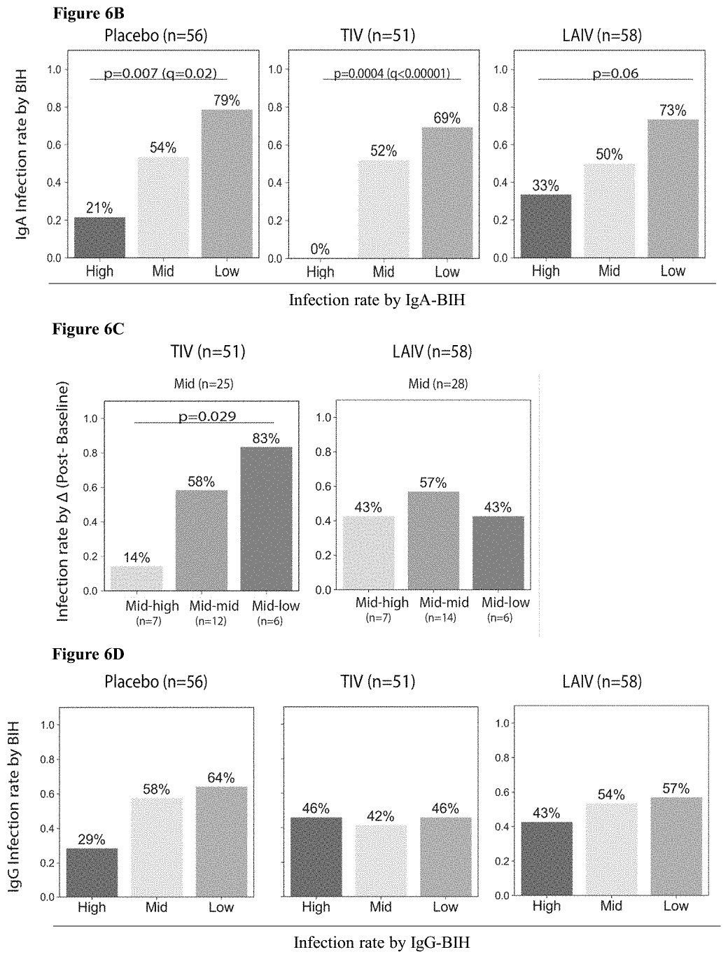

Based on the significant associations between baseline rHA H3N2 IgA levels to the vaccine strain and overall magnitude, it was further asked whether the risk for influenza disease is associated with the subject's baseline immune-history (BIH) rank according to the magnitude of antibodies binding to H3N2 rHA proteins. For that reason, subjects in the placebo and LAIV group were also ranked by their IgA baseline immune history to H3N2 rHA (FIG. 6A). The statistical comparisons between the infection rates in the low and high BIH groups were computed using a logistic regression analysis adjusted for baseline covariates. Significant differences in the infection rates were observed between low and high IgA-BIH quartiles; in the placebo group low-BIH subjects had a 4 times higher risk to be symptomatically ill (79% vs. 21%, p=0.007, q=0.02) while in the TIV group none of the high-BIH subjects had influenza disease (69% vs. 0%, p=0.0004, q<0.00001). Similar trends were observed in the LAIV group, but these were not significant (73% vs. 33%, p=0.06) (FIG. 6B).

Since mid-BIH individuals in the TIV and LAIV groups had a 52% and 50% baseline infection rate respectively (FIG. 6B), it was asked whether ranking these individuals based on their post vaccination antibody levels would also be associated with risk of infection. Therefore, these subjects were further divided into a mid-low-BIH, mid-mid-BIH and mid-high-BIH subgroups based on quartiles. Significant differences were found in the infection rates between the mid-high and mid-low BIH subjects in the TIV group (83% vs. 14%, p=0.0029, q-val not significant). No significant difference in infection rate was observed between the LAIV mid-high and mid-low BIH quartiles (FIG. 6C). When ranking subjects based on their IgG-BIH to H3N2 rHA proteins, it was found that subjects in the low-BIH placebo group were 2.2 times more likely to become infected than subjects in the high-BIH placebo group, but this difference was not-significant (29% vs. 64% in high vs. low-BIH respectively) (FIG. 6D). Insignificant differences in infection rates for IgG levels were also observed in the TIV and LAIV groups (FIG. 6D).

### Example 6: Baseline IgA Levels to the H3N2 A/Wisconsin/67/2005 Vaccine Strain are Correlates of Risk for Influenza Disease

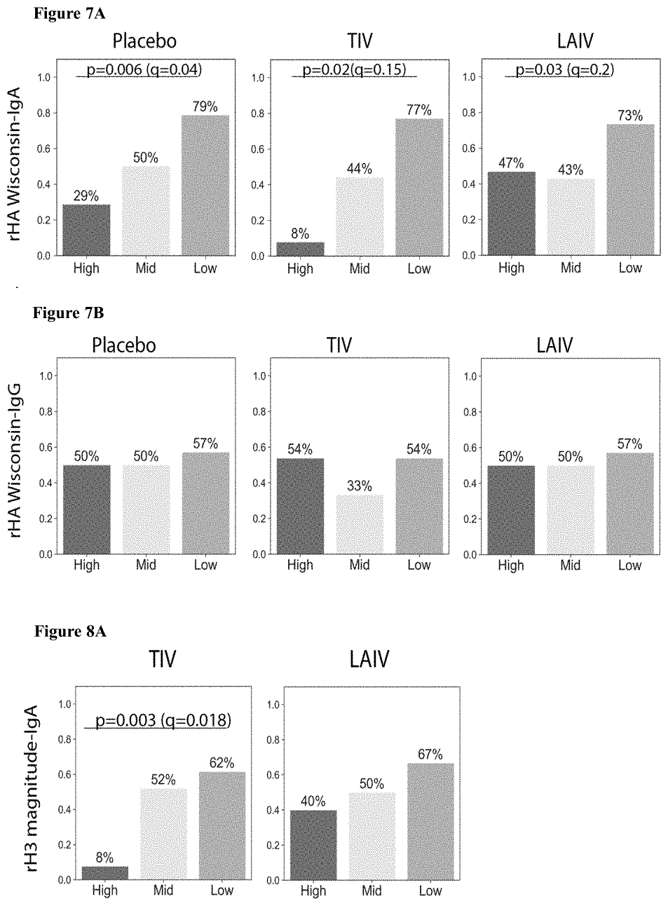

Subjects were also ranked based on their IgG and IgA baseline antibody levels to the A/Wisconsin/67/2005 H3N2 vaccine strain. Subjects were divided into quartiles, as previously described, and the infection rates in the low- and high-BIH groups were compared within each treatment arm. Similar to the findings for overall IgA magnitude to H3N2 rHA proteins (FIG. 6B), it was found that subjects in the placebo group with low-BIH IgA levels had 2.5 times higher infection rate compared to subjects in the placebo high-BIH IgA quartile (79% vs. 29%, p=0.006, q=0.04). The infection rate in the low-BIH subjects in the TIV group was 9.6 times higher than the in TIV high-BIH subjects (77% vs. 8%, p=0.02, q=0.15). A similar trend was observed in the LAIV group (73% vs. 47%, p=0.03, q=0.2,) (FIG. 7A). No significant differences in infection rates were found between the low and high IgG-BIH quartiles in any of the three treatment arms (FIG. 7B).

### Example 7: Post-Vaccination IgA and IgG Antibody Levels are Correlates of Risk for Influenza Disease

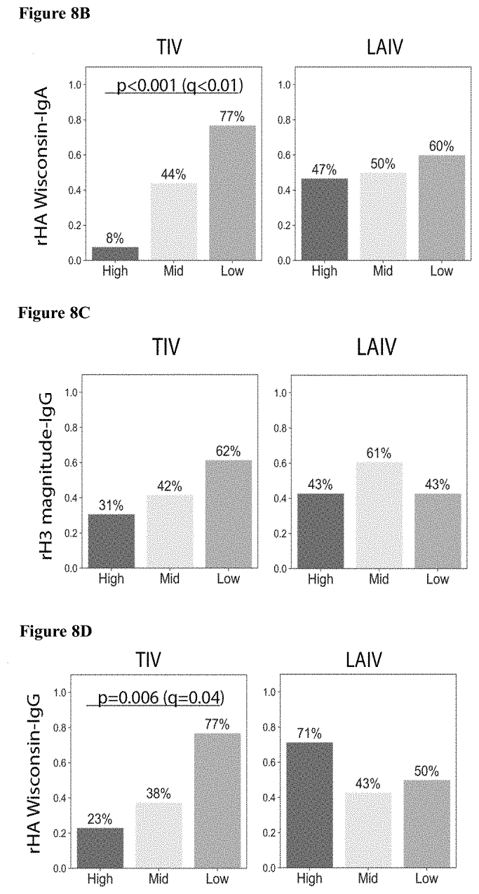

Next, subjects were ranked based on their post-vaccination antibody levels to the H3N2 A/Wisconsin/67/2005 vaccine strain or post-vaccination overall magnitude to H3N2 antigens, to test whether post-vaccination anti-H3N2 antibody profiles were associated with infection rates. Following ranking, the subjects were divided to quartiles, as described above: the quartile with highest post-vaccination antibody levels was termed high-responders, and the quartile with lowest antibody levels was termed low-responders. It was found that ranking subjects by post-vaccination IgA magnitude to H3N2 rHAs was significantly associated with infection rate in the TIV group, with a 7.7 times higher infection rate for low-responders as compared to high-responders (62% vs. 8% p=0.003, q=0.0018, FIG. 8A). However, ranking by post-vaccination IgG magnitude to H3N2 rHAs was not significantly associated with infection rate (FIG. 8C). Post-vaccination antibody levels to A/Wisconsin/67/2005 rHA prtoein were significantly associated with infection risk in the TIV group, for both IgA (77% vs. 8%, p<0.001, q<0.01, FIG. 8B), and IgG (77% vs. 23%, p=0.006, q=0.04, FIG. 8D). Baseline and post-vaccination H3N2 antibody levels to all of the WIV antigens were not associated with infection rates (data not shown).

In summary, it was found that the rise in antibody levels post-vaccination was significantly higher in the TIV group as compared to the LAIV group, with the exception of IgA responses to whole inactivated viruses of the H1N1 and B vaccine strain. This is in agreement with the main findings of the FLUVACS study, which reported significant vaccine efficacy in the TIV group (VE=72%, 95% CI 49 to 84) and non-significant vaccine efficacy in the LAIV group (VE=29%, 95% CI 14 to 55).

Post-vaccination rises were significantly correlated with baseline antibody levels for all three subtypes, highlighting the important role of immune-history in shaping the post-vaccination antibody repertoire. Correlations were stronger in the LAIV group, most likely due to the weak overall responses this vaccine elicited.

The baseline and post-vaccination IgA and IgG levels were compared in case-control individuals and a significant association for baseline and post-vaccination IgA levels with risk of infection were found. This association was stronger for the H3N2 A/Wisconsin/67/2005 vaccine strain, which was also the infection strain in this trial, and H3N2 recombinant HA magnitude in the placebo and TIV group; compared to antibodies to the H1N1 vaccine strain of, which were only baseline correlated in the LAIV group, and antibodies to B, which were only baseline correlated in the placebo group. Surprisingly, IgG binding to H3N2 vaccine strain and magnitude of IgG to H3N2 strains were not associated with risk of infection.

Importantly, it was shown that ranking subjects based on their IgA H3N2 magnitude to recombinant HA proteins at baseline, exhibited an extensive heterogeneity in the baseline immune-history profiles of subjects for both IgG and IgA antibody responses, which is likely driven by both the exposure history, as well as the underlying host immunogenetics. The ranked subjects were divided to low-, mid- and high-baseline immune history groups and individual's IgA and IgG profiles were visualized. While some individuals had broad and potent antibody profiles at baseline, others had almost no influenza specific antibodies to some or all influenza subtypes.

The percentile infection rates were calculated for each of the low-mid-high-baseline immune-history groups and found that subjects with low IgA baseline immune-history against H3N2 strains had significant increase in the infection rates than those with high IgA baseline immune-history in the placebo group (79% vs. 21%) as well as the TIV group (69% vs. 0%). Furthermore, analyzing the post-vaccination antibody levels of the mid-BIH individuals in the TIV and LAIV groups, which both had about 50% infection rate at baseline, showed that low-mid-baseline immune-history subjects had higher infection rates than high-mid-baseline immune-history subjects in the TIV group only.

Taken together, these results demonstrate that individuals with low baseline IgA level, as well as those with moderate IgA level, but fail to generate a robust post-vaccination response, are at higher risk of influenza infection. The finding that IgA immune-history was a correlate of risk, while IgG immune-history not, can be supported by the observations regarding the significant differences between the IgA and IgG profiles of each subject. They imply that serum anti-HA-IgA antibody levels may provide an independent correlate of risk for influenza disease. This is novel serologic evidence for the heretofore unknown contribution of IgA in influenza virus protection.

### Example 8: Obesity Study

To compare antibody repertoires of obese and healthy-weight (HW) subjects, serum samples were obtained from a cohort of 100 obese (BMI≥30) and 89 HW (19≤BMI<25) subjects that were vaccinated with the trivalent seasonal vaccine (TIV) in 2010-2011 (Table 7). Serum samples from each subject were collected pre-vaccination (baseline) and one month (28-35 days) post-vaccination. The cohort included 150 adults and 39 elderly subjects (>65 years of age). The seasonal vaccine strains used in the study were A/California/7/2009 (Cal09, H1N1), A/Perth/16/2009 (H3N2) and B/Brisbane/60/2008. To profile both the IgG and IgA anti-influenza antibody repertoire in the serum of each subject at baseline and post-vaccination, two types of antigen microarrays were utilized in tandem: (1) influenza virus and proteins (influenza VP) microarray; and (2) a Cal09 peptide microarray (SEQ ID NOs: 200-399). The VP microarray was spotted with a panel of 34 whole inactivated viruses and 23 recombinant HA protein (rHA) antigens from A/H1N1, A/H3N2 and B influenza strains spanning 1933 to 2013 (Table 6). Twenty-five out of 34 (74%) of the whole virus strains and 18/23 (78%) of the rHA represented seasonal influenza vaccines spanning 1984 to 2017. This panel provided broad coverage of the antigenic diversity and evolution of influenza strains over 30 years and was used to study the vaccine effect on the level of antibodies to the 2010-2011 vaccine strains and to other strains from the same subtypes. Since obesity was previously correlated with increased rate of complications, hospitalization and mortality during the 2009 A/H1N1 pandemic, and the pandemic Cal09 strain was included in the 2010-11 vaccine, vaccine-induced antibodies to linear epitopes of A/California/07/2009 (Cal09) were further studied using a peptide microarray including 20mer amino acid (aa) peptides spanning the Cal09 HA and NA proteins with 15aa overlap.

TABLE 7
Demographics of all participants

| | | | | Gender | | |
|---|---|---|---|---|---|---|
| group | N | BMI | Age | M/F | Race | |
| Obese | 100 | 30-46.9 | 20-82.6 | 42/58 | Caucasian | 55% |
| | | (34.6 | (54.3 | | African American | 43% |
| | | median) | median) | | Hispanic | 1% |
| | | | | | Asian | 1% |
| Healthy- | 89 | 19-24.9 | 19-87 | 35/54 | Caucasian | 76% |
| Weight | | (22.7 | (57.0 | | African American | 15% |
| | | median) | median) | | Hispanic | 2% |
| | | | | | Asian | 7% |

### Example 9: Baseline Levels of IgG Antibodies, but not IgA Antibodies, Against 2010-2011 Seasonal Vaccine are Higher in Healthy-Weight Subjects

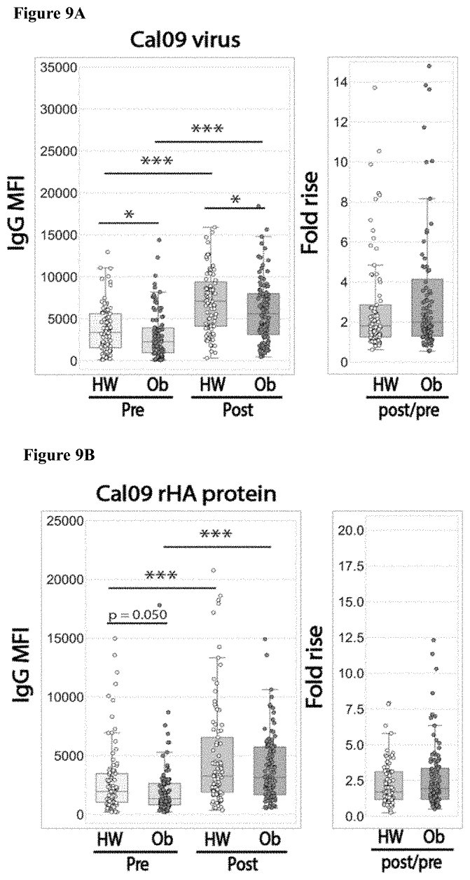

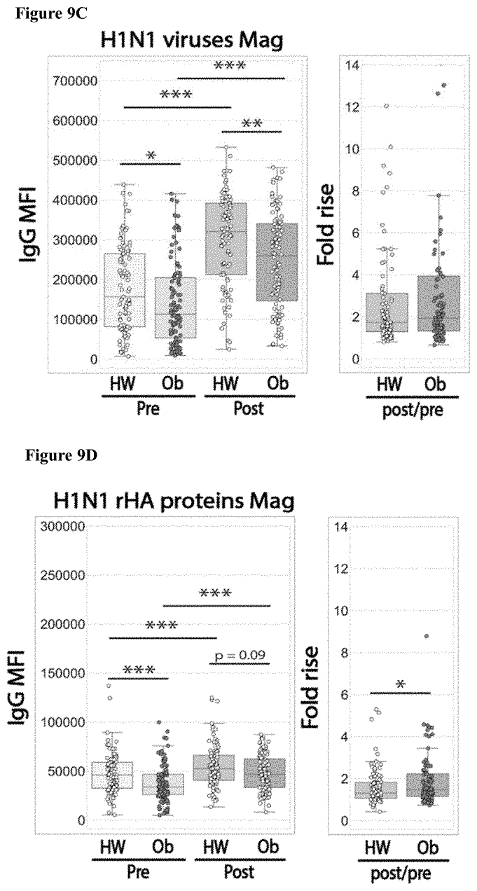

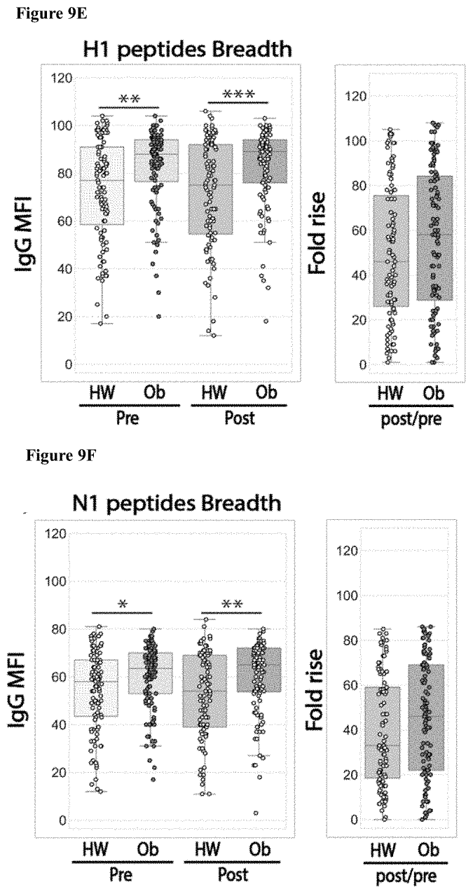

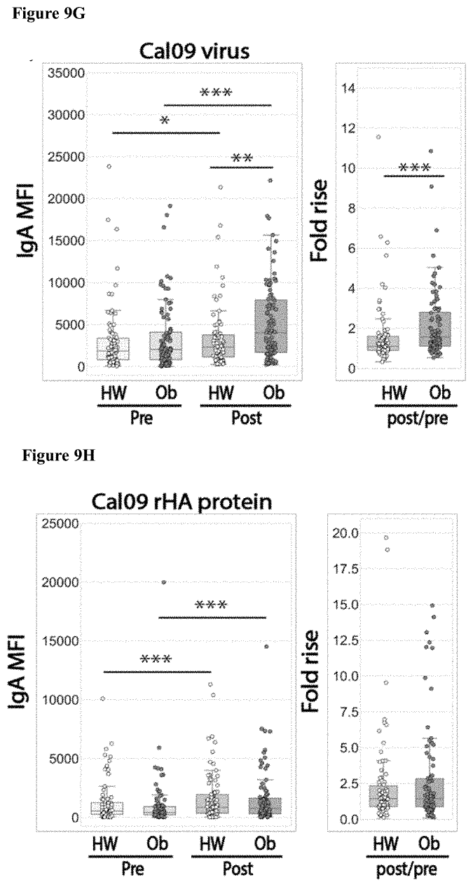

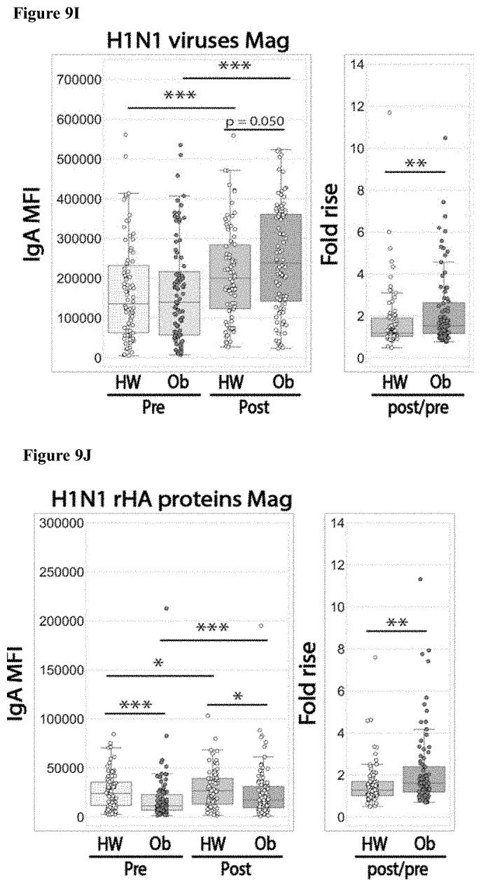

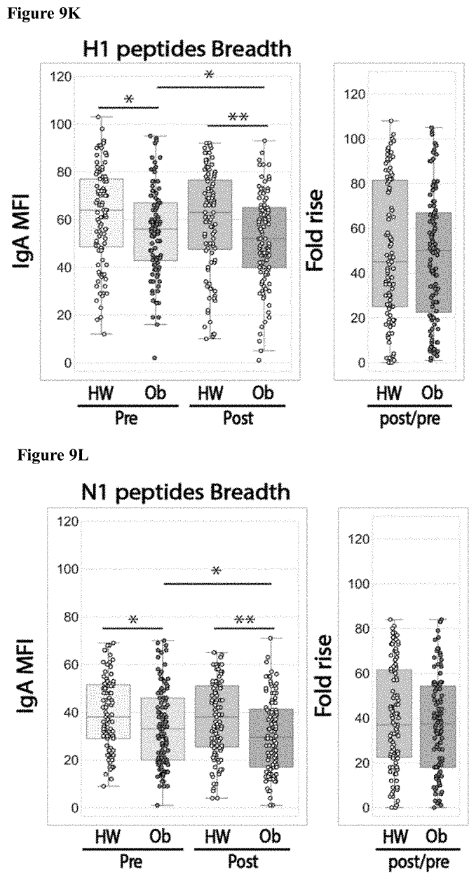

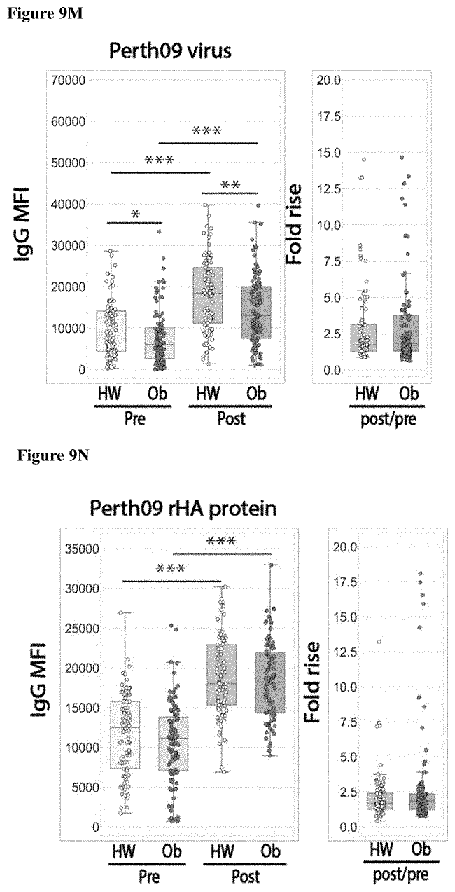

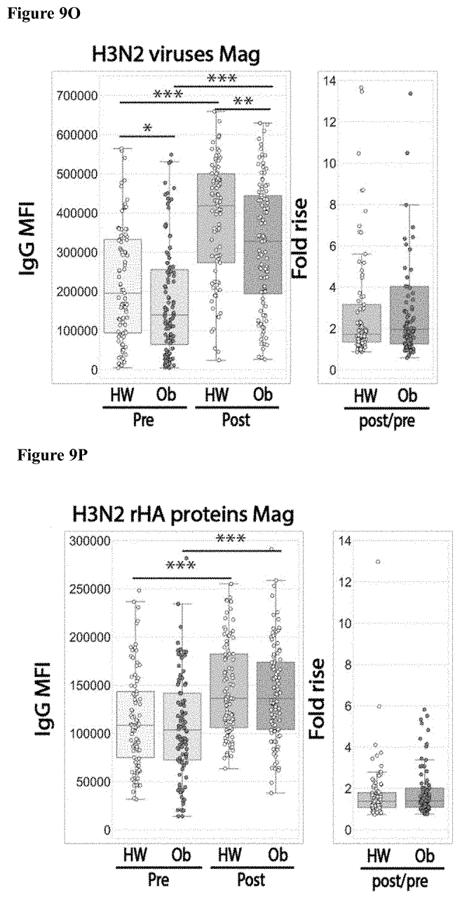

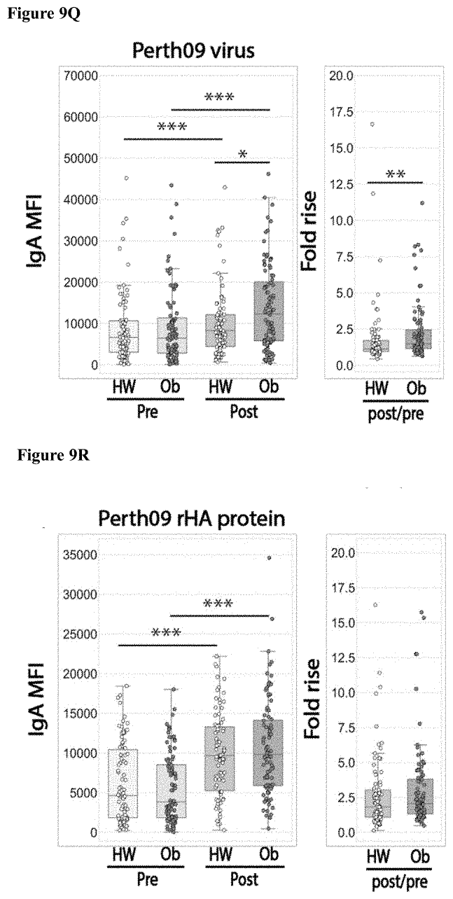

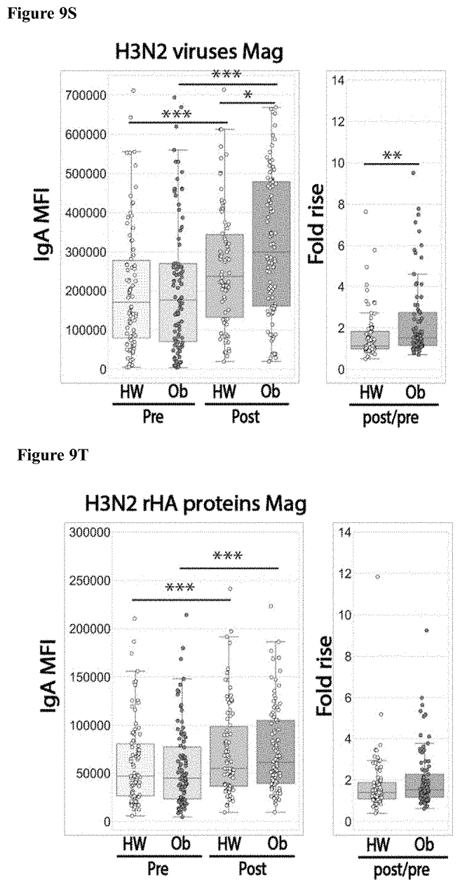

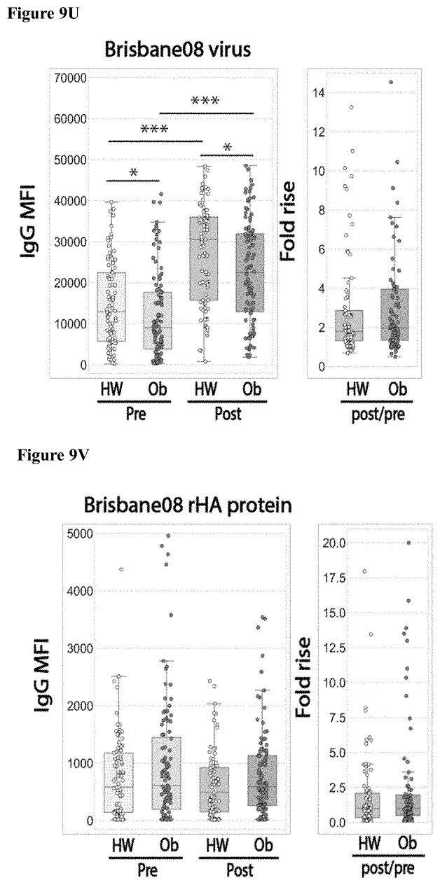

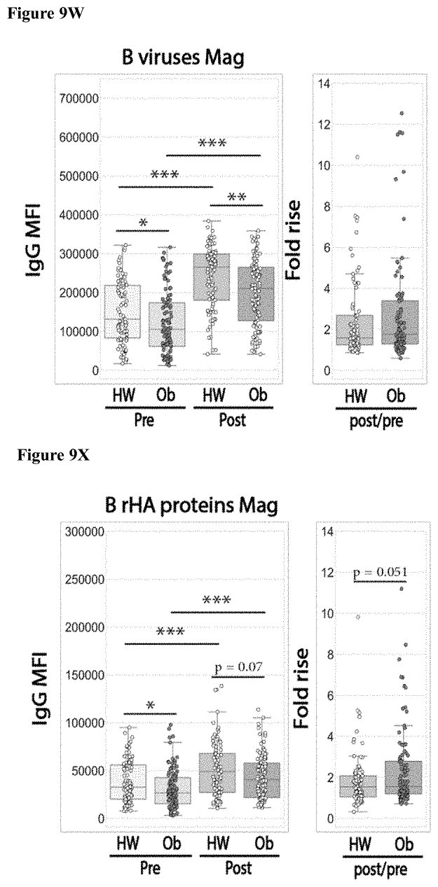

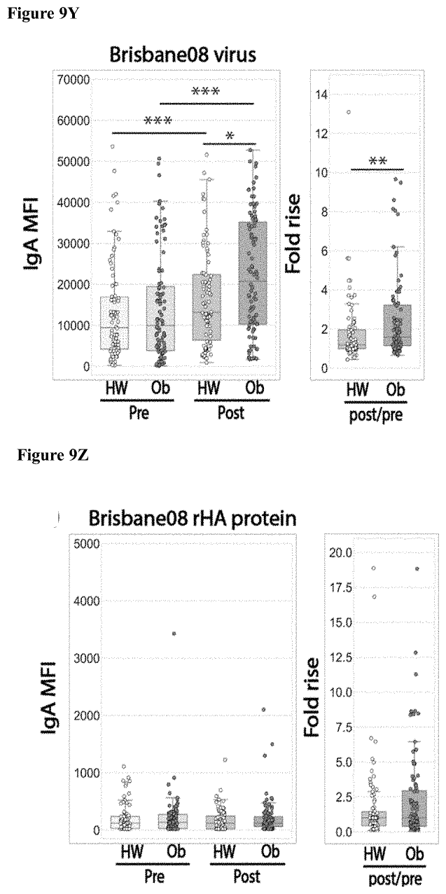

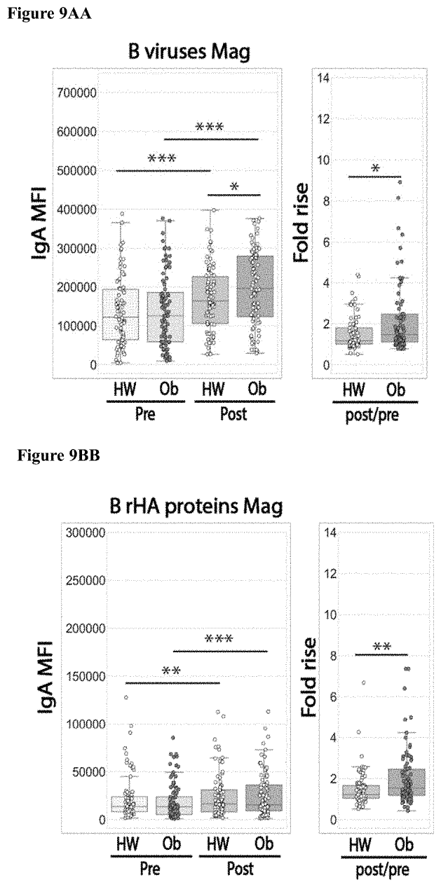

Obese subjects had significantly lower IgG levels to whole virus antigens of all three vaccine strains (p<0.05) at baseline (FIG. 9A, 9M, 9U). While IgG levels to the rHA antigens of the H3N2 and B strains were similar in the obese and HW groups (9N, 9V), the baseline IgG levels to Cal09 rHA were marginally lower in the obese group (p=0.051; FIG. 9B). In contrast to these findings, there was not detected any significant differences between the baseline IgA antibody levels to the three vaccine strains, both for whole viruses and rHA antigens of these strains (FIG. 9E-9F, 9Q-9R, 9Y-9Z).

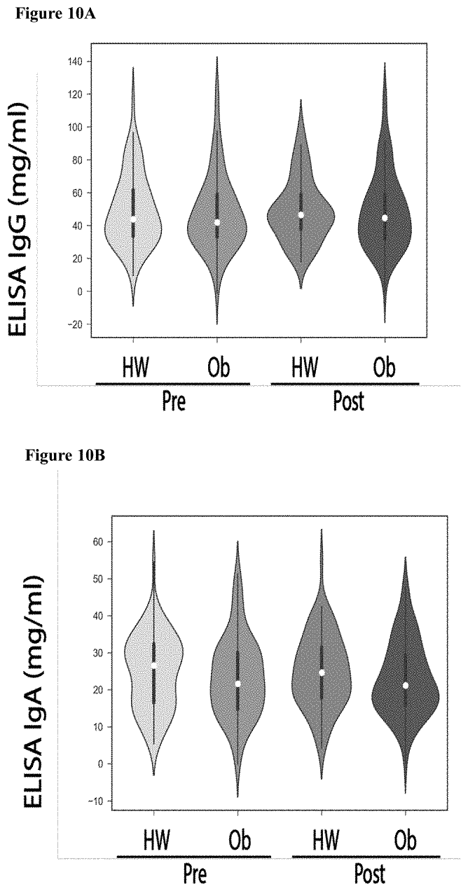

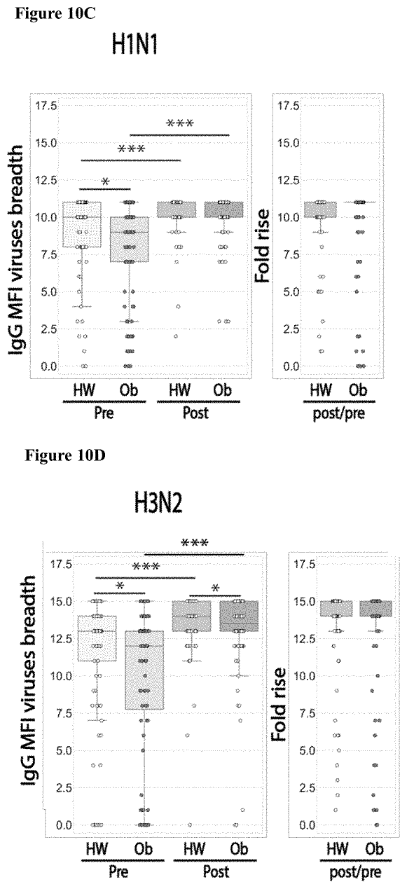

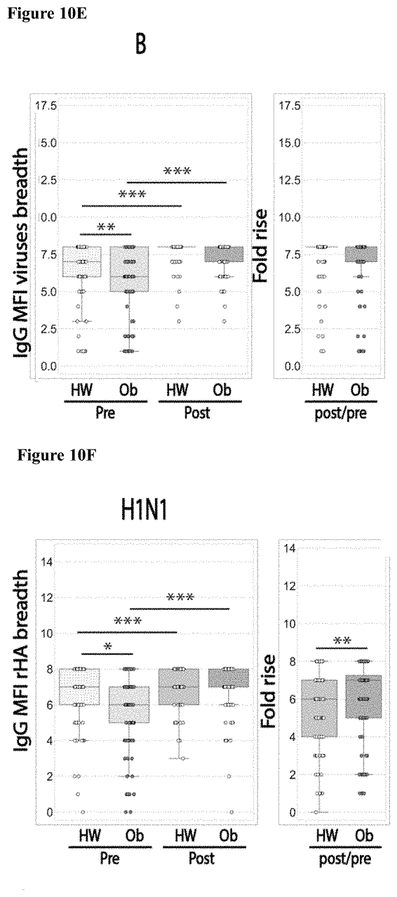

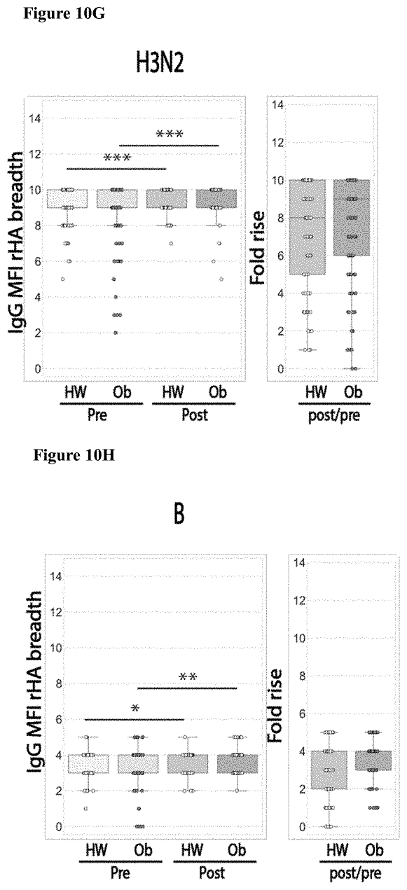

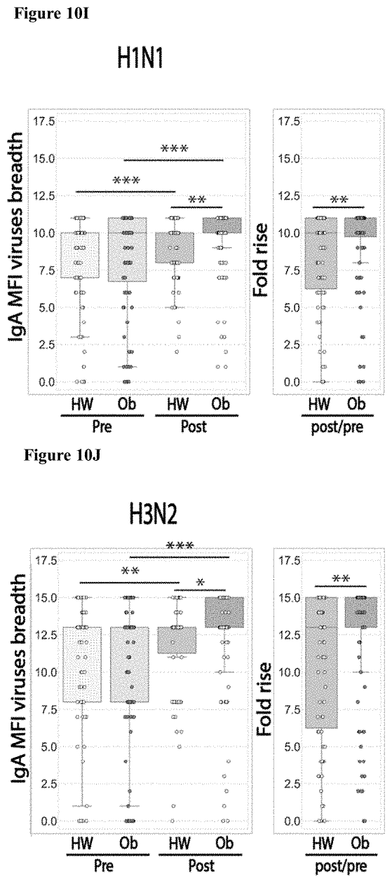

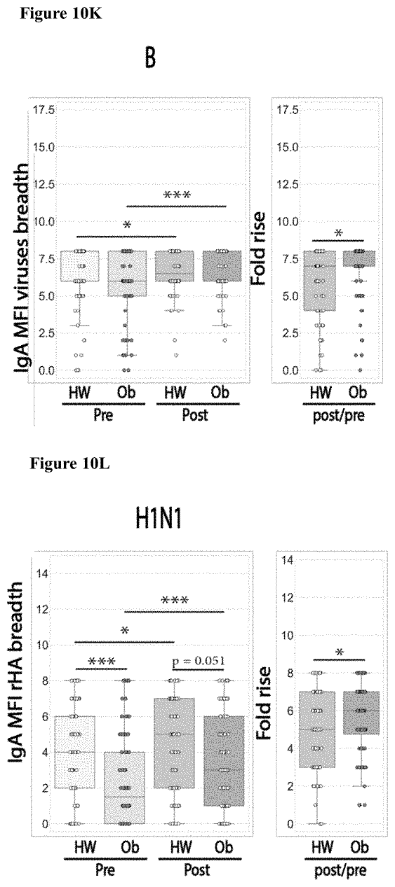

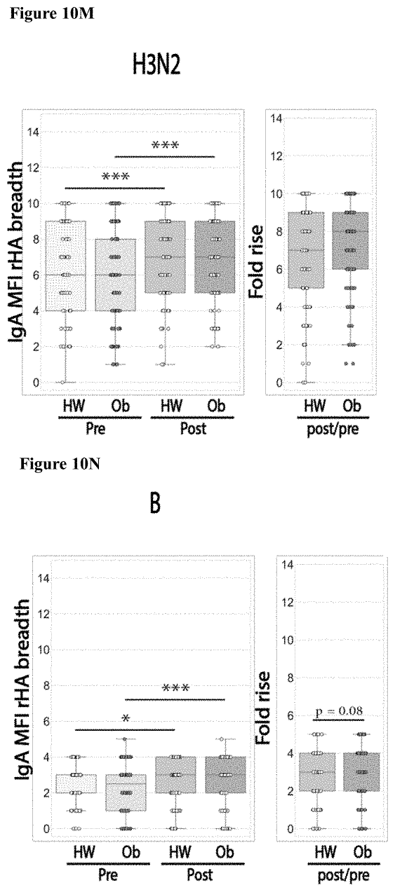

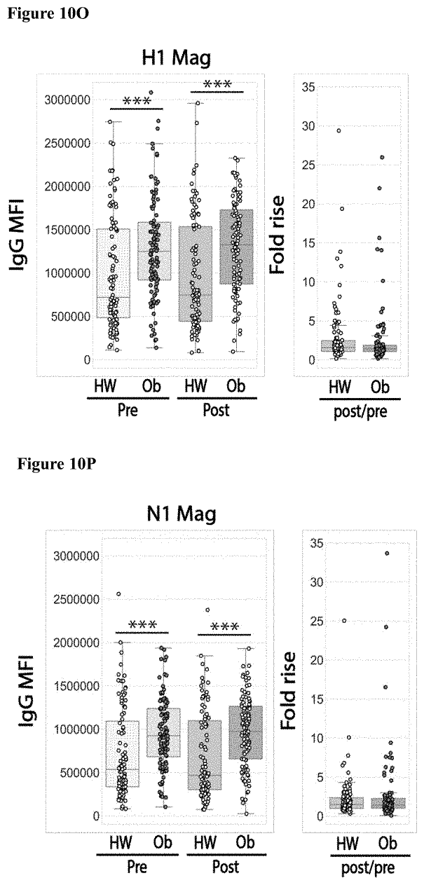

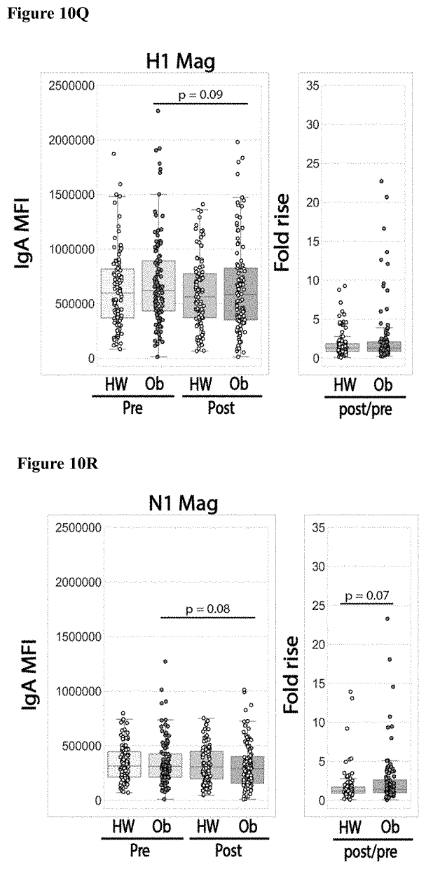

Obese individuals had decreased IgG and higher IgA post-vaccination responses to whole virus antigens of the 2010-2011 seasonal vaccine. Both HW and obese subjects generated a significant rise both IgG and IgA levels against whole virus antigens of the three 2010-2011 vaccine strains one-month post vaccination compared to baseline (FIG. 9A-9B, 9G-9H, 9M-9N, 9Q-9R, 9U-9V, 9Y-9Z). Similarly, a significant increase in IgG and IgA levels against rHA antigens of A/H1N1 and A/H3N2 were observed, but not against influenza B. The rise in IgA binding to the H1N1 Cal09 whole virus was stronger in the obese group (p<0.0005) as compared to the HW group (p=0.02) (FIG. 9G). While both groups generated vaccine-induced IgG and IgA responses, the obese group generated significantly lower IgG levels (H1N1: p=0.01, H3N2: p=0.001, B: p=0.01) and higher IgA levels (H1N1: p=0.002, H3N2: p=0.02, B: p=0.01) against whole virus antigens from 2010-2011 vaccine strains post-vaccination compared with HW. To adjust for differences in baseline antibody response, vaccine-induced fold change of IgG and IgA levels (post-vaccination antibody level divided by the baseline antibody level) were calculated for each individual. IgA fold change against whole virus antigens was significantly higher in the obese group for all the three 2010-2011 vaccine strains (H1N1: p=0.0001, H3N2: p=0.0006, B: p=0.002; FIG. 9G, 9Q 9Y), while no significant difference in fold change in IgG levels was observed between obese and HW (H1N1: p=0.2, H3N2: p=0.8, B: p=0.5; FIG. 9A, 9M, 9U). Post-vaccination IgG and IgA binding to rHA proteins was similar in the two groups (FIG. 9B, 9H, 9N, 9R, 9V, 9Z). No significant differences in total IgG or IgA between the two groups at baseline (p=0.84 and p=0.59, respectively) and post-vaccination (p=0.94 and p=0.42, respectively) were observed (FIG. 10A-10B).

### Example 10: Obese and Healthy-Weight Groups Displayed Different Repertoires of IgG and IgA Antibodies to Conformational and Linear Influenza Epitopes

The influenza VP microarray spotted with whole viruses and rHA proteins of 34 A/H1N1, A/H3N2 and B influenza strains further allowed profiling of antibodies to conformational epitopes. The magnitude and breadth of IgG and IgA antibodies were compared between the obese and HW groups. Magnitude was defined as the sum of antibody levels to all strains from a given subtype, and breadth was defined as the number of strains to which a subject has antibodies. As expected, vaccination induced a significant increase in the magnitude and breadth of both IgG and IgA antibodies to the viral and rHA antigens of all three subtypes (FIG. 9C-D, 9I-9J, 9O-9P, 9S-9T, 9W-9X, 9AA-9BB, 10C-10N). Similar to the observation that obese subjects had significantly lower baseline and post-vaccination levels of IgG antibodies against the seasonal 2010-2011 vaccine strains compared with HW group, the magnitude and breadth of IgG antibodies to whole viruses from all the three subtypes were also lower in the obese group at the two timepoints (FIG. 9C, 90, 9W, 10C-10E). Baseline IgG magnitude against rHA proteins of H1N1 and B, but not H3N2, strains was also lower in the obese group. Post-vaccination IgG magnitude and breadth to rHA antigens did not significantly differ between the two groups (FIG. 9D, 9P, 9X, 10F-10H), in accordance with the IgG levels against rHA proteins of the 2010-2011 vaccine strains.

In contrast to baseline differences in IgG magnitude, IgA magnitude against whole viruses from all three subtypes at baseline did not differ between the two groups (FIG. 9I, 9S, 9AA). However, the obese group had significantly higher magnitude and breadth of IgA response to whole virus A/H1N1 and A/H3N2 antigens one-month post-vaccination (magnitude: A/H3N2; breadth: A/H1N1 and A/H3N2; FIG. 9I, 9S, 9AA, 10I-10K). Baseline IgA magnitude and breadth for H1N1 rHA proteins were significantly decreased in obese subjects (mag: p=0.002, FIG. 9K, 10L), but were not different for H3 and B subtypes (FIG. 9R, 9Z). As a result, the fold change of IgA magnitude to A/H1N1 proteins, but not A/H3N2 or B proteins, was significantly higher in the obese group (FIG. 9K, 9R, 9Z). In addition, the breadth of IgA antibodies to viruses from all the three subtypes and rHA proteins of the A/H1N1 subtype strains (rH1) were also significantly higher in the obese (FIG. 9S-9T, 9AA-9BB, 10I-10K). Overall, obese individuals had significantly decreased magnitude and breadth of IgG antibodies to whole influenza viruses and recombinant proteins from A/H1N1, A/H3N2 and B strains at baseline, and significantly higher magnitude or breadth to these antigens post-vaccination compared to HW controls.

To further compare specific antibody repertoires against linear epitopes in obese and HW, a peptide microarray spotted with 20mer peptides spanning the HA and NA proteins of the A/H1N1 Cal09 vaccine strain with a partial overlap of 15aa was utilized. Here magnitude was defined as the sum of antibody levels to all peptides from the same protein, and breadth was defined as the number of peptides from each protein to which the subject had antibodies. Interestingly, while obese subjects exhibited decreased IgG against whole virus at baseline and post-vaccination compared to HW, they had a higher magnitude and a broader repertoire of IgG antibodies against H1 and N1 Cal09 peptides (FIG. 9E-9F, 100-10P). In contrast, IgA breadth against H1 and N1 peptides was significantly lower in obese subjects at both timepoints, but the magnitude did not significantly differ (FIG. 9K-9L, 10Q-10R). Thus, obesity was associated with a higher level of IgG antibodies to linear peptides and a lower level of IgG to the whole Cal09 virus (FIG. 9A). A similar inverse correlation was observed for the post-vaccination IgA response: a higher level of IgA to the whole virus in the obese (FIG. 911) and a narrower IgA repertoire to linear peptides (FIG. 9K-9L). It was found that the obese group was characterized by weaker and narrower IgG repertoire at baseline to conformational influenza antigens from all 3 subtypes, accompanied by stronger and wider IgG repertoire to linear Cal09 peptides. Post-vaccination, the obese group was characterized by stronger and wider IgA response to conformational influenza antigens and a narrower IgA repertoire to linear peptides. Therefore, the results suggest an inverse correlation between the antibody response to linear peptides and conformational influenza epitopes in general.

### Example 11: Baseline Immune-History Repertoires Exhibit Marked Heterogeneity Across Subjects within Both Groups

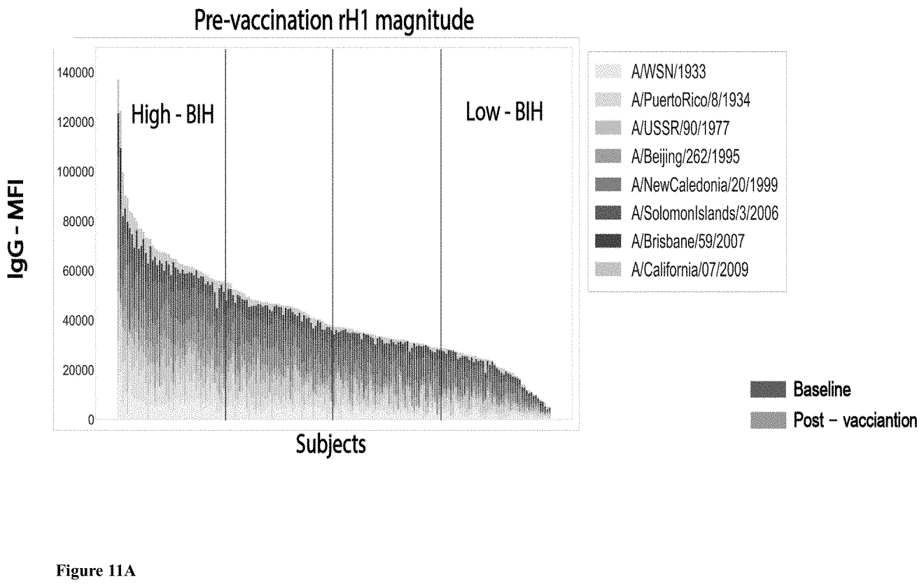

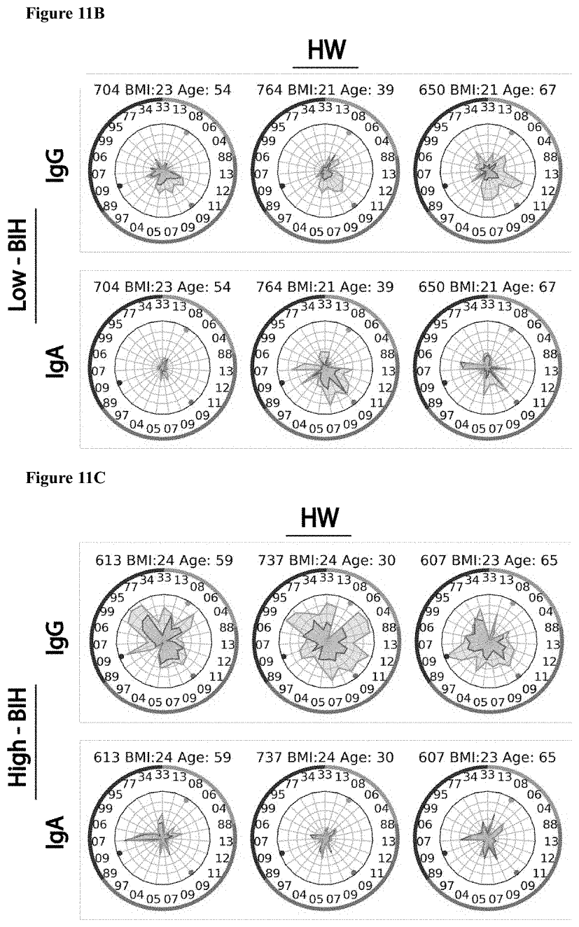

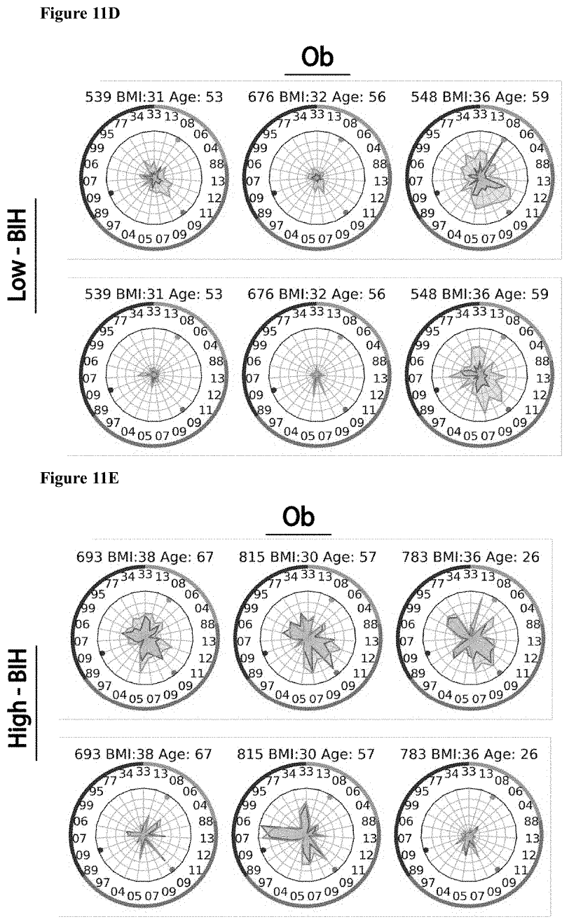

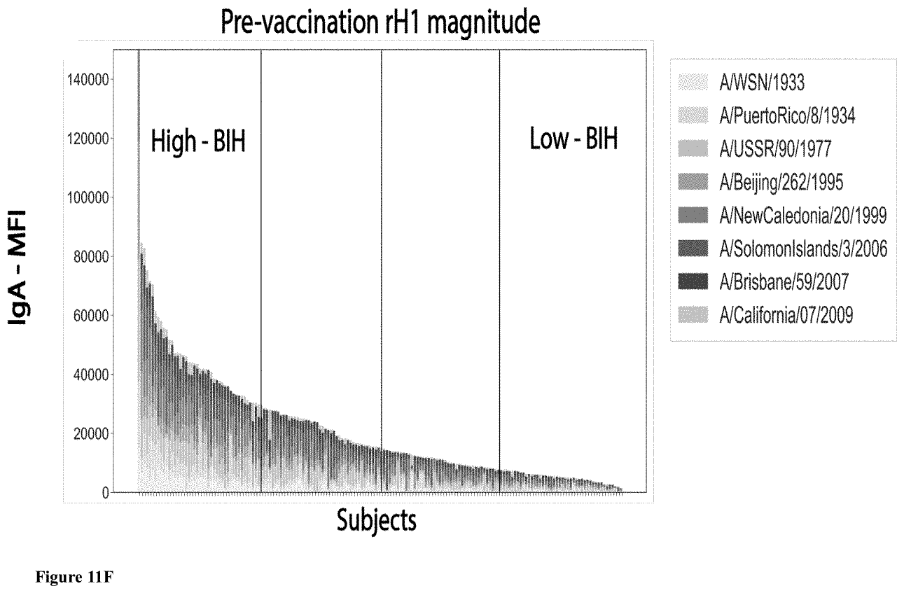

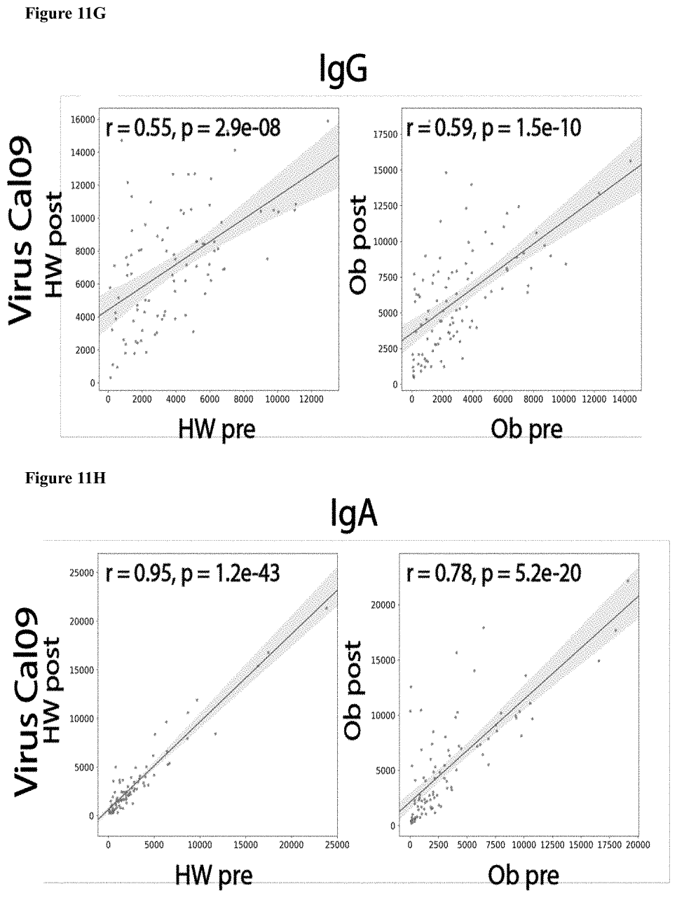

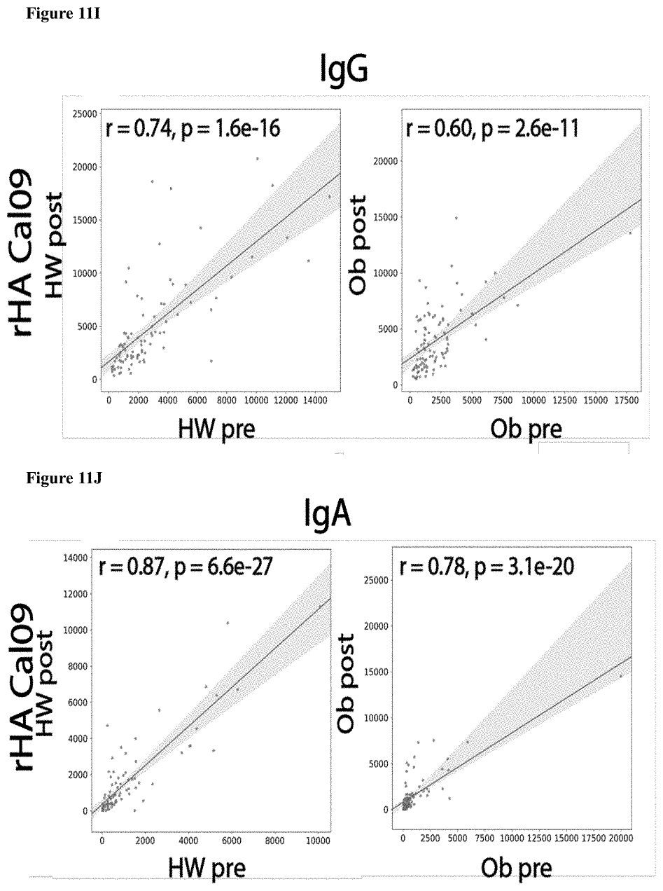

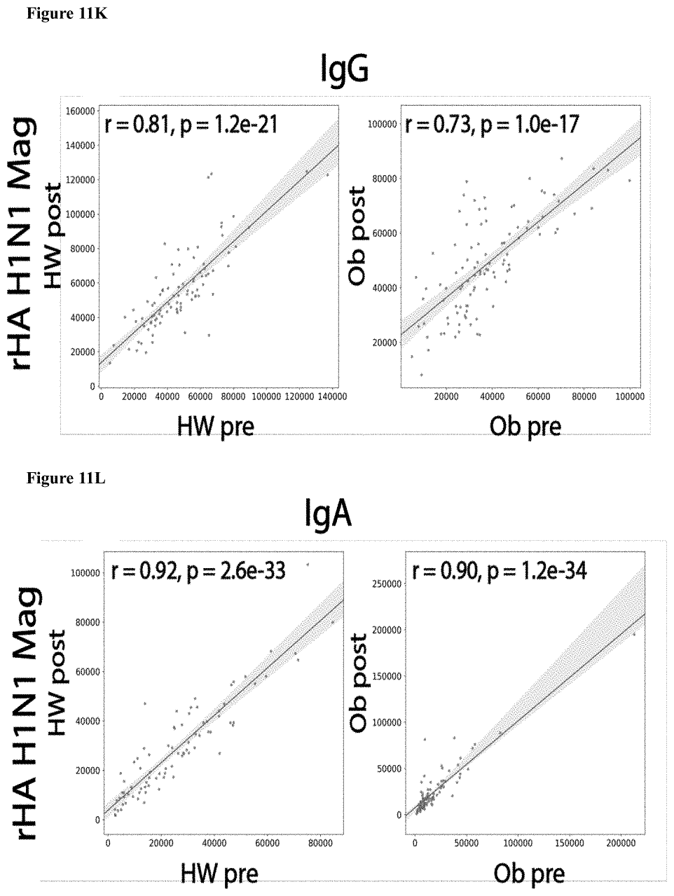

Baseline antibody magnitude and breadth against influenza subtypes could represent the effect of previous exposures to influenza viruses and vaccines on the antibody response, resulting in a baseline immune history (BIH) against influenza virus itself. To assess the effect of the differences in BIH, all the subjects in the cohort (including both HW and obese) were sorted by individual baseline IgG (FIG. 11A-11E) or IgA (FIG. 11F) magnitude to whole viruses or rHA proteins of the H1N1 subtype (rH1) resulting in extensive heterogeneity in baseline magnitude. FIGS. 11A and 11F are examples of ranking according to magnitude of antibodies to H1N1 (rH1) proteins. Following the ranking, the subjects were divided into four BIH quartiles as described in Example 4 above.

To further gain insights into baseline differences between subjects, the BMI of subjects was compared across the lowest (low-BIH group) and highest quartiles (high-BIH group) of the Cal09 BIH distribution. To visualize the BIH of each subject, spider plots were generated for a representative subset of low IgG-BIH and high IgG-BIH subjects, in which the level of antibodies to each of the individual rHA antigens from the three subtypes are plotted at baseline and post-vaccination for both IgG and IgA (FIG. 11B-11E). These plots demonstrate that each subject in the cohort has a unique BIH profile that varies both in the overall magnitude and breadth, but also in the specificity to each subtype and to individual strains within the panel. For example, some subjects with low IgG-BIH to rH1 have low IgG-BIH to all three subtypes (e.g., subjects 704, 539 and 676), while others have stronger levels of IgG to other subtypes (e.g., subject 548). Within the high-BIH group, subjects also presented specific preferences for subtypes and strains. For example, subjects 613, 737 and 815 had a high level of antibodies to strains from all three subtypes, but other subjects had preferences to specific subtypes at baseline (e.g., subject 607 had a low IgG-BIH to subtype B). The antibody response to the vaccine was heavily biased by the individual BIH (FIG. 11G-11L), as can be also observed by comparing the baseline to post-vaccination spider plots. In many subjects the post-vaccination profile was very similar to baseline or an expansion of the baseline profile. However, while some low-BIH subjects hardly developed any vaccine-induced antibody response and remained low post-vaccination (e.g., subject 676), others generated robust vaccine-induced immune responses (e.g., subjects 650 and 548).

### Example 12: IgG and IgA Immune History Profiles are Only Moderately Correlated

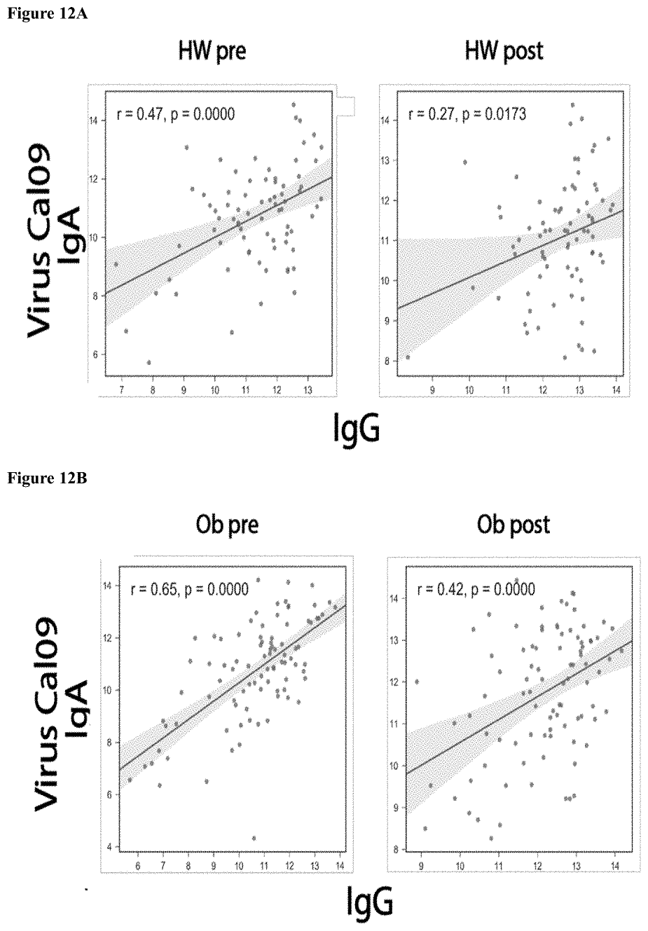

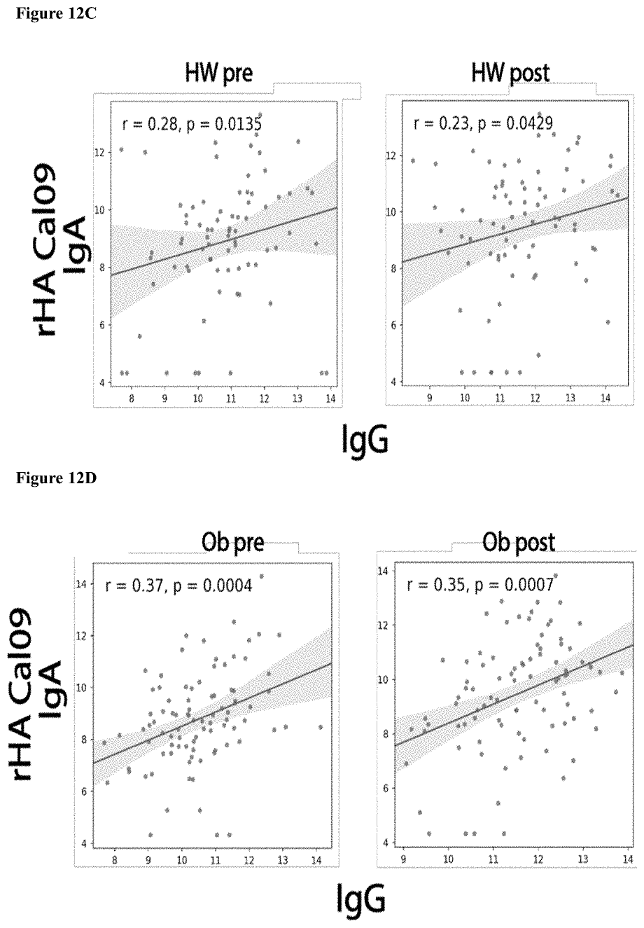

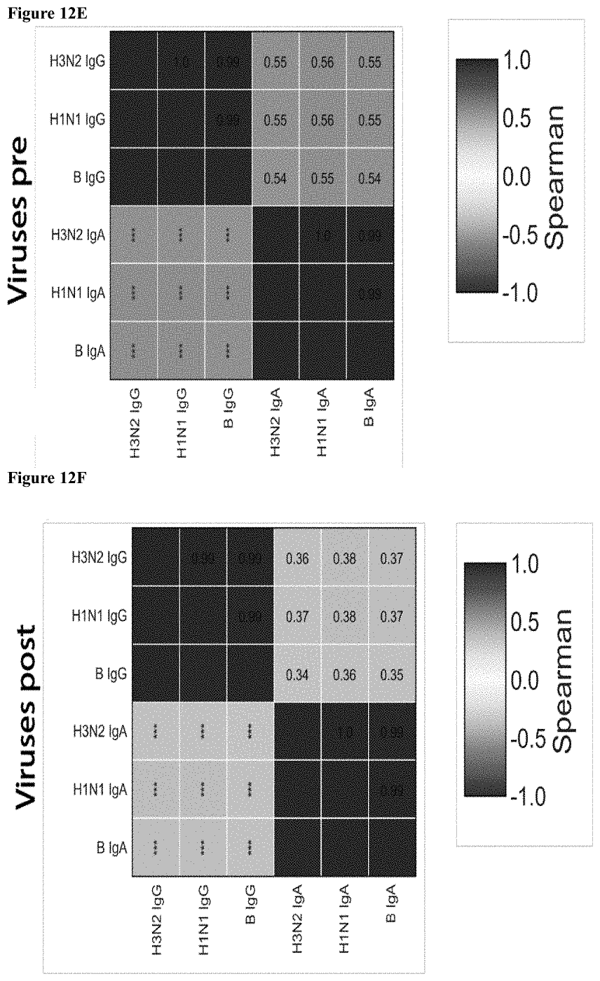

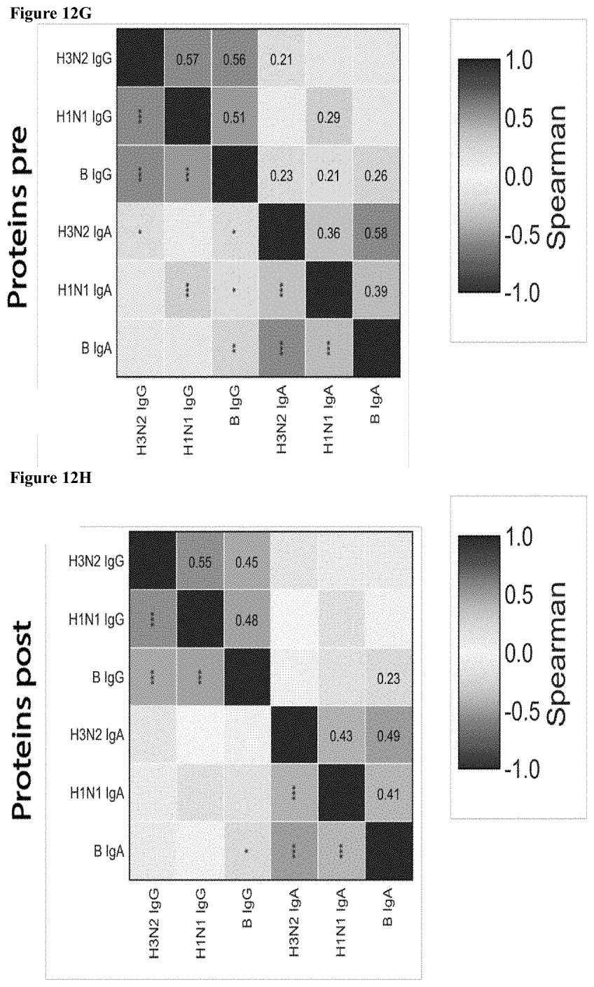

Comparison between the IgG and IgA spider plots for each subject demonstrates that low IgG-BIH to rH1 correlates low IgA-BIH to rH1; however, in some individuals striking differences exist between IgG and IgA profiles, such as a subject with high IgG levels to influenza and low IgA levels and vice versa (e.g., subjects 764, 737 and 783). Sometimes the IgA and IgG levels did not differ in magnitude to subtypes, but did differ in antibody specificity (e.g., subject 815 had high IgG and IgA magnitudes to all the three subtypes, but to different strains, and had high IgA level and low IgG level to Cal09 rHA) (FIG. 11B-11E). Next, the correlations between binding of IgG and IgA antibodies to the whole virus or rHA protein of the Cal09 vaccine strain were computed at both time points within each group. Weak to moderate correlations was found in HW and obese subjects at both time points (0.23≤r≤0.65, FIG. 12A-12D), but the correlations were higher at baseline as compared to post-vaccination in both groups, and higher in the obese group as compared with the HW at both timepoints (FIG. 12A-12D). The correlation between overall IgG and IgA titers as measured by ELISA was also found to be higher in the obese group as compared with the HW group at both baseline (r=0.53 vs. r=0.63 respectively) and post-vaccination (r=0.53 vs. r=0.61 respectively).

The Spearman correlations between the IgG and IgA magnitudes for influenza whole viruses and rHA proteins was computed from each subtype for each serum sample across the entire cohort. While IgG and IgA magnitudes to whole viruses were significantly correlated at baseline (H1N1 r=0.56, H3N2 r=0.55, B r=0.54), and post-vaccination (H1N1 r=0.38; H3N2 r=0.36; B r=0.35) (p<0.0005, FIG. 12E-12F), the correlations between IgA and IgG magnitudes to rHA proteins from the same subtype were significant but weaker at baseline (H1N1 r=0.29; H3N2 r=0.21; B r=0.26), and significant only for subtype B post-vaccination (B r=0.23), FIG. 12G-12H). Overall, the correlations between IgA and IgG magnitude to the same subtype were stronger for viruses and weaker for rHA proteins. IgG-IgA correlations for both viruses and proteins were stronger at baseline than post-vaccination.

### Example 13: Obesity is Associated with Low Magnitude of IgG and IgA Antibodies to H1N1 HA Proteins at Baseline

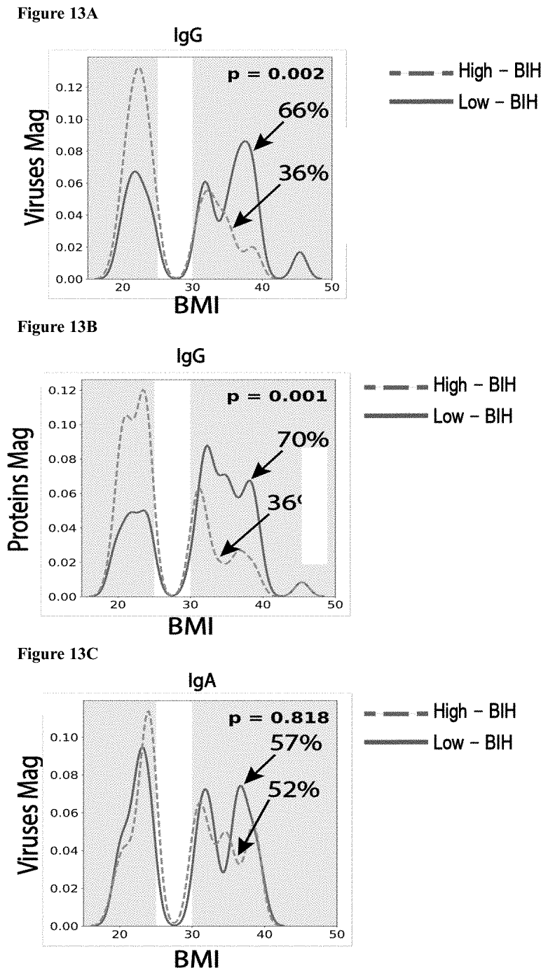

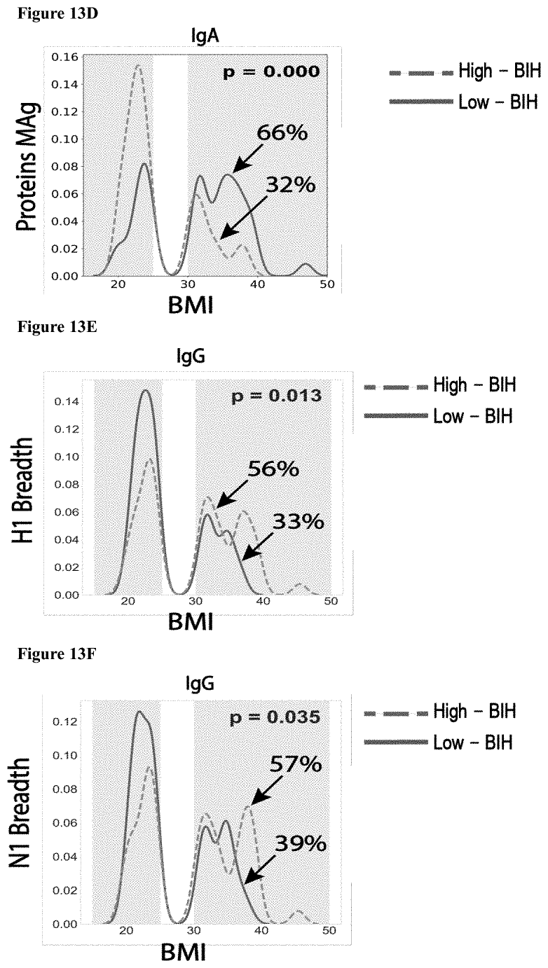

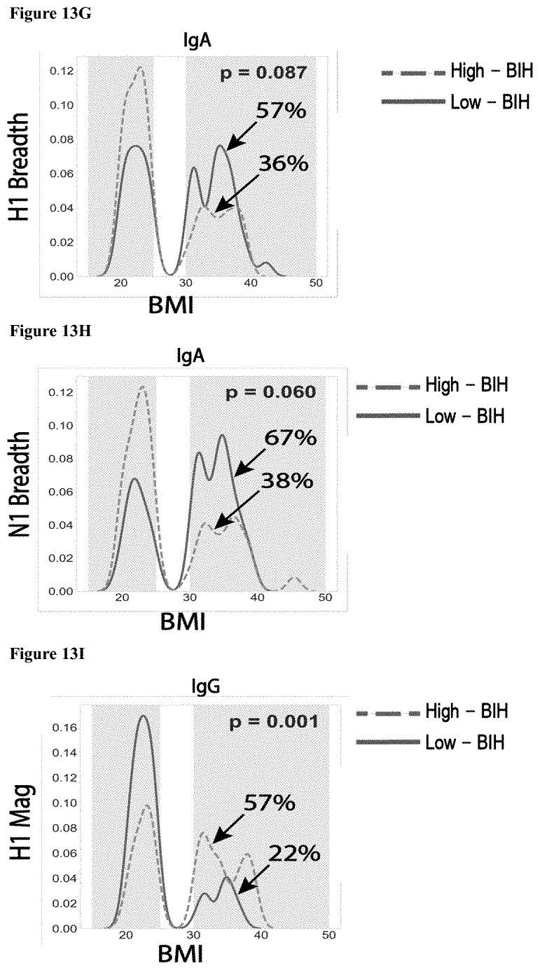

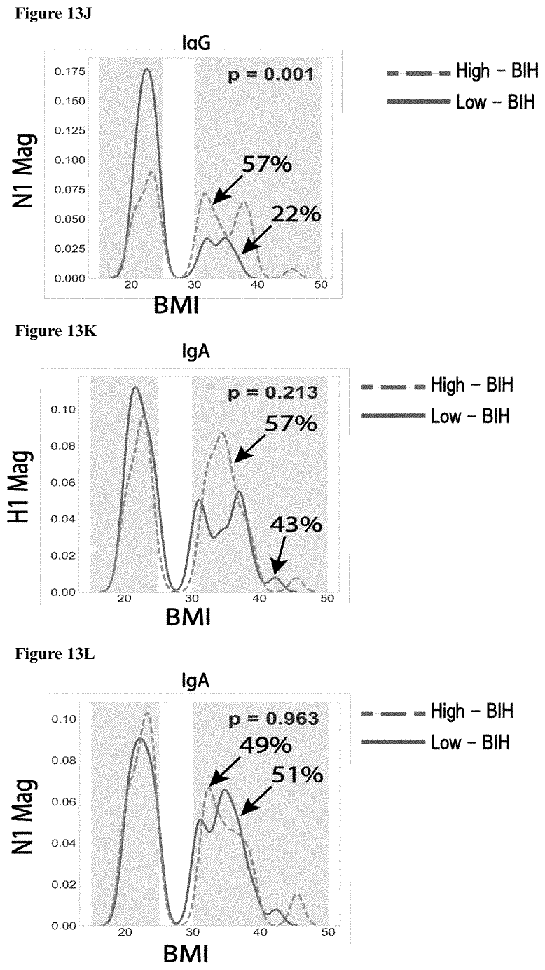

Previous studies reported that obesity was associated with a more severe disease following infection with the pandemic H1N1 Cal09 strain. Based on the observations above that the antibody repertoire to influenza at baseline was different in obese and healthy-weight subjects, it was hypothesized that the baseline antibody repertoire to H1N1 antigens may be associated with obesity. Therefore, the frequency of obese and healthy-weight subjects in the low-BIH and high-BIH groups was measured for both IgG and IgA rankings for whole H1N1 viruses (FIG. 13A, 13C), or rH1 proteins (FIG. 13B, 13D). Statistical significance for the difference between BMI distributions in the low-BIH and high-BIH quartiles was measured using the Wilcoxon rank-sum test. The difference in the frequency of obese and healthy-weight subjects within the low-BIH and high-BIH groups was also compared using Fisher's exact test.

A significantly higher frequency of obese subjects was found in the low IgG-BIH group as compared to the high IgG-BIH group for H1N1 viruses (66% vs. 36%, p=0.006 and rH1 proteins (70% vs. 36%, p=0.001) (FIG. 13A-13B). Similar differences were observed for IgA responses to rH1 proteins (66% obese subjects in low IgA-BIH vs. 32% in high IgA-BIH, p=0.002), but not for IgA to H1N1 viruses (57% vs. 52%, p=0.8) (FIG. 13C-13D).

The distributions of BMI in the low-BIH and high-BIH groups were also compared. Comparisons between the distributions were computed using the Wilcoxon rank-sum test. Significant differences in BMI distributions of low-BIH and high-BIH groups were observed for rH1 proteins for both IgG and IgA based rankings (IgG: p=0.001, IgA: p=0.0001).

Differences in the BMI distributions of the low-BIH and high-BIH groups for H1N1 viruses were significant for IgG, but not for IgA (IgG: p=0.001, IgA: p=0.8). Importantly, the association between BIH and obesity was specific for rH1 proteins, and w no significant associations were found between baseline immune-history rankings for magnitude of H3N2 and B HA proteins with obesity (data not shown).

### Example 14: Obesity is Associated with a Stronger and Broader Repertoire of IgG to Cal09 H1 and N1 Peptides at Baseline, and with a Higher IgG Response to Cal09 Peptides and IgA Response to HIN1 Proteins and Viruses Post Vaccination

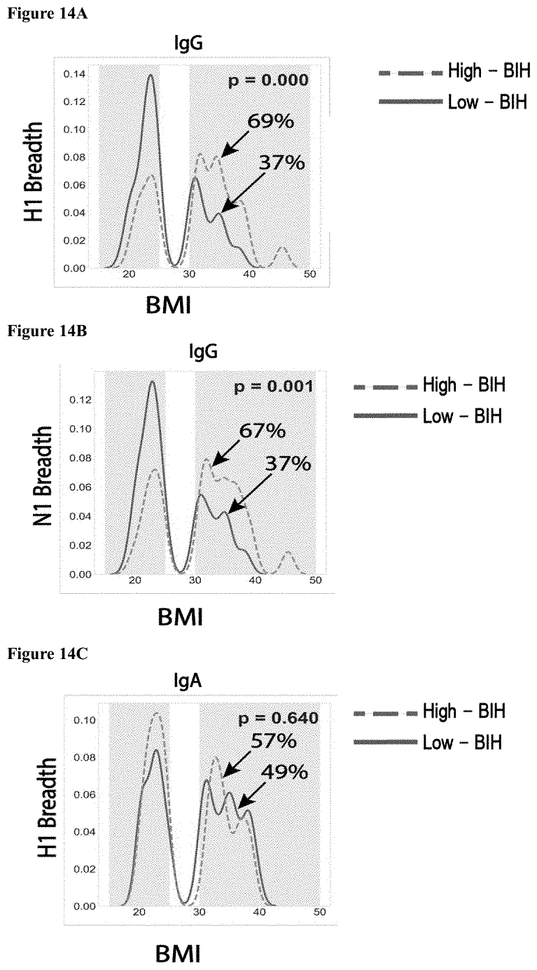

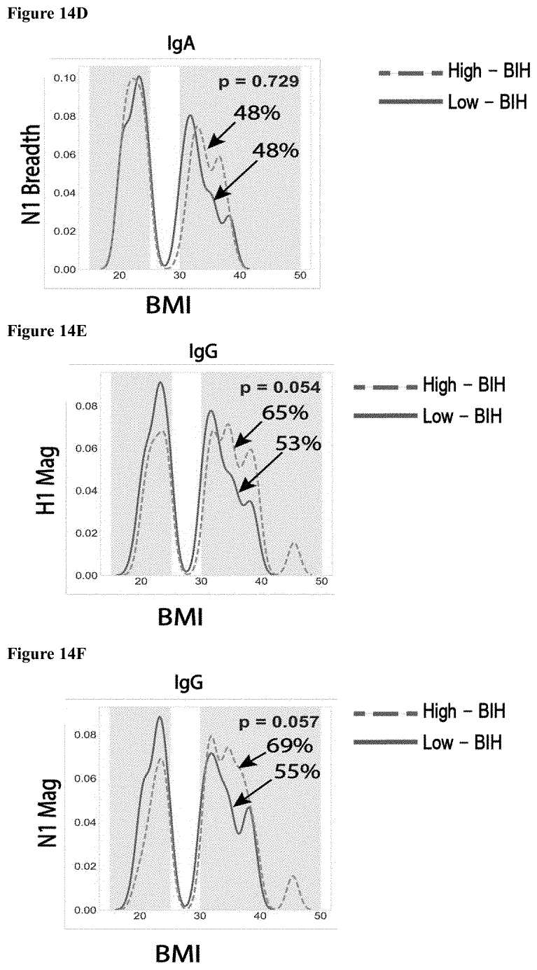

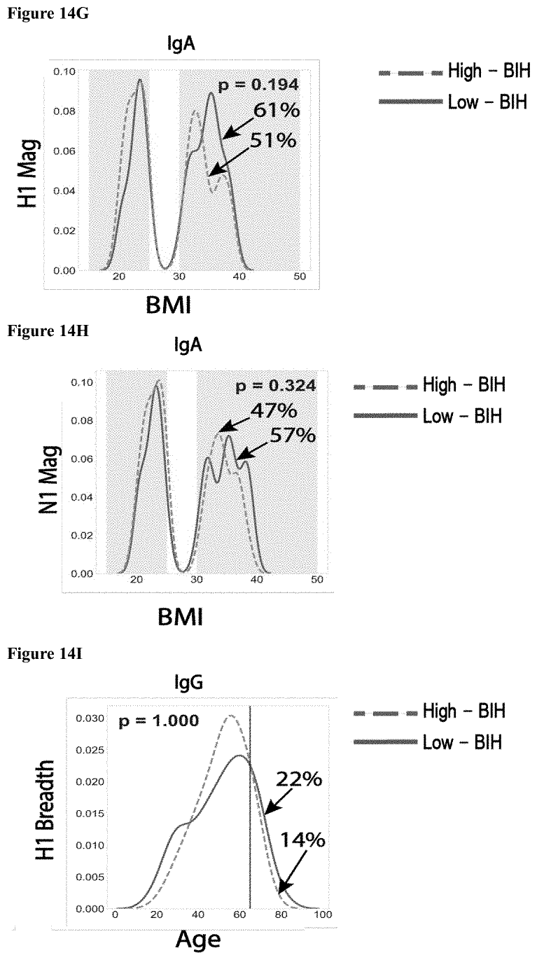

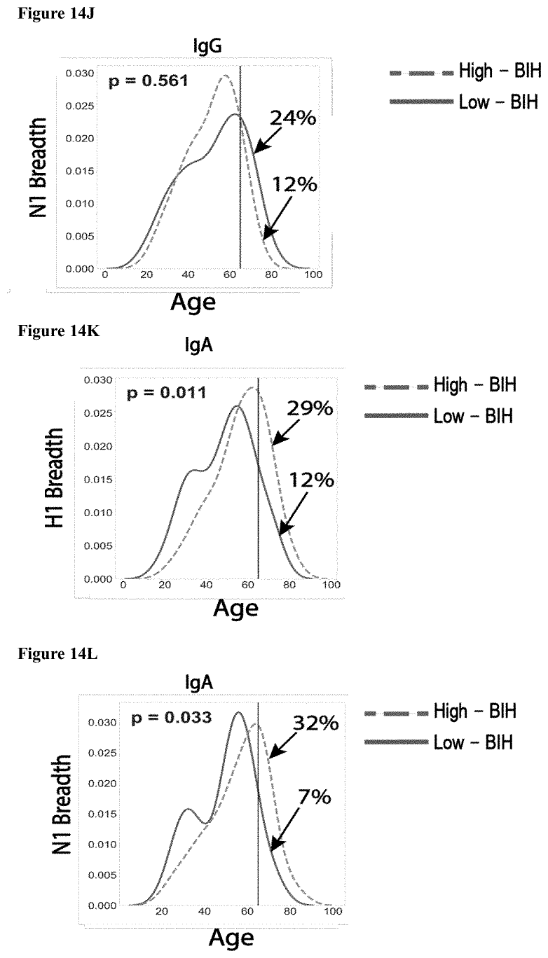

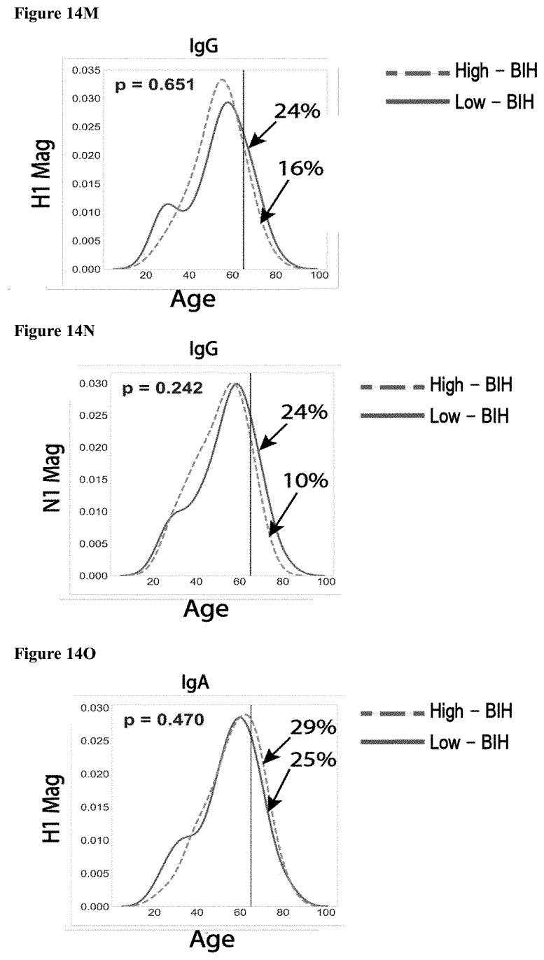

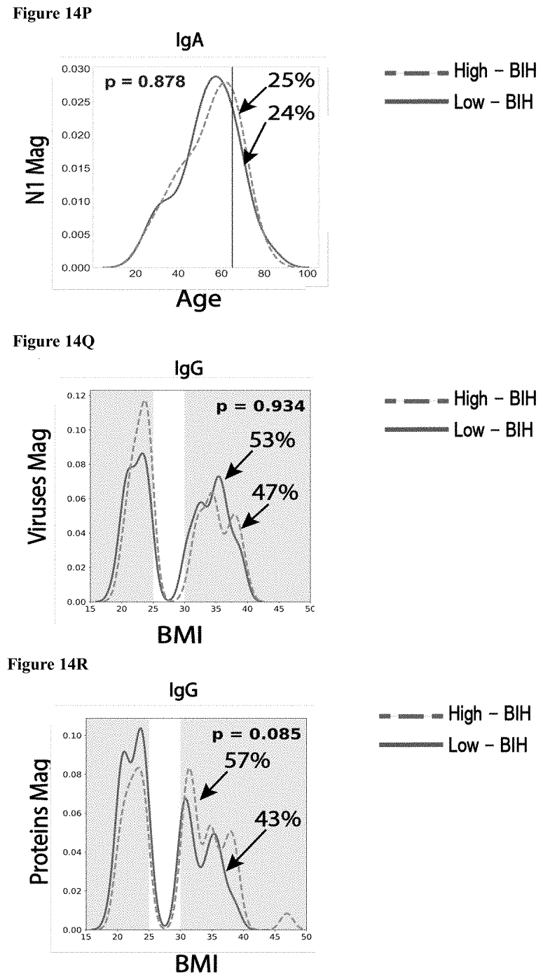

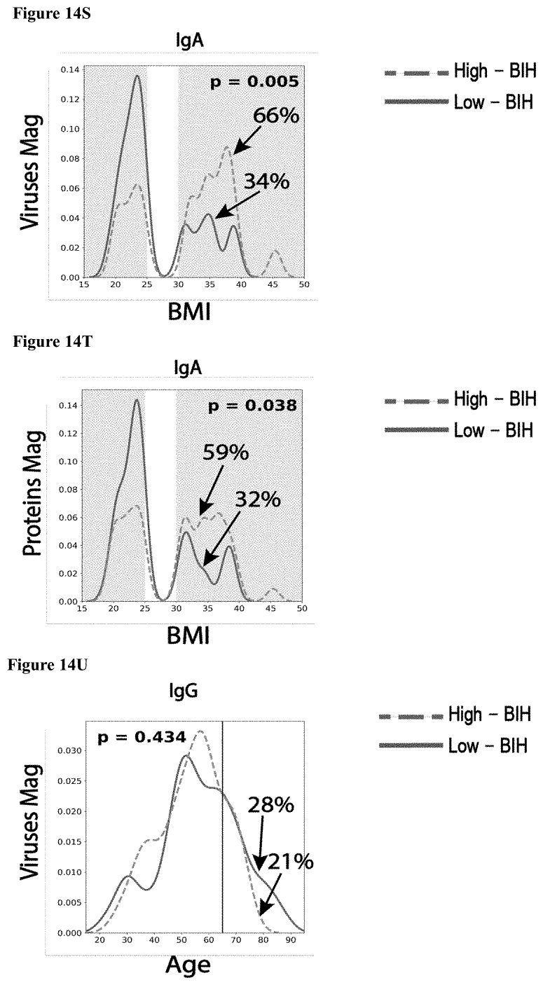

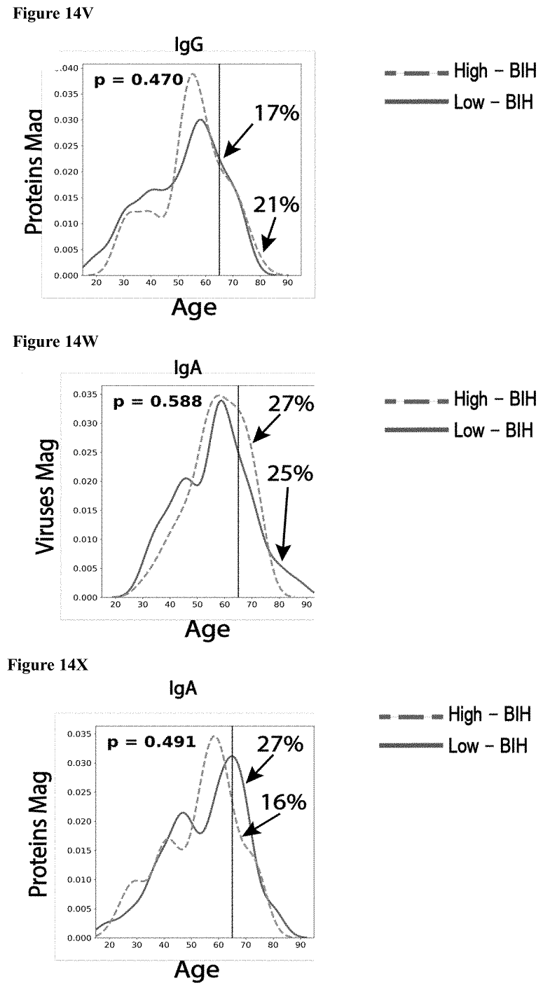

Subjects were also ranked by their baseline repertoires of IgG antibodies to the Cal09 H1 and N1 peptides to define low-BIH and high-BIH groups for magnitude and breadth, based on quartiles (FIG. 13E-13F, 13I-13J). In contrast to the findings on magnitude to whole viruses and protein antigens, obese subjects were found to be less frequent in the low IgG-BIH groups for H1 and N1 peptides, for both magnitude-based ranking (H1: 22% vs. 57%, p=0.0004; N1: 22% vs. 57% obese subjects in low vs. high IgG-BIH, p=0.0004, FIG. 13I-13J) or breadth (H1: 33% vs. 56%, p=0.02; N1: 39% vs. 57%, p=0.07; FIG. 13E-13F). Similar findings were also observed when comparing the distributions of BMI between the low and high peptide IgG-BIH, both for magnitude and breadth groups (mag; H1: p=0.001, N1: p=0.001, breadth; H1: p=0.01, N1: p=0.03). The increased frequency of obese subjects with a stronger and broader IgG repertoire to Cal09 peptides was also observed post-vaccination. Ranking subjects by the magnitude and breadth of their baseline-adjusted post-vaccination IgG responses to Cal09 H1 and N1 peptides demonstrated that obese subjects were more frequent in the high IgG responder groups than HW subjects (FIG. 14A-14B, 14E-14F). Obese subjects were also more frequent in the high IgA responders quartiles for H1N1 whole viruses and rH1 proteins (FIG. 14S-14T). However, the baseline-adjusted post-vaccination IgG responses to H1N1 viruses and rH1 proteins were not associated with obesity (FIG. 14Q-14R).

### Example 15: Obesity is Associated with a Narrower IgA Repertoire to Cal09 H1 and N1 Peptides at Baseline, but does not Affect IgA Response to Cal09 Peptides Post Vaccination

In contrast to the enrichment of obese individuals in high IgG-BIH groups to H1 and N1 peptide magnitude and breadth, obese subjects were more frequent in the low IgA-BIH breadth groups to Cal09 peptides (H1: 57% vs. 36%, p=0.04; N1: 67% vs. 38%, p=0.007; FIG. 13G-13H). However, no significant differences were observed between the frequency of obese individuals in the low and high IgA magnitude BIH groups to both H1 and N1 peptides at baseline (p=0.2 and p=1 respectively, FIG. 13K-13L). The distribution of obese and HW subjects was similar in low and high-IgA responder groups to Cal09 peptides post-vaccination (FIG. 14C-14D, 14G-1411).

### Example 16: The Baseline IgG and IgA Repertoires to H1N1 Antigens and the Breadth of IgA Response to Cal09 Peptides are Also Affected by Age

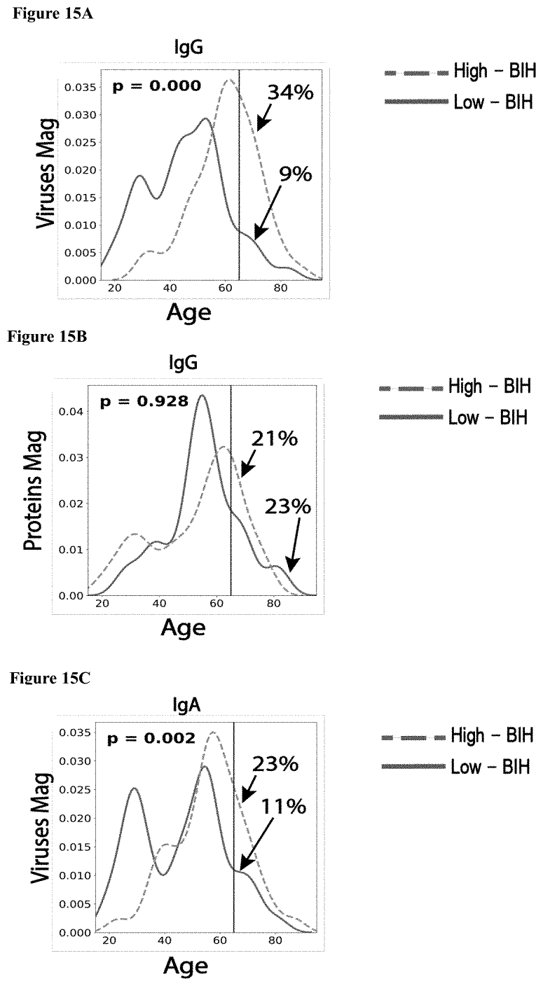

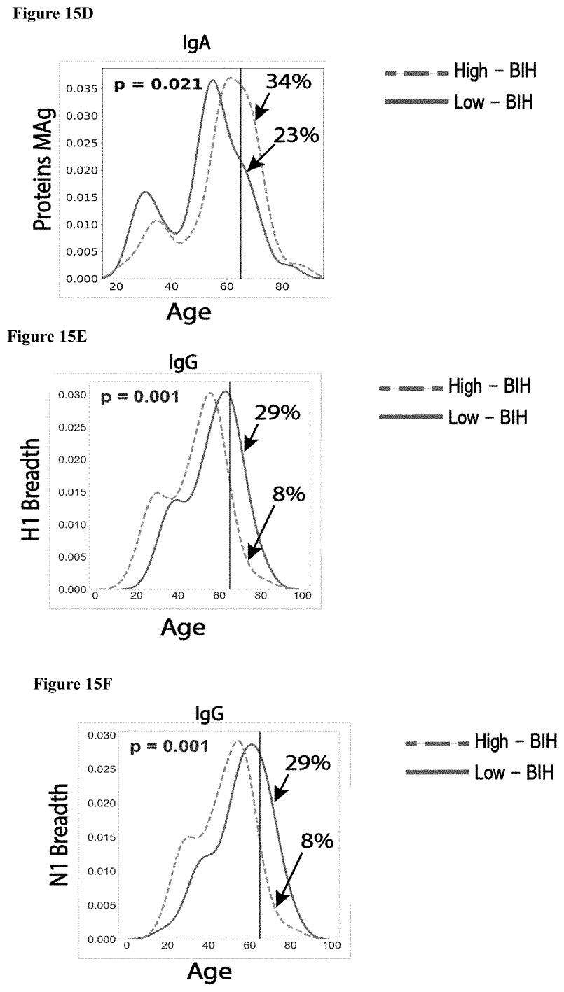

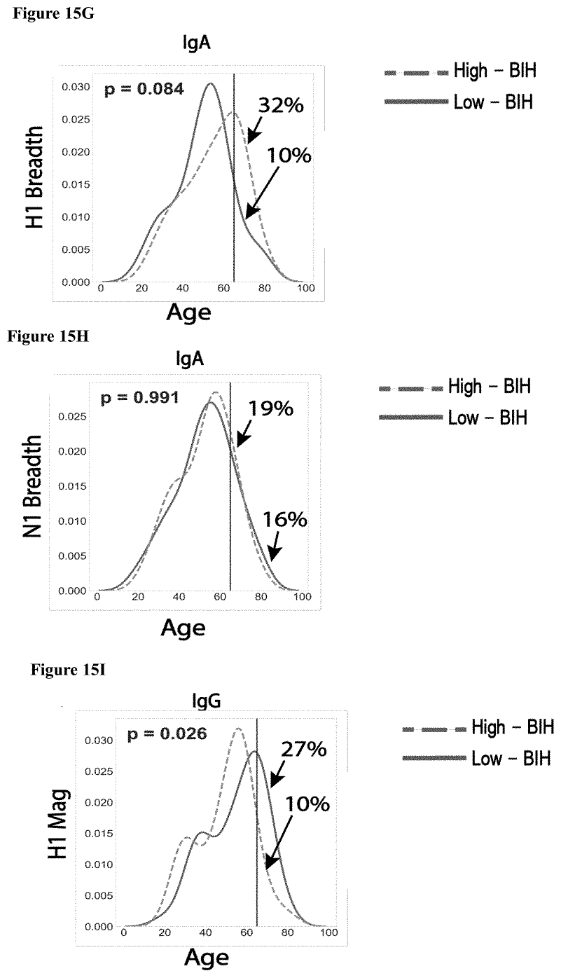

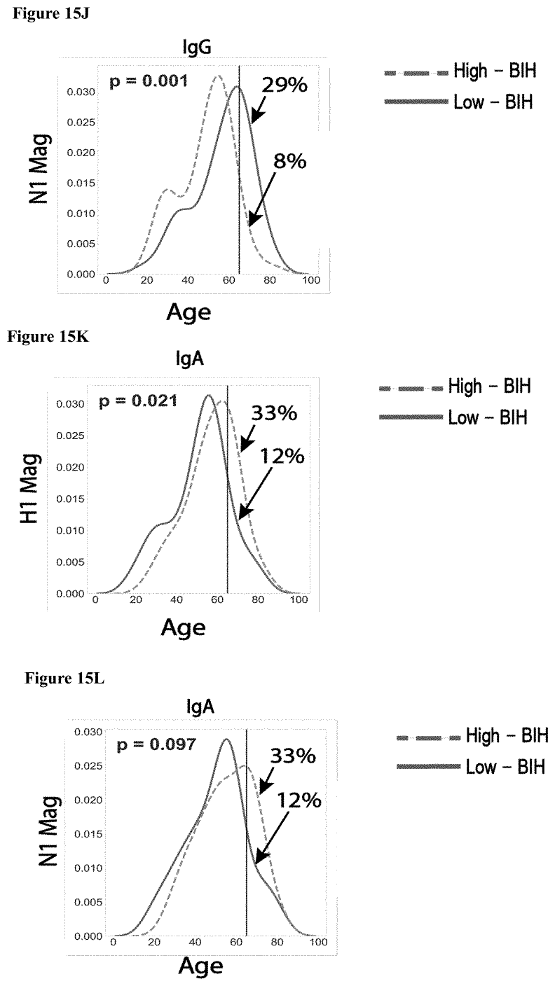

Since immune-history develops over time and is influenced by the number of exposures and vaccinations, we also sought to assess the association between participants' age and their immune-history profiles. In particular, 21.1% of subjects from the cohort were elderly (>=65, Table 7). An association was found between elderly age and high IgG and IgA magnitude to H1N1 whole viruses at baseline (FIG. 15A, 15C). Only 9% of subjects with low IgG-BIH to whole H1N1 viruses were elderly, while elderly subjects were 34% of subjects with high IgG-BIH to whole H1N1 viruses (p=0.004, Fisher's exact test, FIG. 15A). Elderly subjects were also enriched in the high IgA-BIH groups to both whole H1N1 viruses and rH1 proteins (p=0.25 and p=0.34 respectively, FIG. 15C-15D), while the percentage of elderly subjects in low and high IgG-BIH for rH1 proteins was almost equal (FIG. 15B). However, the post-vaccination change in magnitude and breadth of the IgG and IgA repertoires to H1N1 whole viruses and rH1 proteins was not clustered with age (FIG. 14U-14X). Associations between age and baseline immune history rankings by H3N2 and B HA proteins was also tested, and no significant associations were found (IgG: 0.4<p<1, IgA: 0.33<p<0.4, data not shown).

When comparing the frequency of elderly subjects in the low and high baseline breadth and magnitude to H1 and N1 peptides, it was found that in contrast to BIH against H1N1 viruses, elderly subjects were more frequent in the low IgG-BIH breadth and magnitude groups as compared to the high IgG-BIH for both H1 and N1 peptides (breadth/magnitude: H1: 29%/27% vs. 8%/10%, p=0.009/0.03; N1: 29% vs. 8%, p=0.008/0.009; FIG. 15E-15F, 15I-15J). In contrast, the elderly subjects were significantly enriched in the high IgA-BIH magnitude group for H1 peptides only (p=0.01, FIG. 15K-15L). The elderly subjects were also more frequent in the high BIH groups of IgA magnitude to N1 peptides and IgA breadth to H1 peptides (p=0.01, FIG. 15G, 15I). No significant difference in age distribution was observed between the baseline-adjusted post-vaccination IgG responses to H1 and N1 peptides in low and high responders (FIG. 14I-14J, 14M-14N). However, the high-responders groups for IgA breadth were enriched with elderly subjects for both H1 and N1 peptides (H1: p=0.07, N1: p=0.01, FIG. 14K-14L), although responder quartiles for IgA magnitude to Cal09 peptides did not differ (FIG. 140-14P). Thus, old age was associated with high IgG and IgA levels to whole H1N1 viruses, high IgA levels to rH1 proteins, along with lower IgG levels and narrower IgG repertoire to H1 and N1 peptides, and high IgA levels to H1 peptides at baseline. In addition, old age was associated with a broader IgA response to Cal09 peptides post-vaccination.

### Example 17: Obese Subjects with Low-BIH to H1N1 Viruses and Proteins and High-BIH to Cal09 Peptides are Significantly Younger

While no correlation was found between age and BMI (p=0.85), it was found that subjects with low-BIH to H1N1 whole viruses and rH1 proteins were more likely to be obese and young. Furthermore, subjects with high-BIH to H1 and N1 Cal09 peptides were also more likely to be obese and young. To test whether there was an association between obesity and age, the age distributions of obese subjects in the low- and high-BIH groups were compared. It was found that obese subjects in the low IgG- and IgA-BIH groups to whole viruses were significantly younger than obese subjects in the high-BIH groups to viruses (IgG: p=0.000001 and IgA: p=0.002, respectively). Furthermore, obese subjects in the low IgA-BIH to rH1 proteins were younger than the obese subjects in the high IgA-BIH to proteins (p=0.02), while the age of obese subjects in IgG-BIH groups to rH1 proteins was similar (p=0.9). No similar differences were found when comparing HW subjects from the low-BIH and high-BIH groups.

### Example 18: Post-Vaccination IgG and IgA Antibody Levels can be Predicted by Baseline Immune-History Profiles

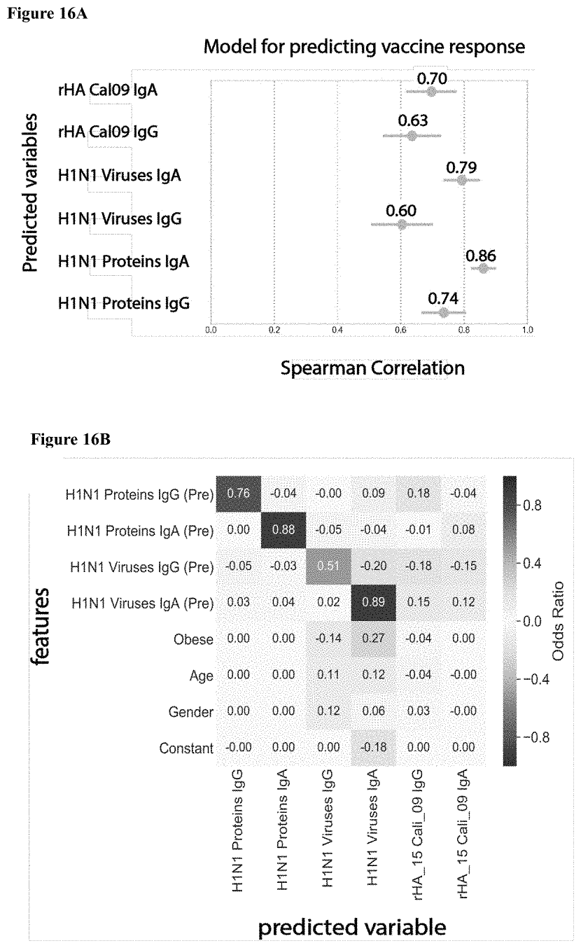

When comparing the baseline and post-vaccination antibody profiles of individual subjects (FIG. 11B-11D, 11G-11L) it was found that overall, the post-vaccination response in many subjects tended to expand their baseline immune-history repertoire. It was therefore asked to what extent could post-vaccination antibody levels be predicted using baseline antibody levels. Generalized linear regression models were trained using IgG and IgA baseline antibody magnitudes to H1N1 whole viruses and rH1 proteins, obesity, age and gender as input features, to predict the post-vaccination IgG and IgA antibody responses to the HA protein of the Cal09 vaccine strain, as well as the overall magnitude for all rH1 proteins and H1N1 viruses included in the VP array. Models were trained using leave-one-out cross validation. To assess the performance of the models, the Spearman correlation coefficient between the predicted and observed post-vaccination antibody responses was calculated. Significant correlations were found between the predicted and observed antibody levels to all 6 variables (FIG. 16A). The highest predictive power was observed for IgA magnitude to both rH1 proteins (r=0.86) and H1N1 whole viruses (r=0.79). To further gain insight into these models, the weights (beta-coefficients) assigned to each of the input features in each of the models was analyzed (FIG. 16B). As expected, the most informative feature within each of the models were found to always be the baseline level of the variable predicted by the model (e.g., baseline IgA magnitude to rH1 proteins was the most informative feature for predicting IgA rH1 magnitude post-vaccination). However, in some cases other features were also significant. In particular, obesity was negatively associated with post-vaccination IgG levels (r=−0.14) to H1N1 viruses and positively associated with post-vaccination levels to IgA levels to H1N1 viruses (r=0.27). Another interesting finding is that the baseline magnitude of IgG to H1N1 viruses was negatively associated with the post-vaccination IgG response to rH1 proteins (FIG. 16B).

### Example 19: Antibody Profiles to Cal09 H1 and N1 Peptides Predict Obesity

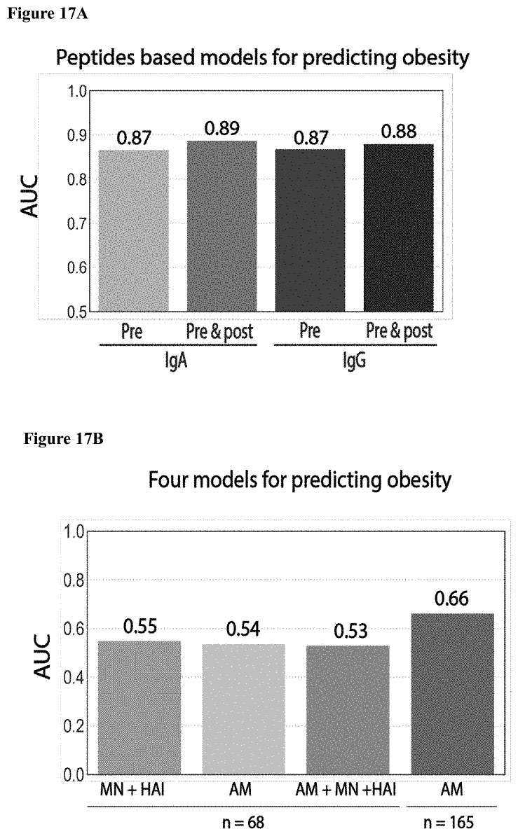

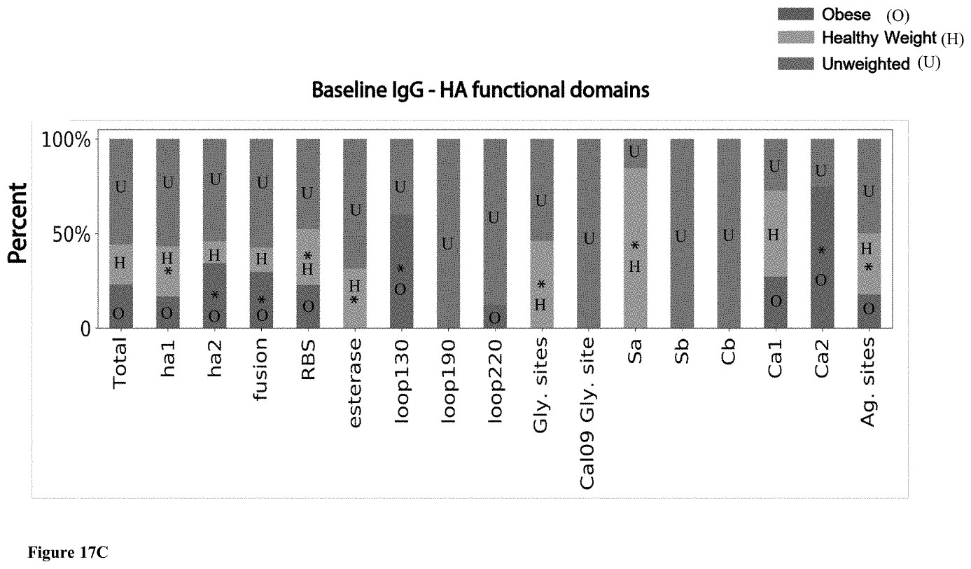

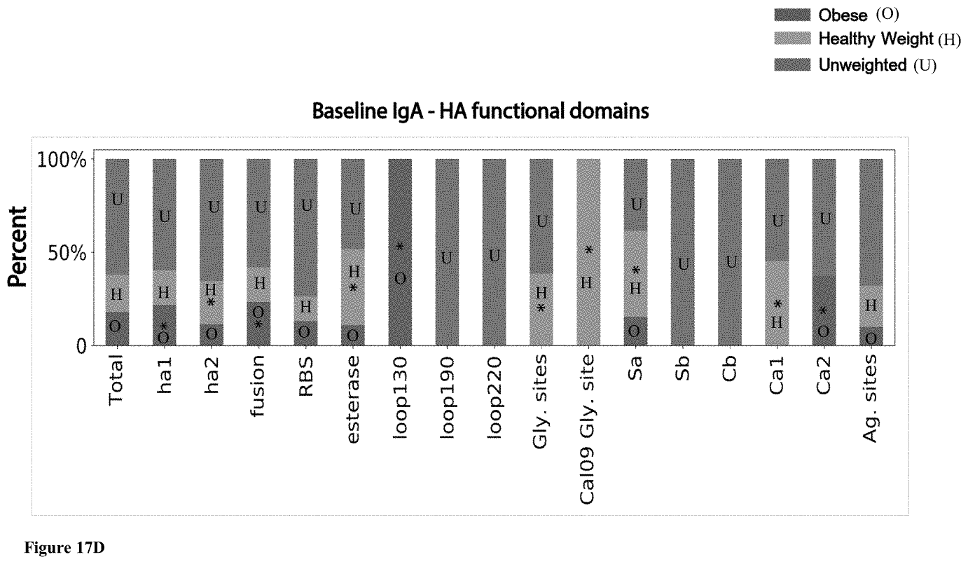

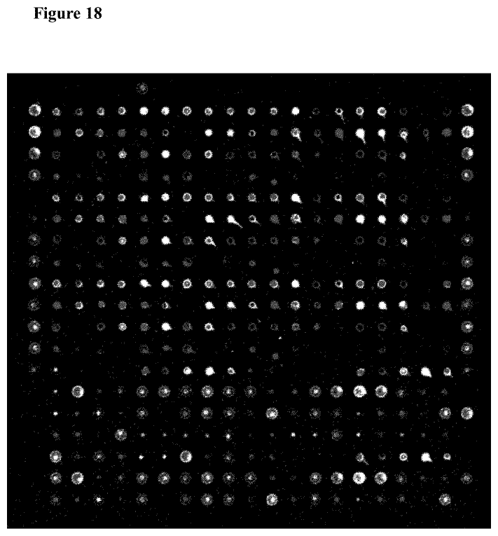

Since significant differences in the overall magnitude and breadth of antibodies to H1 and N1 peptides from the Cal09 vaccine strain in the obese and HW groups were observed, in particular for IgG antibodies, it was next asked whether the antibody profiles to individual peptides can be used to predict whether a subject is obese or HW. Logistic regression models were trained using the level of IgG and IgA antibodies of each subject that bind H1 and N1 peptides, as well as age and gender as input features. Four models were trained with IgG or IgA profiles to H1 and N1 peptides, either from baseline or baseline and post-vaccination timepoints, using leave-one-out cross validation. Performance was measured using the area under the receiver operating characteristic curve (AUC). It was found that both baseline and baseline+post-vaccination antibody profiles were highly predictive of obesity status, for both IgG and IgA profiles, with AUC scores ranging from 0.87-0.89 (FIG. 17A). Similar models based on the IgG and IgA magnitude to whole viruses and HA proteins, as well as the widely used HAI and MN titers, were less predictive of obesity status, with AUC scores ranging from 0.55 to 0.66 (FIG. 17B).

### Example 20: IgG and IgA Antibodies of Obese and HW Subjects Target Different Functional Sites in the Cal09 HA Protein at Baseline

The logistic regression models trained for predicting obesity status based on H1/N1 peptide arrays results (FIG. 17A) also assigned weights to individual features (peptides) from both the HA and NA proteins of the Cal09 vaccine strain. The weight of each feature represents its importance (or contribution) to the prediction model. The weights of H1 peptides were used to score individual positions on the Cal09 HA protein based on the maximal weight they were assigned within the model across all peptides that included each position. Each position was assigned a positive or negative weight, where positive weights were associated with obesity status and negative weights were associated with HW status. It was hypothesized that weighted positions would be located in surface exposed and functional sites on the Cal09 HA protein. To test this, whether there was enrichment for positively weighted sites (associated with HW responses) or negatively weighted sites (associated with obese responses) was computed within the two HA subunits (HA1 and HA2) as well as specific functional regions of the HA protein, including the receptor binding site (RBS), antigenic sites (Sa, Sb, Ca1, Ca2, Cb), glycosylation sites, the esterase domain, and the binding loops. It was found that positions targeted by HW subjects were enriched within the HA1 protein for IgG and the HA2 protein for IgA. In contrast, obese subjects targeted positions within HA2 for IgG and HA1 for IgA. When analyzing functional sites, it was found that binding of IgG antibodies to peptides of 5 of 15 domains was significantly higher in the HW: RBS, antigenic sites (overall as well site Sa), esterase domain, and glycosylation sites (FIG. 17C). IgA binding to 5 of 15 domains was also enriched in the HW subjects: Sa and Cal antigenic sites, glycosylation sites, and the esterase domain (FIG. 17D). In contrast, the obese group was enriched with both IgG and IgA antibodies to 3 of 15 sites compared with the HW: the fusion domain, Ca2 antigenic site and loop 130 (FIG. 17C-17D).

### Example 21: Microarrays can be Used to Identify Antibody Profiles that are Associated with a Higher Risk for Seasonal Influenza Morbidity and Mortality by Comparing High-Risk and Low-Risk Groups

Obese people are considered a high-risk group for Cal09 influenza morbidity and mortality. Obesity may also reduce influenza vaccine effectiveness. Therefore, a comparison of the IgG and IgA repertoires of obese and healthy-weight individuals may identify antibody features that are associated with a higher susceptibility to Cal09 infection, or with a lower effectiveness of Cal09 vaccination. While T-cells target only linear epitopes, antibodies can target both linear and conformational epitopes. The antibody response to a vaccination with a conformational antigen can include a large fraction of antibodies to linear epitopes. Nevertheless, antibodies to linear peptides do not always bind the folded whole protein or virus, and vice versa, and therefore antibodies to linear epitopes are often considered as less effective. Herein an in-depth analysis of the influenza specific IgG and IgA antibody repertoires of 189 subjects, of which 100 were obese, were conducted at baseline and one-month post-vaccination with the 2010-2011 Northern Hemisphere trivalent influenza vaccine (TIV), using antigen microarrays spotted with conformational and linear antigens. Conformational antigens included whole viruses and recombinant HA proteins, and the linear antigens included overlapping peptides spanning the sequences of HA and NA proteins of the Cal09 H1N1 vaccine strain. Significant differences between the anti-influenza antibody repertoires were observed pre-vaccination in the HW and obese groups. Obese individuals presented lower baseline levels of IgG to conformational antigens from all the three subtypes, in particular for the magnitude of anti-rH1 IgG, as well as a lower magnitude of IgA to rH1 proteins only. Concurrently, obese subjects had a wider and stronger IgG repertoire and a narrower IgA repertoire to Cal09 H1 and N1 peptides at baseline, compared with the HW group. In line with previous studies, it was found that both groups generated a significant rise in IgG and IgA antibody levels post-vaccination to most of the conformational antigens of the vaccine strains and other influenza strains from subtypes A/H1N1, A/H3N2 and B. However, the antibody response to vaccination, in particular the IgA response, was also significantly different in HW and obese groups. Thirty days post vaccination, obesity was associated with lower IgG levels, as well as higher IgA levels and fold-change to conformational antigens, accompanied with wider IgG responses and narrower IgA responses to linear Cal09 antigens. These findings suggest that an increase in the level of antibodies to conformational antigens is associated with a decrease in the diversity and abundance of antibodies to linear peptides.

By comparing the influenza BIH profiles of subjects, evidence for extensive heterogeneity in both the HW and obese groups was found. Furthermore, low IgG-BIH was not always associated with low IgA-BIH, as evidenced by the moderate correlations between IgG and IgA antibody profiles at baseline. To further assess the impact of this heterogeneity, all subjects were ranked by their BM antibody magnitude levels for both viruses, proteins and peptides. It was found that obese subjects were significantly more likely to belong to the low IgG-BIH groups for whole viruses and rH1 proteins, and to the low IgA-BIH for rH1 proteins. In contrast, obese subjects were more common in the high IgG-BIH groups for both H1 and N1 peptides, but their prevalence in the low and high IgA-BIH groups for Cal09 peptides were not significantly different. Furthermore, these differences were not observed when ranking subjects based on their antibody levels to rHA proteins or influenza subtypes H3N2 and B. Therefore, the reduced ability of obese subjects to develop an effective IgG response to the pandemic Cal09 strain may be associated with a biased IgG repertoire towards linear epitopes. A successful antibody response converts immature antibodies to linear epitopes to more effective and possibly neutralizing antibodies against the whole virus.

On the other hand, the obese group developed stronger and perhaps more effective IgA response to the vaccine rHA and whole virus antigens. The IgA response to influenza vaccination in obese and HW humans has not been previously compared.

The cohort included mainly class 1-2 obese cases (30<=BMI<40) with only 4 cases of severe obesity (BMI>40). While a previous study reported that obese subjects had higher levels of total IgG and in particular IgA antibodies, as measured using a nephelometry assay, no significant differences in the total IgG and IgA levels of the groups was found by ELISA. Therefore, the differences in anti-influenza IgG and IgA repertoires observed between the obese and HW groups were specific to influenza and not due to systemic differences in the overall antibody levels. The levels of serum IgG and IgA to the same influenza antigens were only moderately correlated, with higher correlations pre-vaccination, indicating that the IgG and IgA responses to the vaccine were independent. Correlations between IgG and IgA magnitudes to rHA proteins were weak at baseline and mostly non-significant post-vaccination.

It was also found that baseline immune-history profiles were associated with age: Elderly subjects were more likely to have high IgG- and IgA-BIH to whole H1N1 viruses and high IgA-BIH to rH1 proteins. These findings are in agreement with previous reports that repeated exposures to multiple influenza strains increases the breadth and magnitude of the influenza-specific antibody repertoire in the elderly. In addition, the elderly subjects were more likely to have low IgG-BIH to Cal09 H1 and N1 peptides, and in contrast—high IgA-BIH to Cal09 H1 peptides. The IgG results suggest that following multiple exposures, immature IgG antibodies to linear epitopes hypermutate to increase their affinity to three-dimensional epitopes that exist on the full protein or whole virus. Regarding IgA, the elderly group was enriched in high-IgA groups for both the three-dimensional rH1 proteins and the H1 peptides.

A combined analysis of obesity and age found that obese subjects with low-BIH to whole viruses and HA proteins were overall younger than obese subjects with high-BIH. Furthermore, these subjects had high IgG-BIH to Cal09 peptides. These findings suggest that subjects that become obese early in life are more likely to have weak immune memory to influenza viruses and HA proteins, and strong immune memory to peptide antigens.

The results provided hereinabove support the theory that pre-existing antibodies often, but not always, dominate the immune response to influenza vaccination. In many subjects in the study, the post-vaccination antibody repertoire tended to expand the baseline immune-history repertoire. It was also shown that baseline antibody profiles could be used to predict vaccine-induced responses for both whole viruses and HA proteins. Overall predictions were more accurate for IgA responses. These data suggest that the baseline antibody repertoire heavily biases the post-vaccination repertoire, further highlighting the importance of baseline differences between the HW and obese groups.

Finally, it was found that baseline and post-vaccination peptide antibody profiles can be used to predict whether a subject is obese or HW, with AUC scores ranging from 0.87-0.89. Further analysis of the specific peptides that were assigned significant weights by the regression model, revealed striking differences in the binding patterns of obese and HW subjects to functional sites. In particular, HW subjects had significantly higher IgG responses to peptides from the HA1 protein while obese subjects had significantly higher IgA responses to HA1 peptides. The opposite was true for HA2. Since HA2 contains the stalk domain and is relatively conserved, while HA1 contains the globular head that is more variable, anti-HA2 antibodies may be more cross-reactive with other influenza strains. Efforts to develop a broad universal influenza vaccine are often focused on induction of anti-HA2 Abs. Accordingly, in this study, higher levels of antibodies to Cal09 HA2 peptides at baseline were correlated with broader (higher breadth) but not stronger (higher magnitude) post-vaccination response. Subjects in the HW group that had stronger IgA responses to Cal09 HA2 peptides at baseline, also had broader IgA to H1N1 and H3N2 viruses post vaccinations. In contrast, subjects from the obese group that had stronger IgG responses to Cal09 HA2 peptides at baseline, also had broader IgG responses to H3N2 viruses post vaccination (compare FIGS. 9V-9BB and 10C-10N). Additional differences in antibody repertoires to peptide antigens in HW and obese subjects were also observed in the antigenic sites and the glycosylation sites, which are key targets of neutralizing antibodies to the HA protein and explain the increased risk of influenza H1N1 infection in obese subjects.

In summary, this study demonstrates the power of the combined use of antigen microarrays spotted with different types of antigens: whole viruses, recombinant proteins, and peptides, to compare the immune response of different populations to the same challenge, and to understand the relationship between antibodies to linear and three-dimensional epitopes. The in-depth analysis of the influenza-specific antibody repertoire found striking differences in the antibody repertoires of HW and obese subjects, and in elderlies compared with younger adults, despite the extensive heterogeneity of the baseline immune-history profiles of subjects in both groups. While, as previously reported, obese subjects respond to influenza vaccination, it was found that their IgG antibody repertoire is biased towards responses to linear peptides and not whole viruses and rHA proteins. This may suggest that lack of protection in the obese group may be due to the increase in IgG antibodies to linear peptides, which may not be protective. On the other hand, their IgG repertoire to Cal09 H1 and N1 peptides is broader and may be more cross-reactive to other strains. The IgA repertoire of the obese group was mirror-like: more IgA antibodies to whole viruses and rHA proteins, and narrower IgA repertoire to peptides. Different antibody repertoires were also identified in the elderly group compared with younger adults: both IgG and IgA repertoires of the elderly were biased to target conformational sites, while IgG antibodies to peptides were depleted.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. An array comprising a plurality of probes each immobilized at a discrete location on said array, wherein said plurality of probes comprises a probe from a first influenza strain or subtype and a probe from a second influenza strain or subtype.

2. The array of claim 1, wherein said plurality of probes comprises at least two probes from each strain or subtype of influenza.

3. The array of claim 1, wherein said influenza subtype is selected from the group consisting of: H1N1 influenza, H3N2 influenza, and influenza B or is selected from the subtypes listed in Table 1 and Table 5.

4. (canceled)

5. The array of claim 1, wherein said plurality of probes
a. are selected from a whole virus, a lysed virus, a virus-like particle (VLP), a whole recombinant protein and a peptide;
b. comprises a probe from an influenza subtype or strain from a first year and a probe from said influenza subtype or strain from a second year, optionally wherein said probe from an influenza subtype from a first year and said probe from an influenza subtype from a second year are from the same protein, from the same region of a protein or both;
c. comprises a peptide probe from each of said influenza strains or subtypes;
d. comprises a peptide probe from a hemagglutinin (HA) protein from each of said influenza strains or subtypes;
e. comprises a peptide probe comprises between 10 and 60 consecutive amino acids from an influenza protein;
f. comprises a recombinant protein from each of said influenza strains or subtypes, optionally wherein said recombinants protein is a recombinant surface protein;
g. comprises an inactivated form of each of said influenza strains or subtypes; or
h. a combination thereof.

6. (canceled)

7. (canceled)

8. (canceled)

9. (canceled)

10. The array of claim 5, wherein said plurality of probes comprises a peptide probe from a neuraminidase (NA) protein from each of said influenza strains or subtypes.

11. (canceled)

12. (canceled)

13. (canceled)

14. (canceled)

15. The array of claim 1, wherein said plurality of probes comprises a virus-like particle (VLP) of each of said influenza strains or subtypes or a lysate from a cell infected by each of said influenza strains or subtypes.

16. (canceled)

17. The array of claim 1, wherein said array is a human array and said plurality of probes are selected from Table 1 and SEQ ID NOs: 1-1390 or said array is a non-human array and said plurality of probes are selected from Table 5 and SEQ ID NO: 598-995.

18. (canceled)

19. (canceled)

20. (canceled)

21. (canceled)

22. (canceled)

23. (canceled)

24. A method of determining the suitability of a subject in need thereof to receive an influenza vaccination, for determining risk of symptomatic infection of a subject by an influenza virus, or for predicting effectiveness of an influenza vaccine in a subject in need thereof, the method comprising:
a. providing a biological sample from said subject comprising antibodies;
b. contacting said sample to an array of claim 1 in conditions sufficient for antibody binding to said probes;
c. detecting the binding of said antibodies to discrete locations on said array indicating the presence in said sample of antibodies to probes located at said detected discrete locations; and
d. generating an influenza immune score from said detected binding, wherein the magnitude of said immune score is proportional to said subject's suitability to receive an influenza vaccine and to effectiveness of an influenza vaccine in said subject;
thereby determining the suitability of a subject to receive an influenza vaccination, determining risk of symptomatic infection of a subject by an influenza virus or predicting effectiveness of an influenza vaccine in a subject.

25. The method of claim 24, wherein said subject has previously been vaccinated against influenza or previously been infected by an influenza virus.

26. The method of claim 24, wherein said influenza is selected from the group consisting of: H1N1 influenza, H3N2 influenza, and influenza B, said influenza subtype is selected from the subtypes listed in Table 1 and Table 5 or both.

27. (canceled)

28. The method of claim 24, wherein said biological sample is a peripheral blood sample, a plasma sample or a serum sample.

29. The method of claim 24, wherein said detecting comprises contacting said array with bound antibodies with labeled secondary antibodies against said antibodies in said biological sample, optionally wherein said secondary antibodies are directed against IgG, IgA, or both and wherein antibodies against IgG and IgA comprise distinct labels.

30. (canceled)

31. The method of claim 29, wherein said influenza immune score is generated from IgA binding.

32. The method of claim 24, wherein said influenza is a specific influenza strain or subtype and said immune score is generated from detected binding to probes from said specific strain or subtype.

33. The method of claim 29, wherein said detecting further comprises scanning said array with a detector configured to detect said labeled secondary antibodies and producing an output of the discrete locations where antibody was detected.

34. The method of claim 24, wherein
a. a higher immune score indicates a greater suitability to receive an influenza vaccination, decreased risk of symptomatic infection by an influenza or greater likelihood of effectiveness of an influenza vaccine, and wherein a lower immune score indicates a lesser suitability to receive an influenza vaccination, increased risk of symptomatic infection by an influenza or a lower likelihood of effectiveness of an influenza vaccine; or
b. an immune score above a predetermined threshold indicates the subject is suitable to receive an influenza vaccination, is at reduced risk of symptomatic infection by an influenza or the influenza vaccine is likely to be effective.

35. (canceled)

36. The method of claim 24, wherein said subject is a human subject, and said plurality of probes is selected from Table 1 and SEQ ID NO: 1-1390 or wherein said subject is a non-human subject and said plurality of probes is selected from Table 5 and SEQ ID NO: 598-995.

37. A method of predicting the effectiveness of an influenza vaccine, the method comprising:
a. providing a solution comprising antibodies from immune cells contacted by said influenza vaccine;
b. contacting said solution to an array of claim 1 in conditions sufficient for antibody binding to said probes;
c. detecting the binding of said antibodies to discrete locations on said array indicating the presence in said solution of antibodies to probes located at said detected discrete locations; and
d. generating an influenza immune score from said detected binding, wherein the magnitude of said immune score is proportional to said influenza vaccine's effectiveness;
thereby predicting the effectiveness of an influenza vaccine.

38. A kit comprising the array of claim 1, and labeled secondary antibodies configured for detection of antibodies bound to said array.

39. A system comprising the array of claim 1, and a detector configured to detect binding of antibodies to probes immobilized on said array, optionally wherein said detector is configured to detect labeled secondary antibodies.

40. (canceled)


SEQUENCE LISTING (1391 sequences)

SEQ ID NO: 1 - protein, 19 aa, Artificial sequence
  Synthetic
KTIIALSYILCLVFAQKLP

SEQ ID NO: 2 - protein, 19 aa, Artificial sequence
  Synthetic
LSYILCLVFAQKLPGNDNS

SEQ ID NO: 3 - protein, 19 aa, Artificial sequence
  Synthetic
CLVFAQKLPGNDNSTATLC

SEQ ID NO: 4 - protein, 19 aa, Artificial sequence
  Synthetic
QKLPGNDNSTATLCLGHHA

SEQ ID NO: 5 - protein, 19 aa, Artificial sequence
  Synthetic
NDNSTATLCLGHHAVPNGT

SEQ ID NO: 6 - protein, 19 aa, Artificial sequence
  Synthetic
ATLCLGHHAVPNGTIVKTI

SEQ ID NO: 7 - protein, 19 aa, Artificial sequence
  Synthetic
GHHAVPNGTIVKTITNDQI

SEQ ID NO: 8 - protein, 19 aa, Artificial sequence
  Synthetic
PNGTIVKTITNDQIEVTNA

SEQ ID NO: 9 - protein, 19 aa, Artificial sequence
  Synthetic
VKTITNDQIEVTNATELVQ

SEQ ID NO: 10 - protein, 19 aa, Artificial sequence
  Synthetic
NDQIEVTNATELVQSSSTG

SEQ ID NO: 11 - protein, 19 aa, Artificial sequence
  Synthetic
VTNATELVQSSSTGGICDS

SEQ ID NO: 12 - protein, 19 aa, Artificial sequence
  Synthetic
ELVQSSSTGGICDSPHQIL

SEQ ID NO: 13 - protein, 19 aa, Artificial sequence
  Synthetic
SSTGGICDSPHQILDGENC

SEQ ID NO: 14 - protein, 19 aa, Artificial sequence
  Synthetic
ICDSPHQILDGENCTLIDA

SEQ ID NO: 15 - protein, 19 aa, Artificial sequence
  Synthetic
HQILDGENCTLIDALLGDP

SEQ ID NO: 16 - protein, 19 aa, Artificial sequence
  Synthetic
GENCTLIDALLGDPQCDGF

SEQ ID NO: 17 - protein, 19 aa, Artificial sequence
  Synthetic
LIDALLGDPQCDGFQNKKW

SEQ ID NO: 18 - protein, 19 aa, Artificial sequence
  Synthetic
LGDPQCDGFQNKKWDLFVE

SEQ ID NO: 19 - protein, 19 aa, Artificial sequence
  Synthetic
CDGFQNKKWDLFVERSKAY

SEQ ID NO: 20 - protein, 19 aa, Artificial sequence
  Synthetic
NKKWDLFVERSKAYSNCYP

SEQ ID NO: 21 - protein, 19 aa, Artificial sequence
  Synthetic
LFVERSKAYSNCYPYDVPD

SEQ ID NO: 22 - protein, 19 aa, Artificial sequence
  Synthetic
SKAYSNCYPYDVPDYASLR

SEQ ID NO: 23 - protein, 19 aa, Artificial sequence
  Synthetic
NCYPYDVPDYASLRSLVAS

SEQ ID NO: 24 - protein, 19 aa, Artificial sequence
  Synthetic
DVPDYASLRSLVASSGTLE

SEQ ID NO: 25 - protein, 19 aa, Artificial sequence
  Synthetic
ASLRSLVASSGTLEFNDES

SEQ ID NO: 26 - protein, 19 aa, Artificial sequence
  Synthetic
LVASSGTLEFNDESFNWTG

SEQ ID NO: 27 - protein, 19 aa, Artificial sequence
  Synthetic
GTLEFNDESFNWTGVTQNG

SEQ ID NO: 28 - protein, 19 aa, Artificial sequence
  Synthetic
NDESFNWTGVTQNGTSSSC

SEQ ID NO: 29 - protein, 19 aa, Artificial sequence
  Synthetic
NWTGVTQNGTSSSCKRRSN

SEQ ID NO: 30 - protein, 19 aa, Artificial sequence
  Synthetic
TQNGTSSSCKRRSNNSFFS

SEQ ID NO: 31 - protein, 19 aa, Artificial sequence
  Synthetic
SSSCKRRSNNSFFSRLNWL

SEQ ID NO: 32 - protein, 19 aa, Artificial sequence
  Synthetic
RRSNNSFFSRLNWLTHLKF

SEQ ID NO: 33 - protein, 19 aa, Artificial sequence
  Synthetic
SFFSRLNWLTHLKFKYPAL

SEQ ID NO: 34 - protein, 19 aa, Artificial sequence
  Synthetic
LNWLTHLKFKYPALNVTMP

SEQ ID NO: 35 - protein, 19 aa, Artificial sequence
  Synthetic
HLKFKYPALNVTMPNNEKF

SEQ ID NO: 36 - protein, 19 aa, Artificial sequence
  Synthetic
YPALNVTMPNNEKFDKLYI

SEQ ID NO: 37 - protein, 19 aa, Artificial sequence
  Synthetic
VTMPNNEKFDKLYIWGVHH

SEQ ID NO: 38 - protein, 19 aa, Artificial sequence
  Synthetic
NEKFDKLYIWGVHHPVTDN

SEQ ID NO: 39 - protein, 19 aa, Artificial sequence
  Synthetic
KLYIWGVHHPVTDNDQIFL

SEQ ID NO: 40 - protein, 19 aa, Artificial sequence
  Synthetic
GVHHPVTDNDQIFLYAQAS

SEQ ID NO: 41 - protein, 19 aa, Artificial sequence
  Synthetic
VTDNDQIFLYAQASGRITV

SEQ ID NO: 42 - protein, 19 aa, Artificial sequence
  Synthetic
QIFLYAQASGRITVSTKRS

SEQ ID NO: 43 - protein, 19 aa, Artificial sequence
  Synthetic
AQASGRITVSTKRSQQTVI

SEQ ID NO: 44 - protein, 19 aa, Artificial sequence
  Synthetic
RITVSTKRSQQTVIPNIGS

SEQ ID NO: 45 - protein, 19 aa, Artificial sequence
  Synthetic
TKRSQQTVIPNIGSRPRIR

SEQ ID NO: 46 - protein, 19 aa, Artificial sequence
  Synthetic
QTVIPNIGSRPRIRNIPSR

SEQ ID NO: 47 - protein, 19 aa, Artificial sequence
  Synthetic
NIGSRPRIRNIPSRISIYW

SEQ ID NO: 48 - protein, 19 aa, Artificial sequence
  Synthetic
PRIRNIPSRISIYWTIVKP

SEQ ID NO: 49 - protein, 19 aa, Artificial sequence
  Synthetic
IPSRISIYWTIVKPGDILL

SEQ ID NO: 50 - protein, 19 aa, Artificial sequence
  Synthetic
SIYWTIVKPGDILLINSTG

SEQ ID NO: 51 - protein, 19 aa, Artificial sequence
  Synthetic
IVKPGDILLINSTGNLIAP

SEQ ID NO: 52 - protein, 19 aa, Artificial sequence
  Synthetic
DILLINSTGNLIAPRGYFK

SEQ ID NO: 53 - protein, 19 aa, Artificial sequence
  Synthetic
NSTGNLIAPRGYFKIRSGK

SEQ ID NO: 54 - protein, 19 aa, Artificial sequence
  Synthetic
LIAPRGYFKIRSGKSSIMR

SEQ ID NO: 55 - protein, 19 aa, Artificial sequence
  Synthetic
GYFKIRSGKSSIMRSDAPI

SEQ ID NO: 56 - protein, 19 aa, Artificial sequence
  Synthetic
RSGKSSIMRSDAPIGKCNS

SEQ ID NO: 57 - protein, 19 aa, Artificial sequence
  Synthetic
SIMRSDAPIGKCNSECITP

SEQ ID NO: 58 - protein, 19 aa, Artificial sequence
  Synthetic
DAPIGKCNSECITPNGSIP

SEQ ID NO: 59 - protein, 18 aa, Artificial sequence
  Synthetic
CNSECITPNGSIPNDKPF

SEQ ID NO: 60 - protein, 19 aa, Artificial sequence
  Synthetic
CITPNGSIPNDKPFQNVNR

SEQ ID NO: 61 - protein, 19 aa, Artificial sequence
  Synthetic
GSIPNDKPFQNVNRITYGA

SEQ ID NO: 62 - protein, 19 aa, Artificial sequence
  Synthetic
DKPFQNVNRITYGACPRYV

SEQ ID NO: 63 - protein, 19 aa, Artificial sequence
  Synthetic
NVNRITYGACPRYVKQNTL

SEQ ID NO: 64 - protein, 19 aa, Artificial sequence
  Synthetic
TYGACPRYVKQNTLKLATG

SEQ ID NO: 65 - protein, 19 aa, Artificial sequence
  Synthetic
PRYVKQNTLKLATGMRNVP

SEQ ID NO: 66 - protein, 19 aa, Artificial sequence
  Synthetic
QNTLKLATGMRNVPEKQTR

SEQ ID NO: 67 - protein, 19 aa, Artificial sequence
  Synthetic
LATGMRNVPEKQTRGIFGA

SEQ ID NO: 68 - protein, 19 aa, Artificial sequence
  Synthetic
RNVPEKQTRGIFGAIAGFI

SEQ ID NO: 69 - protein, 19 aa, Artificial sequence
  Synthetic
KQTRGIFGAIAGFIENGWE

SEQ ID NO: 70 - protein, 19 aa, Artificial sequence
  Synthetic
IFGAIAGFIENGWEGMVDG

SEQ ID NO: 71 - protein, 19 aa, Artificial sequence
  Synthetic
AGFIENGWEGMVDGWYGFR

SEQ ID NO: 72 - protein, 19 aa, Artificial sequence
  Synthetic
NGWEGMVDGWYGFRHQNSE

SEQ ID NO: 73 - protein, 19 aa, Artificial sequence
  Synthetic
MVDGWYGFRHQNSEGIGQA

SEQ ID NO: 74 - protein, 19 aa, Artificial sequence
  Synthetic
YGFRHQNSEGIGQAADLKS

SEQ ID NO: 75 - protein, 19 aa, Artificial sequence
  Synthetic
QNSEGIGQAADLKSTQAAI

SEQ ID NO: 76 - protein, 19 aa, Artificial sequence
  Synthetic
IGQAADLKSTQAAINQING

SEQ ID NO: 77 - protein, 19 aa, Artificial sequence
  Synthetic
DLKSTQAAINQINGKLNRL

SEQ ID NO: 78 - protein, 19 aa, Artificial sequence
  Synthetic
QAAINQINGKLNRLIGKTN

SEQ ID NO: 79 - protein, 19 aa, Artificial sequence
  Synthetic
QINGKLNRLIGKTNEKFHQ

SEQ ID NO: 80 - protein, 19 aa, Artificial sequence
  Synthetic
LNRLIGKTNEKFHQIEKEF

SEQ ID NO: 81 - protein, 19 aa, Artificial sequence
  Synthetic
GKTNEKFHQIEKEFSEVEG

SEQ ID NO: 82 - protein, 18 aa, Artificial sequence
  Synthetic
FHQIEKEFSEVEGRIQDL

SEQ ID NO: 83 - protein, 19 aa, Artificial sequence
  Synthetic
EKEFSEVEGRIQDLEKYVE

SEQ ID NO: 84 - protein, 19 aa, Artificial sequence
  Synthetic
EVEGRIQDLEKYVEDTKID

SEQ ID NO: 85 - protein, 19 aa, Artificial sequence
  Synthetic
IQDLEKYVEDTKIDLWSYN

SEQ ID NO: 86 - protein, 19 aa, Artificial sequence
  Synthetic
KYVEDTKIDLWSYNAELLV

SEQ ID NO: 87 - protein, 19 aa, Artificial sequence
  Synthetic
TKIDLWSYNAELLVALENQ

SEQ ID NO: 88 - protein, 19 aa, Artificial sequence
  Synthetic
WSYNAELLVALENQHTIDL

SEQ ID NO: 89 - protein, 19 aa, Artificial sequence
  Synthetic
ELLVALENQHTIDLTDSEM

SEQ ID NO: 90 - protein, 19 aa, Artificial sequence
  Synthetic
LENQHTIDLTDSEMNKLFE

SEQ ID NO: 91 - protein, 19 aa, Artificial sequence
  Synthetic
TIDLTDSEMNKLFERTKKQ

SEQ ID NO: 92 - protein, 19 aa, Artificial sequence
  Synthetic
DSEMNKLFERTKKQLRENA

SEQ ID NO: 93 - protein, 19 aa, Artificial sequence
  Synthetic
KLFERTKKQLRENAEDMGN

SEQ ID NO: 94 - protein, 19 aa, Artificial sequence
  Synthetic
TKKQLRENAEDMGNGCFKI

SEQ ID NO: 95 - protein, 19 aa, Artificial sequence
  Synthetic
RENAEDMGNGCFKIYHKCD

SEQ ID NO: 96 - protein, 19 aa, Artificial sequence
  Synthetic
DMGNGCFKIYHKCDNACIG

SEQ ID NO: 97 - protein, 19 aa, Artificial sequence
  Synthetic
CFKIYHKCDNACIGSIRNG

SEQ ID NO: 98 - protein, 19 aa, Artificial sequence
  Synthetic
HKCDNACIGSIRNGTYDHD

SEQ ID NO: 99 - protein, 19 aa, Artificial sequence
  Synthetic
ACIGSIRNGTYDHDVYRDE

SEQ ID NO: 100 - protein, 19 aa, Artificial sequence
  Synthetic
IRNGTYDHDVYRDEALNNR

SEQ ID NO: 101 - protein, 19 aa, Artificial sequence
  Synthetic
YDHDVYRDEALNNRFQIKG

SEQ ID NO: 102 - protein, 19 aa, Artificial sequence
  Synthetic
YRDEALNNRFQIKGVELKS

SEQ ID NO: 103 - protein, 19 aa, Artificial sequence
  Synthetic
LNNRFQIKGVELKSGYKDW

SEQ ID NO: 104 - protein, 19 aa, Artificial sequence
  Synthetic
QIKGVELKSGYKDWILWIS

SEQ ID NO: 105 - protein, 19 aa, Artificial sequence
  Synthetic
ELKSGYKDWILWISFAISC

SEQ ID NO: 106 - protein, 19 aa, Artificial sequence
  Synthetic
YKDWILWISFAISCFLLCV

SEQ ID NO: 107 - protein, 19 aa, Artificial sequence
  Synthetic
LWISFAISCFLLCVALLGF

SEQ ID NO: 108 - protein, 19 aa, Artificial sequence
  Synthetic
AISCFLLCVALLGFIMWAC

SEQ ID NO: 109 - protein, 19 aa, Artificial sequence
  Synthetic
LLCVALLGFIMWACQKGNI

SEQ ID NO: 110 - protein, 19 aa, Artificial sequence
  Synthetic
NPNQKIITIGSVSLTISTI

SEQ ID NO: 111 - protein, 19 aa, Artificial sequence
  Synthetic
IITIGSVSLTISTICFFMQ

SEQ ID NO: 112 - protein, 19 aa, Artificial sequence
  Synthetic
SVSLTISTICFFMQIAILI

SEQ ID NO: 113 - protein, 19 aa, Artificial sequence
  Synthetic
ISTICFFMQIAILITTVTL

SEQ ID NO: 114 - protein, 19 aa, Artificial sequence
  Synthetic
FFMQIAILITTVTLHFKQY

SEQ ID NO: 115 - protein, 19 aa, Artificial sequence
  Synthetic
AILITTVTLHFKQYEFNSP

SEQ ID NO: 116 - protein, 19 aa, Artificial sequence
  Synthetic
TVTLHFKQYEFNSPPNNQV

SEQ ID NO: 117 - protein, 19 aa, Artificial sequence
  Synthetic
FKQYEFNSPPNNQVMLCEP

SEQ ID NO: 118 - protein, 19 aa, Artificial sequence
  Synthetic
FNSPPNNQVMLCEPTIIER

SEQ ID NO: 119 - protein, 19 aa, Artificial sequence
  Synthetic
NNQVMLCEPTIIERNITEI

SEQ ID NO: 120 - protein, 19 aa, Artificial sequence
  Synthetic
LCEPTIIERNITEIVYLTN

SEQ ID NO: 121 - protein, 19 aa, Artificial sequence
  Synthetic
IIERNITEIVYLTNTTIEK

SEQ ID NO: 122 - protein, 19 aa, Artificial sequence
  Synthetic
ITEIVYLTNTTIEKEICPK

SEQ ID NO: 123 - protein, 19 aa, Artificial sequence
  Synthetic
YLTNTTIEKEICPKLAEYR

SEQ ID NO: 124 - protein, 19 aa, Artificial sequence
  Synthetic
TIEKEICPKLAEYRNWSKP

SEQ ID NO: 125 - protein, 19 aa, Artificial sequence
  Synthetic
ICPKLAEYRNWSKPQCNIT

SEQ ID NO: 126 - protein, 19 aa, Artificial sequence
  Synthetic
AEYRNWSKPQCNITGFAPF

SEQ ID NO: 127 - protein, 19 aa, Artificial sequence
  Synthetic
WSKPQCNITGFAPFSKDNS

SEQ ID NO: 128 - protein, 19 aa, Artificial sequence
  Synthetic
CNITGFAPFSKDNSIRLSA

SEQ ID NO: 129 - protein, 19 aa, Artificial sequence
  Synthetic
FAPFSKDNSIRLSAGGDIW

SEQ ID NO: 130 - protein, 18 aa, Artificial sequence
  Synthetic
DNSIRLSAGGDIWVTREP

SEQ ID NO: 131 - protein, 19 aa, Artificial sequence
  Synthetic
RLSAGGDIWVTREPYVSCD

SEQ ID NO: 132 - protein, 19 aa, Artificial sequence
  Synthetic
GDIWVTREPYVSCDPDKCY

SEQ ID NO: 133 - protein, 19 aa, Artificial sequence
  Synthetic
TREPYVSCDPDKCYQFALG

SEQ ID NO: 134 - protein, 19 aa, Artificial sequence
  Synthetic
VSCDPDKCYQFALGQGTTL

SEQ ID NO: 135 - protein, 19 aa, Artificial sequence
  Synthetic
DKCYQFALGQGTTLNNVHS

SEQ ID NO: 136 - protein, 19 aa, Artificial sequence
  Synthetic
FALGQGTTLNNVHSNDTVH

SEQ ID NO: 137 - protein, 19 aa, Artificial sequence
  Synthetic
GTTLNNVHSNDTVHDRTPY

SEQ ID NO: 138 - protein, 19 aa, Artificial sequence
  Synthetic
NVHSNDTVHDRTPYRTLLM

SEQ ID NO: 139 - protein, 19 aa, Artificial sequence
  Synthetic
DTVHDRTPYRTLLMNELGV

SEQ ID NO: 140 - protein, 19 aa, Artificial sequence
  Synthetic
RTPYRTLLMNELGVPFHLG

SEQ ID NO: 141 - protein, 19 aa, Artificial sequence
  Synthetic
TLLMNELGVPFHLGTKQVC

SEQ ID NO: 142 - protein, 19 aa, Artificial sequence
  Synthetic
ELGVPFHLGTKQVCIAWSS

SEQ ID NO: 143 - protein, 19 aa, Artificial sequence
  Synthetic
FHLGTKQVCIAWSSSSCHD

SEQ ID NO: 144 - protein, 19 aa, Artificial sequence
  Synthetic
KQVCIAWSSSSCHDGKAWL

SEQ ID NO: 145 - protein, 19 aa, Artificial sequence
  Synthetic
AWSSSSCHDGKAWLHVCVT

SEQ ID NO: 146 - protein, 19 aa, Artificial sequence
  Synthetic
SCHDGKAWLHVCVTGDDKN

SEQ ID NO: 147 - protein, 19 aa, Artificial sequence
  Synthetic
KAWLHVCVTGDDKNATASF

SEQ ID NO: 148 - protein, 19 aa, Artificial sequence
  Synthetic
VCVTGDDKNATASFIYNGR

SEQ ID NO: 149 - protein, 19 aa, Artificial sequence
  Synthetic
DDKNATASFIYNGRLVDSI

SEQ ID NO: 150 - protein, 19 aa, Artificial sequence
  Synthetic
TASFIYNGRLVDSIVSWSK

SEQ ID NO: 151 - protein, 19 aa, Artificial sequence
  Synthetic
YNGRLVDSIVSWSKEILRT

SEQ ID NO: 152 - protein, 19 aa, Artificial sequence
  Synthetic
VDSIVSWSKEILRTQESEC

SEQ ID NO: 153 - protein, 19 aa, Artificial sequence
  Synthetic
SWSKEILRTQESECVCING

SEQ ID NO: 154 - protein, 19 aa, Artificial sequence
  Synthetic
ILRTQESECVCINGTCTVV

SEQ ID NO: 155 - protein, 19 aa, Artificial sequence
  Synthetic
ESECVCINGTCTVVMTDGS

SEQ ID NO: 156 - protein, 19 aa, Artificial sequence
  Synthetic
CINGTCTVVMTDGSASGKA

SEQ ID NO: 157 - protein, 19 aa, Artificial sequence
  Synthetic
CTVVMTDGSASGKADTKIL

SEQ ID NO: 158 - protein, 19 aa, Artificial sequence
  Synthetic
TDGSASGKADTKILFIEEG

SEQ ID NO: 159 - protein, 19 aa, Artificial sequence
  Synthetic
SGKADTKILFIEEGKIVHT

SEQ ID NO: 160 - protein, 19 aa, Artificial sequence
  Synthetic
TKILFIEEGKIVHTSTLSG

SEQ ID NO: 161 - protein, 19 aa, Artificial sequence
  Synthetic
IEEGKIVHTSTLSGSAQHV

SEQ ID NO: 162 - protein, 19 aa, Artificial sequence
  Synthetic
IVHTSTLSGSAQHVEECSC

SEQ ID NO: 163 - protein, 19 aa, Artificial sequence
  Synthetic
TLSGSAQHVEECSCYPRYL

SEQ ID NO: 164 - protein, 19 aa, Artificial sequence
  Synthetic
AQHVEECSCYPRYLGVRCV

SEQ ID NO: 165 - protein, 19 aa, Artificial sequence
  Synthetic
ECSCYPRYLGVRCVCRDNW

SEQ ID NO: 166 - protein, 19 aa, Artificial sequence
  Synthetic
PRYLGVRCVCRDNWKGSNR

SEQ ID NO: 167 - protein, 19 aa, Artificial sequence
  Synthetic
VRCVCRDNWKGSNRPIVDI

SEQ ID NO: 168 - protein, 19 aa, Artificial sequence
  Synthetic
RDNWKGSNRPIVDINIKDY

SEQ ID NO: 169 - protein, 19 aa, Artificial sequence
  Synthetic
GSNRPIVDINIKDYSIVSS

SEQ ID NO: 170 - protein, 19 aa, Artificial sequence
  Synthetic
IVDINIKDYSIVSSYVCSG

SEQ ID NO: 171 - protein, 19 aa, Artificial sequence
  Synthetic
IKDYSIVSSYVCSGLVGDT

SEQ ID NO: 172 - protein, 19 aa, Artificial sequence
  Synthetic
IVSSYVCSGLVGDTPRKND

SEQ ID NO: 173 - protein, 19 aa, Artificial sequence
  Synthetic
VCSGLVGDTPRKNDSSSSS

SEQ ID NO: 174 - protein, 19 aa, Artificial sequence
  Synthetic
VGDTPRKNDSSSSSHCLDP

SEQ ID NO: 175 - protein, 19 aa, Artificial sequence
  Synthetic
RKNDSSSSSHCLDPNNEEG

SEQ ID NO: 176 - protein, 19 aa, Artificial sequence
  Synthetic
SSSSHCLDPNNEEGGHGVK

SEQ ID NO: 177 - protein, 19 aa, Artificial sequence
  Synthetic
CLDPNNEEGGHGVKGWAFD

SEQ ID NO: 178 - protein, 19 aa, Artificial sequence
  Synthetic
NEEGGHGVKGWAFDDGNDV

SEQ ID NO: 179 - protein, 19 aa, Artificial sequence
  Synthetic
HGVKGWAFDDGNDVWMGRT

SEQ ID NO: 180 - protein, 19 aa, Artificial sequence
  Synthetic
WAFDDGNDVWMGRTISEKL

SEQ ID NO: 181 - protein, 19 aa, Artificial sequence
  Synthetic
GNDVWMGRTISEKLRSGYE

SEQ ID NO: 182 - protein, 19 aa, Artificial sequence
  Synthetic
MGRTISEKLRSGYETFKVI

SEQ ID NO: 183 - protein, 19 aa, Artificial sequence
  Synthetic
SEKLRSGYETFKVIEGWSN

SEQ ID NO: 184 - protein, 19 aa, Artificial sequence
  Synthetic
SGYETFKVIEGWSNPNSKL

SEQ ID NO: 185 - protein, 19 aa, Artificial sequence
  Synthetic
FKVIEGWSNPNSKLQINRQ

SEQ ID NO: 186 - protein, 19 aa, Artificial sequence
  Synthetic
GWSNPNSKLQINRQVIVDR

SEQ ID NO: 187 - protein, 19 aa, Artificial sequence
  Synthetic
NSKLQINRQVIVDRGNRSG

SEQ ID NO: 188 - protein, 19 aa, Artificial sequence
  Synthetic
INRQVIVDRGNRSGYSGIF

SEQ ID NO: 189 - protein, 19 aa, Artificial sequence
  Synthetic
IVDRGNRSGYSGIFSVEGK

SEQ ID NO: 190 - protein, 19 aa, Artificial sequence
  Synthetic
NRSGYSGIFSVEGKSCINR

SEQ ID NO: 191 - protein, 19 aa, Artificial sequence
  Synthetic
SGIFSVEGKSCINRCFYVE

SEQ ID NO: 192 - protein, 19 aa, Artificial sequence
  Synthetic
VEGKSCINRCFYVELIRGR

SEQ ID NO: 193 - protein, 19 aa, Artificial sequence
  Synthetic
CINRCFYVELIRGRKEETE

SEQ ID NO: 194 - protein, 19 aa, Artificial sequence
  Synthetic
FYVELIRGRKEETEVLWTS

SEQ ID NO: 195 - protein, 19 aa, Artificial sequence
  Synthetic
IRGRKEETEVLWTSNSIVV

SEQ ID NO: 196 - protein, 19 aa, Artificial sequence
  Synthetic
EETEVLWTSNSIVVFCGTS

SEQ ID NO: 197 - protein, 19 aa, Artificial sequence
  Synthetic
LWTSNSIVVFCGTSGTYGT

SEQ ID NO: 198 - protein, 19 aa, Artificial sequence
  Synthetic
SIVVFCGTSGTYGTGSWPD

SEQ ID NO: 199 - protein, 19 aa, Artificial sequence
  Synthetic
CGTSGTYGTGSWPDGADIN

SEQ ID NO: 200 - protein, 22 aa, Artificial sequence
  Synthetic
MKAILVVLLYTFATANADTLKK

SEQ ID NO: 201 - protein, 20 aa, Artificial sequence
  Synthetic
VVLLYTFATANADTLCIGYH

SEQ ID NO: 202 - protein, 20 aa, Artificial sequence
  Synthetic
TFATANADTLCIGYHANNST

SEQ ID NO: 203 - protein, 20 aa, Artificial sequence
  Synthetic
NADTLCIGYHANNSTDTVDT

SEQ ID NO: 204 - protein, 20 aa, Artificial sequence
  Synthetic
CIGYHANNSTDTVDTVLEKN

SEQ ID NO: 205 - protein, 20 aa, Artificial sequence
  Synthetic
ANNSTDTVDTVLEKNVTVTH

SEQ ID NO: 206 - protein, 20 aa, Artificial sequence
  Synthetic
DTVDTVLEKNVTVTHSVNLL

SEQ ID NO: 207 - protein, 20 aa, Artificial sequence
  Synthetic
VLEKNVTVTHSVNLLEDKHN

SEQ ID NO: 208 - protein, 20 aa, Artificial sequence
  Synthetic
VTVTHSVNLLEDKHNGKLCK

SEQ ID NO: 209 - protein, 20 aa, Artificial sequence
  Synthetic
SVNLLEDKHNGKLCKLRGVA

SEQ ID NO: 210 - protein, 20 aa, Artificial sequence
  Synthetic
EDKHNGKLCKLRGVAPLHLG

SEQ ID NO: 211 - protein, 20 aa, Artificial sequence
  Synthetic
GKLCKLRGVAPLHLGKCNIA

SEQ ID NO: 212 - protein, 20 aa, Artificial sequence
  Synthetic
LRGVAPLHLGKCNIAGWILG

SEQ ID NO: 213 - protein, 20 aa, Artificial sequence
  Synthetic
PLHLGKCNIAGWILGNPECE

SEQ ID NO: 214 - protein, 20 aa, Artificial sequence
  Synthetic
KCNIAGWILGNPECESLSTA

SEQ ID NO: 215 - protein, 20 aa, Artificial sequence
  Synthetic
GWILGNPECESLSTASSWSY

SEQ ID NO: 216 - protein, 20 aa, Artificial sequence
  Synthetic
NPECESLSTASSWSYIVETP

SEQ ID NO: 217 - protein, 20 aa, Artificial sequence
  Synthetic
SLSTASSWSYIVETPSSDNG

SEQ ID NO: 218 - protein, 20 aa, Artificial sequence
  Synthetic
SSWSYIVETPSSDNGTCYPG

SEQ ID NO: 219 - protein, 20 aa, Artificial sequence
  Synthetic
IVETPSSDNGTCYPGDFIDY

SEQ ID NO: 220 - protein, 20 aa, Artificial sequence
  Synthetic
SSDNGTCYPGDFIDYEELRE

SEQ ID NO: 221 - protein, 20 aa, Artificial sequence
  Synthetic
TCYPGDFIDYEELREQLSSV

SEQ ID NO: 222 - protein, 20 aa, Artificial sequence
  Synthetic
DFIDYEELREQLSSVSSFER

SEQ ID NO: 223 - protein, 20 aa, Artificial sequence
  Synthetic
EELREQLSSVSSFERFEIFP

SEQ ID NO: 224 - protein, 20 aa, Artificial sequence
  Synthetic
QLSSVSSFERFEIFPKTSSW

SEQ ID NO: 225 - protein, 20 aa, Artificial sequence
  Synthetic
SSFERFEIFPKTSSWPNHDS

SEQ ID NO: 226 - protein, 20 aa, Artificial sequence
  Synthetic
FEIFPKTSSWPNHDSNKGVT

SEQ ID NO: 227 - protein, 20 aa, Artificial sequence
  Synthetic
KTSSWPNHDSNKGVTAACPH

SEQ ID NO: 228 - protein, 20 aa, Artificial sequence
  Synthetic
PNHDSNKGVTAACPHAGAKS

SEQ ID NO: 229 - protein, 20 aa, Artificial sequence
  Synthetic
NKGVTAACPHAGAKSFYKNL

SEQ ID NO: 230 - protein, 20 aa, Artificial sequence
  Synthetic
AACPHAGAKSFYKNLIWLVK

SEQ ID NO: 231 - protein, 20 aa, Artificial sequence
  Synthetic
AGAKSFYKNLIWLVKKGNSY

SEQ ID NO: 232 - protein, 20 aa, Artificial sequence
  Synthetic
FYKNLIWLVKKGNSYPKLSK

SEQ ID NO: 233 - protein, 20 aa, Artificial sequence
  Synthetic
IWLVKKGNSYPKLSKSYIND

SEQ ID NO: 234 - protein, 20 aa, Artificial sequence
  Synthetic
KGNSYPKLSKSYINDKGKEV

SEQ ID NO: 235 - protein, 20 aa, Artificial sequence
  Synthetic
PKLSKSYINDKGKEVLVLWG

SEQ ID NO: 236 - protein, 20 aa, Artificial sequence
  Synthetic
SYINDKGKEVLVLWGIHHPS

SEQ ID NO: 237 - protein, 20 aa, Artificial sequence
  Synthetic
KGKEVLVLWGIHHPSTSADQ

SEQ ID NO: 238 - protein, 20 aa, Artificial sequence
  Synthetic
LVLWGIHHPSTSADQQSLYQ

SEQ ID NO: 239 - protein, 20 aa, Artificial sequence
  Synthetic
IHHPSTSADQQSLYQNADAY

SEQ ID NO: 240 - protein, 20 aa, Artificial sequence
  Synthetic
TSADQQSLYQNADAYVFVGS

SEQ ID NO: 241 - protein, 20 aa, Artificial sequence
  Synthetic
QSLYQNADAYVFVGSSRYSK

SEQ ID NO: 242 - protein, 20 aa, Artificial sequence
  Synthetic
NADAYVFVGSSRYSKKFKPE

SEQ ID NO: 243 - protein, 20 aa, Artificial sequence
  Synthetic
VFVGSSRYSKKFKPEIAIRP

SEQ ID NO: 244 - protein, 20 aa, Artificial sequence
  Synthetic
SRYSKKFKPEIAIRPKVRDQ

SEQ ID NO: 245 - protein, 20 aa, Artificial sequence
  Synthetic
KFKPEIAIRPKVRDQEGRMN

SEQ ID NO: 246 - protein, 20 aa, Artificial sequence
  Synthetic
IAIRPKVRDQEGRMNYYWTL

SEQ ID NO: 247 - protein, 20 aa, Artificial sequence
  Synthetic
KVRDQEGRMNYYWTLVEPGD

SEQ ID NO: 248 - protein, 20 aa, Artificial sequence
  Synthetic
EGRMNYYWTLVEPGDKITFE

SEQ ID NO: 249 - protein, 20 aa, Artificial sequence
  Synthetic
YYWTLVEPGDKITFEATGNL

SEQ ID NO: 250 - protein, 20 aa, Artificial sequence
  Synthetic
VEPGDKITFEATGNLVVPRY

SEQ ID NO: 251 - protein, 20 aa, Artificial sequence
  Synthetic
KITFEATGNLVVPRYAFAME

SEQ ID NO: 252 - protein, 20 aa, Artificial sequence
  Synthetic
ATGNLVVPRYAFAMERNAGS

SEQ ID NO: 253 - protein, 20 aa, Artificial sequence
  Synthetic
VVPRYAFAMERNAGSGIIIS

SEQ ID NO: 254 - protein, 20 aa, Artificial sequence
  Synthetic
AFAMERNAGSGIIISDTPVH

SEQ ID NO: 255 - protein, 20 aa, Artificial sequence
  Synthetic
RNAGSGIIISDTPVHDCNTT

SEQ ID NO: 256 - protein, 20 aa, Artificial sequence
  Synthetic
GIIISDTPVHDCNTTCQTPK

SEQ ID NO: 257 - protein, 20 aa, Artificial sequence
  Synthetic
DTPVHDCNTTCQTPKGAINT

SEQ ID NO: 258 - protein, 20 aa, Artificial sequence
  Synthetic
DCNTTCQTPKGAINTSLPFQ

SEQ ID NO: 259 - protein, 20 aa, Artificial sequence
  Synthetic
CQTPKGAINTSLPFQNIHPI

SEQ ID NO: 260 - protein, 20 aa, Artificial sequence
  Synthetic
GAINTSLPFQNIHPITIGKC

SEQ ID NO: 261 - protein, 20 aa, Artificial sequence
  Synthetic
SLPFQNIHPITIGKCPKYVK

SEQ ID NO: 262 - protein, 20 aa, Artificial sequence
  Synthetic
NIHPITIGKCPKYVKSTKLR

SEQ ID NO: 263 - protein, 20 aa, Artificial sequence
  Synthetic
TIGKCPKYVKSTKLRLATGL

SEQ ID NO: 264 - protein, 20 aa, Artificial sequence
  Synthetic
PKYVKSTKLRLATGLRNIPS

SEQ ID NO: 265 - protein, 20 aa, Artificial sequence
  Synthetic
STKLRLATGLRNIPSIQSRG

SEQ ID NO: 266 - protein, 20 aa, Artificial sequence
  Synthetic
LATGLRNIPSIQSRGLFGAI

SEQ ID NO: 267 - protein, 20 aa, Artificial sequence
  Synthetic
RNIPSIQSRGLFGAIAGFIE

SEQ ID NO: 268 - protein, 20 aa, Artificial sequence
  Synthetic
IQSRGLFGAIAGFIEGGWTG

SEQ ID NO: 269 - protein, 24 aa, Artificial sequence
  Synthetic
KKLFGAIAGFIEGGWTGMVDGWKK

SEQ ID NO: 270 - protein, 20 aa, Artificial sequence
  Synthetic
AGFIEGGWTGMVDGWYGYHH

SEQ ID NO: 271 - protein, 20 aa, Artificial sequence
  Synthetic
GGWTGMVDGWYGYHHQNEQG

SEQ ID NO: 272 - protein, 20 aa, Artificial sequence
  Synthetic
MVDGWYGYHHQNEQGSGYAA

SEQ ID NO: 273 - protein, 20 aa, Artificial sequence
  Synthetic
YGYHHQNEQGSGYAADLKST

SEQ ID NO: 274 - protein, 20 aa, Artificial sequence
  Synthetic
QNEQGSGYAADLKSTQNAID

SEQ ID NO: 275 - protein, 20 aa, Artificial sequence
  Synthetic
SGYAADLKSTQNAIDEITNK

SEQ ID NO: 276 - protein, 20 aa, Artificial sequence
  Synthetic
DLKSTQNAIDEITNKVNSVI

SEQ ID NO: 277 - protein, 20 aa, Artificial sequence
  Synthetic
QNAIDEITNKVNSVIEKMNT

SEQ ID NO: 278 - protein, 20 aa, Artificial sequence
  Synthetic
EITNKVNSVIEKMNTQFTAV

SEQ ID NO: 279 - protein, 20 aa, Artificial sequence
  Synthetic
VNSVIEKMNTQFTAVGKEFN

SEQ ID NO: 280 - protein, 20 aa, Artificial sequence
  Synthetic
EKMNTQFTAVGKEFNHLEKR

SEQ ID NO: 281 - protein, 20 aa, Artificial sequence
  Synthetic
QFTAVGKEFNHLEKRIENLN

SEQ ID NO: 282 - protein, 18 aa, Artificial sequence
  Synthetic
GKEFNHLEKRIENLNVDD

SEQ ID NO: 283 - protein, 18 aa, Artificial sequence
  Synthetic
HLEKRIENLNVDDGFLDI

SEQ ID NO: 284 - protein, 18 aa, Artificial sequence
  Synthetic
IENLNVDDGFLDIWTYNA

SEQ ID NO: 285 - protein, 18 aa, Artificial sequence
  Synthetic
VDDGFLDIWTYNAELLVL

SEQ ID NO: 286 - protein, 20 aa, Artificial sequence
  Synthetic
GFLDIWTYNAELLVLLENER

SEQ ID NO: 287 - protein, 20 aa, Artificial sequence
  Synthetic
WTYNAELLVLLENERTLDYH

SEQ ID NO: 288 - protein, 20 aa, Artificial sequence
  Synthetic
ELLVLLENERTLDYHDSNVK

SEQ ID NO: 289 - protein, 20 aa, Artificial sequence
  Synthetic
LENERTLDYHDSNVKNLYEK

SEQ ID NO: 290 - protein, 20 aa, Artificial sequence
  Synthetic
TLDYHDSNVKNLYEKVRSQL

SEQ ID NO: 291 - protein, 20 aa, Artificial sequence
  Synthetic
DSNVKNLYEKVRSQLKNNAK

SEQ ID NO: 292 - protein, 20 aa, Artificial sequence
  Synthetic
NLYEKVRSQLKNNAKEIGNG

SEQ ID NO: 293 - protein, 20 aa, Artificial sequence
  Synthetic
VRSQLKNNAKEIGNGCFEFY

SEQ ID NO: 294 - protein, 20 aa, Artificial sequence
  Synthetic
KNNAKEIGNGCFEFYHKCDN

SEQ ID NO: 295 - protein, 20 aa, Artificial sequence
  Synthetic
EIGNGCFEFYHKCDNTCMES

SEQ ID NO: 296 - protein, 20 aa, Artificial sequence
  Synthetic
CFEFYHKCDNTCMESVKNGT

SEQ ID NO: 297 - protein, 20 aa, Artificial sequence
  Synthetic
HKCDNTCMESVKNGTYDYPK

SEQ ID NO: 298 - protein, 20 aa, Artificial sequence
  Synthetic
TCMESVKNGTYDYPKYSEEA

SEQ ID NO: 299 - protein, 20 aa, Artificial sequence
  Synthetic
VKNGTYDYPKYSEEAKLNRE

SEQ ID NO: 300 - protein, 20 aa, Artificial sequence
  Synthetic
YDYPKYSEEAKLNREEIDGV

SEQ ID NO: 301 - protein, 20 aa, Artificial sequence
  Synthetic
YSEEAKLNREEIDGVKLEST

SEQ ID NO: 302 - protein, 20 aa, Artificial sequence
  Synthetic
KLNREEIDGVKLESTRIYQI

SEQ ID NO: 303 - protein, 20 aa, Artificial sequence
  Synthetic
EIDGVKLESTRIYQILAIYS

SEQ ID NO: 304 - protein, 20 aa, Artificial sequence
  Synthetic
KLESTRIYQILAIYSTVASS

SEQ ID NO: 305 - protein, 21 aa, Artificial sequence
  Synthetic
KRIYQILAIYSTVASSLVLVV

SEQ ID NO: 306 - protein, 21 aa, Artificial sequence
  Synthetic
KLAIYSTVASSLVLVVSLGAI

SEQ ID NO: 307 - protein, 21 aa, Artificial sequence
  Synthetic
KTVASSLVLVVSLGAISFWMC

SEQ ID NO: 308 - protein, 21 aa, Artificial sequence
  Synthetic
KLVLVVSLGAISFWMCSNGSL

SEQ ID NO: 309 - protein, 21 aa, Artificial sequence
  Synthetic
KSLGAISFWMCSNGSLQCRIC

SEQ ID NO: 310 - protein, 16 aa, Artificial sequence
  Synthetic
SFWMCSNGSLQCRICI

SEQ ID NO: 311 - protein, 20 aa, Artificial sequence
  Synthetic
MNPNQKIITIGSVCMTIGMA

SEQ ID NO: 312 - protein, 21 aa, Artificial sequence
  Synthetic
KTIGMANLILQIGNIISIWIS

SEQ ID NO: 313 - protein, 21 aa, Artificial sequence
  Synthetic
KNLILQIGNIISIWISHSIQL

SEQ ID NO: 314 - protein, 21 aa, Artificial sequence
  Synthetic
KIGNIISIWISHSIQLGNQNQ

SEQ ID NO: 315 - protein, 21 aa, Artificial sequence
  Synthetic
KSIWISHSIQLGNQNQIETCN

SEQ ID NO: 316 - protein, 21 aa, Artificial sequence
  Synthetic
KHSIQLGNQNQIETCNQSVIT

SEQ ID NO: 317 - protein, 21 aa, Artificial sequence
  Synthetic
KGNQNQIETCNQSVITYENNT

SEQ ID NO: 318 - protein, 21 aa, Artificial sequence
  Synthetic
KIETCNQSVITYENNTWVNQT

SEQ ID NO: 319 - protein, 20 aa, Artificial sequence
  Synthetic
QSVITYENNTWVNQTYVNIS

SEQ ID NO: 320 - protein, 20 aa, Artificial sequence
  Synthetic
YENNTWVNQTYVNISNTNFA

SEQ ID NO: 321 - protein, 21 aa, Artificial sequence
  Synthetic
KWVNQTYVNISNTNFAAGQSV

SEQ ID NO: 322 - protein, 20 aa, Artificial sequence
  Synthetic
YVNISNTNFAAGQSVVSVKL

SEQ ID NO: 323 - protein, 20 aa, Artificial sequence
  Synthetic
NTNFAAGQSVVSVKLAGNSS

SEQ ID NO: 324 - protein, 20 aa, Artificial sequence
  Synthetic
AGQSVVSVKLAGNSSLCPVS

SEQ ID NO: 325 - protein, 20 aa, Artificial sequence
  Synthetic
VSVKLAGNSSLCPVSGWAIY

SEQ ID NO: 326 - protein, 20 aa, Artificial sequence
  Synthetic
AGNSSLCPVSGWAIYSKDNS

SEQ ID NO: 327 - protein, 20 aa, Artificial sequence
  Synthetic
LCPVSGWAIYSKDNSVRIGS

SEQ ID NO: 328 - protein, 20 aa, Artificial sequence
  Synthetic
GWAIYSKDNSVRIGSKGDVF

SEQ ID NO: 329 - protein, 20 aa, Artificial sequence
  Synthetic
SKDNSVRIGSKGDVFVIREP

SEQ ID NO: 330 - protein, 20 aa, Artificial sequence
  Synthetic
VRIGSKGDVFVIREPFISCS

SEQ ID NO: 331 - protein, 20 aa, Artificial sequence
  Synthetic
KGDVFVIREPFISCSPLECR

SEQ ID NO: 332 - protein, 20 aa, Artificial sequence
  Synthetic
VIREPFISCSPLECRTFFLT

SEQ ID NO: 333 - protein, 20 aa, Artificial sequence
  Synthetic
FISCSPLECRTFFLTQGALL

SEQ ID NO: 334 - protein, 20 aa, Artificial sequence
  Synthetic
PLECRTFFLTQGALLNDKHS

SEQ ID NO: 335 - protein, 20 aa, Artificial sequence
  Synthetic
TFFLTQGALLNDKHSNGTIK

SEQ ID NO: 336 - protein, 20 aa, Artificial sequence
  Synthetic
QGALLNDKHSNGTIKDRSPY

SEQ ID NO: 337 - protein, 20 aa, Artificial sequence
  Synthetic
NDKHSNGTIKDRSPYRTLMS

SEQ ID NO: 338 - protein, 20 aa, Artificial sequence
  Synthetic
NGTIKDRSPYRTLMSCPIGE

SEQ ID NO: 339 - protein, 20 aa, Artificial sequence
  Synthetic
DRSPYRTLMSCPIGEVPSPY

SEQ ID NO: 340 - protein, 20 aa, Artificial sequence
  Synthetic
RTLMSCPIGEVPSPYNSRFE

SEQ ID NO: 341 - protein, 20 aa, Artificial sequence
  Synthetic
CPIGEVPSPYNSRFESVAWS

SEQ ID NO: 342 - protein, 20 aa, Artificial sequence
  Synthetic
VPSPYNSRFESVAWSASACH

SEQ ID NO: 343 - protein, 20 aa, Artificial sequence
  Synthetic
NSRFESVAWSASACHDGINW

SEQ ID NO: 344 - protein, 21 aa, Artificial sequence
  Synthetic
KSVAWSASACHDGINWLTIGI

SEQ ID NO: 345 - protein, 20 aa, Artificial sequence
  Synthetic
ASACHDGINWLTIGISGPDN

SEQ ID NO: 346 - protein, 20 aa, Artificial sequence
  Synthetic
DGINWLTIGISGPDNGAVAV

SEQ ID NO: 347 - protein, 20 aa, Artificial sequence
  Synthetic
LTIGISGPDNGAVAVLKYNG

SEQ ID NO: 348 - protein, 20 aa, Artificial sequence
  Synthetic
SGPDNGAVAVLKYNGIITDT

SEQ ID NO: 349 - protein, 20 aa, Artificial sequence
  Synthetic
GAVAVLKYNGIITDTIKSWR

SEQ ID NO: 350 - protein, 20 aa, Artificial sequence
  Synthetic
LKYNGIITDTIKSWRNNILR

SEQ ID NO: 351 - protein, 20 aa, Artificial sequence
  Synthetic
IITDTIKSWRNNILRTQESE

SEQ ID NO: 352 - protein, 20 aa, Artificial sequence
  Synthetic
IKSWRNNILRTQESECACVN

SEQ ID NO: 353 - protein, 20 aa, Artificial sequence
  Synthetic
NNILRTQESECACVNGSCFT

SEQ ID NO: 354 - protein, 20 aa, Artificial sequence
  Synthetic
TQESECACVNGSCFTVMTDG

SEQ ID NO: 355 - protein, 21 aa, Artificial sequence
  Synthetic
KCACVNGSCFTVMTDGPSNGQ

SEQ ID NO: 356 - protein, 20 aa, Artificial sequence
  Synthetic
GSCFTVMTDGPSNGQASYKI

SEQ ID NO: 357 - protein, 20 aa, Artificial sequence
  Synthetic
VMTDGPSNGQASYKIFRIEK

SEQ ID NO: 358 - protein, 20 aa, Artificial sequence
  Synthetic
PSNGQASYKIFRIEKGKIVK

SEQ ID NO: 359 - protein, 20 aa, Artificial sequence
  Synthetic
ASYKIFRIEKGKIVKSVEMN

SEQ ID NO: 360 - protein, 20 aa, Artificial sequence
  Synthetic
FRIEKGKIVKSVEMNAPNYH

SEQ ID NO: 361 - protein, 20 aa, Artificial sequence
  Synthetic
GKIVKSVEMNAPNYHYEECS

SEQ ID NO: 362 - protein, 20 aa, Artificial sequence
  Synthetic
SVEMNAPNYHYEECSCYPDS

SEQ ID NO: 363 - protein, 20 aa, Artificial sequence
  Synthetic
APNYHYEECSCYPDSSEITC

SEQ ID NO: 364 - protein, 20 aa, Artificial sequence
  Synthetic
YEECSCYPDSSEITCVCRDN

SEQ ID NO: 365 - protein, 20 aa, Artificial sequence
  Synthetic
CYPDSSEITCVCRDNWHGSN

SEQ ID NO: 366 - protein, 20 aa, Artificial sequence
  Synthetic
SEITCVCRDNWHGSNRPWVS

SEQ ID NO: 367 - protein, 20 aa, Artificial sequence
  Synthetic
VCRDNWHGSNRPWVSFNQNL

SEQ ID NO: 368 - protein, 20 aa, Artificial sequence
  Synthetic
WHGSNRPWVSFNQNLEYQIG

SEQ ID NO: 369 - protein, 20 aa, Artificial sequence
  Synthetic
RPWVSFNQNLEYQIGYICSG

SEQ ID NO: 370 - protein, 20 aa, Artificial sequence
  Synthetic
FNQNLEYQIGYICSGIFGDN

SEQ ID NO: 371 - protein, 20 aa, Artificial sequence
  Synthetic
EYQIGYICSGIFGDNPRPND

SEQ ID NO: 372 - protein, 20 aa, Artificial sequence
  Synthetic
YICSGIFGDNPRPNDKTGSC

SEQ ID NO: 373 - protein, 20 aa, Artificial sequence
  Synthetic
IFGDNPRPNDKTGSCGPVSS

SEQ ID NO: 374 - protein, 20 aa, Artificial sequence
  Synthetic
PRPNDKTGSCGPVSSNGANG

SEQ ID NO: 375 - protein, 20 aa, Artificial sequence
  Synthetic
KTGSCGPVSSNGANGVKGFS

SEQ ID NO: 376 - protein, 20 aa, Artificial sequence
  Synthetic
GPVSSNGANGVKGFSFKYGN

SEQ ID NO: 377 - protein, 20 aa, Artificial sequence
  Synthetic
NGANGVKGFSFKYGNGVWIG

SEQ ID NO: 378 - protein, 20 aa, Artificial sequence
  Synthetic
VKGFSFKYGNGVWIGRTKSI

SEQ ID NO: 379 - protein, 20 aa, Artificial sequence
  Synthetic
FKYGNGVWIGRTKSISSRNG

SEQ ID NO: 380 - protein, 20 aa, Artificial sequence
  Synthetic
GVWIGRTKSISSRNGFEMIW

SEQ ID NO: 381 - protein, 20 aa, Artificial sequence
  Synthetic
RTKSISSRNGFEMIWDPNGW

SEQ ID NO: 382 - protein, 20 aa, Artificial sequence
  Synthetic
SSRNGFEMIWDPNGWTGTDN

SEQ ID NO: 383 - protein, 20 aa, Artificial sequence
  Synthetic
FEMIWDPNGWTGTDNNFSIK

SEQ ID NO: 384 - protein, 20 aa, Artificial sequence
  Synthetic
DPNGWTGTDNNFSIKQDIVG

SEQ ID NO: 385 - protein, 20 aa, Artificial sequence
  Synthetic
TGTDNNFSIKQDIVGINEWS

SEQ ID NO: 386 - protein, 20 aa, Artificial sequence
  Synthetic
NFSIKQDIVGINEWSGYSGS

SEQ ID NO: 387 - protein, 20 aa, Artificial sequence
  Synthetic
QDIVGINEWSGYSGSFVQHP

SEQ ID NO: 388 - protein, 20 aa, Artificial sequence
  Synthetic
INEWSGYSGSFVQHPELTGL

SEQ ID NO: 389 - protein, 20 aa, Artificial sequence
  Synthetic
GYSGSFVQHPELTGLDCIRP

SEQ ID NO: 390 - protein, 20 aa, Artificial sequence
  Synthetic
FVQHPELTGLDCIRPCFWVE

SEQ ID NO: 391 - protein, 20 aa, Artificial sequence
  Synthetic
ELTGLDCIRPCFWVELIRGR

SEQ ID NO: 392 - protein, 20 aa, Artificial sequence
  Synthetic
DCIRPCFWVELIRGRPKENT

SEQ ID NO: 393 - protein, 20 aa, Artificial sequence
  Synthetic
CFWVELIRGRPKENTIWTSG

SEQ ID NO: 394 - protein, 20 aa, Artificial sequence
  Synthetic
LIRGRPKENTIWTSGSSISF

SEQ ID NO: 395 - protein, 20 aa, Artificial sequence
  Synthetic
PKENTIWTSGSSISFCGVNS

SEQ ID NO: 396 - protein, 21 aa, Artificial sequence
  Synthetic
KIWTSGSSISFCGVNSDTVGW

SEQ ID NO: 397 - protein, 21 aa, Artificial sequence
  Synthetic
KSSISFCGVNSDTVGWSWPDG

SEQ ID NO: 398 - protein, 21 aa, Artificial sequence
  Synthetic
KCGVNSDTVGWSWPDGAELPF

SEQ ID NO: 399 - protein, 19 aa, Artificial sequence
  Synthetic
DTVGWSWPDGAELPFTIDK

SEQ ID NO: 400 - protein, 20 aa, Artificial sequence
  Synthetic
KANLLVLLCALAAADADTIC

SEQ ID NO: 401 - protein, 20 aa, Artificial sequence
  Synthetic
VLLCALAAADADTICIGYHA

SEQ ID NO: 402 - protein, 20 aa, Artificial sequence
  Synthetic
LAAADADTICIGYHANNSTD

SEQ ID NO: 403 - protein, 20 aa, Artificial sequence
  Synthetic
ADTICIGYHANNSTDTVDTV

SEQ ID NO: 404 - protein, 20 aa, Artificial sequence
  Synthetic
IGYHANNSTDTVDTVLEKNV

SEQ ID NO: 405 - protein, 20 aa, Artificial sequence
  Synthetic
NNSTDTVDTVLEKNVTVTHS

SEQ ID NO: 406 - protein, 20 aa, Artificial sequence
  Synthetic
TVDTVLEKNVTVTHSVNLLE

SEQ ID NO: 407 - protein, 20 aa, Artificial sequence
  Synthetic
LEKNVTVTHSVNLLEDSHNG

SEQ ID NO: 408 - protein, 20 aa, Artificial sequence
  Synthetic
TVTHSVNLLEDSHNGKLCRL

SEQ ID NO: 409 - protein, 20 aa, Artificial sequence
  Synthetic
VNLLEDSHNGKLCRLKGIAP

SEQ ID NO: 410 - protein, 20 aa, Artificial sequence
  Synthetic
DSHNGKLCRLKGIAPLQLGK

SEQ ID NO: 411 - protein, 19 aa, Artificial sequence
  Synthetic
LCRLKGIAPLQLGKCNIAG

SEQ ID NO: 412 - protein, 20 aa, Artificial sequence
  Synthetic
KGIAPLQLGKCNIAGWLLGN

SEQ ID NO: 413 - protein, 20 aa, Artificial sequence
  Synthetic
LQLGKCNIAGWLLGNPECDP

SEQ ID NO: 414 - protein, 20 aa, Artificial sequence
  Synthetic
CNIAGWLLGNPECDPLLPVR

SEQ ID NO: 415 - protein, 20 aa, Artificial sequence
  Synthetic
WLLGNPECDPLLPVRSWSYI

SEQ ID NO: 416 - protein, 20 aa, Artificial sequence
  Synthetic
PECDPLLPVRSWSYIVETPN

SEQ ID NO: 417 - protein, 20 aa, Artificial sequence
  Synthetic
LLPVRSWSYIVETPNSENGI

SEQ ID NO: 418 - protein, 20 aa, Artificial sequence
  Synthetic
SWSYIVETPNSENGICYPGD

SEQ ID NO: 419 - protein, 20 aa, Artificial sequence
  Synthetic
VETPNSENGICYPGDFIDYE

SEQ ID NO: 420 - protein, 20 aa, Artificial sequence
  Synthetic
SENGICYPGDFIDYEELREQ

SEQ ID NO: 421 - protein, 20 aa, Artificial sequence
  Synthetic
CYPGDFIDYEELREQLSSVS

SEQ ID NO: 422 - protein, 20 aa, Artificial sequence
  Synthetic
FIDYEELREQLSSVSSFERF

SEQ ID NO: 423 - protein, 20 aa, Artificial sequence
  Synthetic
ELREQLSSVSSFERFEIFPK

SEQ ID NO: 424 - protein, 20 aa, Artificial sequence
  Synthetic
LSSVSSFERFEIFPKESSWP

SEQ ID NO: 425 - protein, 20 aa, Artificial sequence
  Synthetic
SFERFEIFPKESSWPNHNTN

SEQ ID NO: 426 - protein, 20 aa, Artificial sequence
  Synthetic
EIFPKESSWPNHNTNGVTAA

SEQ ID NO: 427 - protein, 20 aa, Artificial sequence
  Synthetic
ESSWPNHNTNGVTAACSHEG

SEQ ID NO: 428 - protein, 20 aa, Artificial sequence
  Synthetic
NHNTNGVTAACSHEGKSSFY

SEQ ID NO: 429 - protein, 20 aa, Artificial sequence
  Synthetic
GVTAACSHEGKSSFYRNLLW

SEQ ID NO: 430 - protein, 20 aa, Artificial sequence
  Synthetic
CSHEGKSSFYRNLLWLTEKE

SEQ ID NO: 431 - protein, 20 aa, Artificial sequence
  Synthetic
KSSFYRNLLWLTEKEGSYPK

SEQ ID NO: 432 - protein, 20 aa, Artificial sequence
  Synthetic
RNLLWLTEKEGSYPKLKNSY

SEQ ID NO: 433 - protein, 20 aa, Artificial sequence
  Synthetic
LTEKEGSYPKLKNSYVNKKG

SEQ ID NO: 434 - protein, 20 aa, Artificial sequence
  Synthetic
GSYPKLKNSYVNKKGKEVLV

SEQ ID NO: 435 - protein, 20 aa, Artificial sequence
  Synthetic
LKNSYVNKKGKEVLVLWGIH

SEQ ID NO: 436 - protein, 20 aa, Artificial sequence
  Synthetic
VNKKGKEVLVLWGIHHPPNS

SEQ ID NO: 437 - protein, 20 aa, Artificial sequence
  Synthetic
KEVLVLWGIHHPPNSKEQQN

SEQ ID NO: 438 - protein, 20 aa, Artificial sequence
  Synthetic
LWGIHHPPNSKEQQNLYQNE

SEQ ID NO: 439 - protein, 20 aa, Artificial sequence
  Synthetic
HPPNSKEQQNLYQNENAYVS

SEQ ID NO: 440 - protein, 20 aa, Artificial sequence
  Synthetic
KEQQNLYQNENAYVSVVTSN

SEQ ID NO: 441 - protein, 20 aa, Artificial sequence
  Synthetic
LYQNENAYVSVVTSNYNRRF

SEQ ID NO: 442 - protein, 20 aa, Artificial sequence
  Synthetic
NAYVSVVTSNYNRRFTPEIA

SEQ ID NO: 443 - protein, 20 aa, Artificial sequence
  Synthetic
VVTSNYNRRFTPEIAERPKV

SEQ ID NO: 444 - protein, 20 aa, Artificial sequence
  Synthetic
YNRRFTPEIAERPKVRDQAG

SEQ ID NO: 445 - protein, 20 aa, Artificial sequence
  Synthetic
TPEIAERPKVRDQAGRMNYY

SEQ ID NO: 446 - protein, 20 aa, Artificial sequence
  Synthetic
ERPKVRDQAGRMNYYWTLLK

SEQ ID NO: 447 - protein, 20 aa, Artificial sequence
  Synthetic
RDQAGRMNYYWTLLKPGDTI

SEQ ID NO: 448 - protein, 20 aa, Artificial sequence
  Synthetic
RMNYYWTLLKPGDTIIFEAN

SEQ ID NO: 449 - protein, 20 aa, Artificial sequence
  Synthetic
WTLLKPGDTIIFEANGNLIA

SEQ ID NO: 450 - protein, 20 aa, Artificial sequence
  Synthetic
PGDTIIFEANGNLIAPMYAF

SEQ ID NO: 451 - protein, 20 aa, Artificial sequence
  Synthetic
IFEANGNLIAPMYAFALSRG

SEQ ID NO: 452 - protein, 20 aa, Artificial sequence
  Synthetic
GNLIAPMYAFALSRGFGSGI

SEQ ID NO: 453 - protein, 20 aa, Artificial sequence
  Synthetic
PMYAFALSRGFGSGIITSNA

SEQ ID NO: 454 - protein, 20 aa, Artificial sequence
  Synthetic
ALSRGFGSGIITSNASMHEC

SEQ ID NO: 455 - protein, 20 aa, Artificial sequence
  Synthetic
FGSGIITSNASMHECNTKCQ

SEQ ID NO: 456 - protein, 20 aa, Artificial sequence
  Synthetic
ITSNASMHECNTKCQTPLGA

SEQ ID NO: 457 - protein, 20 aa, Artificial sequence
  Synthetic
SMHECNTKCQTPLGAINSSL

SEQ ID NO: 458 - protein, 20 aa, Artificial sequence
  Synthetic
NTKCQTPLGAINSSLPYQNI

SEQ ID NO: 459 - protein, 20 aa, Artificial sequence
  Synthetic
TPLGAINSSLPYQNIHPVTI

SEQ ID NO: 460 - protein, 20 aa, Artificial sequence
  Synthetic
INSSLPYQNIHPVTIGECPK

SEQ ID NO: 461 - protein, 20 aa, Artificial sequence
  Synthetic
PYQNIHPVTIGECPKYVRSA

SEQ ID NO: 462 - protein, 20 aa, Artificial sequence
  Synthetic
HPVTIGECPKYVRSAKLRMV

SEQ ID NO: 463 - protein, 20 aa, Artificial sequence
  Synthetic
GECPKYVRSAKLRMVTGLRN

SEQ ID NO: 464 - protein, 20 aa, Artificial sequence
  Synthetic
YVRSAKLRMVTGLRNIPSIQ

SEQ ID NO: 465 - protein, 19 aa, Artificial sequence
  Synthetic
LRMVTGLRNIPSIQSRGLF

SEQ ID NO: 466 - protein, 20 aa, Artificial sequence
  Synthetic
TGLRNIPSIQSRGLFGAIAG

SEQ ID NO: 467 - protein, 20 aa, Artificial sequence
  Synthetic
IPSIQSRGLFGAIAGFIEGG

SEQ ID NO: 468 - protein, 20 aa, Artificial sequence
  Synthetic
SRGLFGAIAGFIEGGWTGMI

SEQ ID NO: 469 - protein, 20 aa, Artificial sequence
  Synthetic
GAIAGFIEGGWTGMIDGWYG

SEQ ID NO: 470 - protein, 20 aa, Artificial sequence
  Synthetic
FIEGGWTGMIDGWYGYHHQN

SEQ ID NO: 471 - protein, 20 aa, Artificial sequence
  Synthetic
WTGMIDGWYGYHHQNEQGSG

SEQ ID NO: 472 - protein, 20 aa, Artificial sequence
  Synthetic
DGWYGYHHQNEQGSGYAADQ

SEQ ID NO: 473 - protein, 20 aa, Artificial sequence
  Synthetic
YHHQNEQGSGYAADQKSTQN

SEQ ID NO: 474 - protein, 20 aa, Artificial sequence
  Synthetic
EQGSGYAADQKSTQNAINGI

SEQ ID NO: 475 - protein, 20 aa, Artificial sequence
  Synthetic
YAADQKSTQNAINGITNKVN

SEQ ID NO: 476 - protein, 20 aa, Artificial sequence
  Synthetic
KSTQNAINGITNKVNTVIEK

SEQ ID NO: 477 - protein, 20 aa, Artificial sequence
  Synthetic
AINGITNKVNTVIEKMNIQF

SEQ ID NO: 478 - protein, 20 aa, Artificial sequence
  Synthetic
TNKVNTVIEKMNIQFTAVGK

SEQ ID NO: 479 - protein, 20 aa, Artificial sequence
  Synthetic
TVIEKMNIQFTAVGKEFNKL

SEQ ID NO: 480 - protein, 20 aa, Artificial sequence
  Synthetic
MNIQFTAVGKEFNKLEKRME

SEQ ID NO: 481 - protein, 20 aa, Artificial sequence
  Synthetic
TAVGKEFNKLEKRMENLNKK

SEQ ID NO: 482 - protein, 20 aa, Artificial sequence
  Synthetic
EFNKLEKRMENLNKKVDDGF

SEQ ID NO: 483 - protein, 20 aa, Artificial sequence
  Synthetic
EKRMENLNKKVDDGFLDIWT

SEQ ID NO: 484 - protein, 20 aa, Artificial sequence
  Synthetic
NLNKKVDDGFLDIWTYNAEL

SEQ ID NO: 485 - protein, 20 aa, Artificial sequence
  Synthetic
VDDGFLDIWTYNAELLVLLE

SEQ ID NO: 486 - protein, 20 aa, Artificial sequence
  Synthetic
LDIWTYNAELLVLLENERTL

SEQ ID NO: 487 - protein, 20 aa, Artificial sequence
  Synthetic
YNAELLVLLENERTLDFHDS

SEQ ID NO: 488 - protein, 20 aa, Artificial sequence
  Synthetic
LVLLENERTLDFHDSNVKNL

SEQ ID NO: 489 - protein, 20 aa, Artificial sequence
  Synthetic
NERTLDFHDSNVKNLYEKVK

SEQ ID NO: 490 - protein, 20 aa, Artificial sequence
  Synthetic
DFHDSNVKNLYEKVKSQLKN

SEQ ID NO: 491 - protein, 20 aa, Artificial sequence
  Synthetic
NVKNLYEKVKSQLKNNAKEI

SEQ ID NO: 492 - protein, 20 aa, Artificial sequence
  Synthetic
YEKVKSQLKNNAKEIGNGCF

SEQ ID NO: 493 - protein, 20 aa, Artificial sequence
  Synthetic
SQLKNNAKEIGNGCFEFYHK

SEQ ID NO: 494 - protein, 20 aa, Artificial sequence
  Synthetic
NAKEIGNGCFEFYHKCDNEC

SEQ ID NO: 495 - protein, 20 aa, Artificial sequence
  Synthetic
GNGCFEFYHKCDNECMESVR

SEQ ID NO: 496 - protein, 20 aa, Artificial sequence
  Synthetic
EFYHKCDNECMESVRNGTYD

SEQ ID NO: 497 - protein, 20 aa, Artificial sequence
  Synthetic
CDNECMESVRNGTYDYPKYS

SEQ ID NO: 498 - protein, 20 aa, Artificial sequence
  Synthetic
MESVRNGTYDYPKYSEESKL

SEQ ID NO: 499 - protein, 20 aa, Artificial sequence
  Synthetic
NGTYDYPKYSEESKLNREKV

SEQ ID NO: 500 - protein, 20 aa, Artificial sequence
  Synthetic
YPKYSEESKLNREKVDGVKL

SEQ ID NO: 501 - protein, 20 aa, Artificial sequence
  Synthetic
EESKLNREKVDGVKLESMGI

SEQ ID NO: 502 - protein, 20 aa, Artificial sequence
  Synthetic
NREKVDGVKLESMGIYQILA

SEQ ID NO: 503 - protein, 20 aa, Artificial sequence
  Synthetic
DGVKLESMGIYQILAIYSTV

SEQ ID NO: 504 - protein, 20 aa, Artificial sequence
  Synthetic
ESMGIYQILAIYSTVASSLV

SEQ ID NO: 505 - protein, 20 aa, Artificial sequence
  Synthetic
YQILAIYSTVASSLVLLVSL

SEQ ID NO: 506 - protein, 20 aa, Artificial sequence
  Synthetic
IYSTVASSLVLLVSLGAISF

SEQ ID NO: 507 - protein, 20 aa, Artificial sequence
  Synthetic
ASSLVLLVSLGAISFWMCSN

SEQ ID NO: 508 - protein, 20 aa, Artificial sequence
  Synthetic
LLVSLGAISFWMCSNGSLQC

SEQ ID NO: 509 - protein, 19 aa, Artificial sequence
  Synthetic
GAISFWMCSNGSLQCRICI

SEQ ID NO: 510 - protein, 20 aa, Artificial sequence
  Synthetic
NPNQKIITIGSICLVVGLIS

SEQ ID NO: 511 - protein, 20 aa, Artificial sequence
  Synthetic
IITIGSICLVVGLISLILQI

SEQ ID NO: 512 - protein, 20 aa, Artificial sequence
  Synthetic
SICLVVGLISLILQIGNIIS

SEQ ID NO: 513 - protein, 20 aa, Artificial sequence
  Synthetic
VGLISLILQIGNIISIWISH

SEQ ID NO: 514 - protein, 20 aa, Artificial sequence
  Synthetic
LILQIGNIISIWISHSIQTG

SEQ ID NO: 515 - protein, 20 aa, Artificial sequence
  Synthetic
GNIISIWISHSIQTGSQNHT

SEQ ID NO: 516 - protein, 20 aa, Artificial sequence
  Synthetic
IWISHSIQTGSQNHTGICNQ

SEQ ID NO: 517 - protein, 20 aa, Artificial sequence
  Synthetic
SIQTGSQNHTGICNQNIITY

SEQ ID NO: 518 - protein, 20 aa, Artificial sequence
  Synthetic
SQNHTGICNQNIITYKNSTW

SEQ ID NO: 519 - protein, 20 aa, Artificial sequence
  Synthetic
GICNQNIITYKNSTWVKDTT

SEQ ID NO: 520 - protein, 20 aa, Artificial sequence
  Synthetic
NIITYKNSTWVKDTTSVILT

SEQ ID NO: 521 - protein, 20 aa, Artificial sequence
  Synthetic
KNSTWVKDTTSVILTGNSSL

SEQ ID NO: 522 - protein, 20 aa, Artificial sequence
  Synthetic
VKDTTSVILTGNSSLCPIRG

SEQ ID NO: 523 - protein, 20 aa, Artificial sequence
  Synthetic
SVILTGNSSLCPIRGWAIYS

SEQ ID NO: 524 - protein, 20 aa, Artificial sequence
  Synthetic
GNSSLCPIRGWAIYSKDNSI

SEQ ID NO: 525 - protein, 20 aa, Artificial sequence
  Synthetic
CPIRGWAIYSKDNSIRIGSK

SEQ ID NO: 526 - protein, 20 aa, Artificial sequence
  Synthetic
WAIYSKDNSIRIGSKGDVFV

SEQ ID NO: 527 - protein, 20 aa, Artificial sequence
  Synthetic
KDNSIRIGSKGDVFVIREPF

SEQ ID NO: 528 - protein, 20 aa, Artificial sequence
  Synthetic
RIGSKGDVFVIREPFISCSH

SEQ ID NO: 529 - protein, 20 aa, Artificial sequence
  Synthetic
GDVFVIREPFISCSHLECRT

SEQ ID NO: 530 - protein, 20 aa, Artificial sequence
  Synthetic
IREPFISCSHLECRTFFLTQ

SEQ ID NO: 531 - protein, 20 aa, Artificial sequence
  Synthetic
ISCSHLECRTFFLTQGALLN

SEQ ID NO: 532 - protein, 20 aa, Artificial sequence
  Synthetic
LECRTFFLTQGALLNDKHSS

SEQ ID NO: 533 - protein, 20 aa, Artificial sequence
  Synthetic
FFLTQGALLNDKHSSGTVKD

SEQ ID NO: 534 - protein, 20 aa, Artificial sequence
  Synthetic
GALLNDKHSSGTVKDRSPYR

SEQ ID NO: 535 - protein, 20 aa, Artificial sequence
  Synthetic
DKHSSGTVKDRSPYRALMSC

SEQ ID NO: 536 - protein, 20 aa, Artificial sequence
  Synthetic
GTVKDRSPYRALMSCPVGEA

SEQ ID NO: 537 - protein, 20 aa, Artificial sequence
  Synthetic
RSPYRALMSCPVGEAPSPYN

SEQ ID NO: 538 - protein, 20 aa, Artificial sequence
  Synthetic
ALMSCPVGEAPSPYNSRFES

SEQ ID NO: 539 - protein, 20 aa, Artificial sequence
  Synthetic
PVGEAPSPYNSRFESVAWSA

SEQ ID NO: 540 - protein, 20 aa, Artificial sequence
  Synthetic
PSPYNSRFESVAWSASACHD

SEQ ID NO: 541 - protein, 20 aa, Artificial sequence
  Synthetic
SRFESVAWSASACHDGMGWL

SEQ ID NO: 542 - protein, 20 aa, Artificial sequence
  Synthetic
VAWSASACHDGMGWLTIGIS

SEQ ID NO: 543 - protein, 20 aa, Artificial sequence
  Synthetic
SACHDGMGWLTIGISGPDNG

SEQ ID NO: 544 - protein, 20 aa, Artificial sequence
  Synthetic
GMGWLTIGISGPDNGAVAVL

SEQ ID NO: 545 - protein, 20 aa, Artificial sequence
  Synthetic
TIGISGPDNGAVAVLKYNGI

SEQ ID NO: 546 - protein, 20 aa, Artificial sequence
  Synthetic
GPDNGAVAVLKYNGIITETI

SEQ ID NO: 547 - protein, 20 aa, Artificial sequence
  Synthetic
AVAVLKYNGIITETIKSWRK

SEQ ID NO: 548 - protein, 19 aa, Artificial sequence
  Synthetic
YNGIITETIKSWRKKILRT

SEQ ID NO: 549 - protein, 20 aa, Artificial sequence
  Synthetic
ITETIKSWRKKILRTQESEC

SEQ ID NO: 550 - protein, 19 aa, Artificial sequence
  Synthetic
SWRKKILRTQESECACVNG

SEQ ID NO: 551 - protein, 20 aa, Artificial sequence
  Synthetic
KILRTQESECACVNGSCFTI

SEQ ID NO: 552 - protein, 20 aa, Artificial sequence
  Synthetic
QESECACVNGSCFTIMTDGP

SEQ ID NO: 553 - protein, 20 aa, Artificial sequence
  Synthetic
ACVNGSCFTIMTDGPSDGLA

SEQ ID NO: 554 - protein, 20 aa, Artificial sequence
  Synthetic
SCFTIMTDGPSDGLASYKIF

SEQ ID NO: 555 - protein, 20 aa, Artificial sequence
  Synthetic
MTDGPSDGLASYKIFKIEKG

SEQ ID NO: 556 - protein, 20 aa, Artificial sequence
  Synthetic
SDGLASYKIFKIEKGKVTKS

SEQ ID NO: 557 - protein, 20 aa, Artificial sequence
  Synthetic
SYKIFKIEKGKVTKSIELNA

SEQ ID NO: 558 - protein, 20 aa, Artificial sequence
  Synthetic
KIEKGKVTKSIELNAPNSHY

SEQ ID NO: 559 - protein, 19 aa, Artificial sequence
  Synthetic
VTKSIELNAPNSHYEECSC

SEQ ID NO: 560 - protein, 20 aa, Artificial sequence
  Synthetic
IELNAPNSHYEECSCYPDTG

SEQ ID NO: 561 - protein, 20 aa, Artificial sequence
  Synthetic
PNSHYEECSCYPDTGKVMCV

SEQ ID NO: 562 - protein, 20 aa, Artificial sequence
  Synthetic
EECSCYPDTGKVMCVCRDNW

SEQ ID NO: 563 - protein, 20 aa, Artificial sequence
  Synthetic
YPDTGKVMCVCRDNWHGSNR

SEQ ID NO: 564 - protein, 20 aa, Artificial sequence
  Synthetic
KVMCVCRDNWHGSNRPWVSF

SEQ ID NO: 565 - protein, 20 aa, Artificial sequence
  Synthetic
CRDNWHGSNRPWVSFDQNLD

SEQ ID NO: 566 - protein, 20 aa, Artificial sequence
  Synthetic
HGSNRPWVSFDQNLDYQIGY

SEQ ID NO: 567 - protein, 20 aa, Artificial sequence
  Synthetic
PWVSFDQNLDYQIGYICSGV

SEQ ID NO: 568 - protein, 20 aa, Artificial sequence
  Synthetic
DQNLDYQIGYICSGVFGDNP

SEQ ID NO: 569 - protein, 20 aa, Artificial sequence
  Synthetic
YQIGYICSGVFGDNPRPEDG

SEQ ID NO: 570 - protein, 20 aa, Artificial sequence
  Synthetic
ICSGVFGDNPRPEDGTGSCG

SEQ ID NO: 571 - protein, 20 aa, Artificial sequence
  Synthetic
FGDNPRPEDGTGSCGPVYVD

SEQ ID NO: 572 - protein, 20 aa, Artificial sequence
  Synthetic
RPEDGTGSCGPVYVDGANGV

SEQ ID NO: 573 - protein, 20 aa, Artificial sequence
  Synthetic
TGSCGPVYVDGANGVKGFSY

SEQ ID NO: 574 - protein, 20 aa, Artificial sequence
  Synthetic
PVYVDGANGVKGFSYRYGNG

SEQ ID NO: 575 - protein, 20 aa, Artificial sequence
  Synthetic
GANGVKGFSYRYGNGVWIGR

SEQ ID NO: 576 - protein, 20 aa, Artificial sequence
  Synthetic
KGFSYRYGNGVWIGRTKSHS

SEQ ID NO: 577 - protein, 20 aa, Artificial sequence
  Synthetic
RYGNGVWIGRTKSHSSRHGF

SEQ ID NO: 578 - protein, 20 aa, Artificial sequence
  Synthetic
VWIGRTKSHSSRHGFEMIWD

SEQ ID NO: 579 - protein, 20 aa, Artificial sequence
  Synthetic
TKSHSSRHGFEMIWDPNGWT

SEQ ID NO: 580 - protein, 20 aa, Artificial sequence
  Synthetic
SRHGFEMIWDPNGWTETDSK

SEQ ID NO: 581 - protein, 20 aa, Artificial sequence
  Synthetic
EMIWDPNGWTETDSKFSVRQ

SEQ ID NO: 582 - protein, 20 aa, Artificial sequence
  Synthetic
PNGWTETDSKFSVRQDVVAM

SEQ ID NO: 583 - protein, 20 aa, Artificial sequence
  Synthetic
ETDSKFSVRQDVVAMTDWSG

SEQ ID NO: 584 - protein, 20 aa, Artificial sequence
  Synthetic
FSVRQDVVAMTDWSGYSGSF

SEQ ID NO: 585 - protein, 20 aa, Artificial sequence
  Synthetic
DVVAMTDWSGYSGSFVQHPE

SEQ ID NO: 586 - protein, 20 aa, Artificial sequence
  Synthetic
TDWSGYSGSFVQHPELTGLD

SEQ ID NO: 587 - protein, 20 aa, Artificial sequence
  Synthetic
YSGSFVQHPELTGLDCMRPC

SEQ ID NO: 588 - protein, 20 aa, Artificial sequence
  Synthetic
VQHPELTGLDCMRPCFWVEL

SEQ ID NO: 589 - protein, 20 aa, Artificial sequence
  Synthetic
LTGLDCMRPCFWVELIRGRP

SEQ ID NO: 590 - protein, 20 aa, Artificial sequence
  Synthetic
CMRPCFWVELIRGRPKEKTI

SEQ ID NO: 591 - protein, 20 aa, Artificial sequence
  Synthetic
FWVELIRGRPKEKTIWTSAS

SEQ ID NO: 592 - protein, 20 aa, Artificial sequence
  Synthetic
IRGRPKEKTIWTSASSISFC

SEQ ID NO: 593 - protein, 20 aa, Artificial sequence
  Synthetic
KEKTIWTSASSISFCGVNSD

SEQ ID NO: 594 - protein, 20 aa, Artificial sequence
  Synthetic
WTSASSISFCGVNSDTVDWS

SEQ ID NO: 595 - protein, 20 aa, Artificial sequence
  Synthetic
SISFCGVNSDTVDWSWPDGA

SEQ ID NO: 596 - protein, 20 aa, Artificial sequence
  Synthetic
GVNSDTVDWSWPDGAELPFS

SEQ ID NO: 597 - protein, 18 aa, Artificial sequence
  Synthetic
TVDWSWPDGAELPFSIDK

SEQ ID NO: 598 - protein, 20 aa, Artificial sequence
  Synthetic
MNTQILVFALIAIIPTNADK

SEQ ID NO: 599 - protein, 20 aa, Artificial sequence
  Synthetic
LVFALIAIIPTNADKICLGH

SEQ ID NO: 600 - protein, 20 aa, Artificial sequence
  Synthetic
IAIIPTNADKICLGHHAVSN

SEQ ID NO: 601 - protein, 20 aa, Artificial sequence
  Synthetic
TNADKICLGHHAVSNGTKVN

SEQ ID NO: 602 - protein, 20 aa, Artificial sequence
  Synthetic
ICLGHHAVSNGTKVNTLTER

SEQ ID NO: 603 - protein, 20 aa, Artificial sequence
  Synthetic
HAVSNGTKVNTLTERGVEVV

SEQ ID NO: 604 - protein, 20 aa, Artificial sequence
  Synthetic
GTKVNTLTERGVEVVNATET

SEQ ID NO: 605 - protein, 20 aa, Artificial sequence
  Synthetic
TLTERGVEVVNATETVERTN

SEQ ID NO: 606 - protein, 20 aa, Artificial sequence
  Synthetic
GVEVVNATETVERTNIPRIC

SEQ ID NO: 607 - protein, 20 aa, Artificial sequence
  Synthetic
NATETVERTNIPRICSKGKR

SEQ ID NO: 608 - protein, 20 aa, Artificial sequence
  Synthetic
VERTNIPRICSKGKRTVDLG

SEQ ID NO: 609 - protein, 20 aa, Artificial sequence
  Synthetic
IPRICSKGKRTVDLGQCGLL

SEQ ID NO: 610 - protein, 20 aa, Artificial sequence
  Synthetic
SKGKRTVDLGQCGLLGTITG

SEQ ID NO: 611 - protein, 20 aa, Artificial sequence
  Synthetic
TVDLGQCGLLGTITGPPQCD

SEQ ID NO: 612 - protein, 20 aa, Artificial sequence
  Synthetic
QCGLLGTITGPPQCDQFLEF

SEQ ID NO: 613 - protein, 20 aa, Artificial sequence
  Synthetic
GTITGPPQCDQFLEFSADLI

SEQ ID NO: 614 - protein, 20 aa, Artificial sequence
  Synthetic
PPQCDQFLEFSADLIIERRE

SEQ ID NO: 615 - protein, 20 aa, Artificial sequence
  Synthetic
QFLEFSADLIIERREGSDVC

SEQ ID NO: 616 - protein, 20 aa, Artificial sequence
  Synthetic
SADLIIERREGSDVCYPGKF

SEQ ID NO: 617 - protein, 20 aa, Artificial sequence
  Synthetic
IERREGSDVCYPGKFVNEEA

SEQ ID NO: 618 - protein, 20 aa, Artificial sequence
  Synthetic
GSDVCYPGKFVNEEALRQIL

SEQ ID NO: 619 - protein, 20 aa, Artificial sequence
  Synthetic
YPGKFVNEEALRQILRESGG

SEQ ID NO: 620 - protein, 20 aa, Artificial sequence
  Synthetic
VNEEALRQILRESGGIDKEA

SEQ ID NO: 621 - protein, 20 aa, Artificial sequence
  Synthetic
LRQILRESGGIDKEAMGFTY

SEQ ID NO: 622 - protein, 20 aa, Artificial sequence
  Synthetic
RESGGIDKEAMGFTYSGIRT

SEQ ID NO: 623 - protein, 20 aa, Artificial sequence
  Synthetic
IDKEAMGFTYSGIRTNGATS

SEQ ID NO: 624 - protein, 20 aa, Artificial sequence
  Synthetic
MGFTYSGIRTNGATSSCRRS

SEQ ID NO: 625 - protein, 20 aa, Artificial sequence
  Synthetic
SGIRTNGATSSCRRSGSSFY

SEQ ID NO: 626 - protein, 20 aa, Artificial sequence
  Synthetic
NGATSSCRRSGSSFYAEMKW

SEQ ID NO: 627 - protein, 20 aa, Artificial sequence
  Synthetic
SCRRSGSSFYAEMKWLLSNT

SEQ ID NO: 628 - protein, 20 aa, Artificial sequence
  Synthetic
GSSFYAEMKWLLSNTDNAAF

SEQ ID NO: 629 - protein, 20 aa, Artificial sequence
  Synthetic
AEMKWLLSNTDNAAFPQMTK

SEQ ID NO: 630 - protein, 20 aa, Artificial sequence
  Synthetic
LLSNTDNAAFPQMTKSYKNT

SEQ ID NO: 631 - protein, 20 aa, Artificial sequence
  Synthetic
DNAAFPQMTKSYKNTRKNPA

SEQ ID NO: 632 - protein, 20 aa, Artificial sequence
  Synthetic
PQMTKSYKNTRKNPALIVWG

SEQ ID NO: 633 - protein, 20 aa, Artificial sequence
  Synthetic
SYKNTRKNPALIVWGIHHSG

SEQ ID NO: 634 - protein, 20 aa, Artificial sequence
  Synthetic
RKNPALIVWGIHHSGSTAEQ

SEQ ID NO: 635 - protein, 20 aa, Artificial sequence
  Synthetic
LIVWGIHHSGSTAEQTKLYG

SEQ ID NO: 636 - protein, 20 aa, Artificial sequence
  Synthetic
IHHSGSTAEQTKLYGSGNKL

SEQ ID NO: 637 - protein, 20 aa, Artificial sequence
  Synthetic
STAEQTKLYGSGNKLVTVGS

SEQ ID NO: 638 - protein, 20 aa, Artificial sequence
  Synthetic
TKLYGSGNKLVTVGSSNYQQ

SEQ ID NO: 639 - protein, 20 aa, Artificial sequence
  Synthetic
SGNKLVTVGSSNYQQSFVPS

SEQ ID NO: 640 - protein, 20 aa, Artificial sequence
  Synthetic
VTVGSSNYQQSFVPSPGART

SEQ ID NO: 641 - protein, 20 aa, Artificial sequence
  Synthetic
SNYQQSFVPSPGARTQVNGQ

SEQ ID NO: 642 - protein, 20 aa, Artificial sequence
  Synthetic
SFVPSPGARTQVNGQSGRID

SEQ ID NO: 643 - protein, 20 aa, Artificial sequence
  Synthetic
PGARTQVNGQSGRIDFHWLM

SEQ ID NO: 644 - protein, 20 aa, Artificial sequence
  Synthetic
QVNGQSGRIDFHWLMLNPND

SEQ ID NO: 645 - protein, 20 aa, Artificial sequence
  Synthetic
SGRIDFHWLMLNPNDTVTFS

SEQ ID NO: 646 - protein, 20 aa, Artificial sequence
  Synthetic
FHWLMLNPNDTVTFSFNGAF

SEQ ID NO: 647 - protein, 20 aa, Artificial sequence
  Synthetic
LNPNDTVTFSFNGAFIAPDR

SEQ ID NO: 648 - protein, 20 aa, Artificial sequence
  Synthetic
TVTFSFNGAFIAPDRASFLR

SEQ ID NO: 649 - protein, 20 aa, Artificial sequence
  Synthetic
FNGAFIAPDRASFLRGKSMG

SEQ ID NO: 650 - protein, 20 aa, Artificial sequence
  Synthetic
IAPDRASFLRGKSMGIQSGV

SEQ ID NO: 651 - protein, 20 aa, Artificial sequence
  Synthetic
ASFLRGKSMGIQSGVQVDAD

SEQ ID NO: 652 - protein, 20 aa, Artificial sequence
  Synthetic
GKSMGIQSGVQVDADCEGDC

SEQ ID NO: 653 - protein, 20 aa, Artificial sequence
  Synthetic
IQSGVQVDADCEGDCYYSGG

SEQ ID NO: 654 - protein, 20 aa, Artificial sequence
  Synthetic
QVDADCEGDCYYSGGTIISN

SEQ ID NO: 655 - protein, 20 aa, Artificial sequence
  Synthetic
CEGDCYYSGGTIISNLPFQN

SEQ ID NO: 656 - protein, 20 aa, Artificial sequence
  Synthetic
YYSGGTIISNLPFQNIDSRA

SEQ ID NO: 657 - protein, 20 aa, Artificial sequence
  Synthetic
TIISNLPFQNIDSRAVGKCP

SEQ ID NO: 658 - protein, 20 aa, Artificial sequence
  Synthetic
LPFQNIDSRAVGKCPRYVKQ

SEQ ID NO: 659 - protein, 20 aa, Artificial sequence
  Synthetic
IDSRAVGKCPRYVKQRSLLL

SEQ ID NO: 660 - protein, 20 aa, Artificial sequence
  Synthetic
VGKCPRYVKQRSLLLATGMK

SEQ ID NO: 661 - protein, 20 aa, Artificial sequence
  Synthetic
RYVKQRSLLLATGMKNVPEI

SEQ ID NO: 662 - protein, 20 aa, Artificial sequence
  Synthetic
RSLLLATGMKNVPEIPKGRG

SEQ ID NO: 663 - protein, 20 aa, Artificial sequence
  Synthetic
ATGMKNVPEIPKGRGLFGAI

SEQ ID NO: 664 - protein, 20 aa, Artificial sequence
  Synthetic
NVPEIPKGRGLFGAIAGFIE

SEQ ID NO: 665 - protein, 20 aa, Artificial sequence
  Synthetic
PKGRGLFGAIAGFIENGWEG

SEQ ID NO: 666 - protein, 20 aa, Artificial sequence
  Synthetic
LFGAIAGFIENGWEGLIDGW

SEQ ID NO: 667 - protein, 20 aa, Artificial sequence
  Synthetic
AGFIENGWEGLIDGWYGFRH

SEQ ID NO: 668 - protein, 20 aa, Artificial sequence
  Synthetic
NGWEGLIDGWYGFRHQNAQG

SEQ ID NO: 669 - protein, 20 aa, Artificial sequence
  Synthetic
LIDGWYGFRHQNAQGEGTAA

SEQ ID NO: 670 - protein, 20 aa, Artificial sequence
  Synthetic
YGFRHQNAQGEGTAADYKST

SEQ ID NO: 671 - protein, 20 aa, Artificial sequence
  Synthetic
QNAQGEGTAADYKSTQSAID

SEQ ID NO: 672 - protein, 20 aa, Artificial sequence
  Synthetic
EGTAADYKSTQSAIDQITGK

SEQ ID NO: 673 - protein, 20 aa, Artificial sequence
  Synthetic
DYKSTQSAIDQITGKLNRLI

SEQ ID NO: 674 - protein, 20 aa, Artificial sequence
  Synthetic
QSAIDQITGKLNRLIEKTNQ

SEQ ID NO: 675 - protein, 20 aa, Artificial sequence
  Synthetic
QITGKLNRLIEKTNQQFELI

SEQ ID NO: 676 - protein, 20 aa, Artificial sequence
  Synthetic
LNRLIEKTNQQFELIDNEFT

SEQ ID NO: 677 - protein, 20 aa, Artificial sequence
  Synthetic
EKTNQQFELIDNEFTEVEKQ

SEQ ID NO: 678 - protein, 20 aa, Artificial sequence
  Synthetic
QFELIDNEFTEVEKQIGNVI

SEQ ID NO: 679 - protein, 20 aa, Artificial sequence
  Synthetic
DNEFTEVEKQIGNVINWTRD

SEQ ID NO: 680 - protein, 20 aa, Artificial sequence
  Synthetic
EVEKQIGNVINWTRDSITEV

SEQ ID NO: 681 - protein, 20 aa, Artificial sequence
  Synthetic
IGNVINWTRDSITEVWSYNA

SEQ ID NO: 682 - protein, 20 aa, Artificial sequence
  Synthetic
NWTRDSITEVWSYNAELLVA

SEQ ID NO: 683 - protein, 20 aa, Artificial sequence
  Synthetic
SITEVWSYNAELLVAMENQH

SEQ ID NO: 684 - protein, 20 aa, Artificial sequence
  Synthetic
WSYNAELLVAMENQHTIDLA

SEQ ID NO: 685 - protein, 20 aa, Artificial sequence
  Synthetic
ELLVAMENQHTIDLADSEMD

SEQ ID NO: 686 - protein, 20 aa, Artificial sequence
  Synthetic
MENQHTIDLADSEMDKLYER

SEQ ID NO: 687 - protein, 20 aa, Artificial sequence
  Synthetic
TIDLADSEMDKLYERVKRQL

SEQ ID NO: 688 - protein, 20 aa, Artificial sequence
  Synthetic
DSEMDKLYERVKRQLRENAE

SEQ ID NO: 689 - protein, 20 aa, Artificial sequence
  Synthetic
KLYERVKRQLRENAEEDGTG

SEQ ID NO: 690 - protein, 20 aa, Artificial sequence
  Synthetic
VKRQLRENAEEDGTGCFEIF

SEQ ID NO: 691 - protein, 20 aa, Artificial sequence
  Synthetic
RENAEEDGTGCFEIFHKCDD

SEQ ID NO: 692 - protein, 20 aa, Artificial sequence
  Synthetic
EDGTGCFEIFHKCDDDCMAS

SEQ ID NO: 693 - protein, 20 aa, Artificial sequence
  Synthetic
CFEIFHKCDDDCMASIRNNT

SEQ ID NO: 694 - protein, 20 aa, Artificial sequence
  Synthetic
HKCDDDCMASIRNNTYDHSK

SEQ ID NO: 695 - protein, 20 aa, Artificial sequence
  Synthetic
DCMASIRNNTYDHSKYREEA

SEQ ID NO: 696 - protein, 20 aa, Artificial sequence
  Synthetic
IRNNTYDHSKYREEAMQNRI

SEQ ID NO: 697 - protein, 20 aa, Artificial sequence
  Synthetic
YDHSKYREEAMQNRIQIDPV

SEQ ID NO: 698 - protein, 20 aa, Artificial sequence
  Synthetic
YREEAMQNRIQIDPVKLSSG

SEQ ID NO: 699 - protein, 20 aa, Artificial sequence
  Synthetic
MQNRIQIDPVKLSSGYKDVI

SEQ ID NO: 700 - protein, 20 aa, Artificial sequence
  Synthetic
QIDPVKLSSGYKDVILWFSF

SEQ ID NO: 701 - protein, 20 aa, Artificial sequence
  Synthetic
KLSSGYKDVILWFSFGASCF

SEQ ID NO: 702 - protein, 20 aa, Artificial sequence
  Synthetic
YKDVILWFSFGASCFILLAI

SEQ ID NO: 703 - protein, 20 aa, Artificial sequence
  Synthetic
LWFSFGASCFILLAIAMGLV

SEQ ID NO: 704 - protein, 20 aa, Artificial sequence
  Synthetic
GASCFILLAIAMGLVFICVK

SEQ ID NO: 705 - protein, 20 aa, Artificial sequence
  Synthetic
ILLAIAMGLVFICVKNGNMR

SEQ ID NO: 706 - protein, 20 aa, Artificial sequence
  Synthetic
AMGLVFICVKNGNMRCTICI

SEQ ID NO: 707 - protein, 15 aa, Artificial sequence
  Synthetic
FICVKNGNMRCTICI

SEQ ID NO: 708 - protein, 20 aa, Artificial sequence
  Synthetic
MNPNQKILCTSATAIIIGAI

SEQ ID NO: 709 - protein, 20 aa, Artificial sequence
  Synthetic
KILCTSATAIIIGAIAVLIG

SEQ ID NO: 710 - protein, 20 aa, Artificial sequence
  Synthetic
SATAIIIGAIAVLIGIANLG

SEQ ID NO: 711 - protein, 20 aa, Artificial sequence
  Synthetic
IIGAIAVLIGIANLGLNIGL

SEQ ID NO: 712 - protein, 20 aa, Artificial sequence
  Synthetic
AVLIGIANLGLNIGLHLKPS

SEQ ID NO: 713 - protein, 20 aa, Artificial sequence
  Synthetic
IANLGLNIGLHLKPSCNCSH

SEQ ID NO: 714 - protein, 20 aa, Artificial sequence
  Synthetic
LNIGLHLKPSCNCSHSQPET

SEQ ID NO: 715 - protein, 20 aa, Artificial sequence
  Synthetic
HLKPSCNCSHSQPETTNTSQ

SEQ ID NO: 716 - protein, 20 aa, Artificial sequence
  Synthetic
CNCSHSQPETTNTSQTIINN

SEQ ID NO: 717 - protein, 20 aa, Artificial sequence
  Synthetic
SQPETTNTSQTIINNYYNET

SEQ ID NO: 718 - protein, 20 aa, Artificial sequence
  Synthetic
TNTSQTIINNYYNETNITNI

SEQ ID NO: 719 - protein, 20 aa, Artificial sequence
  Synthetic
TIINNYYNETNITNIQMEER

SEQ ID NO: 720 - protein, 20 aa, Artificial sequence
  Synthetic
YYNETNITNIQMEERTSRNF

SEQ ID NO: 721 - protein, 20 aa, Artificial sequence
  Synthetic
NITNIQMEERTSRNFNNLTK

SEQ ID NO: 722 - protein, 20 aa, Artificial sequence
  Synthetic
QMEERTSRNFNNLTKGLCTI

SEQ ID NO: 723 - protein, 20 aa, Artificial sequence
  Synthetic
TSRNFNNLTKGLCTINSWHI

SEQ ID NO: 724 - protein, 20 aa, Artificial sequence
  Synthetic
NNLTKGLCTINSWHIYGKDN

SEQ ID NO: 725 - protein, 20 aa, Artificial sequence
  Synthetic
GLCTINSWHIYGKDNAVRIG

SEQ ID NO: 726 - protein, 20 aa, Artificial sequence
  Synthetic
NSWHIYGKDNAVRIGESSDV

SEQ ID NO: 727 - protein, 20 aa, Artificial sequence
  Synthetic
YGKDNAVRIGESSDVLVTRE

SEQ ID NO: 728 - protein, 20 aa, Artificial sequence
  Synthetic
AVRIGESSDVLVTREPYVSC

SEQ ID NO: 729 - protein, 20 aa, Artificial sequence
  Synthetic
ESSDVLVTREPYVSCDPDEC

SEQ ID NO: 730 - protein, 20 aa, Artificial sequence
  Synthetic
LVTREPYVSCDPDECRFYAL

SEQ ID NO: 731 - protein, 20 aa, Artificial sequence
  Synthetic
PYVSCDPDECRFYALSQGTT

SEQ ID NO: 732 - protein, 20 aa, Artificial sequence
  Synthetic
DPDECRFYALSQGTTIRGKH

SEQ ID NO: 733 - protein, 20 aa, Artificial sequence
  Synthetic
RFYALSQGTTIRGKHSNGTI

SEQ ID NO: 734 - protein, 20 aa, Artificial sequence
  Synthetic
SQGTTIRGKHSNGTIHDRSQ

SEQ ID NO: 735 - protein, 20 aa, Artificial sequence
  Synthetic
IRGKHSNGTIHDRSQYRALI

SEQ ID NO: 736 - protein, 20 aa, Artificial sequence
  Synthetic
SNGTIHDRSQYRALISWPLS

SEQ ID NO: 737 - protein, 20 aa, Artificial sequence
  Synthetic
HDRSQYRALISWPLSSPPTV

SEQ ID NO: 738 - protein, 20 aa, Artificial sequence
  Synthetic
YRALISWPLSSPPTVYNSRV

SEQ ID NO: 739 - protein, 20 aa, Artificial sequence
  Synthetic
SWPLSSPPTVYNSRVECIGW

SEQ ID NO: 740 - protein, 20 aa, Artificial sequence
  Synthetic
SPPTVYNSRVECIGWSSTSC

SEQ ID NO: 741 - protein, 20 aa, Artificial sequence
  Synthetic
YNSRVECIGWSSTSCHDGKS

SEQ ID NO: 742 - protein, 20 aa, Artificial sequence
  Synthetic
ECIGWSSTSCHDGKSRMSIC

SEQ ID NO: 743 - protein, 20 aa, Artificial sequence
  Synthetic
SSTSCHDGKSRMSICISGPN

SEQ ID NO: 744 - protein, 20 aa, Artificial sequence
  Synthetic
HDGKSRMSICISGPNNNASA

SEQ ID NO: 745 - protein, 20 aa, Artificial sequence
  Synthetic
RMSICISGPNNNASAVVWYN

SEQ ID NO: 746 - protein, 20 aa, Artificial sequence
  Synthetic
ISGPNNNASAVVWYNRRPVA

SEQ ID NO: 747 - protein, 20 aa, Artificial sequence
  Synthetic
NNASAVVWYNRRPVAEINTW

SEQ ID NO: 748 - protein, 20 aa, Artificial sequence
  Synthetic
VVWYNRRPVAEINTWARNIL

SEQ ID NO: 749 - protein, 20 aa, Artificial sequence
  Synthetic
RRPVAEINTWARNILRTQES

SEQ ID NO: 750 - protein, 20 aa, Artificial sequence
  Synthetic
EINTWARNILRTQESECVCH

SEQ ID NO: 751 - protein, 20 aa, Artificial sequence
  Synthetic
ARNILRTQESECVCHNGVCP

SEQ ID NO: 752 - protein, 20 aa, Artificial sequence
  Synthetic
RTQESECVCHNGVCPVVFTD

SEQ ID NO: 753 - protein, 20 aa, Artificial sequence
  Synthetic
ECVCHNGVCPVVFTDGSATG

SEQ ID NO: 754 - protein, 20 aa, Artificial sequence
  Synthetic
NGVCPVVFTDGSATGPADTR

SEQ ID NO: 755 - protein, 19 aa, Artificial sequence
  Synthetic
VVFTDGSATGPADTRIYYF

SEQ ID NO: 756 - protein, 20 aa, Artificial sequence
  Synthetic
GSATGPADTRIYYFKEGKIL

SEQ ID NO: 757 - protein, 20 aa, Artificial sequence
  Synthetic
PADTRIYYFKEGKILKWESL

SEQ ID NO: 758 - protein, 20 aa, Artificial sequence
  Synthetic
IYYFKEGKILKWESLTGTAK

SEQ ID NO: 759 - protein, 20 aa, Artificial sequence
  Synthetic
EGKILKWESLTGTAKHIEEC

SEQ ID NO: 760 - protein, 20 aa, Artificial sequence
  Synthetic
KWESLTGTAKHIEECSCYGE

SEQ ID NO: 761 - protein, 20 aa, Artificial sequence
  Synthetic
TGTAKHIEECSCYGERTGIT

SEQ ID NO: 762 - protein, 20 aa, Artificial sequence
  Synthetic
HIEECSCYGERTGITCTCKD

SEQ ID NO: 763 - protein, 20 aa, Artificial sequence
  Synthetic
SCYGERTGITCTCKDNWQGS

SEQ ID NO: 764 - protein, 20 aa, Artificial sequence
  Synthetic
RTGITCTCKDNWQGSNRPVI

SEQ ID NO: 765 - protein, 20 aa, Artificial sequence
  Synthetic
CTCKDNWQGSNRPVIQIDPV

SEQ ID NO: 766 - protein, 20 aa, Artificial sequence
  Synthetic
NWQGSNRPVIQIDPVAMTHT

SEQ ID NO: 767 - protein, 20 aa, Artificial sequence
  Synthetic
NRPVIQIDPVAMTHTSQYIC

SEQ ID NO: 768 - protein, 20 aa, Artificial sequence
  Synthetic
QIDPVAMTHTSQYICSPVLT

SEQ ID NO: 769 - protein, 20 aa, Artificial sequence
  Synthetic
AMTHTSQYICSPVLTDNPRP

SEQ ID NO: 770 - protein, 20 aa, Artificial sequence
  Synthetic
SQYICSPVLTDNPRPNDPNI

SEQ ID NO: 771 - protein, 20 aa, Artificial sequence
  Synthetic
SPVLTDNPRPNDPNIGKCND

SEQ ID NO: 772 - protein, 20 aa, Artificial sequence
  Synthetic
DNPRPNDPNIGKCNDPYPGN

SEQ ID NO: 773 - protein, 20 aa, Artificial sequence
  Synthetic
NDPNIGKCNDPYPGNNNNGV

SEQ ID NO: 774 - protein, 20 aa, Artificial sequence
  Synthetic
GKCNDPYPGNNNNGVKGFSY

SEQ ID NO: 775 - protein, 20 aa, Artificial sequence
  Synthetic
PYPGNNNNGVKGFSYLDGAN

SEQ ID NO: 776 - protein, 20 aa, Artificial sequence
  Synthetic
NNNGVKGFSYLDGANTWLGR

SEQ ID NO: 777 - protein, 20 aa, Artificial sequence
  Synthetic
KGFSYLDGANTWLGRTISTA

SEQ ID NO: 778 - protein, 20 aa, Artificial sequence
  Synthetic
LDGANTWLGRTISTASRSGY

SEQ ID NO: 779 - protein, 20 aa, Artificial sequence
  Synthetic
TWLGRTISTASRSGYEMLKV

SEQ ID NO: 780 - protein, 20 aa, Artificial sequence
  Synthetic
TISTASRSGYEMLKVPNALT

SEQ ID NO: 781 - protein, 19 aa, Artificial sequence
  Synthetic
SRSGYEMLKVPNALTDDRS

SEQ ID NO: 782 - protein, 20 aa, Artificial sequence
  Synthetic
EMLKVPNALTDDRSKPIQGQ

SEQ ID NO: 783 - protein, 20 aa, Artificial sequence
  Synthetic
PNALTDDRSKPIQGQTIVLN

SEQ ID NO: 784 - protein, 20 aa, Artificial sequence
  Synthetic
DDRSKPIQGQTIVLNADWSG

SEQ ID NO: 785 - protein, 20 aa, Artificial sequence
  Synthetic
PIQGQTIVLNADWSGYSGSF

SEQ ID NO: 786 - protein, 20 aa, Artificial sequence
  Synthetic
TIVLNADWSGYSGSFMDYWA

SEQ ID NO: 787 - protein, 20 aa, Artificial sequence
  Synthetic
ADWSGYSGSFMDYWAEGDCY

SEQ ID NO: 788 - protein, 20 aa, Artificial sequence
  Synthetic
YSGSFMDYWAEGDCYRACFY

SEQ ID NO: 789 - protein, 20 aa, Artificial sequence
  Synthetic
MDYWAEGDCYRACFYVELIR

SEQ ID NO: 790 - protein, 20 aa, Artificial sequence
  Synthetic
EGDCYRACFYVELIRGRPKE

SEQ ID NO: 791 - protein, 20 aa, Artificial sequence
  Synthetic
RACFYVELIRGRPKEDKVWW

SEQ ID NO: 792 - protein, 20 aa, Artificial sequence
  Synthetic
VELIRGRPKEDKVWWTSNSI

SEQ ID NO: 793 - protein, 20 aa, Artificial sequence
  Synthetic
GRPKEDKVWWTSNSIVSMCS

SEQ ID NO: 794 - protein, 20 aa, Artificial sequence
  Synthetic
DKVWWTSNSIVSMCSSTEFL

SEQ ID NO: 795 - protein, 20 aa, Artificial sequence
  Synthetic
TSNSIVSMCSSTEFLGQWNW

SEQ ID NO: 796 - protein, 20 aa, Artificial sequence
  Synthetic
VSMCSSTEFLGQWNWPDGAK

SEQ ID NO: 797 - protein, 20 aa, Artificial sequence
  Synthetic
STEFLGQWNWPDGAKIEYFL

SEQ ID NO: 798 - protein, 15 aa, Artificial sequence
  Synthetic
GQWNWPDGAKIEYFL

SEQ ID NO: 799 - protein, 22 aa, Artificial sequence
  Synthetic
MEKIVLLFAIVSLVKSDQICKK

SEQ ID NO: 800 - protein, 22 aa, Artificial sequence
  Synthetic
LLFAIVSLVKSDQICIGYHAKK

SEQ ID NO: 801 - protein, 22 aa, Artificial sequence
  Synthetic
VSLVKSDQICIGYHANNSTEKK

SEQ ID NO: 802 - protein, 22 aa, Artificial sequence
  Synthetic
SDQICIGYHANNSTEQVDTIKK

SEQ ID NO: 803 - protein, 22 aa, Artificial sequence
  Synthetic
IGYHANNSTEQVDTIMEKNVKK

SEQ ID NO: 804 - protein, 22 aa, Artificial sequence
  Synthetic
NNSTEQVDTIMEKNVTVTHAKK

SEQ ID NO: 805 - protein, 22 aa, Artificial sequence
  Synthetic
QVDTIMEKNVTVTHAQDILEKK

SEQ ID NO: 806 - protein, 22 aa, Artificial sequence
  Synthetic
MEKNVTVTHAQDILEKKHNGKK

SEQ ID NO: 807 - protein, 22 aa, Artificial sequence
  Synthetic
TVTHAQDILEKKHNGKLCDLKK

SEQ ID NO: 808 - protein, 22 aa, Artificial sequence
  Synthetic
QDILEKKHNGKLCDLDGVKPKK

SEQ ID NO: 809 - protein, 22 aa, Artificial sequence
  Synthetic
KKHNGKLCDLDGVKPLILRDKK

SEQ ID NO: 810 - protein, 22 aa, Artificial sequence
  Synthetic
KLCDLDGVKPLILRDCSVAGKK

SEQ ID NO: 811 - protein, 22 aa, Artificial sequence
  Synthetic
DGVKPLILRDCSVAGWLLGNKK

SEQ ID NO: 812 - protein, 22 aa, Artificial sequence
  Synthetic
LILRDCSVAGWLLGNPMCDEKK

SEQ ID NO: 813 - protein, 22 aa, Artificial sequence
  Synthetic
CSVAGWLLGNPMCDEFINVPKK

SEQ ID NO: 814 - protein, 22 aa, Artificial sequence
  Synthetic
WLLGNPMCDEFINVPEWSYIKK

SEQ ID NO: 815 - protein, 22 aa, Artificial sequence
  Synthetic
PMCDEFINVPEWSYIVEKANKK

SEQ ID NO: 816 - protein, 22 aa, Artificial sequence
  Synthetic
FINVPEWSYIVEKANPVNDLKK

SEQ ID NO: 817 - protein, 22 aa, Artificial sequence
  Synthetic
EWSYIVEKANPVNDLCYPGDKK

SEQ ID NO: 818 - protein, 22 aa, Artificial sequence
  Synthetic
VEKANPVNDLCYPGDFNDYEKK

SEQ ID NO: 819 - protein, 22 aa, Artificial sequence
  Synthetic
PVNDLCYPGDFNDYEELKHLKK

SEQ ID NO: 820 - protein, 22 aa, Artificial sequence
  Synthetic
CYPGDFNDYEELKHLLSRINKK

SEQ ID NO: 821 - protein, 22 aa, Artificial sequence
  Synthetic
FNDYEELKHLLSRINHFEKIKK

SEQ ID NO: 822 - protein, 22 aa, Artificial sequence
  Synthetic
ELKHLLSRINHFEKIQIIPKKK

SEQ ID NO: 823 - protein, 22 aa, Artificial sequence
  Synthetic
LSRINHFEKIQIIPKSSWSSKK

SEQ ID NO: 824 - protein, 22 aa, Artificial sequence
  Synthetic
HFEKIQIIPKSSWSSHEASLKK

SEQ ID NO: 825 - protein, 22 aa, Artificial sequence
  Synthetic
QIIPKSSWSSHEASLGVSSAKK

SEQ ID NO: 826 - protein, 22 aa, Artificial sequence
  Synthetic
SSWSSHEASLGVSSACPYQGKK

SEQ ID NO: 827 - protein, 22 aa, Artificial sequence
  Synthetic
HEASLGVSSACPYQGESSFFKK

SEQ ID NO: 828 - protein, 22 aa, Artificial sequence
  Synthetic
GVSSACPYQGESSFFRNVVWKK

SEQ ID NO: 829 - protein, 22 aa, Artificial sequence
  Synthetic
CPYQGESSFFRNVVWLIKKNKK

SEQ ID NO: 830 - protein, 22 aa, Artificial sequence
  Synthetic
ESSFFRNVVWLIKKNSTYPTKK

SEQ ID NO: 831 - protein, 22 aa, Artificial sequence
  Synthetic
RNVVWLIKKNSTYPTIKRSYKK

SEQ ID NO: 832 - protein, 22 aa, Artificial sequence
  Synthetic
LIKKNSTYPTIKRSYNNTNQKK

SEQ ID NO: 833 - protein, 22 aa, Artificial sequence
  Synthetic
STYPTIKRSYNNTNQEDLLVKK

SEQ ID NO: 834 - protein, 22 aa, Artificial sequence
  Synthetic
IKRSYNNTNQEDLLVLWGIHKK

SEQ ID NO: 835 - protein, 22 aa, Artificial sequence
  Synthetic
NNTNQEDLLVLWGIHHPNDAKK

SEQ ID NO: 836 - protein, 22 aa, Artificial sequence
  Synthetic
EDLLVLWGIHHPNDAAEQTKKK

SEQ ID NO: 837 - protein, 22 aa, Artificial sequence
  Synthetic
LWGIHHPNDAAEQTKLYQNPKK

SEQ ID NO: 838 - protein, 22 aa, Artificial sequence
  Synthetic
HPNDAAEQTKLYQNPTTYISKK

SEQ ID NO: 839 - protein, 22 aa, Artificial sequence
  Synthetic
AEQTKLYQNPTTYISVGTSTKK

SEQ ID NO: 840 - protein, 22 aa, Artificial sequence
  Synthetic
LYQNPTTYISVGTSTLNQRLKK

SEQ ID NO: 841 - protein, 22 aa, Artificial sequence
  Synthetic
TTYISVGTSTLNQRLVPRIAKK

SEQ ID NO: 842 - protein, 22 aa, Artificial sequence
  Synthetic
VGTSTLNQRLVPRIATRSKVKK

SEQ ID NO: 843 - protein, 22 aa, Artificial sequence
  Synthetic
LNQRLVPRIATRSKVNGQSGKK

SEQ ID NO: 844 - protein, 22 aa, Artificial sequence
  Synthetic
VPRIATRSKVNGQSGRMEFFKK

SEQ ID NO: 845 - protein, 22 aa, Artificial sequence
  Synthetic
TRSKVNGQSGRMEFFWTILKKK

SEQ ID NO: 846 - protein, 22 aa, Artificial sequence
  Synthetic
NGQSGRMEFFWTILKPNDAIKK

SEQ ID NO: 847 - protein, 22 aa, Artificial sequence
  Synthetic
RMEFFWTILKPNDAINFESNKK

SEQ ID NO: 848 - protein, 22 aa, Artificial sequence
  Synthetic
WTILKPNDAINFESNGNFIAKK

SEQ ID NO: 849 - protein, 22 aa, Artificial sequence
  Synthetic
PNDAINFESNGNFIAPEYAYKK

SEQ ID NO: 850 - protein, 22 aa, Artificial sequence
  Synthetic
NFESNGNFIAPEYAYKIVKKKK

SEQ ID NO: 851 - protein, 22 aa, Artificial sequence
  Synthetic
GNFIAPEYAYKIVKKGDSTIKK

SEQ ID NO: 852 - protein, 22 aa, Artificial sequence
  Synthetic
PEYAYKIVKKGDSTIMKSELKK

SEQ ID NO: 853 - protein, 22 aa, Artificial sequence
  Synthetic
KIVKKGDSTIMKSELEYGNCKK

SEQ ID NO: 854 - protein, 22 aa, Artificial sequence
  Synthetic
GDSTIMKSELEYGNCNTKCQKK

SEQ ID NO: 855 - protein, 22 aa, Artificial sequence
  Synthetic
MKSELEYGNCNTKCQTPMGAKK

SEQ ID NO: 856 - protein, 22 aa, Artificial sequence
  Synthetic
EYGNCNTKCQTPMGAINSSMKK

SEQ ID NO: 857 - protein, 22 aa, Artificial sequence
  Synthetic
NTKCQTPMGAINSSMPFHNIKK

SEQ ID NO: 858 - protein, 22 aa, Artificial sequence
  Synthetic
TPMGAINSSMPFHNIHPLTIKK

SEQ ID NO: 859 - protein, 22 aa, Artificial sequence
  Synthetic
INSSMPFHNIHPLTIGECPKKK

SEQ ID NO: 860 - protein, 22 aa, Artificial sequence
  Synthetic
PFHNIHPLTIGECPKYVKSNKK

SEQ ID NO: 861 - protein, 22 aa, Artificial sequence
  Synthetic
HPLTIGECPKYVKSNRLVLAKK

SEQ ID NO: 862 - protein, 22 aa, Artificial sequence
  Synthetic
GECPKYVKSNRLVLATGLRNKK

SEQ ID NO: 863 - protein, 22 aa, Artificial sequence
  Synthetic
YVKSNRLVLATGLRNSPQREKK

SEQ ID NO: 864 - protein, 22 aa, Artificial sequence
  Synthetic
RLVLATGLRNSPQRETRGLFKK

SEQ ID NO: 865 - protein, 22 aa, Artificial sequence
  Synthetic
TGLRNSPQRETRGLFGAIAGKK

SEQ ID NO: 866 - protein, 22 aa, Artificial sequence
  Synthetic
SPQRETRGLFGAIAGFIEGGKK

SEQ ID NO: 867 - protein, 22 aa, Artificial sequence
  Synthetic
TRGLFGAIAGFIEGGWQGMVKK

SEQ ID NO: 868 - protein, 22 aa, Artificial sequence
  Synthetic
GAIAGFIEGGWQGMVDGWYGKK

SEQ ID NO: 869 - protein, 22 aa, Artificial sequence
  Synthetic
FIEGGWQGMVDGWYGYHHSNKK

SEQ ID NO: 870 - protein, 22 aa, Artificial sequence
  Synthetic
WQGMVDGWYGYHHSNEQGSGKK

SEQ ID NO: 871 - protein, 22 aa, Artificial sequence
  Synthetic
DGWYGYHHSNEQGSGYAADKKK

SEQ ID NO: 872 - protein, 22 aa, Artificial sequence
  Synthetic
YHHSNEQGSGYAADKESTQKKK

SEQ ID NO: 873 - protein, 22 aa, Artificial sequence
  Synthetic
EQGSGYAADKESTQKAIDGVKK

SEQ ID NO: 874 - protein, 22 aa, Artificial sequence
  Synthetic
YAADKESTQKAIDGVTNKVNKK

SEQ ID NO: 875 - protein, 22 aa, Artificial sequence
  Synthetic
ESTQKAIDGVTNKVNSIIDKKK

SEQ ID NO: 876 - protein, 22 aa, Artificial sequence
  Synthetic
AIDGVTNKVNSIIDKMNTQFKK

SEQ ID NO: 877 - protein, 22 aa, Artificial sequence
  Synthetic
TNKVNSIIDKMNTQFEAVGRKK

SEQ ID NO: 878 - protein, 22 aa, Artificial sequence
  Synthetic
SIIDKMNTQFEAVGREFNNLKK

SEQ ID NO: 879 - protein, 22 aa, Artificial sequence
  Synthetic
MNTQFEAVGREFNNLERRIEKK

SEQ ID NO: 880 - protein, 22 aa, Artificial sequence
  Synthetic
EAVGREFNNLERRIENLNKKKK

SEQ ID NO: 881 - protein, 22 aa, Artificial sequence
  Synthetic
EFNNLERRIENLNKKMEDGFKK

SEQ ID NO: 882 - protein, 22 aa, Artificial sequence
  Synthetic
ERRIENLNKKMEDGFLDVWTKK

SEQ ID NO: 883 - protein, 22 aa, Artificial sequence
  Synthetic
NLNKKMEDGFLDVWTYNAELKK

SEQ ID NO: 884 - protein, 22 aa, Artificial sequence
  Synthetic
MEDGFLDVWTYNAELLVLMEKK

SEQ ID NO: 885 - protein, 22 aa, Artificial sequence
  Synthetic
LDVWTYNAELLVLMENERTLKK

SEQ ID NO: 886 - protein, 22 aa, Artificial sequence
  Synthetic
YNAELLVLMENERTLDFHDSKK

SEQ ID NO: 887 - protein, 22 aa, Artificial sequence
  Synthetic
LVLMENERTLDFHDSNVKNLKK

SEQ ID NO: 888 - protein, 22 aa, Artificial sequence
  Synthetic
NERTLDFHDSNVKNLYDKVRKK

SEQ ID NO: 889 - protein, 22 aa, Artificial sequence
  Synthetic
DFHDSNVKNLYDKVRLQLRDKK

SEQ ID NO: 890 - protein, 22 aa, Artificial sequence
  Synthetic
NVKNLYDKVRLQLRDNAKELKK

SEQ ID NO: 891 - protein, 22 aa, Artificial sequence
  Synthetic
YDKVRLQLRDNAKELGNGCFKK

SEQ ID NO: 892 - protein, 22 aa, Artificial sequence
  Synthetic
LQLRDNAKELGNGCFEFYHKKK

SEQ ID NO: 893 - protein, 22 aa, Artificial sequence
  Synthetic
NAKELGNGCFEFYHKCDNECKK

SEQ ID NO: 894 - protein, 22 aa, Artificial sequence
  Synthetic
GNGCFEFYHKCDNECMESVRKK

SEQ ID NO: 895 - protein, 22 aa, Artificial sequence
  Synthetic
EFYHKCDNECMESVRNGTYDKK

SEQ ID NO: 896 - protein, 22 aa, Artificial sequence
  Synthetic
CDNECMESVRNGTYDYPQYSKK

SEQ ID NO: 897 - protein, 22 aa, Artificial sequence
  Synthetic
MESVRNGTYDYPQYSEEARLKK

SEQ ID NO: 898 - protein, 22 aa, Artificial sequence
  Synthetic
NGTYDYPQYSEEARLKREEIKK

SEQ ID NO: 899 - protein, 22 aa, Artificial sequence
  Synthetic
YPQYSEEARLKREEISGVKLKK

SEQ ID NO: 900 - protein, 22 aa, Artificial sequence
  Synthetic
EEARLKREEISGVKLESIGIKK

SEQ ID NO: 901 - protein, 22 aa, Artificial sequence
  Synthetic
KREEISGVKLESIGIYQILSKK

SEQ ID NO: 902 - protein, 22 aa, Artificial sequence
  Synthetic
SGVKLESIGIYQILSIYSTVKK

SEQ ID NO: 903 - protein, 22 aa, Artificial sequence
  Synthetic
ESIGIYQILSIYSTVASSLAKK

SEQ ID NO: 904 - protein, 22 aa, Artificial sequence
  Synthetic
YQILSIYSTVASSLALAIMVKK

SEQ ID NO: 905 - protein, 22 aa, Artificial sequence
  Synthetic
IYSTVASSLALAIMVAGLSLKK

SEQ ID NO: 906 - protein, 22 aa, Artificial sequence
  Synthetic
ASSLALAIMVAGLSLWMCSNKK

SEQ ID NO: 907 - protein, 22 aa, Artificial sequence
  Synthetic
LAIMVAGLSLWMCSNGSLQCKK

SEQ ID NO: 908 - protein, 21 aa, Artificial sequence
  Synthetic
AGLSLWMCSNGSLQCRICIKK

SEQ ID NO: 909 - protein, 22 aa, Artificial sequence
  Synthetic
MNPNQKIITIGSICMVTGIVKK

SEQ ID NO: 910 - protein, 22 aa, Artificial sequence
  Synthetic
KIITIGSICMVTGIVSLMLQKK

SEQ ID NO: 911 - protein, 22 aa, Artificial sequence
  Synthetic
GSICMVTGIVSLMLQIGNMIKK

SEQ ID NO: 912 - protein, 22 aa, Artificial sequence
  Synthetic
VTGIVSLMLQIGNMISIWVSKK

SEQ ID NO: 913 - protein, 22 aa, Artificial sequence
  Synthetic
SLMLQIGNMISIWVSHSIHTKK

SEQ ID NO: 914 - protein, 22 aa, Artificial sequence
  Synthetic
IGNMISIWVSHSIHTGNQHQKK

SEQ ID NO: 915 - protein, 22 aa, Artificial sequence
  Synthetic
SIWVSHSIHTGNQHQSEPISKK

SEQ ID NO: 916 - protein, 22 aa, Artificial sequence
  Synthetic
HSIHTGNQHQSEPISNTNFLKK

SEQ ID NO: 917 - protein, 22 aa, Artificial sequence
  Synthetic
GNQHQSEPISNTNFLTEKAVKK

SEQ ID NO: 918 - protein, 22 aa, Artificial sequence
  Synthetic
SEPISNTNFLTEKAVASVKLKK

SEQ ID NO: 919 - protein, 22 aa, Artificial sequence
  Synthetic
NTNFLTEKAVASVKLAGNSSKK

SEQ ID NO: 920 - protein, 22 aa, Artificial sequence
  Synthetic
TEKAVASVKLAGNSSLCPINKK

SEQ ID NO: 921 - protein, 22 aa, Artificial sequence
  Synthetic
ASVKLAGNSSLCPINGWAVYKK

SEQ ID NO: 922 - protein, 22 aa, Artificial sequence
  Synthetic
AGNSSLCPINGWAVYSKDNSKK

SEQ ID NO: 923 - protein, 22 aa, Artificial sequence
  Synthetic
LCPINGWAVYSKDNSIRIGSKK

SEQ ID NO: 924 - protein, 22 aa, Artificial sequence
  Synthetic
GWAVYSKDNSIRIGSKGDVFKK

SEQ ID NO: 925 - protein, 22 aa, Artificial sequence
  Synthetic
SKDNSIRIGSKGDVFVIREPKK

SEQ ID NO: 926 - protein, 22 aa, Artificial sequence
  Synthetic
IRIGSKGDVFVIREPFISCSKK

SEQ ID NO: 927 - protein, 22 aa, Artificial sequence
  Synthetic
KGDVFVIREPFISCSHLECRKK

SEQ ID NO: 928 - protein, 22 aa, Artificial sequence
  Synthetic
VIREPFISCSHLECRTFFLTKK

SEQ ID NO: 929 - protein, 22 aa, Artificial sequence
  Synthetic
FISCSHLECRTFFLTQGALLKK

SEQ ID NO: 930 - protein, 22 aa, Artificial sequence
  Synthetic
HLECRTFFLTQGALLNDKHSKK

SEQ ID NO: 931 - protein, 22 aa, Artificial sequence
  Synthetic
TFFLTQGALLNDKHSNGTVKKK

SEQ ID NO: 932 - protein, 22 aa, Artificial sequence
  Synthetic
QGALLNDKHSNGTVKDRSPHKK

SEQ ID NO: 933 - protein, 22 aa, Artificial sequence
  Synthetic
NDKHSNGTVKDRSPHRTLMSKK

SEQ ID NO: 934 - protein, 22 aa, Artificial sequence
  Synthetic
NGTVKDRSPHRTLMSCPVGEKK

SEQ ID NO: 935 - protein, 22 aa, Artificial sequence
  Synthetic
DRSPHRTLMSCPVGEAPSPYKK

SEQ ID NO: 936 - protein, 22 aa, Artificial sequence
  Synthetic
RTLMSCPVGEAPSPYNSRFEKK

SEQ ID NO: 937 - protein, 22 aa, Artificial sequence
  Synthetic
CPVGEAPSPYNSRFESVAWSKK

SEQ ID NO: 938 - protein, 22 aa, Artificial sequence
  Synthetic
APSPYNSRFESVAWSASACHKK

SEQ ID NO: 939 - protein, 22 aa, Artificial sequence
  Synthetic
NSRFESVAWSASACHDGTSWKK

SEQ ID NO: 940 - protein, 22 aa, Artificial sequence
  Synthetic
SVAWSASACHDGTSWLTIGIKK

SEQ ID NO: 941 - protein, 22 aa, Artificial sequence
  Synthetic
ASACHDGTSWLTIGISGPDNKK

SEQ ID NO: 942 - protein, 22 aa, Artificial sequence
  Synthetic
DGTSWLTIGISGPDNGAVAVKK

SEQ ID NO: 943 - protein, 22 aa, Artificial sequence
  Synthetic
LTIGISGPDNGAVAVLKYNGKK

SEQ ID NO: 944 - protein, 22 aa, Artificial sequence
  Synthetic
SGPDNGAVAVLKYNGIITDTKK

SEQ ID NO: 945 - protein, 22 aa, Artificial sequence
  Synthetic
GAVAVLKYNGIITDTIKSWRKK

SEQ ID NO: 946 - protein, 22 aa, Artificial sequence
  Synthetic
LKYNGIITDTIKSWRNNILRKK

SEQ ID NO: 947 - protein, 22 aa, Artificial sequence
  Synthetic
IITDTIKSWRNNILRTQESEKK

SEQ ID NO: 948 - protein, 22 aa, Artificial sequence
  Synthetic
IKSWRNNILRTQESECACVNKK

SEQ ID NO: 949 - protein, 22 aa, Artificial sequence
  Synthetic
NNILRTQESECACVNGSCFTKK

SEQ ID NO: 950 - protein, 22 aa, Artificial sequence
  Synthetic
TQESECACVNGSCFTVMTDGKK

SEQ ID NO: 951 - protein, 22 aa, Artificial sequence
  Synthetic
CACVNGSCFTVMTDGPSNGQKK

SEQ ID NO: 952 - protein, 22 aa, Artificial sequence
  Synthetic
GSCFTVMTDGPSNGQASHKIKK

SEQ ID NO: 953 - protein, 22 aa, Artificial sequence
  Synthetic
VMTDGPSNGQASHKIFKMEKKK

SEQ ID NO: 954 - protein, 22 aa, Artificial sequence
  Synthetic
PSNGQASHKIFKMEKGKVVKKK

SEQ ID NO: 955 - protein, 22 aa, Artificial sequence
  Synthetic
ASHKIFKMEKGKVVKSVELDKK

SEQ ID NO: 956 - protein, 22 aa, Artificial sequence
  Synthetic
FKMEKGKVVKSVELDAPNYHKK

SEQ ID NO: 957 - protein, 22 aa, Artificial sequence
  Synthetic
GKVVKSVELDAPNYHYEECSKK

SEQ ID NO: 958 - protein, 22 aa, Artificial sequence
  Synthetic
SVELDAPNYHYEECSCYPNAKK

SEQ ID NO: 959 - protein, 22 aa, Artificial sequence
  Synthetic
APNYHYEECSCYPNAGEITCKK

SEQ ID NO: 960 - protein, 22 aa, Artificial sequence
  Synthetic
YEECSCYPNAGEITCVCRDNKK

SEQ ID NO: 961 - protein, 22 aa, Artificial sequence
  Synthetic
CYPNAGEITCVCRDNWHGSNKK

SEQ ID NO: 962 - protein, 22 aa, Artificial sequence
  Synthetic
GEITCVCRDNWHGSNRPWVSKK

SEQ ID NO: 963 - protein, 22 aa, Artificial sequence
  Synthetic
VCRDNWHGSNRPWVSFNQNLKK

SEQ ID NO: 964 - protein, 22 aa, Artificial sequence
  Synthetic
WHGSNRPWVSFNQNLEYQIGKK

SEQ ID NO: 965 - protein, 22 aa, Artificial sequence
  Synthetic
RPWVSFNQNLEYQIGYICSGKK

SEQ ID NO: 966 - protein, 22 aa, Artificial sequence
  Synthetic
FNQNLEYQIGYICSGVFGDNKK

SEQ ID NO: 967 - protein, 22 aa, Artificial sequence
  Synthetic
EYQIGYICSGVFGDNPRPNDKK

SEQ ID NO: 968 - protein, 22 aa, Artificial sequence
  Synthetic
YICSGVFGDNPRPNDGTGSCKK

SEQ ID NO: 969 - protein, 22 aa, Artificial sequence
  Synthetic
VFGDNPRPNDGTGSCGPVSSKK

SEQ ID NO: 970 - protein, 22 aa, Artificial sequence
  Synthetic
PRPNDGTGSCGPVSSNGAYGKK

SEQ ID NO: 971 - protein, 22 aa, Artificial sequence
  Synthetic
GTGSCGPVSSNGAYGVKGFSKK

SEQ ID NO: 972 - protein, 22 aa, Artificial sequence
  Synthetic
GPVSSNGAYGVKGFSFKYGNKK

SEQ ID NO: 973 - protein, 22 aa, Artificial sequence
  Synthetic
NGAYGVKGFSFKYGNGVWIGKK

SEQ ID NO: 974 - protein, 22 aa, Artificial sequence
  Synthetic
VKGFSFKYGNGVWIGRTKSTKK

SEQ ID NO: 975 - protein, 22 aa, Artificial sequence
  Synthetic
FKYGNGVWIGRTKSTNSRSGKK

SEQ ID NO: 976 - protein, 22 aa, Artificial sequence
  Synthetic
GVWIGRTKSTNSRSGFEMIWKK

SEQ ID NO: 977 - protein, 22 aa, Artificial sequence
  Synthetic
RTKSTNSRSGFEMIWDPNGWKK

SEQ ID NO: 978 - protein, 22 aa, Artificial sequence
  Synthetic
NSRSGFEMIWDPNGWTETDSKK

SEQ ID NO: 979 - protein, 22 aa, Artificial sequence
  Synthetic
FEMIWDPNGWTETDSSFSVKKK

SEQ ID NO: 980 - protein, 22 aa, Artificial sequence
  Synthetic
DPNGWTETDSSFSVKQDIVAKK

SEQ ID NO: 981 - protein, 22 aa, Artificial sequence
  Synthetic
TETDSSFSVKQDIVAITDWSKK

SEQ ID NO: 982 - protein, 22 aa, Artificial sequence
  Synthetic
SFSVKQDIVAITDWSGYSGSKK

SEQ ID NO: 983 - protein, 22 aa, Artificial sequence
  Synthetic
QDIVAITDWSGYSGSFVQHPKK

SEQ ID NO: 984 - protein, 22 aa, Artificial sequence
  Synthetic
ITDWSGYSGSFVQHPELTGLKK

SEQ ID NO: 985 - protein, 22 aa, Artificial sequence
  Synthetic
GYSGSFVQHPELTGLDCIRPKK

SEQ ID NO: 986 - protein, 22 aa, Artificial sequence
  Synthetic
FVQHPELTGLDCIRPCFWVEKK

SEQ ID NO: 987 - protein, 22 aa, Artificial sequence
  Synthetic
ELTGLDCIRPCFWVELIRGRKK

SEQ ID NO: 988 - protein, 22 aa, Artificial sequence
  Synthetic
DCIRPCFWVELIRGRPKESTKK

SEQ ID NO: 989 - protein, 22 aa, Artificial sequence
  Synthetic
CFWVELIRGRPKESTIWTSGKK

SEQ ID NO: 990 - protein, 22 aa, Artificial sequence
  Synthetic
LIRGRPKESTIWTSGSSISFKK

SEQ ID NO: 991 - protein, 22 aa, Artificial sequence
  Synthetic
PKESTIWTSGSSISFCGVNSKK

SEQ ID NO: 992 - protein, 22 aa, Artificial sequence
  Synthetic
IWTSGSSISFCGVNSDTVGWKK

SEQ ID NO: 993 - protein, 22 aa, Artificial sequence
  Synthetic
SSISFCGVNSDTVGWSWPDGKK

SEQ ID NO: 994 - protein, 22 aa, Artificial sequence
  Synthetic
CGVNSDTVGWSWPDGAELPFKK

SEQ ID NO: 995 - protein, 21 aa, Artificial sequence
  Synthetic
DTVGWSWPDGAELPFTIDKKK

SEQ ID NO: 996 - protein, 20 aa, Artificial sequence
  Synthetic
MKTIIALSHILCLVFAQKLP

SEQ ID NO: 997 - protein, 20 aa, Artificial sequence
  Synthetic
ALSHILCLVFAQKLPGNDNS

SEQ ID NO: 998 - protein, 20 aa, Artificial sequence
  Synthetic
LCLVFAQKLPGNDNSTATLC

SEQ ID NO: 999 - protein, 20 aa, Artificial sequence
  Synthetic
AQKLPGNDNSTATLCLGHHA

SEQ ID NO: 1000 - protein, 20 aa, Artificial sequence
  Synthetic
GNDNSTATLCLGHHAVPNGT

SEQ ID NO: 1001 - protein, 20 aa, Artificial sequence
  Synthetic
TATLCLGHHAVPNGTIVKTI

SEQ ID NO: 1002 - protein, 20 aa, Artificial sequence
  Synthetic
LGHHAVPNGTIVKTITNDQI

SEQ ID NO: 1003 - protein, 20 aa, Artificial sequence
  Synthetic
VPNGTIVKTITNDQIEVTNA

SEQ ID NO: 1004 - protein, 20 aa, Artificial sequence
  Synthetic
IVKTITNDQIEVTNATELVQ

SEQ ID NO: 1005 - protein, 20 aa, Artificial sequence
  Synthetic
TNDQIEVTNATELVQNSSIG

SEQ ID NO: 1006 - protein, 20 aa, Artificial sequence
  Synthetic
EVTNATELVQNSSIGEICDS

SEQ ID NO: 1007 - protein, 20 aa, Artificial sequence
  Synthetic
TELVQNSSIGEICDSPHQIL

SEQ ID NO: 1008 - protein, 20 aa, Artificial sequence
  Synthetic
NSSIGEICDSPHQILDGENC

SEQ ID NO: 1009 - protein, 20 aa, Artificial sequence
  Synthetic
EICDSPHQILDGENCTLIDA

SEQ ID NO: 1010 - protein, 20 aa, Artificial sequence
  Synthetic
PHQILDGENCTLIDALLGDP

SEQ ID NO: 1011 - protein, 20 aa, Artificial sequence
  Synthetic
DGENCTLIDALLGDPQCDGF

SEQ ID NO: 1012 - protein, 20 aa, Artificial sequence
  Synthetic
TLIDALLGDPQCDGFQNKKW

SEQ ID NO: 1013 - protein, 20 aa, Artificial sequence
  Synthetic
LLGDPQCDGFQNKKWDLFVE

SEQ ID NO: 1014 - protein, 20 aa, Artificial sequence
  Synthetic
QCDGFQNKKWDLFVERSKAY

SEQ ID NO: 1015 - protein, 20 aa, Artificial sequence
  Synthetic
QNKKWDLFVERSKAYSNCYP

SEQ ID NO: 1016 - protein, 20 aa, Artificial sequence
  Synthetic
DLFVERSKAYSNCYPYDVPD

SEQ ID NO: 1017 - protein, 20 aa, Artificial sequence
  Synthetic
RSKAYSNCYPYDVPDYASLR

SEQ ID NO: 1018 - protein, 20 aa, Artificial sequence
  Synthetic
SNCYPYDVPDYASLRSLVAS

SEQ ID NO: 1019 - protein, 20 aa, Artificial sequence
  Synthetic
YDVPDYASLRSLVASSGTLE

SEQ ID NO: 1020 - protein, 20 aa, Artificial sequence
  Synthetic
YASLRSLVASSGTLEFNNES

SEQ ID NO: 1021 - protein, 20 aa, Artificial sequence
  Synthetic
SLVASSGTLEFNNESFNWTG

SEQ ID NO: 1022 - protein, 20 aa, Artificial sequence
  Synthetic
SGTLEFNNESFNWTGVTQNG

SEQ ID NO: 1023 - protein, 20 aa, Artificial sequence
  Synthetic
FNNESFNWTGVTQNGTSSAC

SEQ ID NO: 1024 - protein, 20 aa, Artificial sequence
  Synthetic
FNWTGVTQNGTSSACIRRSN

SEQ ID NO: 1025 - protein, 20 aa, Artificial sequence
  Synthetic
VTQNGTSSACIRRSNNSFFS

SEQ ID NO: 1026 - protein, 20 aa, Artificial sequence
  Synthetic
TSSACIRRSNNSFFSRLNWL

SEQ ID NO: 1027 - protein, 20 aa, Artificial sequence
  Synthetic
IRRSNNSFFSRLNWLTQLNF

SEQ ID NO: 1028 - protein, 20 aa, Artificial sequence
  Synthetic
NSFFSRLNWLTQLNFKYPAL

SEQ ID NO: 1029 - protein, 20 aa, Artificial sequence
  Synthetic
RLNWLTQLNFKYPALNVTMP

SEQ ID NO: 1030 - protein, 20 aa, Artificial sequence
  Synthetic
TQLNFKYPALNVTMPNNEQF

SEQ ID NO: 1031 - protein, 20 aa, Artificial sequence
  Synthetic
KYPALNVTMPNNEQFDKLYI

SEQ ID NO: 1032 - protein, 20 aa, Artificial sequence
  Synthetic
NVTMPNNEQFDKLYIWGVHH

SEQ ID NO: 1033 - protein, 20 aa, Artificial sequence
  Synthetic
NNEQFDKLYIWGVHHPVTDK

SEQ ID NO: 1034 - protein, 20 aa, Artificial sequence
  Synthetic
DKLYIWGVHHPVTDKDQIFL

SEQ ID NO: 1035 - protein, 20 aa, Artificial sequence
  Synthetic
WGVHHPVTDKDQIFLYAQSS

SEQ ID NO: 1036 - protein, 20 aa, Artificial sequence
  Synthetic
PVTDKDQIFLYAQSSGRITV

SEQ ID NO: 1037 - protein, 20 aa, Artificial sequence
  Synthetic
DQIFLYAQSSGRITVSTKRS

SEQ ID NO: 1038 - protein, 20 aa, Artificial sequence
  Synthetic
YAQSSGRITVSTKRSQQAVI

SEQ ID NO: 1039 - protein, 20 aa, Artificial sequence
  Synthetic
GRITVSTKRSQQAVIPNIGY

SEQ ID NO: 1040 - protein, 20 aa, Artificial sequence
  Synthetic
STKRSQQAVIPNIGYRPRIR

SEQ ID NO: 1041 - protein, 20 aa, Artificial sequence
  Synthetic
QQAVIPNIGYRPRIRNIPSR

SEQ ID NO: 1042 - protein, 20 aa, Artificial sequence
  Synthetic
PNIGYRPRIRNIPSRISIYW

SEQ ID NO: 1043 - protein, 20 aa, Artificial sequence
  Synthetic
RPRIRNIPSRISIYWTIVKP

SEQ ID NO: 1044 - protein, 20 aa, Artificial sequence
  Synthetic
NIPSRISIYWTIVKPGDILL

SEQ ID NO: 1045 - protein, 20 aa, Artificial sequence
  Synthetic
ISIYWTIVKPGDILLINSTG

SEQ ID NO: 1046 - protein, 20 aa, Artificial sequence
  Synthetic
TIVKPGDILLINSTGNLIAP

SEQ ID NO: 1047 - protein, 20 aa, Artificial sequence
  Synthetic
GDILLINSTGNLIAPRGYFK

SEQ ID NO: 1048 - protein, 20 aa, Artificial sequence
  Synthetic
INSTGNLIAPRGYFKIRSGK

SEQ ID NO: 1049 - protein, 20 aa, Artificial sequence
  Synthetic
NLIAPRGYFKIRSGKSSIMR

SEQ ID NO: 1050 - protein, 20 aa, Artificial sequence
  Synthetic
RGYFKIRSGKSSIMRSDAPI

SEQ ID NO: 1051 - protein, 20 aa, Artificial sequence
  Synthetic
IRSGKSSIMRSDAPIGKCNS

SEQ ID NO: 1052 - protein, 20 aa, Artificial sequence
  Synthetic
SSIMRSDAPIGKCNSECITP

SEQ ID NO: 1053 - protein, 20 aa, Artificial sequence
  Synthetic
SDAPIGKCNSECITPNGSIP

SEQ ID NO: 1054 - protein, 20 aa, Artificial sequence
  Synthetic
GKCNSECITPNGSIPNDKPF

SEQ ID NO: 1055 - protein, 20 aa, Artificial sequence
  Synthetic
ECITPNGSIPNDKPFQNVNR

SEQ ID NO: 1056 - protein, 20 aa, Artificial sequence
  Synthetic
NGSIPNDKPFQNVNRITYGA

SEQ ID NO: 1057 - protein, 20 aa, Artificial sequence
  Synthetic
NDKPFQNVNRITYGACPRYV

SEQ ID NO: 1058 - protein, 20 aa, Artificial sequence
  Synthetic
QNVNRITYGACPRYVKQSTL

SEQ ID NO: 1059 - protein, 20 aa, Artificial sequence
  Synthetic
ITYGACPRYVKQSTLKLATG

SEQ ID NO: 1060 - protein, 20 aa, Artificial sequence
  Synthetic
CPRYVKQSTLKLATGMRNVP

SEQ ID NO: 1061 - protein, 20 aa, Artificial sequence
  Synthetic
KQSTLKLATGMRNVPEKQTR

SEQ ID NO: 1062 - protein, 20 aa, Artificial sequence
  Synthetic
KLATGMRNVPEKQTRGIFGA

SEQ ID NO: 1063 - protein, 20 aa, Artificial sequence
  Synthetic
MRNVPEKQTRGIFGAIAGFI

SEQ ID NO: 1064 - protein, 20 aa, Artificial sequence
  Synthetic
EKQTRGIFGAIAGFIENGWE

SEQ ID NO: 1065 - protein, 20 aa, Artificial sequence
  Synthetic
GIFGAIAGFIENGWEGMVDG

SEQ ID NO: 1066 - protein, 20 aa, Artificial sequence
  Synthetic
IAGFIENGWEGMVDGWYGFR

SEQ ID NO: 1067 - protein, 20 aa, Artificial sequence
  Synthetic
ENGWEGMVDGWYGFRHQNSE

SEQ ID NO: 1068 - protein, 20 aa, Artificial sequence
  Synthetic
GMVDGWYGFRHQNSEGRGQA

SEQ ID NO: 1069 - protein, 20 aa, Artificial sequence
  Synthetic
WYGFRHQNSEGRGQAADLKS

SEQ ID NO: 1070 - protein, 20 aa, Artificial sequence
  Synthetic
HQNSEGRGQAADLKSTQAAI

SEQ ID NO: 1071 - protein, 20 aa, Artificial sequence
  Synthetic
GRGQAADLKSTQAAIDQING

SEQ ID NO: 1072 - protein, 20 aa, Artificial sequence
  Synthetic
ADLKSTQAAIDQINGKLNRL

SEQ ID NO: 1073 - protein, 20 aa, Artificial sequence
  Synthetic
TQAAIDQINGKLNRLIGKTN

SEQ ID NO: 1074 - protein, 20 aa, Artificial sequence
  Synthetic
DQINGKLNRLIGKTNEKFHQ

SEQ ID NO: 1075 - protein, 20 aa, Artificial sequence
  Synthetic
KLNRLIGKTNEKFHQIEKEF

SEQ ID NO: 1076 - protein, 20 aa, Artificial sequence
  Synthetic
IGKTNEKFHQIEKEFSEVEG

SEQ ID NO: 1077 - protein, 20 aa, Artificial sequence
  Synthetic
EKFHQIEKEFSEVEGRIQDL

SEQ ID NO: 1078 - protein, 20 aa, Artificial sequence
  Synthetic
IEKEFSEVEGRIQDLEKYVE

SEQ ID NO: 1079 - protein, 20 aa, Artificial sequence
  Synthetic
SEVEGRIQDLEKYVEDTKID

SEQ ID NO: 1080 - protein, 20 aa, Artificial sequence
  Synthetic
RIQDLEKYVEDTKIDLWSYN

SEQ ID NO: 1081 - protein, 20 aa, Artificial sequence
  Synthetic
EKYVEDTKIDLWSYNAELLV

SEQ ID NO: 1082 - protein, 20 aa, Artificial sequence
  Synthetic
DTKIDLWSYNAELLVALENQ

SEQ ID NO: 1083 - protein, 20 aa, Artificial sequence
  Synthetic
LWSYNAELLVALENQHTIDL

SEQ ID NO: 1084 - protein, 20 aa, Artificial sequence
  Synthetic
AELLVALENQHTIDLTDSEM

SEQ ID NO: 1085 - protein, 20 aa, Artificial sequence
  Synthetic
ALENQHTIDLTDSEMNKLFE

SEQ ID NO: 1086 - protein, 20 aa, Artificial sequence
  Synthetic
HTIDLTDSEMNKLFEKTKKQ

SEQ ID NO: 1087 - protein, 20 aa, Artificial sequence
  Synthetic
TDSEMNKLFEKTKKQLRENA

SEQ ID NO: 1088 - protein, 20 aa, Artificial sequence
  Synthetic
NKLFEKTKKQLRENAEDMGN

SEQ ID NO: 1089 - protein, 20 aa, Artificial sequence
  Synthetic
KTKKQLRENAEDMGNGCFKI

SEQ ID NO: 1090 - protein, 20 aa, Artificial sequence
  Synthetic
LRENAEDMGNGCFKIYHKCD

SEQ ID NO: 1091 - protein, 20 aa, Artificial sequence
  Synthetic
EDMGNGCFKIYHKCDNACIG

SEQ ID NO: 1092 - protein, 20 aa, Artificial sequence
  Synthetic
GCFKIYHKCDNACIGSIRNG

SEQ ID NO: 1093 - protein, 20 aa, Artificial sequence
  Synthetic
YHKCDNACIGSIRNGTYDHD

SEQ ID NO: 1094 - protein, 20 aa, Artificial sequence
  Synthetic
NACIGSIRNGTYDHDVYRDE

SEQ ID NO: 1095 - protein, 20 aa, Artificial sequence
  Synthetic
SIRNGTYDHDVYRDEALNNR

SEQ ID NO: 1096 - protein, 20 aa, Artificial sequence
  Synthetic
TYDHDVYRDEALNNRFQIKG

SEQ ID NO: 1097 - protein, 20 aa, Artificial sequence
  Synthetic
VYRDEALNNRFQIKGVELKS

SEQ ID NO: 1098 - protein, 20 aa, Artificial sequence
  Synthetic
ALNNRFQIKGVELKSGYKDW

SEQ ID NO: 1099 - protein, 20 aa, Artificial sequence
  Synthetic
FQIKGVELKSGYKDWILWIS

SEQ ID NO: 1100 - protein, 20 aa, Artificial sequence
  Synthetic
VELKSGYKDWILWISFAISC

SEQ ID NO: 1101 - protein, 24 aa, Artificial sequence
  Synthetic
KKGYKDWILWISFAISCFLLCVKK

SEQ ID NO: 1102 - protein, 25 aa, Artificial sequence
  Synthetic
KKKILWISFAISCFLLCVALLGFKK

SEQ ID NO: 1103 - protein, 25 aa, Artificial sequence
  Synthetic
KKKFAISCFLLCVALLGFIMWACKK

SEQ ID NO: 1104 - protein, 20 aa, Artificial sequence
  Synthetic
FLLCVALLGFIMWACQKGNI

SEQ ID NO: 1105 - protein, 20 aa, Artificial sequence
  Synthetic
ALLGFIMWACQKGNIRCNIC

SEQ ID NO: 1106 - protein, 16 aa, Artificial sequence
  Synthetic
IMWACQKGNIRCNICI

SEQ ID NO: 1107 - protein, 22 aa, Artificial sequence
  Synthetic
KKCFFMQIAILITTVTLHFKQY

SEQ ID NO: 1108 - protein, 22 aa, Artificial sequence
  Synthetic
KKTTVTLHFKQYEFNSPPNNQV

SEQ ID NO: 1109 - protein, 22 aa, Artificial sequence
  Synthetic
KKHFKQYEFNSPPNNQVMLCEP

SEQ ID NO: 1110 - protein, 22 aa, Artificial sequence
  Synthetic
KKEFNSPPNNQVMLCEPTIIER

SEQ ID NO: 1111 - protein, 22 aa, Artificial sequence
  Synthetic
KKPNNQVMLCEPTIIERNITEI

SEQ ID NO: 1112 - protein, 22 aa, Artificial sequence
  Synthetic
KKMLCEPTIIERNITEIVYLTN

SEQ ID NO: 1113 - protein, 22 aa, Artificial sequence
  Synthetic
KKTIIERNITEIVYLTNTTIEK

SEQ ID NO: 1114 - protein, 22 aa, Artificial sequence
  Synthetic
KKNITEIVYLTNTTIEKEICPK

SEQ ID NO: 1115 - protein, 22 aa, Artificial sequence
  Synthetic
KKVYLTNTTIEKEICPKPAEYR

SEQ ID NO: 1116 - protein, 22 aa, Artificial sequence
  Synthetic
KKTTIEKEICPKPAEYRNWSKP

SEQ ID NO: 1117 - protein, 22 aa, Artificial sequence
  Synthetic
KKEICPKPAEYRNWSKPQCGIT

SEQ ID NO: 1118 - protein, 22 aa, Artificial sequence
  Synthetic
KKPAEYRNWSKPQCGITGFAPF

SEQ ID NO: 1119 - protein, 22 aa, Artificial sequence
  Synthetic
KKNWSKPQCGITGFAPFSKDNS

SEQ ID NO: 1120 - protein, 22 aa, Artificial sequence
  Synthetic
KKQCGITGFAPFSKDNSIRLSA

SEQ ID NO: 1121 - protein, 22 aa, Artificial sequence
  Synthetic
KKGFAPFSKDNSIRLSAGGDIW

SEQ ID NO: 1122 - protein, 22 aa, Artificial sequence
  Synthetic
KKSKDNSIRLSAGGDIWVTREP

SEQ ID NO: 1123 - protein, 22 aa, Artificial sequence
  Synthetic
KKIRLSAGGDIWVTREPYVSCD

SEQ ID NO: 1124 - protein, 22 aa, Artificial sequence
  Synthetic
KKGGDIWVTREPYVSCDPDKCY

SEQ ID NO: 1125 - protein, 22 aa, Artificial sequence
  Synthetic
KKVTREPYVSCDPDKCYQFALG

SEQ ID NO: 1126 - protein, 22 aa, Artificial sequence
  Synthetic
KKYVSCDPDKCYQFALGQGTTL

SEQ ID NO: 1127 - protein, 22 aa, Artificial sequence
  Synthetic
KKPDKCYQFALGQGTTLNNVHS

SEQ ID NO: 1128 - protein, 22 aa, Artificial sequence
  Synthetic
KKQFALGQGTTLNNVHSNNTVR

SEQ ID NO: 1129 - protein, 22 aa, Artificial sequence
  Synthetic
KKQGTTLNNVHSNNTVRDRTPY

SEQ ID NO: 1130 - protein, 22 aa, Artificial sequence
  Synthetic
KKNNVHSNNTVRDRTPYRTLLM

SEQ ID NO: 1131 - protein, 22 aa, Artificial sequence
  Synthetic
KKNNTVRDRTPYRTLLMNELGV

SEQ ID NO: 1132 - protein, 22 aa, Artificial sequence
  Synthetic
KKDRTPYRTLLMNELGVPFHLG

SEQ ID NO: 1133 - protein, 22 aa, Artificial sequence
  Synthetic
KKRTLLMNELGVPFHLGTKQVC

SEQ ID NO: 1134 - protein, 22 aa, Artificial sequence
  Synthetic
KKNELGVPFHLGTKQVCIAWSS

SEQ ID NO: 1135 - protein, 22 aa, Artificial sequence
  Synthetic
KKPFHLGTKQVCIAWSSSSCHD

SEQ ID NO: 1136 - protein, 22 aa, Artificial sequence
  Synthetic
KKTKQVCIAWSSSSCHDGKAWL

SEQ ID NO: 1137 - protein, 22 aa, Artificial sequence
  Synthetic
KKIAWSSSSCHDGKAWLHVCIT

SEQ ID NO: 1138 - protein, 22 aa, Artificial sequence
  Synthetic
KKSSCHDGKAWLHVCITGDDKN

SEQ ID NO: 1139 - protein, 22 aa, Artificial sequence
  Synthetic
KKGKAWLHVCITGDDKNATASF

SEQ ID NO: 1140 - protein, 22 aa, Artificial sequence
  Synthetic
KKHVCITGDDKNATASFIYNGR

SEQ ID NO: 1141 - protein, 22 aa, Artificial sequence
  Synthetic
KKGDDKNATASFIYNGRLVDSV

SEQ ID NO: 1142 - protein, 22 aa, Artificial sequence
  Synthetic
KKATASFIYNGRLVDSVVSWSK

SEQ ID NO: 1143 - protein, 22 aa, Artificial sequence
  Synthetic
KKIYNGRLVDSVVSWSKEILRT

SEQ ID NO: 1144 - protein, 22 aa, Artificial sequence
  Synthetic
KKLVDSVVSWSKEILRTQESEC

SEQ ID NO: 1145 - protein, 22 aa, Artificial sequence
  Synthetic
KKVSWSKEILRTQESECVCING

SEQ ID NO: 1146 - protein, 22 aa, Artificial sequence
  Synthetic
KKQESECVCINGTCTVVMTDGS

SEQ ID NO: 1147 - protein, 22 aa, Artificial sequence
  Synthetic
KKVCINGTCTVVMTDGSASGKA

SEQ ID NO: 1148 - protein, 22 aa, Artificial sequence
  Synthetic
KKTCTVVMTDGSASGKADTKIL

SEQ ID NO: 1149 - protein, 22 aa, Artificial sequence
  Synthetic
KKMTDGSASGKADTKILFIKEG

SEQ ID NO: 1150 - protein, 22 aa, Artificial sequence
  Synthetic
KKASGKADTKILFIKEGKIVHT

SEQ ID NO: 1151 - protein, 22 aa, Artificial sequence
  Synthetic
KKDTKILFIKEGKIVHTSTLSG

SEQ ID NO: 1152 - protein, 22 aa, Artificial sequence
  Synthetic
KKFIKEGKIVHTSTLSGSAQHV

SEQ ID NO: 1153 - protein, 22 aa, Artificial sequence
  Synthetic
KKKIVHTSTLSGSAQHVEECSC

SEQ ID NO: 1154 - protein, 22 aa, Artificial sequence
  Synthetic
KKSTLSGSAQHVEECSCYPRYP

SEQ ID NO: 1155 - protein, 22 aa, Artificial sequence
  Synthetic
KKSAQHVEECSCYPRYPGVRCV

SEQ ID NO: 1156 - protein, 22 aa, Artificial sequence
  Synthetic
KKEECSCYPRYPGVRCVCRDNW

SEQ ID NO: 1157 - protein, 22 aa, Artificial sequence
  Synthetic
KKYPRYPGVRCVCRDNWKGSNR

SEQ ID NO: 1158 - protein, 22 aa, Artificial sequence
  Synthetic
KKGVRCVCRDNWKGSNRPIVDI

SEQ ID NO: 1159 - protein, 22 aa, Artificial sequence
  Synthetic
KKCRDNWKGSNRPIVDINIKDH

SEQ ID NO: 1160 - protein, 22 aa, Artificial sequence
  Synthetic
KKKGSNRPIVDINIKDHSIVSS

SEQ ID NO: 1161 - protein, 22 aa, Artificial sequence
  Synthetic
KKPIVDINIKDHSIVSSYVCSG

SEQ ID NO: 1162 - protein, 22 aa, Artificial sequence
  Synthetic
KKNIKDHSIVSSYVCSGLVGDT

SEQ ID NO: 1163 - protein, 22 aa, Artificial sequence
  Synthetic
KKSIVSSYVCSGLVGDTPRKTD

SEQ ID NO: 1164 - protein, 22 aa, Artificial sequence
  Synthetic
KKYVCSGLVGDTPRKTDSSSSS

SEQ ID NO: 1165 - protein, 22 aa, Artificial sequence
  Synthetic
KKLVGDTPRKTDSSSSSHCLDP

SEQ ID NO: 1166 - protein, 22 aa, Artificial sequence
  Synthetic
KKPRKTDSSSSSHCLDPNNEEG

SEQ ID NO: 1167 - protein, 22 aa, Artificial sequence
  Synthetic
KKSSSSSHCLDPNNEEGGHGVK

SEQ ID NO: 1168 - protein, 22 aa, Artificial sequence
  Synthetic
KKHCLDPNNEEGGHGVKGWAFD

SEQ ID NO: 1169 - protein, 22 aa, Artificial sequence
  Synthetic
KKNNEEGGHGVKGWAFDDGNDV

SEQ ID NO: 1170 - protein, 22 aa, Artificial sequence
  Synthetic
KKGHGVKGWAFDDGNDVWMGRT

SEQ ID NO: 1171 - protein, 22 aa, Artificial sequence
  Synthetic
KKGWAFDDGNDVWMGRTINETS

SEQ ID NO: 1172 - protein, 22 aa, Artificial sequence
  Synthetic
KKDGNDVWMGRTINETSRLGYE

SEQ ID NO: 1173 - protein, 22 aa, Artificial sequence
  Synthetic
KKWMGRTINETSRLGYETFKVI

SEQ ID NO: 1174 - protein, 22 aa, Artificial sequence
  Synthetic
KKINETSRLGYETFKVIEGWSN

SEQ ID NO: 1175 - protein, 22 aa, Artificial sequence
  Synthetic
KKRLGYETFKVIEGWSNPKSKL

SEQ ID NO: 1176 - protein, 22 aa, Artificial sequence
  Synthetic
KKTFKVIEGWSNPKSKLQINRQ

SEQ ID NO: 1177 - protein, 22 aa, Artificial sequence
  Synthetic
KKEGWSNPKSKLQINRQVIVDR

SEQ ID NO: 1178 - protein, 22 aa, Artificial sequence
  Synthetic
KKPKSKLQINRQVIVDRGDRSG

SEQ ID NO: 1179 - protein, 22 aa, Artificial sequence
  Synthetic
KKQINRQVIVDRGDRSGYSGIF

SEQ ID NO: 1180 - protein, 22 aa, Artificial sequence
  Synthetic
KKVIVDRGDRSGYSGIFSVEGK

SEQ ID NO: 1181 - protein, 22 aa, Artificial sequence
  Synthetic
KKGDRSGYSGIFSVEGKSCINR

SEQ ID NO: 1182 - protein, 22 aa, Artificial sequence
  Synthetic
KKYSGIFSVEGKSCINRCFYVE

SEQ ID NO: 1183 - protein, 22 aa, Artificial sequence
  Synthetic
KKSVEGKSCINRCFYVELIRGR

SEQ ID NO: 1184 - protein, 22 aa, Artificial sequence
  Synthetic
KKSCINRCFYVELIRGRKEETE

SEQ ID NO: 1185 - protein, 22 aa, Artificial sequence
  Synthetic
KKCFYVELIRGRKEETEVLWTS

SEQ ID NO: 1186 - protein, 22 aa, Artificial sequence
  Synthetic
KKLIRGRKEETEVLWTSNSIVV

SEQ ID NO: 1187 - protein, 23 aa, Artificial sequence
  Synthetic
KKKVLWTSNSIVVFCGTSGTYGT

SEQ ID NO: 1188 - protein, 23 aa, Artificial sequence
  Synthetic
KKKNSIVVFCGTSGTYGTGSWPD

SEQ ID NO: 1189 - protein, 23 aa, Artificial sequence
  Synthetic
KKKFCGTSGTYGTGSWPDGADLN

SEQ ID NO: 1190 - protein, 21 aa, Artificial sequence
  Synthetic
KKGTYGTGSWPDGADLNLMPI

SEQ ID NO: 1191 - protein, 20 aa, Artificial sequence
  Synthetic
MKAIIVLLMVVTSNADRICT

SEQ ID NO: 1192 - protein, 20 aa, Artificial sequence
  Synthetic
VLLMVVTSNADRICTGITSS

SEQ ID NO: 1193 - protein, 20 aa, Artificial sequence
  Synthetic
VTSNADRICTGITSSNSPHV

SEQ ID NO: 1194 - protein, 20 aa, Artificial sequence
  Synthetic
DRICTGITSSNSPHVVKTAT

SEQ ID NO: 1195 - protein, 20 aa, Artificial sequence
  Synthetic
GITSSNSPHVVKTATQGEVN

SEQ ID NO: 1196 - protein, 20 aa, Artificial sequence
  Synthetic
NSPHVVKTATQGEVNVTGVI

SEQ ID NO: 1197 - protein, 20 aa, Artificial sequence
  Synthetic
VKTATQGEVNVTGVIPLTTT

SEQ ID NO: 1198 - protein, 20 aa, Artificial sequence
  Synthetic
QGEVNVTGVIPLTTTPTKSY

SEQ ID NO: 1199 - protein, 20 aa, Artificial sequence
  Synthetic
VTGVIPLTTTPTKSYFANLK

SEQ ID NO: 1200 - protein, 20 aa, Artificial sequence
  Synthetic
PLTTTPTKSYFANLKGTRTR

SEQ ID NO: 1201 - protein, 20 aa, Artificial sequence
  Synthetic
PTKSYFANLKGTRTRGKLCP

SEQ ID NO: 1202 - protein, 20 aa, Artificial sequence
  Synthetic
FANLKGTRTRGKLCPDCLNC

SEQ ID NO: 1203 - protein, 20 aa, Artificial sequence
  Synthetic
GTRTRGKLCPDCLNCTDLDV

SEQ ID NO: 1204 - protein, 20 aa, Artificial sequence
  Synthetic
GKLCPDCLNCTDLDVALGRP

SEQ ID NO: 1205 - protein, 20 aa, Artificial sequence
  Synthetic
DCLNCTDLDVALGRPMCVGT

SEQ ID NO: 1206 - protein, 20 aa, Artificial sequence
  Synthetic
TDLDVALGRPMCVGTTPSAK

SEQ ID NO: 1207 - protein, 20 aa, Artificial sequence
  Synthetic
ALGRPMCVGTTPSAKASILH

SEQ ID NO: 1208 - protein, 20 aa, Artificial sequence
  Synthetic
MCVGTTPSAKASILHEVRPV

SEQ ID NO: 1209 - protein, 20 aa, Artificial sequence
  Synthetic
TPSAKASILHEVRPVTSGCF

SEQ ID NO: 1210 - protein, 20 aa, Artificial sequence
  Synthetic
ASILHEVRPVTSGCFPIMHD

SEQ ID NO: 1211 - protein, 20 aa, Artificial sequence
  Synthetic
EVRPVTSGCFPIMHDRTKIR

SEQ ID NO: 1212 - protein, 20 aa, Artificial sequence
  Synthetic
TSGCFPIMHDRTKIRQLPNL

SEQ ID NO: 1213 - protein, 20 aa, Artificial sequence
  Synthetic
PIMHDRTKIRQLPNLLRGYE

SEQ ID NO: 1214 - protein, 20 aa, Artificial sequence
  Synthetic
RTKIRQLPNLLRGYENIRLS

SEQ ID NO: 1215 - protein, 20 aa, Artificial sequence
  Synthetic
QLPNLLRGYENIRLSTQNVI

SEQ ID NO: 1216 - protein, 20 aa, Artificial sequence
  Synthetic
LRGYENIRLSTQNVIDAEKA

SEQ ID NO: 1217 - protein, 20 aa, Artificial sequence
  Synthetic
NIRLSTQNVIDAEKAPGGPY

SEQ ID NO: 1218 - protein, 20 aa, Artificial sequence
  Synthetic
TQNVIDAEKAPGGPYRLGTS

SEQ ID NO: 1219 - protein, 20 aa, Artificial sequence
  Synthetic
DAEKAPGGPYRLGTSGSCPN

SEQ ID NO: 1220 - protein, 20 aa, Artificial sequence
  Synthetic
PGGPYRLGTSGSCPNATSKI

SEQ ID NO: 1221 - protein, 20 aa, Artificial sequence
  Synthetic
RLGTSGSCPNATSKIGFFAT

SEQ ID NO: 1222 - protein, 24 aa, Artificial sequence
  Synthetic
KKGSCPNATSKIGFFATMAWAVKK

SEQ ID NO: 1223 - protein, 20 aa, Artificial sequence
  Synthetic
ATSKIGFFATMAWAVPKDNY

SEQ ID NO: 1224 - protein, 20 aa, Artificial sequence
  Synthetic
GFFATMAWAVPKDNYKNATN

SEQ ID NO: 1225 - protein, 20 aa, Artificial sequence
  Synthetic
MAWAVPKDNYKNATNPLTVE

SEQ ID NO: 1226 - protein, 20 aa, Artificial sequence
  Synthetic
PKDNYKNATNPLTVEVPYIC

SEQ ID NO: 1227 - protein, 20 aa, Artificial sequence
  Synthetic
KNATNPLTVEVPYICTEGED

SEQ ID NO: 1228 - protein, 20 aa, Artificial sequence
  Synthetic
PLTVEVPYICTEGEDQITVW

SEQ ID NO: 1229 - protein, 20 aa, Artificial sequence
  Synthetic
VPYICTEGEDQITVWGFHSD

SEQ ID NO: 1230 - protein, 20 aa, Artificial sequence
  Synthetic
TEGEDQITVWGFHSDNKTQM

SEQ ID NO: 1231 - protein, 20 aa, Artificial sequence
  Synthetic
QITVWGFHSDNKTQMKSLYG

SEQ ID NO: 1232 - protein, 20 aa, Artificial sequence
  Synthetic
GFHSDNKTQMKSLYGDSNPQ

SEQ ID NO: 1233 - protein, 20 aa, Artificial sequence
  Synthetic
NKTQMKSLYGDSNPQKFTSS

SEQ ID NO: 1234 - protein, 20 aa, Artificial sequence
  Synthetic
KSLYGDSNPQKFTSSANGVT

SEQ ID NO: 1235 - protein, 20 aa, Artificial sequence
  Synthetic
DSNPQKFTSSANGVTTHYVS

SEQ ID NO: 1236 - protein, 20 aa, Artificial sequence
  Synthetic
KFTSSANGVTTHYVSQIGDF

SEQ ID NO: 1237 - protein, 20 aa, Artificial sequence
  Synthetic
ANGVTTHYVSQIGDFPDQTE

SEQ ID NO: 1238 - protein, 20 aa, Artificial sequence
  Synthetic
THYVSQIGDFPDQTEDGGLP

SEQ ID NO: 1239 - protein, 20 aa, Artificial sequence
  Synthetic
QIGDFPDQTEDGGLPQSGRI

SEQ ID NO: 1240 - protein, 20 aa, Artificial sequence
  Synthetic
PDQTEDGGLPQSGRIVVDYM

SEQ ID NO: 1241 - protein, 20 aa, Artificial sequence
  Synthetic
DGGLPQSGRIVVDYMMQKPG

SEQ ID NO: 1242 - protein, 20 aa, Artificial sequence
  Synthetic
QSGRIVVDYMMQKPGKTGTI

SEQ ID NO: 1243 - protein, 20 aa, Artificial sequence
  Synthetic
VVDYMMQKPGKTGTIVYQRG

SEQ ID NO: 1244 - protein, 20 aa, Artificial sequence
  Synthetic
MQKPGKTGTIVYQRGVLLPQ

SEQ ID NO: 1245 - protein, 20 aa, Artificial sequence
  Synthetic
KTGTIVYQRGVLLPQKVWCA

SEQ ID NO: 1246 - protein, 20 aa, Artificial sequence
  Synthetic
VYQRGVLLPQKVWCASGRSK

SEQ ID NO: 1247 - protein, 20 aa, Artificial sequence
  Synthetic
VLLPQKVWCASGRSKVIKGS

SEQ ID NO: 1248 - protein, 20 aa, Artificial sequence
  Synthetic
KVWCASGRSKVIKGSLPLIG

SEQ ID NO: 1249 - protein, 20 aa, Artificial sequence
  Synthetic
SGRSKVIKGSLPLIGEADCL

SEQ ID NO: 1250 - protein, 20 aa, Artificial sequence
  Synthetic
VIKGSLPLIGEADCLHEKYG

SEQ ID NO: 1251 - protein, 20 aa, Artificial sequence
  Synthetic
LPLIGEADCLHEKYGGLNKS

SEQ ID NO: 1252 - protein, 20 aa, Artificial sequence
  Synthetic
EADCLHEKYGGLNKSKPYYT

SEQ ID NO: 1253 - protein, 20 aa, Artificial sequence
  Synthetic
HEKYGGLNKSKPYYTGEHAK

SEQ ID NO: 1254 - protein, 20 aa, Artificial sequence
  Synthetic
GLNKSKPYYTGEHAKAIGNC

SEQ ID NO: 1255 - protein, 20 aa, Artificial sequence
  Synthetic
KPYYTGEHAKAIGNCPIWVK

SEQ ID NO: 1256 - protein, 20 aa, Artificial sequence
  Synthetic
GEHAKAIGNCPIWVKTPLKL

SEQ ID NO: 1257 - protein, 20 aa, Artificial sequence
  Synthetic
AIGNCPIWVKTPLKLANGTK

SEQ ID NO: 1258 - protein, 20 aa, Artificial sequence
  Synthetic
PIWVKTPLKLANGTKYRPPA

SEQ ID NO: 1259 - protein, 20 aa, Artificial sequence
  Synthetic
TPLKLANGTKYRPPAKLLKE

SEQ ID NO: 1260 - protein, 20 aa, Artificial sequence
  Synthetic
ANGTKYRPPAKLLKERGFFG

SEQ ID NO: 1261 - protein, 20 aa, Artificial sequence
  Synthetic
YRPPAKLLKERGFFGAIAGF

SEQ ID NO: 1262 - protein, 20 aa, Artificial sequence
  Synthetic
KLLKERGFFGAIAGFLEGGW

SEQ ID NO: 1263 - protein, 24 aa, Artificial sequence
  Synthetic
KKRGFFGAIAGFLEGGWEGMIAKK

SEQ ID NO: 1264 - protein, 20 aa, Artificial sequence
  Synthetic
AIAGFLEGGWEGMIAGWHGY

SEQ ID NO: 1265 - protein, 20 aa, Artificial sequence
  Synthetic
LEGGWEGMIAGWHGYTSHGA

SEQ ID NO: 1266 - protein, 20 aa, Artificial sequence
  Synthetic
EGMIAGWHGYTSHGAHGVAV

SEQ ID NO: 1267 - protein, 20 aa, Artificial sequence
  Synthetic
GWHGYTSHGAHGVAVAADLK

SEQ ID NO: 1268 - protein, 20 aa, Artificial sequence
  Synthetic
TSHGAHGVAVAADLKSTQEA

SEQ ID NO: 1269 - protein, 20 aa, Artificial sequence
  Synthetic
HGVAVAADLKSTQEAINKIT

SEQ ID NO: 1270 - protein, 20 aa, Artificial sequence
  Synthetic
AADLKSTQEAINKITKNLNS

SEQ ID NO: 1271 - protein, 20 aa, Artificial sequence
  Synthetic
STQEAINKITKNLNSLSELE

SEQ ID NO: 1272 - protein, 20 aa, Artificial sequence
  Synthetic
INKITKNLNSLSELEVKNLQ

SEQ ID NO: 1273 - protein, 20 aa, Artificial sequence
  Synthetic
KNLNSLSELEVKNLQRLSGA

SEQ ID NO: 1274 - protein, 20 aa, Artificial sequence
  Synthetic
LSELEVKNLQRLSGAMDELH

SEQ ID NO: 1275 - protein, 20 aa, Artificial sequence
  Synthetic
VKNLQRLSGAMDELHNEILE

SEQ ID NO: 1276 - protein, 20 aa, Artificial sequence
  Synthetic
RLSGAMDELHNEILELDEKV

SEQ ID NO: 1277 - protein, 20 aa, Artificial sequence
  Synthetic
MDELHNEILELDEKVDDLRA

SEQ ID NO: 1278 - protein, 20 aa, Artificial sequence
  Synthetic
NEILELDEKVDDLRADTISS

SEQ ID NO: 1279 - protein, 20 aa, Artificial sequence
  Synthetic
LDEKVDDLRADTISSQIELA

SEQ ID NO: 1280 - protein, 24 aa, Artificial sequence
  Synthetic
KKDDLRADTISSQIELAVLLSNKK

SEQ ID NO: 1281 - protein, 24 aa, Artificial sequence
  Synthetic
KKDTISSQIELAVLLSNEGIINKK

SEQ ID NO: 1282 - protein, 20 aa, Artificial sequence
  Synthetic
QIELAVLLSNEGIINSEDEH

SEQ ID NO: 1283 - protein, 20 aa, Artificial sequence
  Synthetic
VLLSNEGIINSEDEHLLALE

SEQ ID NO: 1284 - protein, 20 aa, Artificial sequence
  Synthetic
EGIINSEDEHLLALERKLKK

SEQ ID NO: 1285 - protein, 20 aa, Artificial sequence
  Synthetic
SEDEHLLALERKLKKMLGPS

SEQ ID NO: 1286 - protein, 20 aa, Artificial sequence
  Synthetic
LLALERKLKKMLGPSAVDIG

SEQ ID NO: 1287 - protein, 20 aa, Artificial sequence
  Synthetic
RKLKKMLGPSAVDIGNGCFE

SEQ ID NO: 1288 - protein, 20 aa, Artificial sequence
  Synthetic
MLGPSAVDIGNGCFETKHKC

SEQ ID NO: 1289 - protein, 20 aa, Artificial sequence
  Synthetic
AVDIGNGCFETKHKCNQTCL

SEQ ID NO: 1290 - protein, 20 aa, Artificial sequence
  Synthetic
NGCFETKHKCNQTCLDRIAA

SEQ ID NO: 1291 - protein, 20 aa, Artificial sequence
  Synthetic
TKHKCNQTCLDRIAAGTFNA

SEQ ID NO: 1292 - protein, 20 aa, Artificial sequence
  Synthetic
NQTCLDRIAAGTFNAGEFSL

SEQ ID NO: 1293 - protein, 20 aa, Artificial sequence
  Synthetic
DRIAAGTFNAGEFSLPTFDS

SEQ ID NO: 1294 - protein, 20 aa, Artificial sequence
  Synthetic
GTFNAGEFSLPTFDSLNITA

SEQ ID NO: 1295 - protein, 20 aa, Artificial sequence
  Synthetic
GEFSLPTFDSLNITAASLND

SEQ ID NO: 1296 - protein, 20 aa, Artificial sequence
  Synthetic
PTFDSLNITAASLNDDGLDN

SEQ ID NO: 1297 - protein, 20 aa, Artificial sequence
  Synthetic
LNITAASLNDDGLDNHTILL

SEQ ID NO: 1298 - protein, 20 aa, Artificial sequence
  Synthetic
ASLNDDGLDNHTILLYYSTA

SEQ ID NO: 1299 - protein, 20 aa, Artificial sequence
  Synthetic
DGLDNHTILLYYSTAASSLA

SEQ ID NO: 1300 - protein, 25 aa, Artificial sequence
  Synthetic
KKKHTILLYYSTAASSLAVTLMLKK

SEQ ID NO: 1301 - protein, 25 aa, Artificial sequence
  Synthetic
KKKYYSTAASSLAVTLMLAIFIVKK

SEQ ID NO: 1302 - protein, 24 aa, Artificial sequence
  Synthetic
KKASSLAVTLMLAIFIVYMVSRKK

SEQ ID NO: 1303 - protein, 24 aa, Artificial sequence
  Synthetic
KKVTLMLAIFIVYMVSRDNVSCKK

SEQ ID NO: 1304 - protein, 23 aa, Artificial sequence
  Synthetic
KKAIFIVYMVSRDNVSCSICLKK

SEQ ID NO: 1305 - protein, 22 aa, Artificial sequence
  Synthetic
KKMLPSTIQTLTLFLTSGGVLL

SEQ ID NO: 1306 - protein, 22 aa, Artificial sequence
  Synthetic
KKSLYVSASLSYLLYSDILLKF

SEQ ID NO: 1307 - protein, 22 aa, Artificial sequence
  Synthetic
KKASLSYLLYSDILLKFSRTEI

SEQ ID NO: 1308 - protein, 22 aa, Artificial sequence
  Synthetic
KKLLYSDILLKFSRTEITAPIM

SEQ ID NO: 1309 - protein, 22 aa, Artificial sequence
  Synthetic
KKILLKFSRTEITAPIMPLDCA

SEQ ID NO: 1310 - protein, 22 aa, Artificial sequence
  Synthetic
KKSRTEITAPIMPLDCANASNV

SEQ ID NO: 1311 - protein, 22 aa, Artificial sequence
  Synthetic
KKTAPIMPLDCANASNVQAVNR

SEQ ID NO: 1312 - protein, 22 aa, Artificial sequence
  Synthetic
KKPLDCANASNVQAVNRSATKG

SEQ ID NO: 1313 - protein, 22 aa, Artificial sequence
  Synthetic
KKNASNVQAVNRSATKGVTLLL

SEQ ID NO: 1314 - protein, 22 aa, Artificial sequence
  Synthetic
KKQAVNRSATKGVTLLLPEPEW

SEQ ID NO: 1315 - protein, 22 aa, Artificial sequence
  Synthetic
KKSATKGVTLLLPEPEWTYPRL

SEQ ID NO: 1316 - protein, 22 aa, Artificial sequence
  Synthetic
KKVTLLLPEPEWTYPRLSCPGS

SEQ ID NO: 1317 - protein, 22 aa, Artificial sequence
  Synthetic
KKPEPEWTYPRLSCPGSTFQKA

SEQ ID NO: 1318 - protein, 22 aa, Artificial sequence
  Synthetic
KKTYPRLSCPGSTFQKALLISP

SEQ ID NO: 1319 - protein, 22 aa, Artificial sequence
  Synthetic
KKSCPGSTFQKALLISPHRFGE

SEQ ID NO: 1320 - protein, 22 aa, Artificial sequence
  Synthetic
KKTFQKALLISPHRFGETKGNS

SEQ ID NO: 1321 - protein, 22 aa, Artificial sequence
  Synthetic
KKLLISPHRFGETKGNSAPLII

SEQ ID NO: 1322 - protein, 22 aa, Artificial sequence
  Synthetic
KKHRFGETKGNSAPLIIREPFI

SEQ ID NO: 1323 - protein, 22 aa, Artificial sequence
  Synthetic
KKTKGNSAPLIIREPFIACGPK

SEQ ID NO: 1324 - protein, 22 aa, Artificial sequence
  Synthetic
KKAPLIIREPFIACGPKECKHF

SEQ ID NO: 1325 - protein, 22 aa, Artificial sequence
  Synthetic
KKREPFIACGPKECKHFALTHY

SEQ ID NO: 1326 - protein, 22 aa, Artificial sequence
  Synthetic
KKACGPKECKHFALTHYAAQPG

SEQ ID NO: 1327 - protein, 22 aa, Artificial sequence
  Synthetic
KKECKHFALTHYAAQPGGYYNG

SEQ ID NO: 1328 - protein, 22 aa, Artificial sequence
  Synthetic
KKALTHYAAQPGGYYNGTREDR

SEQ ID NO: 1329 - protein, 22 aa, Artificial sequence
  Synthetic
KKAAQPGGYYNGTREDRNKLRH

SEQ ID NO: 1330 - protein, 22 aa, Artificial sequence
  Synthetic
KKGYYNGTREDRNKLRHLISVK

SEQ ID NO: 1331 - protein, 22 aa, Artificial sequence
  Synthetic
KKTREDRNKLRHLISVKLGKIP

SEQ ID NO: 1332 - protein, 22 aa, Artificial sequence
  Synthetic
KKNKLRHLISVKLGKIPTVENS

SEQ ID NO: 1333 - protein, 22 aa, Artificial sequence
  Synthetic
KKLISVKLGKIPTVENSIFHMA

SEQ ID NO: 1334 - protein, 22 aa, Artificial sequence
  Synthetic
KKLGKIPTVENSIFHMAAWSGS

SEQ ID NO: 1335 - protein, 22 aa, Artificial sequence
  Synthetic
KKTVENSIFHMAAWSGSACHDG

SEQ ID NO: 1336 - protein, 22 aa, Artificial sequence
  Synthetic
KKIFHMAAWSGSACHDGREWTY

SEQ ID NO: 1337 - protein, 22 aa, Artificial sequence
  Synthetic
KKAWSGSACHDGREWTYIGVDG

SEQ ID NO: 1338 - protein, 22 aa, Artificial sequence
  Synthetic
KKACHDGREWTYIGVDGPDSNA

SEQ ID NO: 1339 - protein, 22 aa, Artificial sequence
  Synthetic
KKREWTYIGVDGPDSNALLKIK

SEQ ID NO: 1340 - protein, 22 aa, Artificial sequence
  Synthetic
KKIGVDGPDSNALLKIKYGEAY

SEQ ID NO: 1341 - protein, 22 aa, Artificial sequence
  Synthetic
KKPDSNALLKIKYGEAYTDTYH

SEQ ID NO: 1342 - protein, 22 aa, Artificial sequence
  Synthetic
KKLLKIKYGEAYTDTYHSYAKN

SEQ ID NO: 1343 - protein, 22 aa, Artificial sequence
  Synthetic
KKYGEAYTDTYHSYAKNILRTQ

SEQ ID NO: 1344 - protein, 22 aa, Artificial sequence
  Synthetic
KKTDTYHSYAKNILRTQESACN

SEQ ID NO: 1345 - protein, 22 aa, Artificial sequence
  Synthetic
KKSYAKNILRTQESACNCIGGD

SEQ ID NO: 1346 - protein, 22 aa, Artificial sequence
  Synthetic
KKILRTQESACNCIGGDCYLMI

SEQ ID NO: 1347 - protein, 23 aa, Artificial sequence
  Synthetic
KKKCIGGDCYLMITDGPASGISE

SEQ ID NO: 1348 - protein, 22 aa, Artificial sequence
  Synthetic
KKCYLMITDGPASGISECRFLK

SEQ ID NO: 1349 - protein, 22 aa, Artificial sequence
  Synthetic
KKTDGPASGISECRFLKIREGR

SEQ ID NO: 1350 - protein, 22 aa, Artificial sequence
  Synthetic
KKSGISECRFLKIREGRIIKEI

SEQ ID NO: 1351 - protein, 22 aa, Artificial sequence
  Synthetic
KKCRFLKIREGRIIKEIFPTGR

SEQ ID NO: 1352 - protein, 22 aa, Artificial sequence
  Synthetic
KKIREGRIIKEIFPTGRVKHTE

SEQ ID NO: 1353 - protein, 22 aa, Artificial sequence
  Synthetic
KKIIKEIFPTGRVKHTEECTCG

SEQ ID NO: 1354 - protein, 22 aa, Artificial sequence
  Synthetic
KKFPTGRVKHTEECTCGFASNK

SEQ ID NO: 1355 - protein, 22 aa, Artificial sequence
  Synthetic
KKVKHTEECTCGFASNKTIECA

SEQ ID NO: 1356 - protein, 22 aa, Artificial sequence
  Synthetic
KKECTCGFASNKTIECACRDNS

SEQ ID NO: 1357 - protein, 22 aa, Artificial sequence
  Synthetic
KKFASNKTIECACRDNSYTAKR

SEQ ID NO: 1358 - protein, 22 aa, Artificial sequence
  Synthetic
KKTIECACRDNSYTAKRPFVKL

SEQ ID NO: 1359 - protein, 22 aa, Artificial sequence
  Synthetic
KKCRDNSYTAKRPFVKLNVETD

SEQ ID NO: 1360 - protein, 22 aa, Artificial sequence
  Synthetic
KKYTAKRPFVKLNVETDTAEIR

SEQ ID NO: 1361 - protein, 22 aa, Artificial sequence
  Synthetic
KKNVETDTAEIRLMCTETYLDT

SEQ ID NO: 1362 - protein, 22 aa, Artificial sequence
  Synthetic
KKTAEIRLMCTETYLDTPRPND

SEQ ID NO: 1363 - protein, 22 aa, Artificial sequence
  Synthetic
KKLMCTETYLDTPRPNDGSITG

SEQ ID NO: 1364 - protein, 22 aa, Artificial sequence
  Synthetic
KKTYLDTPRPNDGSITGPCESN

SEQ ID NO: 1365 - protein, 22 aa, Artificial sequence
  Synthetic
KKPRPNDGSITGPCESNGDKGS

SEQ ID NO: 1366 - protein, 22 aa, Artificial sequence
  Synthetic
KKGSITGPCESNGDKGSGGIKG

SEQ ID NO: 1367 - protein, 22 aa, Artificial sequence
  Synthetic
KKPCESNGDKGSGGIKGGFVHQ

SEQ ID NO: 1368 - protein, 22 aa, Artificial sequence
  Synthetic
KKGDKGSGGIKGGFVHQRMASK

SEQ ID NO: 1369 - protein, 22 aa, Artificial sequence
  Synthetic
KKGGIKGGFVHQRMASKIGRWY

SEQ ID NO: 1370 - protein, 22 aa, Artificial sequence
  Synthetic
KKGFVHQRMASKIGRWYSRTMS

SEQ ID NO: 1371 - protein, 22 aa, Artificial sequence
  Synthetic
KKRMASKIGRWYSRTMSKTKRM

SEQ ID NO: 1372 - protein, 22 aa, Artificial sequence
  Synthetic
KKIGRWYSRTMSKTKRMGMGLY

SEQ ID NO: 1373 - protein, 22 aa, Artificial sequence
  Synthetic
KKSRTMSKTKRMGMGLYVKYDG

SEQ ID NO: 1374 - protein, 22 aa, Artificial sequence
  Synthetic
KKKTKRMGMGLYVKYDGDPWTD

SEQ ID NO: 1375 - protein, 22 aa, Artificial sequence
  Synthetic
KKGMGLYVKYDGDPWTDSEALA

SEQ ID NO: 1376 - protein, 22 aa, Artificial sequence
  Synthetic
KKVKYDGDPWTDSEALALSGVM

SEQ ID NO: 1377 - protein, 22 aa, Artificial sequence
  Synthetic
KKDPWTDSEALALSGVMVSMEE

SEQ ID NO: 1378 - protein, 22 aa, Artificial sequence
  Synthetic
KKSEALALSGVMVSMEEPGWYS

SEQ ID NO: 1379 - protein, 22 aa, Artificial sequence
  Synthetic
KKLSGVMVSMEEPGWYSFGFEI

SEQ ID NO: 1380 - protein, 22 aa, Artificial sequence
  Synthetic
KKVSMEEPGWYSFGFEIKDKKC

SEQ ID NO: 1381 - protein, 22 aa, Artificial sequence
  Synthetic
KKPGWYSFGFEIKDKKCDVPCI

SEQ ID NO: 1382 - protein, 22 aa, Artificial sequence
  Synthetic
KKFGFEIKDKKCDVPCIGIEMV

SEQ ID NO: 1383 - protein, 22 aa, Artificial sequence
  Synthetic
KKKDKKCDVPCIGIEMVHDGGK

SEQ ID NO: 1384 - protein, 22 aa, Artificial sequence
  Synthetic
KKDVPCIGIEMVHDGGKTTWHS

SEQ ID NO: 1385 - protein, 22 aa, Artificial sequence
  Synthetic
KKGIEMVHDGGKTTWHSAATAI

SEQ ID NO: 1386 - protein, 22 aa, Artificial sequence
  Synthetic
KKHDGGKTTWHSAATAIYCLMG

SEQ ID NO: 1387 - protein, 23 aa, Artificial sequence
  Synthetic
KKKTTWHSAATAIYCLMGSGQLL

SEQ ID NO: 1388 - protein, 23 aa, Artificial sequence
  Synthetic
KKKAATAIYCLMGSGQLLWDTVT

SEQ ID NO: 1389 - protein, 23 aa, Artificial sequence
  Synthetic
KKKYCLMGSGQLLWDTVTGVNMT

SEQ ID NO: 1390 - protein, 18 aa, Artificial sequence
  Synthetic
KKSGQLLWDTVTGVNMTL

SEQ ID NO: 1391 - protein, 6 aa, Artificial sequence
  Synthetic
HHHHHH